SEQUENCE LISTING (421 sequences)

SEQ ID NO: 1 - protein, 717 aa, unidentified
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 2 - protein, 757 aa, unidentified
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 3 - protein, 766 aa, unidentified
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKTFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 4 - protein, 830 aa, unidentified
MPKIKKPTEISLLRKEVFPDLHFAKDRMRAASLVLKNEGREAAIEYLRVNHEDKPPNFMPPAKTPYVALSRPLEQWPIAQASIAIQKYIFGLTKDEFSATKKLLYGDKSTPNTESRKRWFEVTGVPNFGYMSAQGLNAIFSGALARYEGVVQKVENRNKKRFEKLSEKNQLLIEEGQPVKDYVPDTAYHTPETLQKLAENNHVRVEDLGDMIDRLVHPPGIHRSIYGYQQVPPFAYDPDNPKGIILPKAYAGYTRKPHDIIEAMPNRLNIPEGQAGYIPEHQRDKLKKGGRVKRLRTTRVRVDATETVRAKAEALNAEKARLRGKEAILAVFQIEEDWALIDMRGLLRNVYMRKLIAAGELTPTTLLGYFTETLTLDPRRTEATFCYHLRSEGALHAEYVRHGKNTRELLLDLTKDNEKIALVTIDLGQRNPLAAAIFRVGRDASGDLTENSLEPVSRMLLPQAYLDQIKAYRDAYDSFRQNIWDTALASLTPEQQRQILAYEAYTPDDSKENVLRLLLGGNVMPDDLPWEDMTKNTHYISDRYLADGGDPSKVWFVPGPRKRKKNAPPLKKPPKPRELVKRSDHNISHLSEFRPQLLKETRDAFEKAKIDTERGHVGYQKLSTRKDQLCKEILNWLEAEAVRLTRCKTMVLGLEDLNGPFFNQGKGKVRGWVSFFRQKQENRWIVNGFRKNALARAHDKGKYILELWPSWTSQTCPKCKHVHADNRHGDDFVCLQCGARLHADAEVATWNLAVVAIQGHSLPGPVREKSNDRKKSGSARKSKKANESGKVVGAWAAQATPKRATSKKETGTARNPVYNPLETQASCPAP

SEQ ID NO: 5 - protein, 733 aa, unidentified
QAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKERKKFEGINERRSKEGMPLLEPRLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVGKEEETNFVTSNGPRKRTDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRDVQTPGTARKSGRSLKSQDNLSEPKRVLQSKTRKKITSTETQNEPLATDLKT

SEQ ID NO: 6 - protein, 78 aa, synthetic construct
DTVPYKEAAGLNLIIKNAVNTYKGVQVKVDNKNKNNLAKINRKNEIAKLNGEQEISFEEIKAFDDKGYLLQKPSPNKS

SEQ ID NO: 7 - protein, 19 aa, synthetic construct
AVNTYKGVQVKVDNKNKNN

SEQ ID NO: 8 - protein, 21 aa, synthetic construct
LAKINRKNEIAKLNGEQEISF

SEQ ID NO: 9 - protein, 22 aa, synthetic construct
LAKINRKNEIAKLNGEQEISFE

SEQ ID NO: 10 - protein, 17 aa, synthetic construct
INRKNEIAKLNGEQEIS

SEQ ID NO: 11 - protein, 15 aa, synthetic construct
NRKNEIAKLNGEQEI

SEQ ID NO: 12 - protein, 11 aa, synthetic construct
NEIAKLNGEQE

SEQ ID NO: 13 - protein, 9 aa, synthetic construct
NGEQEISFE

SEQ ID NO: 14 - protein, 41 aa, synthetic construct
PKWQYTFLSKKENKRRKLSKRIKNVSPILGIICIKKDWCVF

SEQ ID NO: 15 - protein, 28 aa, synthetic construct
SEKAQVSNKSEIYFTSTDKGKTKDVMKS

SEQ ID NO: 16 - protein, 27 aa, synthetic construct
EKAQVSNKSEIYFTSTDKGKTKDVMKS

SEQ ID NO: 17 - protein, 17 aa, synthetic construct
IYFTSTDKGKTKDVMKS

SEQ ID NO: 18 - protein, 23 aa, synthetic construct
VSNKSEIYFTSTDKGKTKDVMKS

SEQ ID NO: 19 - skipped

SEQ ID NO: 20 - protein, 9 aa, synthetic construct
KGKTKDVMK

SEQ ID NO: 21 - protein, 30 aa, synthetic construct
NLVKKNNFFGGSGKREPGWDNFYKPKKENR

SEQ ID NO: 22 - protein, 29 aa, synthetic construct
TCPKCKYCDSKNRNGEKFNCLKCGIELNA

SEQ ID NO: 23 - protein, 22 aa, synthetic construct
RARLESINASRADEGLPEIKAE

SEQ ID NO: 24 - protein, 28 aa, synthetic construct
SVSRDQVFFTPAPKKGAKKKAPVEVMRK

SEQ ID NO: 25 - protein, 15 aa, synthetic construct
KKGAKKKAPVEVMRK

SEQ ID NO: 26 - protein, 22 aa, synthetic construct
EKKFNEINHKRSLEGLPIITPD

SEQ ID NO: 27 - protein, 19 aa, synthetic construct
GGDESRVYFEINNKRIKRS

SEQ ID NO: 28 - protein, 6 aa, synthetic construct
KRIKRS

SEQ ID NO: 29 - protein, 22 aa, synthetic construct
FEKLSEKNQLLIEEGQPVKDYV

SEQ ID NO: 30 - protein, 36 aa, synthetic construct
GGDPSKVWFVPGPRKRKKNAPPLKKPPKPRELVKRS

SEQ ID NO: 31 - protein, 23 aa, synthetic construct
RKRKKNAPPLKKPPKPRELVKRS

SEQ ID NO: 32 - protein, 22 aa, synthetic construct
RKKFEGINERRSKEGMPLLEPR

SEQ ID NO: 33 - protein, 18 aa, synthetic construct
GKEEETNFVTSNGPRKRT

SEQ ID NO: 34 - protein, 5 aa, synthetic construct
PRKRT

SEQ ID NO: 35-40 - skipped (6 entries)

SEQ ID NO: 41 - protein, 78 aa, synthetic construct
CTDYFSVQGLNLIFQNARKRYIGVQTKVTNRNEKRHKKLKRINAKRIAEGLPELTSDEPESALDETGHLIDPPGLNTN

SEQ ID NO: 42 - protein, 78 aa, synthetic construct
PDRGLPVQAINKIAKAAVNRAFGVVRKVENRNEKRRSRDNRIAEHNRENGLTEVVREAPEVATNADGFLLHPPGIDPS

SEQ ID NO: 43 - protein, 99 aa, synthetic construct
PNFGYMSAQGLNAIFSGALARYEGVVQKVENRNKKRFEKLSEKNQLLIEEGQPVKDYVPDTAYHTPETLQKLAENNHVRVEDLGDMIDRLVHPPGIHRS

SEQ ID NO: 44 - protein, 81 aa, synthetic construct
SEDDLMALEAQLLETIMGNAISLHGGVLKKIDNANVKAAKRLSGRNEARLNKGLQELPPEQEGSAYGADGLLVNPPGLNLN

SEQ ID NO: 45 - protein, 78 aa, synthetic construct
SNYNYTSVQGLNLIFKNAKAIYDGTLVKANNKNKKLEKKFNEINHKRSLEGLPIITPDFEEPFDENGHLNNPPGINRN

SEQ ID NO: 46 - protein, 90 aa, synthetic construct
LTFPMNQVQMLNGIFNRAFSVYLGVEKKVANRNAEAREKLKNRGQEDQFVEEFAYSYCPVDFKDGMFVHDSAEEGETQGCLLHPPGIDPK

SEQ ID NO: 47 - protein, 67 aa, synthetic construct
NIEYQNVAGLNLIFNNVKNTYNGVILKVKNRNEKLKKKAIKNNYEFEEIKTFNDDGCLINKPGINNV

SEQ ID NO: 48 - protein, 4 aa, synthetic construct
GSSG

SEQ ID NO: 49 - skipped

SEQ ID NO: 50 - protein, 70 aa, synthetic construct
PEWQHPLLNRRKNRRRRDWYSASLNKPKATCSKRSGTPNRKNSRTDQIQSGRFKGAIPVLMRFQDEWVII

SEQ ID NO: 51 - protein, 83 aa, synthetic construct
PGHQRFADTGQNNSGKANPNKKGRMRKYYGHGTKYTQPGEYQEVFRKGHREGNKRRYWEEDFRSEAHDCILYVIHIGDDWVVC

SEQ ID NO: 52 - protein, 63 aa, synthetic construct
PEHQRDKLKKGGRVKRLRTTRVRVDATETVRAKAEALNAEKARLRGKEAILAVFQIEEDWALI

SEQ ID NO: 53 - protein, 59 aa, synthetic construct
PEPHREGLTGRKDRRMRRYYETERGTKLKRPPLTAKGRADKANEALLVVVRIDSDWVVM

SEQ ID NO: 54 - protein, 43 aa, synthetic construct
PEHQRKNLKKKGRVRLYRRTPPKTKALASILAVLQIGKDWVLF

SEQ ID NO: 55 - protein, 70 aa, synthetic construct
PEWQRPLLNRHKGRRHRSWYANSLNKPRKSRTEEAKDRQNAGKRTALIEAERLKGVLPVLMRFKEDWLII

SEQ ID NO: 56 - protein, 72 aa, synthetic construct
PAWQREQGLVKPGGRRRRLSGSESNMRQKVDPSTGPRRSTRSGTVNRSNQRTGRNGDPLLVEIRMKEDWVLL

SEQ ID NO: 57 - protein, 34 aa, synthetic construct
DEYGKLISKRRKERINKDDAILCVSNFGDDWIIF

SEQ ID NO: 58 - protein, 46 aa, synthetic construct
PPWDRENLSVKKHRRKRASWARSRGGAIDDNMLLAVVRVADDWALL

SEQ ID NO: 59 - protein, 41 aa, synthetic construct
PLHDREKLTSNKHRRMKLPKSLRAQGALPVCFRVFDDWAVV

SEQ ID NO: 60 - protein, 59 aa, synthetic construct
PEWMRTAGEKTNPRTQKKFMHPGLSTRKNKRMRLPRSVRSAPLGALLVTIHLGEDWLVL

SEQ ID NO: 61 - protein, 69 aa, synthetic construct
NKIQRFNFVHGKNSGKVKFSDKTGRVKRYHHSKYKDATKPYKFLEESKKVSALDSILAIITIGDDWVVF

SEQ ID NO: 62 - protein, 85 aa, synthetic construct
PWFQRMDIPEGQIGHVNKIQRFNFVHGKNSGKVKFSDKTGRVKRYHHSKYKDATKPYKFLEESKKVSALDSILAIITIGDDWVVF

SEQ ID NO: 63 - protein, 68 aa, synthetic construct
PEKHRSQLSMAKHKRRRAWYALSQNKPRPPKDGSKGRRSVRDLADLKAASLADAIPLVSRVGFDWVVI

SEQ ID NO: 64 - protein, 48 aa, synthetic construct
PYWQRPFLSKRRNRRVRAGWGKQVSSIQAWLTGALLVIVRLGNEAFLA

SEQ ID NO: 65 - protein, 76 aa, synthetic construct
PGHQRKESTTEGPKINFRKGRIRRSYTALYAKRDSRRVRQGKLALPSYRHHMMRLNSNAESAILAVIFFGKDWVVF

SEQ ID NO: 66 - protein, 34 aa, synthetic construct
TDLFLARGGDPKKCMFTSEPKKKKNSKQVLYKIR

SEQ ID NO: 67 - protein, 27 aa, synthetic construct
ATAYLEKGGDRKVATLKPKNRPEMLRR

SEQ ID NO: 68 - protein, 35 aa, synthetic construct
SDDFLRRGGDPNIVHFDRQPKKGKVSKKSQRIKRS

SEQ ID NO: 69 - protein, 34 aa, synthetic construct
KQFLKKNGGNKSLIEYIPYQKKKSKKTPKAVLRS

SEQ ID NO: 70 - protein, 35 aa, synthetic construct
SEALLSNSVSRDQVFFTPAPKKGAKKKAPVEVMRK

SEQ ID NO: 71 - protein, 43 aa, synthetic construct
SDRYLADGGDPSKVWFVPGPRKRKKNAPPLKKPPKPRELVKRS

SEQ ID NO: 72 - protein, 32 aa, synthetic construct
SDHILDHGGDPDTVFFMATKRGQNKPTLHKRK

SEQ ID NO: 73 - protein, 43 aa, synthetic construct
SDLFLRMGGDRSRVFFEVPPKKAKKAPKKPPKKPAGPRIVKRT

SEQ ID NO: 74 - protein, 36 aa, synthetic construct
ANLYLERGGDPSKVFFTRPLKKDSKSKKPRKPTKRT

SEQ ID NO: 75 - protein, 27 aa, synthetic construct
VDAVLRKGGDESLVYFKNKEGETLKFR

SEQ ID NO: 76 - protein, 31 aa, synthetic construct
ADHVQAAGGDVEWFFFKTCGKGKEIKTVRRS

SEQ ID NO: 77 - protein, 27 aa, synthetic construct
AQAFVAKGGDPKVVTIKPNGKPKMFRK

SEQ ID NO: 78 - protein, 30 aa, synthetic construct
SDQLLQQGADPASLFFDGERDGKPCRHKKK

SEQ ID NO: 79 - protein, 29 aa, synthetic construct
ADMLISHGRDDDAFLSHGEFPTLEKRKKF

SEQ ID NO: 80 - protein, 20 aa, synthetic construct
SEALSAKGSPLALHDGAPIK

SEQ ID NO: 81 - protein, 26 aa, synthetic construct
RDCLKNKNIDESEFTFEKNGKKLDPT

SEQ ID NO: 82 - protein, 34 aa, synthetic construct
SDLAKERGVDDTLYMFPFFKGKGKKRKTEIRKRW

SEQ ID NO: 83 - protein, 29 aa, synthetic construct
SRWLRDHGWNASDFTQITKGRKKVERLWS

SEQ ID NO: 84 - protein, 40 aa, synthetic construct
SDLYLRRGGDPSRVFFVPQPSTPKKNAKKPPAPRKPVKRT

SEQ ID NO: 85 - protein, 32 aa, synthetic construct
SDHLINHGGDTSAVFFQTIRKGTKKLETIKRK

SEQ ID NO: 86 - protein, 21 aa, synthetic construct
ADELIKFENKDAFYPSGKKKF

SEQ ID NO: 87 - protein, 33 aa, synthetic construct
SEAILAQGVSRDQVFFTPNPKKGSKEPVEVMRK

SEQ ID NO: 88 - protein, 32 aa, synthetic construct
ADEFINNGGDKSLVYFTAKDKKSKKEKLVKLS

SEQ ID NO: 89 - protein, 26 aa, synthetic construct
SDLYLKNGGDESRVYFEINNKRIKRS

SEQ ID NO: 90 - protein, 32 aa, synthetic construct
AQHLMSLGFGESATHVRYRPKRKASERTILKY

SEQ ID NO: 91 - protein, 25 aa, synthetic construct
SDALIEVGKEEETNFVTSNGPRKRT

SEQ ID NO: 92 - protein, 33 aa, synthetic construct
SDCLVALGKTDDVFFVPKAKKGKKETGIAVKRK

SEQ ID NO: 93 - protein, 25 aa, synthetic construct
SDYLIDNNITNDVYRTVNKAKYKTN

SEQ ID NO: 94 - protein, 30 aa, synthetic construct
SDYLIQIGRGTETEKTITTKKGKEKILTIR

SEQ ID NO: 95 - protein, 36 aa, synthetic construct
SDLYIERGGDPRDVHQQVETKPKGKRKSEIRILKIR

SEQ ID NO: 96 - protein, 31 aa, synthetic construct
SDYIVDHGGDPEKVFFETKSKKDKTKRYKRR

SEQ ID NO: 97 - protein, 32 aa, synthetic construct
SDHLLAKGVTDQVFFEKKSKGKKKGTETVKRK

SEQ ID NO: 98 - protein, 43 aa, synthetic construct
FNKHVENGGDPNVLLFTPTEDKKNKGKKSKNKKGEYGDRVPHS

SEQ ID NO: 99 - protein, 53 aa, synthetic construct
SNYLLNNGLSPQEGASDQDWVVPSSTREVARVAYRISDSKGKGKKKPEIFKAS

SEQ ID NO: 100 - protein, 38 aa, synthetic construct
AKWVMRHGDPSLVEVLEYRKDNEIKLDKNGVPKKVKLT

SEQ ID NO: 101 - protein, 35 aa, synthetic construct
DLYIERGGDPRDVHQQVETKPKGKRKSEIRILKIR

SEQ ID NO: 102 - skipped

SEQ ID NO: 103 - protein, 29 aa, synthetic construct
DLDVKKKFNGRGIRDIGWDNFFSSRKENR

SEQ ID NO: 104 - protein, 29 aa, synthetic construct
TCPDCGFCSKENRDGINFTCRKCGVSYHA

SEQ ID NO: 105 - protein, 698 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQGSSGDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 106 - protein, 704 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEGSSGDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 107 - protein, 712 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDGSSGSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 108 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSESGSTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 109 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSESSSTSTDKGKTKDSMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 110 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEAAATSTDKGKTKDAMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 111 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSESSFTSTDKGKTKDSMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 112 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEAAFTSTDKGKTKDAMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 113 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYGTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 114 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSAYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 115 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDAGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 116 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGATKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 117 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTADVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 118 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMASDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 119 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDSGSTSDVMSSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 120 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDAGATADVMASDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 121 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDAGATADVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 122 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKRVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 123 - protein, 699 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNGSSGEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 124 - protein, 694 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNGSSGEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDGSSGSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 125 - protein, 680 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNGSSGEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQGSSGDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 126 - protein, 733 aa, synthetic construct
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNGSSGDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 127 - protein, 746 aa, synthetic construct
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPGSSGDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 128 - protein, 739 aa, synthetic construct
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKAGSSGEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 129 - protein, 728 aa, synthetic construct
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKAGSSGEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPGSSGDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 130 - protein, 715 aa, synthetic construct
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKAGSSGEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNGSSGDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 131 - protein, 753 aa, synthetic construct
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSNKSEIYFTSTDKGKTKDVMKSDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 132 - protein, 751 aa, synthetic construct
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGSSGDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKTFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 133 - protein, 751 aa, synthetic construct; identical to SEQ ID NO: 132

SEQ ID NO: 134 - protein, 748 aa, synthetic construct
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLGSSGFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKTFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 135 - protein, 733 aa, synthetic construct
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLGSSGFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGSSGDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKTFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 136 - protein, 746 aa, synthetic construct
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLGSSGFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNGSSGDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKTFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 137 - protein, 771 aa, synthetic construct
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGVSNKSEIYFTSTDKGKTKDVMKSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKTFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 138 - protein, 746 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPEWQHPLLNRRKNRRRRDWYSASLNKPKATCSKRSGTPNRKNSRTDQIQSGRFKGAIPVLMRFQDEWVIIDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 139 - protein, 759 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPGHQRFADTGQNNSGKANPNKKGRMRKYYGHGTKYTQPGEYQEVFRKGHREGNKRRYWEEDFRSEAHDCILYVIHIGDDWVVCDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 140 - protein, 739 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPEHQRDKLKKGGRVKRLRTTRVRVDATETVRAKAEALNAEKARLRGKEAILAVFQIEEDWALIDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 141 - protein, 735 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPEPHREGLTGRKDRRMRRYYETERGTKLKRPPLTAKGRADKANEALLVVVRIDSDWVVMDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 142 - protein, 719 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPEHQRKNLKKKGRVRLYRRTPPKTKALASILAVLQIGKDWVLFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 143 - protein, 746 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPEWQRPLLNRHKGRRHRSWYANSLNKPRKSRTEEAKDRQNAGKRTALIEAERLKGVLPVLMRFKEDWLIIDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 144 - protein, 748 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPAWQREQGLVKPGGRRRRLSGSESNMRQKVDPSTGPRRSTRSGTVNRSNQRTGRNGDPLLVEIRMKEDWVLLDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 145 - protein, 710 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVDEYGKLISKRRKERINKDDAILCVSNFGDDWIIFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 146 - protein, 722 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPPWDRENLSVKKHRRKRASWARSRGGAIDDNMLLAVVRVADDWALLDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 147 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPLHDREKLTSNKHRRMKLPKSLRAQGALPVCFRVFDDWAVVDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 148 - protein, 735 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPEWMRTAGEKTNPRTQKKFMHPGLSTRKNKRMRLPRSVRSAPLGALLVTIHLGEDWLVLDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 149 - protein, 745 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVNKIQRFNFVHGKNSGKVKFSDKTGRVKRYHHSKYKDATKPYKFLEESKKVSALDSILAIITIGDDWVVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 150 - protein, 761 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPWFQRMDIPEGQIGHVNKIQRFNFVHGKNSGKVKFSDKTGRVKRYHHSKYKDATKPYKFLEESKKVSALDSILAIITIGDDWVVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 151 - protein, 744 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPEKHRSQLSMAKHKRRRAWYALSQNKPRPPKDGSKGRRSVRDLADLKAASLADAIPLVSRVGFDWVVIDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 152 - protein, 724 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPYWQRPFLSKRRNRRVRAGWGKQVSSIQAWLTGALLVIVRLGNEAFLADMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 153 - protein, 752 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPGHQRKESTTEGPKINFRKGRIRRSYTALYAKRDSRRVRQGKLALPSYRHHMMRLNSNAESAILAVIFFGKDWVVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 154 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLCTDYFSVQGLNLIFQNARKRYIGVQTKVTNRNEKRHKKLKRINAKRIAEGLPELTSDEPESALDETGHLIDPPGLNTNIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 155 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLPDRGLPVQAINKIAKAAVNRAFGVVRKVENRNEKRRSRDNRIAEHNRENGLTEVVREAPEVATNADGFLLHPPGIDPSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 156 - protein, 738 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLPNFGYMSAQGLNAIFSGALARYEGVVQKVENRNKKRFEKLSEKNQLLIEEGQPVKDYVPDTAYHTPETLQKLAENNHVRVEDLGDMIDRLVHPPGIHRSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 157 - protein, 720 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLSEDDLMALEAQLLETIMGNAISLHGGVLKKIDNANVKAAKRLSGRNEARLNKGLQELPPEQEGSAYGADGLLVNPPGLNLNIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 158 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLSNYNYTSVQGLNLIFKNAKAIYDGTLVKANNKNKKLEKKFNEINHKRSLEGLPIITPDFEEPFDENGHLNNPPGINRNIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 159 - protein, 729 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLLTFPMNQVQMLNGIFNRAFSVYLGVEKKVANRNAEAREKLKNRGQEDQFVEEFAYSYCPVDFKDGMFVHDSAEEGETQGCLLHPPGIDPKIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 160 - protein, 706 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLNIEYQNVAGLNLIFNNVKNTYNGVILKVKNRNEKLKKKAIKNNYEFEEIKTFNDDGCLINKPGINNVIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 161 - protein, 723 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFITDLFLARGGDPKKCMFTSEPKKKKNSKQVLYKIRDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 162 - protein, 716 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFIATAYLEKGGDRKVATLKPKNRPEMLRRDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 163 - protein, 724 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISDDFLRRGGDPNIVHFDRQPKKGKVSKKSQRIKRSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 164 - protein, 723 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFIKQFLKKNGGNKSLIEYIPYQKKKSKKTPKAVLRSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 165 - protein, 724 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEALLSNSVSRDQVFFTPAPKKGAKKKAPVEVMRKDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 166 - protein, 732 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISDRYLADGGDPSKVWFVPGPRKRKKNAPPLKKPPKPRELVKRSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 167 - protein, 721 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISDHILDHGGDPDTVFFMATKRGQNKPTLHKRKDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 168 - protein, 732 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISDLFLRMGGDRSRVFFEVPPKKAKKAPKKPPKKPAGPRIVKRTDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 169 - protein, 725 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFIANLYLERGGDPSKVFFTRPLKKDSKSKKPRKPTKRTDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 170 - protein, 716 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFIVDAVLRKGGDESLVYFKNKEGETLKFRDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 171 - protein, 720 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFIADHVQAAGGDVEWFFFKTCGKGKEIKTVRRSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 172 - protein, 716 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFIAQAFVAKGGDPKVVTIKPNGKPKMFRKDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 173 - protein, 719 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISDQLLQQGADPASLFFDGERDGKPCRHKKKDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 174 - protein, 718 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFIADMLISHGRDDDAFLSHGEFPTLEKRKKFDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 175 - protein, 709 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEALSAKGSPLALHDGAPIKDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 176 - protein, 715 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFIRDCLKNKNIDESEFTFEKNGKKLDPTDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 177 - protein, 723 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISDLAKERGVDDTLYMFPFFKGKGKKRKTEIRKRWDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 178 - protein, 718 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISRWLRDHGWNASDFTQITKGRKKVERLWSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 179 - protein, 729 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISDLYLRRGGDPSRVFFVPQPSTPKKNAKKPPAPRKPVKRTDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 180 - protein, 721 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISDHLINHGGDTSAVFFQTIRKGTKKLETIKRKDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 181 - protein, 710 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFIADELIKFENKDAFYPSGKKKFDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 182 - protein, 722 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEAILAQGVSRDQVFFTPNPKKGSKEPVEVMRKDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 183 - protein, 721 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFIADEFINNGGDKSLVYFTAKDKKSKKEKLVKLSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 184 - protein, 715 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISDLYLKNGGDESRVYFEINNKRIKRSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 185 - protein, 721 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFIAQHLMSLGFGESATHVRYRPKRKASERTILKYDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 186 - protein, 714 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISDALIEVGKEEETNFVTSNGPRKRTDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 187 - protein, 722 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISDCLVALGKTDDVFFVPKAKKGKKETGIAVKRKDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 188 - protein, 714 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISDYLIDNNITNDVYRTVNKAKYKTNDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 189 - protein, 719 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISDYLIQIGRGTETEKTITTKKGKEKILTIRDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 190 - protein, 725 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISDLYIERGGDPRDVHQQVETKPKGKRKSEIRILKIRDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 191 - protein, 720 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISDYIVDHGGDPEKVFFETKSKKDKTKRYKRRDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 192 - protein, 721 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISDHLLAKGVTDQVFFEKKSKGKKKGTETVKRKDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 193 - protein, 732 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFIFNKHVENGGDPNVLLFTPTEDKKNKGKKSKNKKGEYGDRVPHSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 194 - protein, 742 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISNYLLNNGLSPQEGASDQDWVVPSSTREVARVAYRISDSKGKGKKKPEIFKASDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 195 - protein, 727 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFIAKWVMRHGDPSLVEVLEYRKDNEIKLDKNGVPKKVKLTDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 196 - protein, 716 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIEDLDVKKKFNGRGIRDIGWDNFFSSRKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 197 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPDCGFCSKENRDGINFTCRKCGVSYHADIDVATENLATVAITAQSMPKPTCERSGDAKKPVRARKAKAPEFHDKLAPSYTVVLREAV

SEQ ID NO: 198 - protein, 712 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDGSSGSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 199 - protein, 707 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLGSSGEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDGSSGSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 200 - protein, 697 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNGSSGLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDGSSGSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 201 - protein, 712 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNAIAKLNGAQAISFAEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDGSSGSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 202 - protein, 700 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNGSSGEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 203 - protein, 699 aa, synthetic construct; identical to SEQ ID NO: 123

SEQ ID NO: 204 - protein, 698 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQGSSGDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 205 - protein, 704 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEGSSGDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 206 - protein, 693 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLGSSGEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQGSSGDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 207 - protein, 683 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNGSSGLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQGSSGDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 208 - protein, 698 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNAIAKLNGAQAISFAEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQGSSGDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 209 - protein, 26 aa, synthetic construct
GSGGSPAGSPTSTEEGTSESATPGSG

SEQ ID NO: 210 - protein, 681 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNGSSGEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQGSSGDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 211 - protein, 680 aa, synthetic construct; identical to SEQ ID NO: 125

SEQ ID NO: 212 - protein, 685 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKGSSGFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQGSSGDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 213 - protein, 685 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKIGSSFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQGSSGDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 214 - protein, 689 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKGSISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQGSSGDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 215 - protein, 712 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLGSSGEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 216 - protein, 702 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNGSSGLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 217 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNAIAKLNGAQAISFAEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 218 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNAIAKLNGLQIISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 219 - protein, 700 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNGSSGEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 220 - protein, 699 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNGSSGEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 221 - protein, 704 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKGSSGFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 222 - protein, 704 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKIGSSFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 223 - protein, 708 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKGSISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 224 - protein, 725 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISDLYIERGGDPRDVHQQVETKPKGKRKSEIRILKIRDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 225 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEVAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 226 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEQAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 227 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEFAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 228 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEWAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 229 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLFLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 230 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLYLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 231 - protein, 717 aa, synthetic construct
MIKPTVSQFLTPGFKLIRNHSRTAGKKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 232 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIQNLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 233 - protein, 798 aa, synthetic construct
MPKIKKPTEISLLRKEVFPDLHFAKDRMRAASLVLKNEGREAAIEYLRVNHEDKPPNFMPPAKTPYVALSRPLEQWPIAQASIAIQKYIFGLTKDEFSATKKLLYGDKSTPNTESRKRWFEVTGVPNFGYMSAQGLNAIFSGALARYEGVVQKVENRNKKRFEKLSEKNQLLIEEGQPVKDYVPDTAYHTPETLQKLAENNHVRVEDLGDMIDRLVHPPGIHRSIYGYQQVPPFAYDPDNPKGIILPKAYAGYTRKPHDIIEAMPNRLNIPEGQAGYIPEHQRDKLKKGGRVKRLRTTRVRVDATETVRAKAEALNAEKARLRGKEAILAVFQIEEDWALIDMRGLLRNVYMRKLIAAGELTPTTLLGYFTETLTLDPRRTEATFCYHLRSEGALHAEYVRHGKNTRELLLDLTKDNEKIALVTIDLGQRNPLAAAIFRVGRDASGDLTENSLEPVSRMLLPQAYLDQIKAYRDAYDSFRQNIWDTALASLTPEQQRQILAYEAYTPDDSKENVLRLLLGGNVMPDDLPWEDMTKNTHYISDRYLADGSSGDHNISHLSEFRPQLLKETRDAFEKAKIDTERGHVGYQKLSTRKDQLCKEILNWLEAEAVRLTRCKTMVLGLEDLNGPFFNQGKGKVRGWVSFFRQKQENRWIVNGFRKNALARAHDKGKYILELWPSWTSQTCPKCKHVHADNRHGDDFVCLQCGARLHADAEVATWNLAVVAIQGHSLPGPVREKSNDRKKSGSARKSKKANESGKVVGAWAAQATPKRATSKKETGTARNPVYNPLETQASCPAP

SEQ ID NO: 234 - protein, 811 aa, synthetic construct
MPKIKKPTEISLLRKEVFPDLHFAKDRMRAASLVLKNEGREAAIEYLRVNHEDKPPNFMPPAKTPYVALSRPLEQWPIAQASIAIQKYIFGLTKDEFSATKKLLYGDKSTPNTESRKRWFEVTGVPNFGYMSAQGLNAIFSGALARYEGVVQKVENRNKKRFEKLSEKNQLLIEEGQPVKDYVPDTAYHTPETLQKLAENNHVRVEDLGDMIDRLVHPPGIHRSIYGYQQVPPFAYDPDNPKGIILPKAYAGYTRKPHDIIEAMPNRLNIPEGQAGYIPEHQRDKLKKGGRVKRLRTTRVRVDATETVRAKAEALNAEKARLRGKEAILAVFQIEEDWALIDMRGLLRNVYMRKLIAAGELTPTTLLGYFTETLTLDPRRTEATFCYHLRSEGALHAEYVRHGKNTRELLLDLTKDNEKIALVTIDLGQRNPLAAAIFRVGRDASGDLTENSLEPVSRMLLPQAYLDQIKAYRDAYDSFRQNIWDTALASLTPEQQRQILAYEAYTPDDSKENVLRLLLGGNVMPDDLPWEDMTKNTHYISDRYLADGGDPSKVWFVPGPGSSGDHNISHLSEFRPQLLKETRDAFEKAKIDTERGHVGYQKLSTRKDQLCKEILNWLEAEAVRLTRCKTMVLGLEDLNGPFFNQGKGKVRGWVSFFRQKQENRWIVNGFRKNALARAHDKGKYILELWPSWTSQTCPKCKHVHADNRHGDDFVCLQCGARLHADAEVATWNLAVVAIQGHSLPGPVREKSNDRKKSGSARKSKKANESGKVVGAWAAQATPKRATSKKETGTARNPVYNPLETQASCPAP

SEQ ID NO: 235 - protein, 812 aa, synthetic construct
MPKIKKPTEISLLRKEVFPDLHFAKDRMRAASLVLKNEGREAAIEYLRVNHEDKPPNFMPPAKTPYVALSRPLEQWPIAQASIAIQKYIFGLTKDEFSATKKLLYGDKSTPNTESRKRWFEVTGVPNFGYMSAQGLNAIFSGALARYEGVVQKVENRNKKRGSSGPDTAYHTPETLQKLAENNHVRVEDLGDMIDRLVHPPGIHRSIYGYQQVPPFAYDPDNPKGIILPKAYAGYTRKPHDIIEAMPNRLNIPEGQAGYIPEHQRDKLKKGGRVKRLRTTRVRVDATETVRAKAEALNAEKARLRGKEAILAVFQIEEDWALIDMRGLLRNVYMRKLIAAGELTPTTLLGYFTETLTLDPRRTEATFCYHLRSEGALHAEYVRHGKNTRELLLDLTKDNEKIALVTIDLGQRNPLAAAIFRVGRDASGDLTENSLEPVSRMLLPQAYLDQIKAYRDAYDSFRQNIWDTALASLTPEQQRQILAYEAYTPDDSKENVLRLLLGGNVMPDDLPWEDMTKNTHYISDRYLADGGDPSKVWFVPGPRKRKKNAPPLKKPPKPRELVKRSDHNISHLSEFRPQLLKETRDAFEKAKIDTERGHVGYQKLSTRKDQLCKEILNWLEAEAVRLTRCKTMVLGLEDLNGPFFNQGKGKVRGWVSFFRQKQENRWIVNGFRKNALARAHDKGKYILELWPSWTSQTCPKCKHVHADNRHGDDFVCLQCGARLHADAEVATWNLAVVAIQGHSLPGPVREKSNDRKKSGSARKSKKANESGKVVGAWAAQATPKRATSKKETGTARNPVYNPLETQASCPAP

SEQ ID NO: 236 - protein, 780 aa, synthetic construct
MPKIKKPTEISLLRKEVFPDLHFAKDRMRAASLVLKNEGREAAIEYLRVNHEDKPPNFMPPAKTPYVALSRPLEQWPIAQASIAIQKYIFGLTKDEFSATKKLLYGDKSTPNTESRKRWFEVTGVPNFGYMSAQGLNAIFSGALARYEGVVQKVENRNKKRGSSGPDTAYHTPETLQKLAENNHVRVEDLGDMIDRLVHPPGIHRSIYGYQQVPPFAYDPDNPKGIILPKAYAGYTRKPHDIIEAMPNRLNIPEGQAGYIPEHQRDKLKKGGRVKRLRTTRVRVDATETVRAKAEALNAEKARLRGKEAILAVFQIEEDWALIDMRGLLRNVYMRKLIAAGELTPTTLLGYFTETLTLDPRRTEATFCYHLRSEGALHAEYVRHGKNTRELLLDLTKDNEKIALVTIDLGQRNPLAAAIFRVGRDASGDLTENSLEPVSRMLLPQAYLDQIKAYRDAYDSFRQNIWDTALASLTPEQQRQILAYEAYTPDDSKENVLRLLLGGNVMPDDLPWEDMTKNTHYISDRYLADGSSGDHNISHLSEFRPQLLKETRDAFEKAKIDTERGHVGYQKLSTRKDQLCKEILNWLEAEAVRLTRCKTMVLGLEDLNGPFFNQGKGKVRGWVSFFRQKQENRWIVNGFRKNALARAHDKGKYILELWPSWTSQTCPKCKHVHADNRHGDDFVCLQCGARLHADAEVATWNLAVVAIQGHSLPGPVREKSNDRKKSGSARKSKKANESGKVVGAWAAQATPKRATSKKETGTARNPVYNPLETQASCPAP

SEQ ID NO: 237 - protein, 793 aa, synthetic construct
MPKIKKPTEISLLRKEVFPDLHFAKDRMRAASLVLKNEGREAAIEYLRVNHEDKPPNFMPPAKTPYVALSRPLEQWPIAQASIAIQKYIFGLTKDEFSATKKLLYGDKSTPNTESRKRWFEVTGVPNFGYMSAQGLNAIFSGALARYEGVVQKVENRNKKRGSSGPDTAYHTPETLQKLAENNHVRVEDLGDMIDRLVHPPGIHRSIYGYQQVPPFAYDPDNPKGIILPKAYAGYTRKPHDIIEAMPNRLNIPEGQAGYIPEHQRDKLKKGGRVKRLRTTRVRVDATETVRAKAEALNAEKARLRGKEAILAVFQIEEDWALIDMRGLLRNVYMRKLIAAGELTPTTLLGYFTETLTLDPRRTEATFCYHLRSEGALHAEYVRHGKNTRELLLDLTKDNEKIALVTIDLGQRNPLAAAIFRVGRDASGDLTENSLEPVSRMLLPQAYLDQIKAYRDAYDSFRQNIWDTALASLTPEQQRQILAYEAYTPDDSKENVLRLLLGGNVMPDDLPWEDMTKNTHYISDRYLADGGDPSKVWFVPGPGSSGDHNISHLSEFRPQLLKETRDAFEKAKIDTERGHVGYQKLSTRKDQLCKEILNWLEAEAVRLTRCKTMVLGLEDLNGPFFNQGKGKVRGWVSFFRQKQENRWIVNGFRKNALARAHDKGKYILELWPSWTSQTCPKCKHVHADNRHGDDFVCLQCGARLHADAEVATWNLAVVAIQGHSLPGPVREKSNDRKKSGSARKSKKANESGKVVGAWAAQATPKRATSKKETGTARNPVYNPLETQASCPAP

SEQ ID NO: 238 - protein, 817 aa, synthetic construct
MPKIKKPTEISLLRKEVFPDLHFAKDRMRAASLVLKNEGREAAIEYLRVNHEDKPPNFMPPAKTPYVALSRPLEQWPIAQASIAIQKYIFGLTKDEFSATKKLLYGDKSTPNTESRKRWFEVTGVPNFGYMSAQGLNAIFSGALARYEGVVQKVENRNKKRFEKLSEKNQLLIEEGQPVKDYVPDTAYHTPETLQKLAENNHVRVEDLGDMIDRLVHPPGIHRSIYGYQQVPPFAYDPDNPKGIILPKAYAGYTRKPHDIIEAMPNRLNIPEGQAGYIPEHQRDKLKKGGRVKRLRTTRVRVDATETVRAKAEALNAEKARLRGKEAILAVFQIEEDWALIDMRGLLRNVYMRKLIAAGELTPTTLLGYFTETLTLDPRRTEATFCYHLRSEGALHAEYVRHGKNTRELLLDLTKDNEKIALVTIDLGQRNPLAAAIFRVGRDASGDLTENSLEPVSRMLLPQAYLDQIKAYRDAYDSFRQNIWDTALASLTPEQQRQILAYEAYTPDDSKENVLRLLLGGNVMPDDLPWEDMTKNTHYISDRYLADVSNKSEIYFTSTDKGKTKDVMKSDHNISHLSEFRPQLLKETRDAFEKAKIDTERGHVGYQKLSTRKDQLCKEILNWLEAEAVRLTRCKTMVLGLEDLNGPFFNQGKGKVRGWVSFFRQKQENRWIVNGFRKNALARAHDKGKYILELWPSWTSQTCPKCKHVHADNRHGDDFVCLQCGARLHADAEVATWNLAVVAIQGHSLPGPVREKSNDRKKSGSARKSKKANESGKVVGAWAAQATPKRATSKKETGTARNPVYNPLETQASCPAP

SEQ ID NO: 239 - protein, 719 aa, synthetic construct
QAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKERKKFEGINERRSKEGMPLLEPRLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVGSSGDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRDVQTPGTARKSGRSLKSQDNLSEPKRVLQSKTRKKITSTETQNEPLATDLKT

SEQ ID NO: 240 - protein, 732 aa, synthetic construct
QAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKERKKFEGINERRSKEGMPLLEPRLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVGKEEETNFVTSNGGSSGDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRDVQTPGTARKSGRSLKSQDNLSEPKRVLQSKTRKKITSTETQNEPLATDLKT

SEQ ID NO: 241 - protein, 715 aa, synthetic construct
QAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKEGSSGLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVGKEEETNFVTSNGPRKRTDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRDVQTPGTARKSGRSLKSQDNLSEPKRVLQSKTRKKITSTETQNEPLATDLKT

SEQ ID NO: 242 - protein, 701 aa, synthetic construct
QAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKEGSSGLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVGSSGDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRDVQTPGTARKSGRSLKSQDNLSEPKRVLQSKTRKKITSTETQNEPLATDLKT

SEQ ID NO: 243 - protein, 714 aa, synthetic construct
QAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKEGSSGLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVGKEEETNFVTSNGGSSGDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRDVQTPGTARKSGRSLKSQDNLSEPKRVLQSKTRKKITSTETQNEPLATDLKT

SEQ ID NO: 244 - protein, 738 aa, synthetic construct
QAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKERKKFEGINERRSKEGMPLLEPRLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVVSNKSEIYFTSTDKGKTKDVMKSDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRDVQTPGTARKSGRSLKSQDNLSEPKRVLQSKTRKKITSTETQNEPLATDLKT

SEQ ID NO: 245-246 - skipped (2 entries)

SEQ ID NO: 247 - protein, 8 aa, synthetic construct
  VARIANT 3: note = Any naturally occurring amino acid
  VARIANT 4: note = W, F, or Y
  VARIANT 5: note = Any naturally occurring amino acid
  VARIANT 6: note = Any naturally occurring amino acid
KRXXXXAF

SEQ ID NO: 248 - skipped

SEQ ID NO: 249 - protein, 6 aa, synthetic construct
  VARIANT 5: note = K or R
  VARIANT 6: note = W, F, or Y
LGKRXX

SEQ ID NO: 250 - skipped

SEQ ID NO: 251 - protein, 20 aa, synthetic construct
  VARIANT 4: note = Any naturally occurring amino acid
  VARIANT 8: note = Any naturally occurring amino acid
  VARIANT 11: note = Any naturally occurring amino acid
  VARIANT 15: note = Any naturally occurring amino acid
  VARIANT 19: note = Any naturally occurring amino acid
GLFXALLXLLXSLWXLLLXA

SEQ ID NO: 252-257 - skipped (6 entries)

SEQ ID NO: 258 - protein, 16 aa, synthetic construct
APKKKRKVGIHGVPAA

SEQ ID NO: 259-264 - skipped (6 entries)

SEQ ID NO: 265 - RNA, 24 nt, synthetic construct
  source 1..24: mol_type = other RNA
atagattgctccttacgaggagac

SEQ ID NO: 266 - RNA, 41 nt, synthetic construct
  source 1..41: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 39: mod_base = OTHER; note = 2'-O-methyl modified nucleotide with phosphorothioate linkage
  modified_base 40: mod_base = OTHER; note = 2'-O-methyl modified nucleotide with phosphorothioate linkage
  modified_base 41: mod_base = OTHER; note = 2'-O-methyl modified nucleotide
atagattgctccttacgaggagacgagcaacggcggaaggt

SEQ ID NO: 267 - RNA, 17 nt, synthetic construct
  source 1..17: mol_type = other RNA
gagcaacggcggaaggt

SEQ ID NO: 268 - RNA, 17 nt, synthetic construct
  source 1..17: mol_type = other RNA
aggtgagatctagggag

SEQ ID NO: 269 - RNA, 41 nt, synthetic construct
  source 1..41: mol_type = other RNA
atagattgctccttacgaggagacctttgcactttccttag

SEQ ID NO: 270 - RNA, 41 nt, synthetic construct
  source 1..41: mol_type = other RNA
atagattgctccttacgaggagacgtatttgtgaagtctta

SEQ ID NO: 271 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 272 - RNA, 41 nt, synthetic construct
  source 1..41: mol_type = other RNA
atagattgctccttacgaggagacaataaagcatagtgcaa

SEQ ID NO: 273 - RNA, 41 nt, synthetic construct
  source 1..41: mol_type = other RNA
atagattgctccttacgaggagactgagatcaacagcacag

SEQ ID NO: 274 - RNA, 41 nt, synthetic construct
  source 1..41: mol_type = other RNA
atagattgctccttacgaggagaccattttagtctgtcttc

SEQ ID NO: 275 - RNA, 41 nt, synthetic construct
  source 1..41: mol_type = other RNA
atagattgctccttacgaggagacggctctgattcctacag

SEQ ID NO: 276 - RNA, 41 nt, synthetic construct
  source 1..41: mol_type = other RNA
atagattgctccttacgaggagacgacttagattatgcatt

SEQ ID NO: 277 - RNA, 41 nt, synthetic construct; identical to SEQ ID NO: 266
  source 1..41: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 39: mod_base = OTHER; note = 2'-O-methyl modified nucleotide with phosphorothioate linkage
  modified_base 40: mod_base = OTHER; note = 2'-O-methyl modified nucleotide with phosphorothioate linkage
  modified_base 41: mod_base = OTHER; note = 2'-O-methyl modified nucleotide

SEQ ID NO: 278 - RNA, 41 nt, synthetic construct
  source 1..41: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 39: mod_base = OTHER; note = 2'-O-methyl modified nucleotide with phosphorothioate linkage
  modified_base 40: mod_base = OTHER; note = 2'-O-methyl modified nucleotide with phosphorothioate linkage
  modified_base 41: mod_base = OTHER; note = 2'-O-methyl modified nucleotide
atagattgctccttacgaggagacatgtcacagagtgggac

SEQ ID NO: 279 - RNA, 41 nt, synthetic construct
  source 1..41: mol_type = other RNA
atagattgctccttacgaggagacaggtgagatctagggag

SEQ ID NO: 280 - RNA, 41 nt, synthetic construct
  source 1..41: mol_type = other RNA
atagattgctccttacgaggagacatccctagaagcagcta

SEQ ID NO: 281 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGGLGPSYTVVLREAV

SEQ ID NO: 282 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNRAKINRKNEIAKLNGEQEISFEEIKAFDDKGYLLQKPSPNKSIYCYQSVSPKPFITSKYRNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 283 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNRIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 284 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNRIAKLNGEQEISFEEIKAFDDKGYLLQKPSPNKSIYCYQSVSPKPFITSKYHNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 285 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNRIAKLNGEQEISFEEIKAFDDKGYLLQKPSPNKSIYCYQSVSPKPFITSKYHNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 286 - protein, 716 aa, synthetic construct
IKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 287 - protein, 715 aa, synthetic construct
KPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 288 - protein, 714 aa, synthetic construct
PTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 289 - protein, 713 aa, synthetic construct
TVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 290 - protein, 716 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREA

SEQ ID NO: 291 - protein, 715 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLRE

SEQ ID NO: 292 - protein, 714 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLR

SEQ ID NO: 293 - protein, 713 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVL

SEQ ID NO: 294 - protein, 717 aa, synthetic construct
MIKPTVSQFLTPGFKLIRNHSRTAWLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWIVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKWVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 295 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQIARQLANWISSMCDVIGIENLVKKNNFFGGSGKREPGWDNFYKPKKENRWWIYAIHKALTELSQNKGKWVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 296 - protein, 717 aa, synthetic construct
MIKPTVSQFLTPGFKLIRNHSRTAWLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVYQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWIYAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 297 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWIVFDMRGLLRTNHWKKYHKPTDSINDLFDYFTGDPVIDTKANVVRFRYKMENGIVNYKPVREKKGKELLENICDQNGSCKLATVDVYQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQIARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 298 - protein, 717 aa, synthetic construct
MIKPTVSQFLTPGFKLIRNHSRTAYLKLKNEGEEACKKFVRENEIPKDECPNFQGGPAIANIIALSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKYVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 299 - protein, 717 aa, synthetic construct
MIKPTVSQFLTPGFKLIRNHSRTAYLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHFVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMFTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 300 - protein, 717 aa, synthetic construct
MIKPTVSQFLTPGFKLIRNHSRTAFLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKYVILLPAMFTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 301 - protein, 717 aa, synthetic construct
MIKPTVSQFLTPGFKLIRNHSRTAFLKLKNEGEEACKKFVRENEIPKDECPNFQGGPAIANIIALSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHFVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 302 - protein, 717 aa, synthetic construct
MIKPTVSQFLTPGFKLIRNHWRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLLTNHWKKYHKPTDSINDLFDYFTGDPVIDTKANVVRFRYKMENGIVNYKPVREKKGKELLENICDQNGSCKLAIVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 303 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSMLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKWRKLSKRIKNVSPILGIIFIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 304 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKMRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLAIVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQWARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 305 - protein, 717 aa, synthetic construct
MIKPTVSQFLTPGFKLIRNHFRTAGLKLKNEGEEACKKFVRENEIPKDECPNFQGGPAIANIIAKSREFTEWEIYQSMLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQYARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 306 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIIIIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQLARQLANWISSMCDVIGIENLVKKNNFFGGSGKREPGWDNFYKPKKENRWWIFAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 307 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSFLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKLRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQLARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 308 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSFLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIIIIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLAWVAITAQSMPKPTCERSGDAKKPVRARKAKAPEFHDKLAPSYTVVLREAV

SEQ ID NO: 309 - protein, 717 aa, synthetic construct
MIKPTVSQFLTPGFKLIRNHWRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQWARQLANWISSMCDVIGIENLVKKNNFFGGSGKREPGWDNFYKPKKENRWWINAIHKALTELSQNKGKRVILLPAMYTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 310 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKLRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWIFAIHKALTELSQNKGKRVILLPAMRTSITCPKCKYCDSKNRNGEKFNCLKCGIELNADIDVATENLAWVAITAQSMPKPTCERSGDAKKPVRARKAKAPEFHDKLAPSYTVVLREAV

SEQ ID NO: 311 - protein, 717 aa, synthetic construct
MIKPTVSQFLTPGFKLIRNHSRTALLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLAVVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMWTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 312 - protein, 717 aa, synthetic construct
MIKPTVSQFLTPGFKLIRNHSRTALLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKLWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQWTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 313 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKWRKLSKRIKNVSPILGIICIKKDWVVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQYARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 314 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSILAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVMYKPVREKKGKELLENICDQNGSCKLATVDVGQNNPVAIGLFELKKVNGELTKTLISRHPTPIWFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 315 - protein, 717 aa, synthetic construct
MIKPTVSQFLTPGFKLIRNHYRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHYVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCILATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 316 - protein, 717 aa, synthetic construct
MIKPTVSQFLTPGFKLIRNHSRTAVLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVFQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSIVCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 317 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKIAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVLYKPVREKKGKELLENICDQNGSCILATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 318 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIIWIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQWTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMWTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 319 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSILAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVLPYQFDRLRIPIGEPGYVPKWQYTFLSKKENKRRKLSKRIKNVSPILGIIYIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 320 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKIAVNTYKGVQVKVDNKNKNNLAKINRKNEIAKLNGEQEISFEEIKAFDDKGYLLQKPSPNKSIYCYQSVSPKPFITSKYHYVNLPEEYIGYYRKSNEPIVSPYQFDRLRIPIGEPGYVPKWQYTFLSKKENKRRKLSKRIKNVSPILGIICIKKDWCVFDMRGLLRTNHWWKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 321 - protein, 717 aa, synthetic construct
MIKPTVSQFLTPGFKLIRNHSRTAILKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVLPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVMYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 322 - protein, 717 aa, synthetic construct
MIKPTVSQFLTPGFKLIRNHSRTAILKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIIYIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIWFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 323 - protein, 698 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNAIAKLNGLQIISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQGSSGDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 324 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYRNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 325 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNRAKINRKNEIAKLNGEQEISFEEIKAFDDKGYLLQKPSPNKSIYCYQSVSPKPFITSKYHNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 326 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNRAKINRKNEIAKLNGEQEISFEEIKAFDDKGYLLQKPSPNKSIYCYQSVSPKPFITSKYRNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 327 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGGLGPSYTVVLREAV

SEQ ID NO: 328 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNRAKINRKNEIAKLNGEQEISFEEIKAFDDKGYLLQKPSPNKSIYCYQSVSPKPFITSKYRNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGGLGPSYTVVLREAV

SEQ ID NO: 329 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 330 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYRNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 331 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNRAKINRKNEIAKLNGEQEISFEEIKAFDDKGYLLQKPSPNKSIYCYQSVSPKPFITSKYHNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 332 - protein, 717 aa, synthetic construct; identical to SEQ ID NO: 282

SEQ ID NO: 333 - protein, 717 aa, synthetic construct
MIKPTVSQFLTPGFKLIRNHYRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWWKYHKPTDSINDLFDYFTGDPVIDTKANVVRFRYKMENGIVLYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 334 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 335 - protein, 716 aa, synthetic construct
MIKPTVSQFLTPGFKLIRNHSRTAGLKLKNEGEEACKKFVRENEIPKDECPNFQPPAKANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 336 - protein, 744 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPEKHRSQLSMAKHKRRRAWYALSQNKPRPPKDGSKGRRSVRDLADLKAASLADAILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 337 - protein, 735 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPEWMRTAGEKTNPRTQKKFMHPGLSTRKNKRMRLPRSVRSAPLGAILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 338 - protein, 737 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPEWQRPHLSMKCKRVRMWYARANWRRKPGRRSVLNEARLKEASAKGAILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 339 - protein, 736 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPEWQREAGTAISPKTGKAVTVPGLSPKKNKRMRRYWRSEKEKAQDAILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 340 - protein, 738 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPEWQRSQLTTQKHRRKRSWYSAQKWKPRTGRTSTFDPDRLNCARAQGAILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 341 - protein, 735 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPEPHREGLTGRKDRRMRRYYETERGTKLKRPPLTAKGRADKANEAILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 342 - protein, 739 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPEWQRLKCSTNKHRRMRQWSNQDYKPKAGRRAKPLEFQAHLTRERAKGAILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 343 - protein, 736 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQREAGTAISPKTGKAVTVPGLSPKKNKRMRRYWRSEKEKAQDAILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 344 - protein, 736 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKTGKAVTVPGLSPKKNKRMRRYWRSEKEKAQDAILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 345 - protein, 736 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKAVTVPGLSPKKNKRMRRYWRSEKEKAQDAILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 346 - protein, 735 aa, synthetic construct
MIKPTVSQFLTPGFKLIRNHSRTAGLKLKNEGEEACKKFVRENEIPKDECPNFQPPAKANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPEWQREAGTAISPKRRKAVTVPGLSPKKNKRMRRYWRSEKEKAQDAILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 347 - protein, 736 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPEWQREAGTAISPKRRKAVTVPGLSPKKNKRMRRYWRSEKEKAQDAILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 348 - RNA, 20 nt, synthetic construct
  source 1..20: mol_type = other RNA
attgctccttacgaggagac

SEQ ID NO: 349 - protein, 5 aa, synthetic construct
GGPAI

SEQ ID NO: 350 - protein, 12 aa, synthetic construct
KWQYTFLSKKEN

SEQ ID NO: 351 - protein, 10 aa, synthetic construct
LSKRIKNVSP

SEQ ID NO: 352 - protein, 26 aa, synthetic construct
KWQYTFLSKKENKRRKLSKRIKNVSP

SEQ ID NO: 353 - protein, 23 aa, synthetic construct
YTFLSKKENKRRKLSKRIKNVSP

SEQ ID NO: 354 - protein, 13 aa, synthetic construct
RRKLSKRIKNVSP

SEQ ID NO: 355 - protein, 10 aa, synthetic construct
LSKRIKNVSP

SEQ ID NO: 356 - protein, 4 aa, synthetic construct
PPAK

SEQ ID NO: 357 - protein, 12 aa, synthetic construct
EWQREAGTAISP

SEQ ID NO: 358 - protein, 29 aa, synthetic construct
AVTVPGLSPKKNKRMRRYWRSEKEKAQDA

SEQ ID NO: 359 - protein, 53 aa, synthetic construct
EKHRSQLSMAKHKRRRAWYALSQNKPRPPKDGSKGRRSVRDLADLKAASLADA

SEQ ID NO: 360 - protein, 44 aa, synthetic construct
EWMRTAGEKTNPRTQKKFMHPGLSTRKNKRMRLPRSVRSAPLGA

SEQ ID NO: 361 - protein, 46 aa, synthetic construct
EWQRPHLSMKCKRVRMWYARANWRRKPGRRSVLNEARLKEASAKGA

SEQ ID NO: 362 - protein, 45 aa, synthetic construct
EWQREAGTAISPKTGKAVTVPGLSPKKNKRMRRYWRSEKEKAQDA

SEQ ID NO: 363 - protein, 47 aa, synthetic construct
EWQRSQLTTQKHRRKRSWYSAQKWKPRTGRTSTFDPDRLNCARAQGA

SEQ ID NO: 364 - protein, 44 aa, synthetic construct
EPHREGLTGRKDRRMRRYYETERGTKLKRPPLTAKGRADKANEA

SEQ ID NO: 365 - protein, 48 aa, synthetic construct
EWQRLKCSTNKHRRMRQWSNQDYKPKAGRRAKPLEFQAHLTRERAKGA

SEQ ID NO: 366 - protein, 42 aa, synthetic construct
REAGTAISPKTGKAVTVPGLSPKKNKRMRRYWRSEKEKAQDA

SEQ ID NO: 367 - protein, 32 aa, synthetic construct
TGKAVTVPGLSPKKNKRMRRYWRSEKEKAQDA

SEQ ID NO: 368 - protein, 29 aa, synthetic construct
AVTVPGLSPKKNKRMRRYWRSEKEKAQDA

SEQ ID NO: 369 - protein, 5 aa, synthetic construct
GSGGS

SEQ ID NO: 370 - protein, 6 aa, synthetic construct
GGSGGS

SEQ ID NO: 371 - protein, 4 aa, synthetic construct
GGGS

SEQ ID NO: 372 - protein, 5 aa, synthetic construct
GGSGG

SEQ ID NO: 373 - protein, 5 aa, synthetic construct
GSGSG

SEQ ID NO: 374 - protein, 5 aa, synthetic construct
GSGGG

SEQ ID NO: 375 - protein, 5 aa, synthetic construct
GGGSG

SEQ ID NO: 376 - protein, 5 aa, synthetic construct
GSSSG

SEQ ID NO: 377 - protein, 6 aa, synthetic construct
HHHHHH

SEQ ID NO: 378 - protein, 4 aa, synthetic construct
GGSG

SEQ ID NO: 379 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFGLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPGPNKSIYCYQSVSPKPFITSKYRNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFLNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALNDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 380 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAQGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKPNEPIVSPYQFDRLRIPIGEPGYVPKWQYTFLSKKKNKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRKALNDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 381 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKKNLAKINRKNEIAKLNGEQEISFEEIKAFDDKGYLLQKPSPNKSIYCYQSVSPKPFITSKYRNVNLPEEYIGYYRKPNEPIVSPYQFDRLRIPIGEPGYVPKWQYTFLSKKKNKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVKKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 382 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKKNLAKINRKNEIAKLNGEQEISFEEIKAFDDKGYLLQKPGPNKSIYCYQSVSPKPFITSKYHNVNLPEEYIGYYRKPNEPIVSPYQFDRLRIPIGEPGYVPKWQYTFLSKKKNKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALNDIEWRLRRESLEFNKLSKSREQLARQLANWISSMCDVIGIENLVKKNNFFGGSGKREPGWDNFYKPKKENRWWINAIHKALTELSQNKGKRVILLPAMRTSITCPKCKYCDKKNRNGEKFNCLKCGIELNADIDVATENLATVAITAQSMPKPTCERSGDAKKPVRARKAKAPEFHGKLAPSYTVVLREAV

SEQ ID NO: 383 - protein, 717 aa, synthetic construct
MIKPTVSQFLTPGFKLIRNHLRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPGPNKSIYCYQSVSPKPFITSKYHNVNLPEEYIGYSRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 384 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFGLPKDKLPEPILKEEWRAQWLSEHGLDTVPYKEAQGLNLIIKNAVNTYKGVQVKVDNKNKNNLAKINRKNEIAKLNGEQEISFEEIKAFDDKGYLLQKPGPNKSIYCYQSVSPKPFITSKYRNVNLPEEYIGYSRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFLNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRKALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 385 - protein, 717 aa, synthetic construct
MIKPTVSQFLTPGFKLIRNHLRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAQGLNLIIKNAVNTYKGVQVKVDNKNKKNLAKINRKNEIAKLNGEQEISFEEIKAFDDKGYLLQKPGPNKSIYCYQSVSPKPFITSKYHNVNLPEEYIGYSRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALNDIEWRLRRESLEFNKLSKSREQLARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 386 - protein, 717 aa, synthetic construct
MIKPTVSQFLTPGFKLIRNHLRTAGRKLKNEGEEACKKFVRENEIPKDECPNFQGGPAIANIIAKSREFTEWEIYRSSLAIQEVIFTLPKDKLPEPILKEEWRAQWLSEHGLDTVPYKEAAGLNLIIKNAVNTYKGVQVKVDNKNKKNRAKINRKNEIAKLNGEQEISFEEIKAFDDKGYLLQKPSPNKSIYCYQSVSPKPFITSKYHNVNLPEEYIGYSRKPNEPIVSPYQFDRLRIPIGEPGYVPKWQRTFLSKKKNKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVKKSDYKWFQDYKPKLSKEVRDALSDIEWRLRRESLEFNKLSKSREQDARRLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 387 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAQGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYSRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFLNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRKALSDIEWRLRRESLEFNKLSKSREQDARRLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 388 - protein, 717 aa, synthetic construct
MIKPTVSQFLTPGFKLIRNHLRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAQGLNLIIKNAVNTYKGVQVKVDNKNKKNLAKINRKNEIAKLNGEQEISFEEIKAFDDKGYLLQKPSPNKSIYCYQSVSPKPFITSKYHNVNLPEEYIGYSRKPNEPIVSPYQFDRLRIPIGEPGYVPKWQRTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFLNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 389 - protein, 717 aa, synthetic construct
MIKPTVSQFLTPGFKLIRNHSRTAGRKLKNEGEEACKKFVRENEIPKDECPNFQGGPAIANIIAKSREFTEWEIYRSSLAIQEVIFGLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKPNEPIVSPYQFDRLRIPIGEPGYVPKWQYTFLSKKKNKRRKLSKRIKNVSPILGIIRIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVKKSDYKWFQDYKPKLSKEVRKALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 390 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKKNLAKINRKNEIAKLNGEQEISFEEIKAFDDKGYLLQKPGPNKSIYCYQSVSPKPFITSKYHNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVKKSDYKWFQDYKPKLSKEVRDALNDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 391 - protein, 717 aa, synthetic construct
MIKPTVSQFLTPGFKLIRNHLRTAGRKLKNEGEEACKKFVRENEIPKDECPNFQGGPAIANIIAKSREFTEWEIYQSSLAIQEVIFGLPKDKLPEPILKEEWRAQWLSEHGLDTVPYKEAAGLNLIIKNAVNTYKGVQVKVDNKNKKNLAKINRKNEIAKLNGEQEISFEEIKAFDDKGYLLQKPSPNKSIYCYQSVSPKPFITSKYRNVNLPEEYIGYSRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 392 - protein, 717 aa, synthetic construct
MIKPTVSQFLTPGFKLIRNHLRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAQGLNLIIKNAVNTYKGVQVKVDNKNKKNLAKINRKNEIAKLNGEQEISFEEIKAFDDKGYLLQKPGPNKSIYCYQSVSPKPFITSKYHNVNLPEEYIGYYRKPNEPIVSPYQFDRLRIPIGEPGYVPKWQYTFLSKKKNKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRKALSDIEWRLRRESLEFNKLSKSREQDARRLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 393 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAQGLNLIIKNAVNTYKGVQVKVDNKNKNNRAKINRKNEIAKLNGEQEISFEEIKAFDDKGYLLQKPGPNKSIYCYQSVSPKPFITSKYHNVNLPEEYIGYSRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 394 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAQGLNLIIKNAVNTYKGVQVKVDNKNKKNLAKINRKNEIAKLNGEQEISFEEIKAFDDKGYLLQKPSPNKSIYCYQSVSPKPFITSKYHNVNLPEEYIGYSRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVKKSDYKWFQDYKPKLSKEVRDALNDIEWRLRRESLEFNKLSKSREQLARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 395 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFGLPKDKLPEPILKEEWRAQWLSEHGLDTVPYKEAQGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYSRKPNEPIVSPYQFDRLRIPIGEPGYVPKWQYTFLSKKKNKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFLNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 396 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFGLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPGPNKSIYCYQSVSPKPFITSKYHNVNLPEEYIGYSRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVKKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 397 - protein, 717 aa, synthetic construct
MIKPTVSQFLTPGFKLIRNHLRTAGRKLKNEGEEACKKFVRENEIPKDECPNFQGGPAIANIIAKSREFTEWEIYRSSLAIQEVIFGLPKDKLPEPILKEEWRAQWLSEHGLDTVPYKEAAGLNLIIKNAVNTYKGVQVKVDNKNKKNLAKINRKNEIAKLNGEQEISFEEIKAFDDKGYLLQKPGPNKSIYCYQSVSPKPFITSKYHNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALNDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDKKNRNGEKFNCLKCGIELNADIDVATENLATVAITAQSMPKPTCERSGDAKKPVRARKAKAPEFHGKLAPSYTVVLREAV

SEQ ID NO: 398 - protein, 717 aa, synthetic construct
MIKPTVSQFLTPGFKLIRNHLRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAQGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYSRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFLNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVKKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 399 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFGLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALNDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 400 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFGLPKDKLPEPILKEEWRAQWLSEHGLDTVPYKEAAGLNLIIKNAVNTYKGVQVKVDNKNKKNLAKINRKNEIAKLNGEQEISFEEIKAFDDKGYLLQKPSPNKSIYCYQSVSPKPFITSKYHNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALNDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 401 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFGLPKDKLPEPILKEEWRAQWLSEHGLDTVPYKEAAGLNLIIKNAVNTYKGVQVKVDNKNKKNLAKINRKNEIAKLNGEQEISFEEIKAFDDKGYLLQKPSPNKSIYCYQSVSPKPFITSKYHNVNLPEEYIGYYRKPNEPIVSPYQFDRLRIPIGEPGYVPKWQYTFLSKKKNKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALNDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 402 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFGLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYSRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALNDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 403 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFGLPKDKLPEPILKEEWRAQWLSEHGLDTVPYKEAAGLNLIIKNAVNTYKGVQVKVDNKNKKNLAKINRKNEIAKLNGEQEISFEEIKAFDDKGYLLQKPSPNKSIYCYQSVSPKPFITSKYHNVNLPEEYIGYSRKPNEPIVSPYQFDRLRIPIGEPGYVPKWQYTFLSKKKNKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALNDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 404 - protein, 717 aa, synthetic construct
MIKPTVSQFLTPGFKLIRNHSRTAGRKLKNEGEEACKKFVRENEIPKDECPNFQGGPAIANIIAKSREFTEWEIYRSSLAIQEVIFGLPKDKLPEPILKEEWRAQWLSEHGLDTVPYKEAAGLNLIIKNAVNTYKGVQVKVDNKNKKNLAKINRKNEIAKLNGEQEISFEEIKAFDDKGYLLQKPSPNKSIYCYQSVSPKPFITSKYHNVNLPEEYIGYSRKPNEPIVSPYQFDRLRIPIGEPGYVPKWQYTFLSKKKNKRRKLSKRIKNVSPILGIIRIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVKKSDYKWFQDYKPKLSKEVRKALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 405 - protein, 717 aa, synthetic construct
MIKPTVSQFLTPGFKLIRNHSRTAGRKLKNEGEEACKKFVRENEIPKDECPNFQGGPAIANIIAKSREFTEWEIYRSSLAIQEVIFGLPKDKLPEPILKEEWRAQWLSEHGLDTVPYKEAAGLNLIIKNAVNTYKGVQVKVDNKNKKNLAKINRKNEIAKLNGEQEISFEEIKAFDDKGYLLQKPSPNKSIYCYQSVSPKPFITSKYHNVNLPEEYIGYSRKPNEPIVSPYQFDRLRIPIGEPGYVPKWQYTFLSKKKNKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVKKSDYKWFQDYKPKLSKEVRKALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 406 - protein, 717 aa, synthetic construct
MIKPTVSQFLTPGFKLIRNHSRTAGRKLKNEGEEACKKFVRENEIPKDECPNFQGGPAIANIIAKSREFTEWEIYRSSLAIQEVIFGLPKDKLPEPILKEEWRAQWLSEHGLDTVPYKEAAGLNLIIKNAVNTYKGVQVKVDNKNKKNLAKINRKNEIAKLNGEQEISFEEIKAFDDKGYLLQKPSPNKSIYCYQSVSPKPFITSKYHNVNLPEEYIGYSRKPNEPIVSPYQFDRLRIPIGEPGYVPKWQYTFLSKKKNKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVKKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 407 - protein, 717 aa, synthetic construct
MIKPTVSQFLTPGFKLIRNHLRTAGRKLKNEGEEACKKFVRENEIPKDECPNFQGGPAIANIIAKSREFTEWEIYRSSLAIQEVIFGLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKPNEPIVSPYQFDRLRIPIGEPGYVPKWQYTFLSKKKNKRRKLSKRIKNVSPILGIIRIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFLNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVKKSDYKWFQDYKPKLSKEVRKALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 408 - protein, 717 aa, synthetic construct
MIKPTVSQFLTPGFKLIRNHSRTAGRKLKNEGEEACKKFVRENEIPKDECPNFQGGPAIANIIAKSREFTEWEIYRSSLAIQEVIFGLPKDKLPEPILKEEWRAQWLSEHGLDTVPYKEAAGLNLIIKNAVNTYKGVQVKVDNKNKKNLAKINRKNEIAKLNGEQEISFEEIKAFDDKGYLLQKPGPNKSIYCYQSVSPKPFITSKYHNVNLPEEYIGYSRKPNEPIVSPYQFDRLRIPIGEPGYVPKWQYTFLSKKKNKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFLNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVKKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 409 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFGLPKDKLPEPILKEEWRAQWLSEHGLDTVPYKEAAGLNLIIKNAVNTYKGVQVKVDNKNKKNLAKINRKNEIAKLNGEQEISFEEIKAFDDKGYLLQKPSPNKSIYCYQSVSPKPFITSKYHNVNLPEEYIGYSRKPNEPIVSPYQFDRLRIPIGEPGYVPKWQYTFLSKKKNKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 410 - protein, 717 aa, synthetic construct; identical to SEQ ID NO: 409

SEQ ID NO: 411 - protein, 717 aa, synthetic construct
MIKPTVSQFLTPGFKLIRNHLRTAGRKLKNEGEEACKKFVRENEIPKDECPNFQGGPAIANIIAKSREFTEWEIYQSSLAIQEVIFGLPKDKLPEPILKEEWRAQWLSEHGLDTVPYKEAAGLNLIIKNAVNTYKGVQVKVDNKNKKNLAKINRKNEIAKLNGEQEISFEEIKAFDDKGYLLQKPSPNKSIYCYQSVSPKPFITSKYHNVNLPEEYIGYSRKPNEPIVSPYQFDRLRIPIGEPGYVPKWQYTFLSKKKNKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 412 - protein, 717 aa, synthetic construct
MIKPTVSQFLTPGFKLIRNHLRTAGRKLKNEGEEACKKFVRENEIPKDECPNFQGGPAIANIIAKSREFTEWEIYQSSLAIQEVIFGLPKDKLPEPILKEEWRAQWLSEHGLDTVPYKEAAGLNLIIKNAVNTYKGVQVKVDNKNKKNLAKINRKNEIAKLNGEQEISFEEIKAFDDKGYLLQKPSPNKSIYCYQSVSPKPFITSKYHNVNLPEEYIGYSRKPNEPIVSPYQFDRLRIPIGEPGYVPKWQYTFLSKKKNKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFLNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVKKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 413 - protein, 717 aa, synthetic construct
MIKPTVSQFLTPGFKLIRNHLRTAGRKLKNEGEEACKKFVRENEIPKDECPNFQGGPAIANIIAKSREFTEWEIYQSSLAIQEVIFGLPKDKLPEPILKEEWRAQWLSEHGLDTVPYKEAQGLNLIIKNAVNTYKGVQVKVDNKNKKNLAKINRKNEIAKLNGEQEISFEEIKAFDDKGYLLQKPSPNKSIYCYQSVSPKPFITSKYHNVNLPEEYIGYSRKPNEPIVSPYQFDRLRIPIGEPGYVPKWQYTFLSKKKNKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFLNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVKKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDKKNRNGEKFNCLKCGIELNADIDVATENLATVAITAQSMPKPTCERSGDAKKPVRARKAKAPEFHGKLAPSYTVVLREAV

SEQ ID NO: 414 - protein, 717 aa, synthetic construct
MIKPTVSQFLTPGFKLIRNHLRTAGRKLKNEGEEACKKFVRENEIPKDECPNFQGGPAIANIIAKSREFTEWEIYQSSLAIQEVIFGLPKDKLPEPILKEEWRAQWLSEHGLDTVPYKEAQGLNLIIKNAVNTYKGVQVKVDNKNKKNLAKINRKNEIAKLNGEQEISFEEIKAFDDKGYLLQKPGPNKSIYCYQSVSPKPFITSKYHNVNLPEEYIGYSRKPNEPIVSPYQFDRLRIPIGEPGYVPKWQYTFLSKKKNKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFLNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVKKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDKKNRNGEKFNCLKCGIELNADIDVATENLATVAITAQSMPKPTCERSGDAKKPVRARKAKAPEFHGKLAPSYTVVLREAV

SEQ ID NO: 415 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFGLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 416 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFGLPKDKLPEPILKEEWRAQWLSEHGLDTVPYKEAAGLNLIIKNAVNTYKGVQVKVDNKNKKNLAKINRKNEIAKLNGEQEISFEEIKAFDDKGYLLQKPSPNKSIYCYQSVSPKPFITSKYHNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 417 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFGLPKDKLPEPILKEEWRAQWLSEHGLDTVPYKEAAGLNLIIKNAVNTYKGVQVKVDNKNKKNLAKINRKNEIAKLNGEQEISFEEIKAFDDKGYLLQKPSPNKSIYCYQSVSPKPFITSKYHNVNLPEEYIGYYRKPNEPIVSPYQFDRLRIPIGEPGYVPKWQYTFLSKKKNKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 418 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFGLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPGPNKSIYCYQSVSPKPFITSKYRNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFLNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 419 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKKNLAKINRKNEIAKLNGEQEISFEEIKAFDDKGYLLQKPGPNKSIYCYQSVSPKPFITSKYHNVNLPEEYIGYYRKPNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVKKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 420 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKPNEPIVSPYQFDRLRIPIGEPGYVPKWQYTFLSKKKNKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMTSGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHGKLAPSYTVVLREAV

SEQ ID NO: 421 - RNA, 40 nt, synthetic construct
  source 1..40: mol_type = other RNA
attgctccttacgaggagacttgccctcctggcgctcctg

(19) United States
(12) Patent Application Publication
(10) Pub. No.: US 2026/0174899 A1
(43) Pub. Date: Jun. 25, 2026

(54) EFFECTOR PROTEINS, COMPOSITIONS, SYSTEMS AND METHODS OF USE THEREOF

(71) Applicant: Mammoth Biosciences, Inc., Brisbane, CA (US)

(72) Inventors: William Douglass WRIGHT, Fairfield, CA (US); Adam L. GARSKE, Palo Alto, CA (US); Aaron DELOUGHERY, San Francisco, CA (US); Shiaki Arnett MINAMI, South San Francisco, CA (US); Lucas Benjamin HARRINGTON, San Francisco, CA (US); Benjamin Julius RAUCH, San Francisco, CA (US); Wang-Ting LU, San Francisco, CA (US); Ishita JAIN, San Francisco, CA (US); Siming XU, Redwood City, CA (US); Susan Lorraine PROVIDENZA, San Carlos, CA (US); Mark DEWITT, Millbrae, CA (US)

(21) Appl. No.: 19/361,915

(22) Filed: Oct. 17, 2025

Related U.S. Application Data
(63) Continuation of application No. PCT/US2024/025555, filed on Apr. 19, 2024.
(60) Provisional application No. 63/497,412, filed on Apr. 20, 2023.
(60) Provisional application No. 63/515,556, filed on Jul. 25, 2023.
(60) Provisional application No. 63/586,756, filed on Sep. 29, 2023.
(60) Provisional application No. 63/598,900, filed on Nov. 14, 2023.

(51) Int. Cl. A61K 48/00; A61K 9/127; A61K 9/51; C12N 9/10; C12N 9/12; C12N 9/22; C12N 9/78; C12N 15/11; C12N 15/86; C12N 15/88

(52) U.S. Cl. CPC A61K 48/005; A61K 9/127; A61K 9/5123; C12N 9/1007; C12N 9/1276; C12N 9/226; C12N 9/78; C12N 15/111; C12N 15/86; C12N 15/88; C07K 2319/80; C12N 2310/20; C12N 2750/14143; C12Y 201/01; C12Y 207/07049; C12Y 305/04

(57) ABSTRACT

Provided herein are compositions, systems, and methods comprising effector proteins and uses thereof. These effector proteins may be characterized as engineered CRISPR-associated (Cas) proteins. Various compositions, systems, and methods of the present disclosure may leverage the activities of these effector proteins for the editing, detecting and/or engineering of nucleic acids.

### CROSS-REFERENCED APPLICATIONS

This application is a continuation of International Patent Application No. PCT/US2024/025555, filed Apr. 19, 2024, which claims the benefit of priority to U.S. Provisional Application No. 63/497,412, filed on Apr. 20, 2023, U.S. Provisional Application No. 63/515,556, filed on Jul. 25, 2023, U.S. Provisional Application No. 63/586,756, filed on Sep. 29, 2023, and U.S. Provisional Application No. 63/598,900, filed on Nov. 14, 2023, the entire contents of each of which are incorporated herein by reference.

### INCORPORATION BY REFERENCE OF SEQUENCE LISTING

The instant application contains a Sequence Listing, which has been submitted via Patent Center. The Sequence Listing titled 203477-777301_US_SL.xml, which was created on Oct. 13, 2025, and is 548,920 bytes in size, is hereby incorporated by reference in its entirety.

### FIELD

The present disclosure relates generally to polypeptides, such as effector proteins, compositions of such polypeptides and guide nucleic acids, systems, and methods of using such polypeptides and compositions, including detecting and editing target nucleic acids.

### BACKGROUND

Clustered Regularly Interspaced Short Palindromic Repeats (CRISPR) and associated proteins (Cas proteins), sometimes referred to as a CRISPR/Cas system, were first identified in certain bacterial species and are now understood to form part of a prokaryotic acquired immune system. CRISPR/Cas systems provide immunity in bacteria and archaea against viruses and plasmids by targeting the nucleic acids of the viruses and plasmids in a sequence-specific manner. Native systems contain a CRISPR array, which includes direct repeats flanking short spacer sequences that, in part, guide Cas proteins to their targets. The discovery of CRISPR/Cas systems has revolutionized the field of genomic manipulation and engineering, and therapeutic applications of these systems are being explored.

### SUMMARY

The present disclosure provides for polypeptides, such as effector proteins, compositions, systems and methods comprising the same, and uses thereof. In some instances, compositions, systems and methods comprise guide nucleic acids or uses thereof. Compositions, systems and methods disclosed herein may leverage nucleic acid modification activities, such as nucleic acid editing. Editing may comprise: insertion, deletion, substitution, or a combination thereof of one or more nucleotides or amino acids. Editing may also comprise cleavage activity, such as cis cleavage activity, nickase activity and/or nuclease activity. In some instances, compositions, systems and methods are useful for the editing the sequence of target nucleic acids. In some instances, compositions, systems and methods are useful for the treatment of a disease or disorder. The disease or disorder may be associated with a target nucleic acid. The disease or disorder may be associated with one or more mutations in the target nucleic acid.

### Certain Embodiments

Provided herein are effector proteins or nucleic acids encoding the effector proteins. In some embodiments, the effector protein comprises an amino acid sequence that is at least 70%, at least 75%, at least 80%, at least 85%, at least 90%, at least 95%, at least 98% or at least 99% identical to any one of the sequences set forth in TABLE 1, wherein the effector protein comprises a deletion of one or more domains, a substitution of one or more domains for a different amino acid sequence, or a combination thereof, and wherein the one or more domains independently comprise an amino acid sequence that is at least 90% identical to any one of the domains identified in TABLE 3. In some embodiments, the effector protein comprises a deletion of one or more domains, a substitution of one or more domains for a different amino acid sequence, or a combination thereof, wherein the one or more domains independently comprise an amino acid sequence that is at least 90% identical to any one of the domains identified in TABLE 3, and wherein the effector protein comprises an amino acid sequence, other than the deletion of one or more domains, the substitution of one or more domains for a different amino acid sequence, or the combination thereof, that is at least 70%, at least 75%, at least 80%, at least 85%, at least 90%, at least 95%, at least 98% or at least 99% identical to any one of the sequences set forth in TABLE 1. In some embodiments, (a) the effector protein comprises an amino acid sequence that is at least 70% identical to SEQ ID NO: 1, (b) the domain comprises an amino acid sequence that is at least 90% identical to any one of SEQ ID NO: 6-22 and 349-355, and (c) the different amino acid sequence comprises an amino acid sequence that is at least 90% identical to any one of SEQ ID NO: 41-104 and 356-368. In some embodiments, the different amino acid sequence comprises any one of the amino acid sequences of SEQ ID NO: 18, 41-104 and 356-368. In some embodiments, (a) the effector protein comprises the amino acid sequence that is at least 70% identical to SEQ ID NO: 1, (b) the domain comprises the amino acid sequence that is at least 90% identical to SEQ ID NO: 6, and (c) the different amino acid sequence comprises the amino acid sequence that is at least 90% identical to any one of SEQ ID NO: 41-47. In some embodiments, (a) the effector protein comprises the amino acid sequence that is at least 70% identical to SEQ ID NO: 1, (b) the domain comprises the amino acid sequence that is at least 90% identical to any one of SEQ ID NO: 7-10, 13, 17-18 and 20, and (c) the different amino acid sequence comprises the amino acid sequence of SEQ ID NO: 48. In some embodiments, the effector protein comprises the amino acid sequence that is at least 70% identical to SEQ ID NO: 1, (b) the domain comprises the amino acid sequence that is at least 90% identical to any one of SEQ ID NO: 11 and 12, and (c) the different amino acid sequence comprises the amino acid sequence of SEQ ID NO: 49. In some embodiments, (a) the effector protein comprises the amino acid sequence that is at least 70% identical to SEQ ID NO: 1, (b) the domain comprises the amino acid sequence that is at least 90% identical to SEQ ID NO: 14, and (c) the different amino acid sequence comprises the amino acid sequence that is at least 90% identical to any one of SEQ ID NO: 50-65. In some embodiments, (a) the effector protein comprises the amino acid sequence that is at least 70% identical to SEQ ID NO: 1, (b) the domain comprises the amino acid sequence that is at least 90% identical to SEQ ID NO: 15, and (c) the different amino acid sequence comprises the amino acid sequence that is at least 90% identical to any one of SEQ ID NO: 66-100. In some embodiments, (a) the effector protein comprises the amino acid sequence that is at least 70% identical to SEQ ID NO: 1, (b) the domain comprises the amino acid sequence that is at least 90% identical to SEQ ID NO: 16, and (c) the different amino acid sequence comprises the amino acid sequence that is at least 90% identical to SEQ ID NO: 101. In some embodiments, (a) the effector protein comprises the amino acid sequence that is at least 70% identical to SEQ ID NO: 1, (b) the domain comprises the amino acid sequence that is at least 90% identical to SEQ ID NO: 19, and (c) the different amino acid sequence comprises the amino acid sequence of SEQ ID NO: 102. In some embodiments, (a) the effector protein comprises the amino acid sequence that is at least 70% identical to SEQ ID NO: 1, (b) the domain comprises the amino acid sequence that is at least 90% identical to SEQ ID NO: 21, and (c) the different amino acid sequence comprises the amino acid sequence that is at least 90% identical to SEQ ID NO: 103. In some embodiments, (a) the effector protein comprises the amino acid sequence that is at least 70% identical to SEQ ID NO: 1, (b) the domain comprises the amino acid sequence that is at least 90% identical to SEQ ID NO: 22, and (c) the different amino acid sequence comprises the amino acid sequence that is at least 90% identical to SEQ ID NO: 104. In some embodiments, (a) the effector protein comprises the amino acid sequence that is at least 70% identical to SEQ ID NO: 1, (b) the domain comprises the amino acid sequence that is at least 90% identical to SEQ ID NO: 349, and (c) the different amino acid sequence comprises the amino acid sequence that is at least 90% identical to SEQ ID NO: 356. In some embodiments, (a) the effector protein comprises the amino acid sequence that is at least 70% identical to SEQ ID NO: 1, (b) the domain comprises the amino acid sequence that is at least 90% identical to SEQ ID NO: 350, and (c) the different amino acid sequence comprises the amino acid sequence that is at least 90% identical to SEQ ID NO: 357. In some embodiments, (a) the effector protein comprises the amino acid sequence that is at least 70% identical to SEQ ID NO: 1, (b) the domain comprises the amino acid sequence that is at least 90% identical to SEQ ID NO: 351, and (c) the different amino acid sequence comprises the amino acid sequence that is at least 90% identical to SEQ ID NO: 358. In some embodiments, (a) the effector protein comprises the amino acid sequence that is at least 70% identical to SEQ ID NO: 1, (b) the domain comprises the amino acid sequence that is at least 90% identical to SEQ ID NO: 352, and (c) the different amino acid sequence comprises the amino acid sequence that is at least 90% identical to any one of SEQ ID NOS: 359-365. In some embodiments, (a) the effector protein comprises the amino acid sequence that is at least 70% identical to SEQ ID NO: 1, (b) the domain comprises the amino acid sequence that is at least 90% identical to SEQ ID NO: 353, and (c) the different amino acid sequence comprises the amino acid sequence that is at least 90% identical to SEQ ID NO: 366. In some embodiments, (a) the effector protein comprises the amino acid sequence that is at least 70% identical to SEQ ID NO: 1, (b) the domain comprises the amino acid sequence that is at least 90% identical to SEQ ID NO: 354, and (c) the different amino acid sequence comprises the amino acid sequence that is at least 90% identical to SEQ ID NO: 367. In some embodiments, (a) the effector protein comprises the amino acid sequence that is at least 70% identical to SEQ ID NO: 1, (b) the domain comprises the amino acid sequence that is at least 90% identical to SEQ ID NO: 355, and (c) the different amino acid sequence comprises the amino acid sequence that is at least 90% identical to SEQ ID NO: 368. In some embodiments, (a) the effector protein comprises an amino acid sequence that is at least 70% identical to SEQ ID NO: 2, (b) the domain comprises an amino acid sequence that is at least 90% identical to any one of SEQ ID NO: 23-25, and (c) the different amino acid sequence comprises an amino acid sequence of SEQ ID NO: 18 or 48. In some embodiments, (a) the effector protein comprises the amino acid sequence that is at least 70% identical to SEQ ID NO: 2, (b) the domain comprises the amino acid sequence that is at least 90% identical to any one of SEQ ID NO: 23-25, and (c) the different amino acid sequence comprises the amino acid sequence of SEQ ID NO: 48. In some embodiments, (a) the effector protein comprises the amino acid sequence that is at least 70% identical to SEQ ID NO: 2, (b) the domain comprises the amino acid sequence that is at least 90% identical to SEQ ID NO: 24, and (c) the different amino acid sequence comprises the amino acid sequence that is at least 90% identical to SEQ ID NO: 18. In some embodiments, (a) the effector protein comprises an amino acid sequence that is at least 70% identical to SEQ ID NO: 3, (b) the domain comprises an amino acid sequence that is at least 90% identical to any one of SEQ ID NO: 26-28, and (c) the different amino acid sequence comprises an amino acid sequence of SEQ ID NO: 18 or 48. In some embodiments, (a) the effector protein comprises the amino acid sequence that is at least 70% identical to SEQ ID NO: 3, (b) the domain comprises the amino acid sequence that is at least 90% identical to any one of SEQ ID NO: 26-28, and (c) the different amino acid sequence comprises the amino acid sequence of SEQ ID NO: 48. In some embodiments, (a) the effector protein comprises the amino acid sequence that is at least 70% identical to SEQ ID NO: 3, (b) the domain comprises the amino acid sequence that is at least 90% identical to SEQ ID NO: 27, and (c) the different amino acid sequence comprises the amino acid sequence that is at least 90% identical to SEQ ID NO: 18. In some embodiments, (a) the effector protein comprises an amino acid sequence that is at least 70% identical to SEQ ID NO: 4, (b) the domain comprises an amino acid sequence that is at least 90% identical to any one of SEQ ID NO: 29-31, and (c) the different amino acid sequence comprises an amino acid sequence of SEQ ID NO: 18 or 48. In some embodiments, (a) the effector protein comprises the amino acid sequence that is at least 70% identical to SEQ ID NO: 4, (b) the domain comprises the amino acid sequence that is at least 90% identical to any one of SEQ ID NO: 29-31, and (c) the different amino acid sequence comprises the amino acid sequence of SEQ ID NO: 48. In some embodiments, (a) the effector protein comprises the amino acid sequence that is at least 70% identical to SEQ ID NO: 4, (b) the domain comprises the amino acid sequence that is at least 90% identical to SEQ ID NO: 30, and (c) the different amino acid sequence comprises the amino acid sequence that is at least 90% identical to SEQ ID NO: 18. In some embodiments, (a) the effector protein comprises an amino acid sequence that is at least 70% identical to SEQ ID NO: 5, (b) the domain comprises an amino acid sequence that is at least 90% identical to any one of SEQ ID NO: 32-34, and (c) the different amino acid sequence comprises an amino acid sequence of SEQ ID NO: 18 or 48. In some embodiments, (a) the effector protein comprises the amino acid sequence that is at least 70% identical to SEQ ID NO: 5, (b) the domain comprises the amino acid sequence that is at least 90% identical to any one of SEQ ID NO: 32-34, and (c) the different amino acid sequence comprises the amino acid sequence of SEQ ID NO: 48. In some embodiments, (a) the effector protein comprises the amino acid sequence that is at least 70% identical to SEQ ID NO: 5, (b) the domain comprises the amino acid sequence that is at least 90% identical to SEQ ID NO: 33, and (c) the different amino acid sequence comprises the amino acid sequence that is at least 90% identical to SEQ ID NO: 18. In some embodiments, the effector protein comprises an amino acid sequence that is at least 85%, at least 90%, at least 95%, at least 98%, at least 99% or 100% identical to any one of the amino acid sequences set forth in TABLE 4. In some embodiments, the effector protein further comprises any one of the amino acid substitutions recited in TABLE 2. In some embodiments, the effector protein comprises a substitution of I471T relative to the amino acid sequence of SEQ ID NO: 1. In some embodiments, the effector protein comprises a substitution of L26R relative to the amino acid sequence of SEQ ID NO: 1. In some embodiments, the effector protein further comprises substitutions of S223P, I471T and D703G relative to the amino acid sequence of SEQ ID NO: 1. In some embodiments, the effector protein further comprises a substitution of S186G relative to the amino acid sequence of SEQ ID NO: 1. In some embodiments, the effector protein comprises substitutions of L26K and H208R relative to the amino acid sequence of SEQ ID NO: 1. In some embodiments, the effector protein comprises substitutions of L26K and D703G relative to the amino acid sequence of SEQ ID NO: 1. In some embodiments, the effector protein comprises substitutions of L26K, L149R and I471T relative to the amino acid sequence of SEQ ID NO: 1. In some embodiments, the effector protein comprises a combination of substitutions relative to the amino acid sequence of SEQ ID NO: 1, wherein the combination is selected from: (a) L26R, I471T, S223P and D703G; (b) L26R, I471T, S223P, D703G and H208R; (c) L26R, I471T, S223P, D703G and L149R; (d) L26R, I471T, S223P, D703G, L149R and H208R; (e) L26R, I471T, S223P, D703G, D704G and A706G; (f) L26R, I471T, S223P, D703G, L149R, H208R, D704G and A706G; (g) I471T, S223P and D703G; (h) I471T, S223P, D703G and H208R; (i) I471T, S223P, D703G and L149R; (j) I471T, S223P, D703G, L149R and H208R; (k) I471T, S223P, D703G, D704G and A706G; (l) I471T, S223P, D703G, L149R, H208R, D704G and A706G; (m) I471T and E157R; (n) I471T, E157R, S223P and D703G; (o) L26R, I471T, E157R, S223P and D703G; (p) L26R, T87G, S186G, H208R, S223P, C405L, I471T, S526N and D703G; (q) L26R, A121Q, S223P, E258K, I471T, D523K, S526N and D703G; (r) L26R, N147K, H208R, S223P, E258K, I471T, M503K and D703G; (s) L26R, N147K, S186G, S223P, E258K, I471T, S526N, D549L, S638K and D703G; (t) S21L, L26R, S186G, Y220S, S223P, I471T and D703G; (u) L26R, T87G, A121Q, S186G, H208R, Y220S, S223P, C405L, I471T, D523K and D703G; (v) S21L, L26R, A121Q, N147K, S186G, Y220S, S223P, I471T, S526N, D549L and D703G; (w) S21L, L26R, Q76R, N147K, L149R, Y220S, S223P, Y251R, E258K, I471T, M503K, Q552R and D703G; (x) L26R, A121Q, Y220S, S223P, C405L, I471T, D523K, Q552R and D703G; (y) S21L, L26R, A121Q, N147K, Y220S, S223P, Y251R, C405L, I471T and D703G; (z) L26R, Q76R, T87G, S223P, E258K, C279R, I471T, M503K, D523K and D703G; (aa) L26R, N147K, S186G, S223P, I471T, M503K, S526N and D703G; (bb) S21L, L26R, T87G, N147K, H208R, Y220S, S223P, I471T and D703G; (cc) S21L, L26R, A121Q, N147K, S186G, S223P, E258K, I471T, D523K, Q552R and D703G; (dd) L26R, A121Q, L149R, S186G, Y220S, S223P, I471T and D703G; (ee) L26R, A121Q, N147K, Y220S, S223P, I471T, M503K, S526N, D549L and D703G; (ff) L26R, T87G, A121Q, Y220S, S223P, E258K, C405L, I471T and D703G; (gg) L26R, T87G, S186G, Y220S, S223P, I471T, M503K and D703G; (hh) S21L, L26R, Q76R, T87G, N147K, S186G, S223P, I471T, S526N, S638K and D703G; (ii) S21L, L26R, A121Q, Y220S, S223P, C405L, I471T, M503K and D703G; (jj) L26R, S223P, I471T and D703G; (kk) L26R, T87G, S223P, I471T, S526N and D703G; (ll) L26R, T87G, N147K, S223P, I471T, S526N and D703G; (mm) L26R, T87G, N147K, S223P, E258K, I471T, S526N and D703G; (nn) L26R, T87G, Y220S, S223P, I471T, S526N and D703G; (oo) L26R, T87G, N147K, Y220S, S223P, E258K, I471T, S526N and D703G; (pp) L26R, Q76R, T87G, N147K, Y220S, S223P, E258K, C279R, I471T, M503K, D523K and D703G; (qq) L26R, Q76R, T87G, N147K, Y220S, S223P, E258K, I471T, M503K, D523K and D703G; (rr) L26R, Q76R, T87G, N147K, Y220S, S223P, E258K, I471T, M503K and D703G; (ss) S21L, L26R, Q76R, T87G, S223P, E258K, C279R, C405L, I471T, M503K, D523K and D703G; (tt) L26R, Q76R, T87G, N147K, S186G, Y220S, S223P, E258K, C405L, I471T, M503K and D703G; (uu) L26R, T87G, Y220S, S223P, I471T and D703G; (vv) L26R, T87G, N147K, Y220S, S223P, E258K, I471T and D703G; (ww) S21L, L26R, T87G, N147K, Y220S, S223P, E258K, I471T and D703G; (xx) S21L, L26R, T87G, N147K, Y220S, S223P, E258K, C405L, I471T, M503K and D703G; (yy) S21L, L26R, T87G, A121Q, N147K, Y220S, S223P, E258K, C405L, I471T, M503K, S638K and D703G; (zz) S21L, L26R, T87G, A121Q, N147K, S186G, Y220S, S223P, E258K, C405L, I471T, M503K, S638K and D703G; (aaa) L26R, T87G, S223P, I471T and D703G; (bbb) L26R, T87G, N147K, S223P, I471T and D703G; (ccc) L26R, T87G, N147K, S223P, E258K, I471T and D703G; (ddd) L26R, T87G, S186G, H208R, S223P, C405L, I471T and D703G; (eee) L26R, N147K, S186G, S223P, I471T, M503K and D703G; and (fff) L26R, S223P, E258K, I471T and D703G. In some embodiments, the effector protein, when in complex with a guide nucleic acid and the guide nucleic acid is hybridized to a target sequence of a double stranded target nucleic acid, nicks the double stranded target nucleic acid. In some embodiments, the effector protein nicks a target strand of the double stranded target nucleic acid. In some embodiments, the effector protein nicks a non-target strand of the double stranded target nucleic acid. In some embodiments, the effector protein comprises cis cleavage activity. In some embodiments, the effector protein further comprises one or more heterologous peptides that are heterologous to the effector protein. In some embodiments, the one or more heterologous peptides are located at N-terminus, C-terminus, or both of the effector protein. In some embodiments, the one or more heterologous peptide independently comprises any one of the amino acid sequences recited in TABLE 5. In some embodiments, the effector protein recognizes any one of protospacer adjacent motif (PAM) sequences set forth in TABLE 6. In some embodiments, the nucleic acid encoding the effector protein comprises a messenger RNA. In some embodiments, the effector protein is covalently linked to a heterologous peptide or protein, optionally via a linker molecule.

Also provided herein is an effector protein or nucleic acids encoding the effector protein, wherein the effector protein is at least 70%, at least 75%, at least 80%, at least 85%, at least 90%, at least 95%, at least 98% or at least 99% identical to SEQ ID NO: 1, and wherein the effector protein comprises any combination of amino acid substitutions described in TABLE 2. Also provided herein is an effector protein or nucleic acids encoding the effector protein, wherein the effector protein comprises any combination of amino acid substitutions described in TABLE 2, and wherein the effector protein comprises an amino acid sequence, other than the amino acid substitutions described in TABLE 2, that is at least 70%, at least 75%, at least 80%, at least 85%, at least 90%, at least 95%, at least 98% or at least 99% identical to SEQ ID NO: 1. In some embodiments, the combination of amino acid substitutions is selected from: (a) L26R, I471T, S223P and D703G; (b) L26R, I471T, S223P, D703G and H208R; (c) L26R, I471T, S223P, D703G and L149R; (d) L26R, I471T, S223P, D703G, L149R and H208R; (e) L26R, I471T, S223P, D703G, D704G and A706G; (f) L26R, I471T, S223P, D703G, L149R, H208R, D704G and A706G; (g) I471T, S223P and D703G; (h) I471T, S223P, D703G and H208R; (i) I471T, S223P, D703G and L149R; (j) I471T, S223P, D703G, L149R and H208R; (k) I471T, S223P, D703G, D704G and A706G; (l) I471T, S223P, D703G, L149R, H208R, D704G and A706G; (m) I471T and E157R; (n) I471T, E157R, S223P and D703G; (o) L26R, I471T, E157R, S223P and D703G; (p) L26R, T87G, S186G, H208R, S223P, C405L, I471T, S526N and D703G; (q) L26R, A121Q, S223P, E258K, I471T, D523K, S526N and D703G; (r) L26R, N147K, H208R, S223P, E258K, I471T, M503K and D703G; (s) L26R, N147K, S186G, S223P, E258K, I471T, S526N, D549L, S638K and D703G; (t) S21L, L26R, S186G, Y220S, S223P, I471T and D703G; (u) L26R, T87G, A121Q, S186G, H208R, Y220S, S223P, C405L, I471T, D523K and D703G; (v) S21L, L26R, A121Q, N147K, S186G, Y220S, S223P, I471T, S526N, D549L and D703G; (w) S21L, L26R, Q76R, N147K, L149R, Y220S, S223P, Y251R, E258K, I471T, M503K, Q552R and D703G; (x) L26R, A121Q, Y220S, S223P, C405L, I471T, D523K, Q552R and D703G; (y) S21L, L26R, A121Q, N147K, Y220S, S223P, Y251R, C405L, I471T and D703G; (z) L26R, Q76R, T87G, S223P, E258K, C279R, I471T, M503K, D523K and D703G; (aa) L26R, N147K, S186G, S223P, I471T, M503K, S526N and D703G; (bb) S21L, L26R, T87G, N147K, H208R, Y220S, S223P, I471T and D703G; (cc) S21L, L26R, A121Q, N147K, S186G, S223P, E258K, I471T, D523K, Q552R and D703G; (dd) L26R, A121Q, L149R, S186G, Y220S, S223P, I471T and D703G; (ee) L26R, A121Q, N147K, Y220S, S223P, I471T, M503K, S526N, D549L and D703G; (ff) L26R, T87G, A121Q, Y220S, S223P, E258K, C405L, I471T and D703G; (gg) L26R, T87G, S186G, Y220S, S223P, I471T, M503K and D703G; (hh) S21L, L26R, Q76R, T87G, N147K, S186G, S223P, I471T, S526N, S638K and D703G; (ii) S21L, L26R, A121Q, Y220S, S223P, C405L, I471T, M503K and D703G; (jj) L26R, S223P, I471T and D703G; (kk) L26R, T87G, S223P, I471T, S526N and D703G; (ll) L26R, T87G, N147K, S223P, I471T, S526N and D703G; (mm) L26R, T87G, N147K, S223P, E258K, I471T, S526N and D703G; (nn) L26R, T87G, Y220S, S223P, I471T, S526N and D703G; (oo) L26R, T87G, N147K, Y220S, S223P, E258K, I471T, S526N and D703G; (pp) L26R, Q76R, T87G, N147K, Y220S, S223P, E258K, C279R, I471T, M503K, D523K and D703G; (qq) L26R, Q76R, T87G, N147K, Y220S, S223P, E258K, I471T, M503K, D523K and D703G; (rr) L26R, Q76R, T87G, N147K, Y220S, S223P, E258K, I471T, M503K and D703G; (ss) S21L, L26R, Q76R, T87G, S223P, E258K, C279R, C405L, I471T, M503K, D523K and D703G; (tt) L26R, Q76R, T87G, N147K, S186G, Y220S, S223P, E258K, C405L, I471T, M503K and D703G; (uu) L26R, T87G, Y220S, S223P, I471T and D703G; (vv) L26R, T87G, N147K, Y220S, S223P, E258K, I471T and D703G; (ww) S21L, L26R, T87G, N147K, Y220S, S223P, E258K, I471T and D703G; (xx) S21L, L26R, T87G, N147K, Y220S, S223P, E258K, C405L, I471T, M503K and D703G; (yy) S21L, L26R, T87G, A121Q, N147K, Y220S, S223P, E258K, C405L, I471T, M503K, S638K and D703G; (zz) S21L, L26R, T87G, A121Q, N147K, S186G, Y220S, S223P, E258K, C405L, I471T, M503K, S638K and D703G; (aaa) L26R, T87G, S223P, I471T and D703G; (bbb) L26R, T87G, N147K, S223P, I471T and D703G; (ccc) L26R, T87G, N147K, S223P, E258K, I471T and D703G; (ddd) L26R, T87G, S186G, H208R, S223P, C405L, I471T and D703G; (eee) L26R, N147K, S186G, S223P, I471T, M503K and D703G; and (fff) L26R, S223P, E258K, I471T and D703G. In some embodiments, the effector protein further comprises a deletion of one or more domains, a substitution of one or more domains for a different amino acid sequence, or a combination thereof, wherein the one or more domains independently comprise an amino acid sequence that is at least 90% identical to any one of the domains identified in TABLE 3. Also, provided herein are compositions, wherein the compositions comprise the effector protein or the nucleic acid encoding the effector protein described herein, and a guide nucleic acid. In some embodiments, the guide the guide nucleic acid comprises a nucleotide sequence that is at least 70%, at least 75%, at least 80%, at least 85%, at least 90%, at least 95% or 100% identical to SEQ ID NO: 348. In some embodiments, the nucleic acid encoding the effector protein comprises a messenger RNA. In some embodiments, the effector protein is covalently linked to a heterologous peptide or protein, optionally via a linker molecule.

Provided herein is an effector protein or nucleic acids encoding the effector protein, wherein the effector protein comprises or consists of any one of the amino acid sequences selected from TABLE 4. In some embodiments, the nucleic acid encoding the effector protein comprises a messenger RNA. In some embodiments, the effector protein is covalently linked to a heterologous peptide or protein, optionally via a linker molecule.

Provided herein is an effector protein or nucleic acids encoding the effector protein, wherein the effector protein comprises an amino acid sequence that is at least 90%, at least 95%, at least 97%, at least 98%, or at least 99% identical to any one of the sequences recited in TABLE 4. In some embodiments, the effector protein comprises an amino acid sequence that is at least 90%, at least 95%, at least 97%, at least 98%, or at least 99% identical to any one of the amino acid sequences recited in TABLE 4, and wherein the amino acid sequence comprises all amino acid differences between an amino acid sequence recited in TABLE 4 and SEQ ID NO: 1. In some embodiments, the amino acid sequence that is at least 90%, at least 95%, at least 97%, at least 98%, or at least 99% identical to any one of the amino acid sequences recited in TABLE 4, other than all the amino acid differences between the amino acid sequence recited in TABLE 4 and SEQ ID NO: 1, is comprised of conservative amino acid substitutions relative to the amino acid sequence recited in TABLE 4. In some embodiments, the one or more amino acid alterations are conservative amino acid substitutions. In some embodiments, the effector protein comprises at least 600, at least 620, at least 640, at least 660, at least 680, or at least 700 contiguous amino acids of any one of the sequences recited in TABLE 4. In some embodiments, the nucleic acid encoding the effector protein comprises a messenger RNA. In some embodiments, the effector protein is covalently linked to a heterologous peptide or protein, optionally via a linker molecule.

Provided herein are systems comprising one or more components, wherein the one or more components individually comprise: the effector protein or a nucleic acid encoding any one of the effector proteins described herein; and a guide nucleic acid or a nucleic acid that encodes the guide nucleic acid, wherein the guide nucleic acid comprises a repeat sequence and a spacer sequence, wherein the repeat sequence, at least partially, interacts with the effector protein, wherein the spacer sequence comprises a nucleic acid sequence that hybridizes to a target sequence in a target nucleic acid. In some embodiments, the repeat sequence comprises a nucleotide sequence that is at least 75%, at least 80%, at least 85%, at least 90%, at least 95%, at least 97%, or at least 98%, at least 99%, or 100% identical to the nucleotide sequence recited in TABLE 7. In some embodiments, the guide nucleic acid is a crRNA. In some embodiments, the spacer sequence comprises a nucleotide sequence in a range of from 10 to 20 linked nucleotides. In some embodiments, the spacer sequence comprises a nucleotide sequence of 14 linked nucleotides. In some embodiments, the spacer sequence comprises a nucleotide sequence that is about 80% to about 95% complementary to the target sequence. In some embodiments, the guide nucleic acid comprises one or more phosphorothioate (PS) backbone modifications, 2′-fluoro (2′-F) sugar modifications, or 2′-O-Methyl(2′OMe) sugar modifications. In some embodiments, the guide nucleic acid comprises PS backbone modification between −3 and −2 positions of a repeat sequence present in the guide nucleic acid, and wherein the repeat sequence comprises at least 24 nucleotides. In some embodiments, the guide nucleic acid further comprises at least one modification between −16 and −12 positions of a repeat sequence present in the guide nucleic acid. In some embodiments, the at least one modification comprises 2′OMe sugar modification at −14 position of the repeat sequence present in the guide nucleic acid. In some embodiments, the at least one modification comprises 2′OMe sugar modification at −16 position of the repeat sequence present in the guide nucleic acid. In some embodiments, the at least one modification comprises PS backbone modification between −13 and −12 positions of a repeat sequence present in the guide nucleic acid. In some embodiments, the at least one modification comprises PS backbone modification between −14 and −13 positions of a repeat sequence present in the guide nucleic acid. In some embodiments, the at least one modification comprises PS backbone modification between −15 and −14 positions of a repeat sequence present in the guide nucleic acid. In some embodiments, the target sequence is within a human gene. In some embodiments, the system further comprises the target nucleic acid, wherein the target nucleic acid is a double stranded DNA comprising a target strand and a non-target strand. In some embodiments, the spacer sequence hybridizes to the target strand and the PAM is located on a non-target strand of the target nucleic acid. In some embodiments, the PAM is located 5′ of a reverse complement of the target sequence on the non-target strand. In some embodiments, the target nucleic acid is isolated from a human cell. In some embodiments, the target nucleic acid is any one of the nucleic acids set forth in TABLE 8. In some embodiments, the target nucleic acid is associated with any one of the diseases or disorders of TABLE 9. In some embodiments, the system described herein further comprising an effector partner, or a nucleic acid encoding the effector partner. In some embodiments, the system comprises a fusion protein, or a nucleic acid encoding the fusion protein, wherein the fusion protein comprises the effector protein and the effector partner fused to each other. In some embodiments, N-terminus of the effector protein is linked to C-terminus of the effector partner. In some embodiments, C-terminus of the effector protein is linked to C-terminus of the effector partner. In some embodiments, the effector protein and the effector partner are directly fused to each other. In some embodiments, the effector protein and the effector partner are fused by a linker. In some embodiments, the system comprises an expression vector, wherein the expression vector encodes the effector protein, the effector partner, the guide nucleic acid, or a combination thereof. In some embodiments, the expression vector is a viral vector or a non-viral vector. In some embodiments, the viral vector is an adeno associated viral (AAV) vector. In some embodiments, the nucleic acids encoding the effector protein, the effector partner, or the combination thereof are messenger RNAs. In some embodiments, the systems described herein comprises a lipid or a lipid nanoparticle. In some embodiments, the system comprises: (a) an LNP, wherein the LNP contains the nucleic acid encoding the effector protein and the guide nucleic acid, wherein the nucleic acid encoding the effector protein comprises a messenger RNA; and (b) optionally, an AAV vector comprising a donor nucleic acid.

Provided herein is a library of nucleic acid expression vectors comprising at least one of the expression vectors described herein.

Provided herein are compositions comprising: any one of the effector proteins described herein; or one or more components of any one of the systems described herein.

Provided herein are pharmaceutical compositions comprising: any one of the effector proteins or the nucleic acids encoding the effector proteins described herein, one or more components of any one of the systems described herein, or any one of the compositions described herein; and a pharmaceutically acceptable excipient.

Provided herein are cells comprising: any one of the effector proteins or the nucleic acids encoding the effector proteins described herein; one or more components of any one of the systems described herein; the library of nucleic acid expression vectors described herein; any one of the compositions described herein; or any one of the pharmaceutical compositions described herein.

Provided herein are method of nicking a target nucleic acid within a human gene or associated with expression of a human gene, the method comprising contacting the target nucleic acid with one or more of: any one of the effector proteins or the nucleic acids encoding the effector proteins described herein; one or more components of any one of the systems described herein; the library of nucleic acid expression vectors described herein; any one of the compositions described herein; or any one of the pharmaceutical compositions described herein, thereby nicking the target nucleic acid. In some embodiments, the method is performed in a cell. In some embodiments, the method is performed in vivo. In some embodiments, the method is performed in vitro. In some embodiments, the target nucleic acid comprises a mutation associated with a disease. In some embodiments, the one or more mutations comprise a point mutation, a single nucleotide polymorphism (SNP), a chromosomal mutation, a copy number mutation, or any combination thereof. In some embodiments, the target nucleic acid is any one of the nucleic acids set forth in TABLE 8. In some embodiments, the target nucleic acid is associated with any one of the diseases set forth in TABLE 9.

Provided herein are method of modifying a target nucleic acid within a human gene or associated with expression of a human gene, the method comprising contacting the target nucleic acid with one or more of: any one of the effector proteins or the nucleic acids encoding the effector proteins described herein; one or more components of any one of the systems described herein; the library of nucleic acid expression vectors described herein; any one of the compositions described herein; or any one of the pharmaceutical compositions described herein, thereby modifying the target nucleic acid. In some embodiments, the method is performed in a cell. In some embodiments, the method is performed in vivo. In some embodiments, the method is performed in vitro. In some embodiments, the target nucleic acid comprises a mutation associated with a disease. In some embodiments, the one or more mutations comprise a point mutation, a single nucleotide polymorphism (SNP), a chromosomal mutation, a copy number mutation, or any combination thereof. In some embodiments, the target nucleic acid is any one of the nucleic acids set forth in TABLE 8. In some embodiments, the target nucleic acid is associated with any one of the diseases set forth in TABLE 9.

Provided herein are cells contacted by: any one of the effector proteins or the nucleic acids encoding the effector proteins described herein; one or more components of any one of the systems described herein; the library of nucleic acid expression vectors described herein; any one of the compositions described herein; any one of the pharmaceutical compositions described herein; or any one of the methods described herein.

Provided herein are cells comprising a target nucleic acid modified by: any one of the effector proteins or the nucleic acids encoding the effector proteins described herein; one or more components of any one of the systems described herein; the library of nucleic acid expression vectors described herein; any one of the compositions described herein; any one of the pharmaceutical compositions described herein; or any one of the methods described herein

Provided herein are the cells described herein, wherein the cell is a eukaryotic cell. In some embodiments, the cell is a mammalian cell. In some embodiments, the cell is a human cell.

Provided herein is a population of cells that comprises at least one cell of any one of the cells described herein.

### INCORPORATION BY REFERENCE

All publications, patents and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows results of a cis cleavage assay for effector proteins as confirmed by polyacrylamide gel electrophoresis (PAGE). In FIG. 1, cleavage resulting in a double strand break or nicking of a target nucleic acid resulted in structural changes of supercoiled double stranded DNA plasmid. Effector proteins that nicked the target nucleic acid yielded a circular, but not supercoiled, target nucleic acid described as “nicked” in FIG. 1. Effector proteins that cleaved both strands of the supercoiled double stranded DNA target nucleic acid resulting in a double strand break yielded a linear target nucleic acid described as described as “linear” in FIG. 1. Effector proteins that did not cleave or nick the supercoiled double stranded DNA target nucleic acid yielded a target nucleic acid described as “uncut” in FIG. 1.

FIGS. 2A-2D show results of a cis cleavage assay for effector proteins as confirmed by polyacrylamide gel electrophoresis (PAGE). In FIGS. 2A-2D, cleavage resulting in a double strand break or nicking of a target nucleic acid resulted in structural changes of supercoiled double stranded DNA plasmid. Effector proteins that nicked the target nucleic acid yielded a circular, but not supercoiled, target nucleic acid described as “nicked” in FIGS. 2A-2D. Effector proteins that cleaved both strands of the supercoiled double stranded DNA target nucleic acid resulting in a double strand break yielded a linear target nucleic acid described as described as “linear” in FIGS. 2A-2D. Effector proteins that did not cleave or nick the supercoiled double stranded DNA target nucleic acid yielded a target nucleic acid described as “uncut” in FIGS. 2A-2D.

FIG. 3 shows schematics of expected fragment size for the restriction enzyme, NcoI, digested cis cleaved target nucleic acid with the WT effector protein comprising the amino acid sequence of SEQ ID NO: 1.

FIGS. 4A-4C shows results of restriction enzyme, NcoI, digested cis cleaved target nucleic acid with each of the effector proteins of SEQ ID NO: 95-100, 102-103, 113, 151, 153, 156-159, 162-163, 165, 167-170, 172, 174 and 176-179.

FIG. 5 shows schematics of expected fragment size for the restriction enzyme, PvuII, digested cis cleaved target nucleic acid with the WT effector protein comprising the amino acid sequence of SEQ ID NO: 1.

FIGS. 6A-6C shows results of restriction enzyme, PvuII, digested cis cleaved target nucleic acid with each of the effector proteins of SEQ ID NO: 95-100, 102-103, 113, 151, 153, 156-159, 162-163, 165, 167-170, 172, 174 and 176-179.

FIGS. 7A-7C show results of change in nuclease activity as a function of change in spacer sequence length of a guide RNA. FIGS. 7A and 7B show results of cis cleavage activity of the WT effector protein (SEQ ID NO: 1) and guide RNAs with different spacer lengths. FIG. 7A shows results of cis cleavage assays for the set of the guide RNA targeting SPI gene. FIG. 7B shows results of cis cleavage assays for the set of the guide RNA targeting B2M gene. FIG. 7C shows results of restriction enzyme, NcoI, digested cis cleaved target nucleic acid shown in FIGS. 7A and 7B.

FIGS. 8A and 8B show quantitative analysis of FIGS. 7A and 7B, respectively, data showing relative quantities of uncut, nicked or cleaved (double stranded break) target nucleic acid.

FIG. 9 shows the editing efficiency (% indels) of CasPhi. 12 I471T, delivered by LNP, in mice is comparable with Cas9.

FIG. 10 shows the editing efficiency (% indels) of CasPhi. 12 variants and six different guide RNAs, delivered by LNP, in mice.

FIGS. 11A-11F show various guide modifications that were tested. The modifications include one or more 2′-O-Methyl(2′OMe) sugar modifications, shown as [symbol] , and one or more phosphorothioate (PS) backbone modifications, shown as [symbol] . FIGS. 11A-11B show positions of unbiased modifications that were tested. FIGS. 11C-11F show positions of combinatorial modifications that were tested.

FIGS. 12A-12B show the effects of introducing chemical modifications to CasPhi.12 guide RNAs.

FIGS. 13A-13C show the effects of introducing chemical modifications to CasPhi.12 guide RNAs.

FIG. 14 shows results of luciferase assay that were performed to determine editing efficiency of LNPs comprising three variants of WT CasPhi. 12 effector protein (SEQ ID NO: 1) in combination with seven guide nucleic acids. The activity of three CasPhi.12 effector protein variants, L26R substitution, I471T substitution and both, are shown from left to right. SpyCas9 was used as a positive control.

FIG. 15 shows % indel activity that was observed for LNPs comprising three variants of WT CasPhi.12 effector protein (SEQ ID NO: 1) in combination with four guide nucleic acids. The three CasPhi.12 effector protein variants included L26R substitution, I471T substitution and both. Cas9 was used as a positive control.

FIG. 16 shows Mod % (indel activity) that was observed in HEK293T mammalian cells with variants of WT CasPhi.12 effector protein (SEQ ID NO: 1) in combination with five guide nucleic acids. Guide nucleic acids represented, from left to right for each variant assayed, are PL37872, PL37893, PL37905, PL37864, and PL37859.

FIG. 17 shows fold change of Mod % of variants of CasPhi. 12 effector protein (SEQ ID NO: 1) relative to that of CasPhi.12 variant L26R,I471T in HEK293T mammalian cells. Guide nucleic acids represented, from left to right for each variant assayed, are PL37859, PL37864, PL37893, and PL37905.

FIG. 18 shows Mod % (indel activity) that was observed in HEK293T mammalian cells with variants of CasPhi. 12 effector protein that included amino acid substitutions and N or C terminal truncations. Guide nucleic acids represented, from left to right for each variant assayed, are PL37872, PL37893, PL37864, PL37905, and PL37859.

FIG. 19 shows Mod % (indel activity) that was observed in HEK293T mammalian cells with variants of effector protein that included one of the amino acid sequences of SEQ ID NO: 271, 379-394 and 396-398.

### DETAILED DESCRIPTION OF THE INVENTION

It is to be understood that both the foregoing general description and the following detailed description are exemplary and explanatory only, and are not restrictive of the disclosure.

The section headings used herein are for organizational purposes only and are not to be construed as limiting the subject matter described.

All documents, or portions of documents, cited in this application, including, but not limited to, patents, patent applications, articles, books and treatises, are hereby expressly incorporated by reference in their entirety for any purpose.

### Definitions

Unless otherwise indicated, all technical terms used herein have the same meaning as commonly understood by one of ordinary skill in the art to which this invention belongs. Unless otherwise indicated or obvious from context, the following terms have the following meanings:

The terms, “a,” “an,” and “the,” as used herein, include plural references unless the context clearly dictates otherwise.

The terms, “or” and “and/or,” as used herein, include any and all combinations of one or more of the associated listed items.

The terms, “including,” “includes,” “included,” and other forms, are not limiting.

The terms, “comprise” and its grammatical equivalents, as used herein, specify the presence of stated features, integers, steps, operations, elements and/or components, but do not preclude the presence or addition of one or more other features, integers, steps, operations, elements, components and/or groups thereof.

The term, “about,” as used herein in reference to a number or range of numbers, is understood to mean the stated number and numbers+/−10% thereof, or 10% below the lower listed limit and 10% above the higher listed limit for the values listed for a range.

The terms, “% identical,” “% identity,” “percent identity,” and grammatical equivalents thereof, as used herein, in the context of an amino acid sequence or nucleotide sequence, refer to the percent of residues that are identical between respective positions of two sequences when the two sequences are aligned for maximum sequence identity. The % identity is calculated by dividing the total number of the aligned residues by the number of the residues that are identical between the respective positions of the at least two sequences and multiplying by 100. Generally, computer programs can be employed for such calculations. Illustrative programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12(1 Pt 1):387-95).

The terms, “% complementary”, “% complementarity”, “percent complementary”, “percent complementarity” and grammatical equivalents thereof, as used interchangeably herein, in the context of two or more nucleic acid molecules, refer to the percent of nucleotides in two nucleotide sequences in said nucleic acid molecules of equal length that can undergo cumulative base pairing at two or more individual corresponding positions in an antiparallel orientation. Accordingly, the terms include nucleic acid sequences that are not completely complementary over their entire length, which indicates that the two or more nucleic acid molecules include one or more mismatches. A “mismatch” is present at any position in the two opposed nucleotides that are not complementary. The % complementary is calculated by dividing the total number of the complementary residues by the total number of the nucleotides in one of the equal length sequences and multiplying by 100. Complete or total complementarity describes nucleotide sequences in 100% of the residues of a nucleotide sequence are complementary to residues in a reference nucleotide sequence. “Partially complementarity” describes nucleotide sequences in which at least 20%, but less than 100%, of the residues of a nucleotide sequence are complementary to residues in a reference nucleotide sequence. In some instances, at least 50%, but less than 100%, of the residues of a nucleotide sequence are complementary to residues in a reference nucleotide sequence. In some instances, at least 70%, 80%, 90% or 95%, but less than 100%, of the residues of a nucleotide sequence are complementary to residues in a reference nucleotide sequence. “Noncomplementary” describes nucleotide sequences in which less than 20% of the residues of a nucleotide sequence are complementary to residues in a reference nucleotide sequence.

The term, “percent similarity,” or “% similarity,” as used herein, in the context of an amino acid sequence, refers to a value that is calculated by dividing a similarity score by the length of the alignment. The similarity of two amino acid sequences can be calculated by using a BLOSUM62 similarity matrix (Henikoff and Henikoff, Proc. Natl. Acad. Sci. USA., 89:10915-10919 (1992)) that is transformed so that any value≥1 is replaced with +1 and any value≤0 is replaced with 0. For example, an Ile (I) to Leu (L) substitution is scored at +2.0 by the BLOSUM62 similarity matrix, which in the transformed matrix is scored at +1. This transformation allows the calculation of percent similarity, rather than a similarity score. Alternately, when comparing two full protein sequences, the proteins can be aligned using pairwise MUSCLE alignment. Then, the % similarity can be scored at each residue and divided by the length of the alignment. For determining % similarity over a protein domain or motif, a multilevel consensus sequence (or PROSITE motif sequence) can be used to identify how strongly each domain or motif is conserved. In calculating the similarity of a domain or motif, the second and third levels of the multilevel sequence are treated as equivalent to the top level. Additionally, if a substitution could be treated as conservative with any of the amino acids in that position of the multilevel consensus sequence, +1 point is assigned. For example, given the multilevel consensus sequence: RLG and YCK, the test sequence QIQ would receive three points. This is because in the transformed BLOSUM62 matrix, each combination is scored as: Q-R: +1; Q-Y: +0; I-L: +1; I-C: +0; Q-G: +0; Q-K: +1. For each position, the highest score is used when calculating similarity. The % similarity can also be calculated using commercially available programs, such as the Geneious Prime software given the parameters matrix=BLOSUM62 and threshold≥1.

The terms, “amplification,” “amplifying,” and grammatical equivalents thereof, as used herein, refer to a process by which a nucleic acid molecule is enzymatically copied to generate a plurality of nucleic acid molecules containing the same sequence as the original nucleic acid molecule or a distinguishable portion thereof.

The terms, “bind,” “binding,” “interact” and “interacting,” as used herein, refer to a non-covalent interaction between macromolecules (e.g., between two polypeptides, between a polypeptide and a nucleic acid; between a polypeptide/guide nucleic acid complex and a target nucleic acid; and the like). While in a state of noncovalent interaction, the macromolecules are said to be “associated” or “interacting” or “binding” (e.g., when a molecule X is said to interact with a molecule Y, it is meant the molecule X binds to molecule Y in a non-covalent manner). Non-limiting examples of non-covalent interactions are ionic bonds, hydrogen bonds, van der Waals and hydrophobic interactions. Not all components of a binding interaction need be sequence-specific (e.g., contacts with phosphate residues in a DNA backbone), but some portions of a binding interaction may be sequence-specific.

The term, “base editor,” as used herein, refers to a polypeptide or fusion protein comprising a base editing activity. The polypeptide with base editing activity may be referred to as an effector partner. The base editor can differ from a naturally occurring base editing enzyme. It is understood that any reference to a base editor herein also refers to a base editing enzyme variant. The base editor is functional when the effector protein is coupled to a guide nucleic acid. The guide nucleic acid imparts sequence specific activity to the base editor. By way of non-limiting example, the effector protein may comprise a catalytically inactive effector protein (e.g., a catalytically inactive variant of an effector protein described herein). Also, by way of non-limiting example, the base editing enzyme may comprise deaminase activity.

The term, “catalytically inactive effector protein,” as used herein, refers to an effector protein that is modified relative to a naturally-occurring effector protein to have a reduced or eliminated catalytic activity relative to that of the naturally-occurring effector protein, but retains its ability to interact with a guide nucleic acid. The catalytic activity that is reduced or eliminated is often a nuclease activity. The naturally-occurring effector protein may be a wildtype protein. In some instances, the catalytically inactive effector protein is referred to as a catalytically inactive variant of an effector protein.

The term, “cis cleavage,” as used herein, refers to cleavage (hydrolysis of a phosphodiester bond) of a target nucleic acid by a complex of an effector protein and a guide nucleic acid (e.g., an RNP complex), wherein at least a portion of the guide nucleic acid is hybridized to at least a portion of the target nucleic acid. Cleavage may occur within or directly adjacent to the portion of the target nucleic acid that is hybridized to the portion of the guide nucleic acid.

The term, “codon optimized,” as used herein, refers to a mutation of a nucleotide sequence encoding a polypeptide, such as a nucleotide sequence encoding an effector protein, to mimic the codon preferences of the intended host organism or cell while encoding the same polypeptide. Thus, the codons can be changed, but the encoded polypeptide remains unchanged. For example, if the intended target cell was a human cell, a human codon-optimized nucleotide sequence encoding an effector protein could be used. As another non-limiting example, if the intended host cell were a mouse cell, then a mouse codon-optimized nucleotide sequence encoding an effector protein could be generated. As another non-limiting example, if the intended host cell were a eukaryotic cell, then a eukaryote codon-optimized nucleotide sequence encoding an effector protein could be generated. As another non-limiting example, if the intended host cell were a prokaryotic cell, then a prokaryote codon-optimized nucleotide sequence encoding an effector protein could be generated. Codon usage tables are readily available, for example, at the “Codon Usage Database” available at www.kazusa.or.jp/codon.

The terms, “complementary” and “complementarity,” as used herein, in the context of a nucleic acid molecule or nucleotide sequence, refer to the characteristic of a polynucleotide having nucleotides that can undergo cumulative base pairing with their Watson-Crick counterparts (C with G; or A with T) in a reference nucleic acid in antiparallel orientation. For example, when every nucleotide in a polynucleotide or a specified portion thereof forms a base pair with every nucleotide in an equal length sequence of a reference nucleic acid, that polynucleotide is said to be 100% complementary to the nucleotide sequence of the reference nucleic acid. In a double stranded DNA or RNA sequence, the upper (sense) strand sequence is, in general, understood as going in the direction from its 5′- to 3′-end, and the complementary sequence is thus understood as the nucleotide sequence of the lower (antisense) strand in the same direction as the upper strand. Following the same logic, the reverse sequence is understood as the nucleotide sequence of the upper strand in the direction from its 3′- to its 5′-end, while the “reverse complement” sequence or the “reverse complementary” sequence is understood as the nucleotide sequence of the lower strand in the direction of its 5′- to its 3′-end. Each nucleotide in a double stranded DNA or RNA molecule that is paired with its Watson-Crick counterpart can be referred to as its complementary nucleotide. The complementarity of modified or artificial base pairs can be based on other types of hydrogen bonding and/or hydrophobicity of bases and/or shape complementarity between bases.

The term, “cleavage assay,” as used herein, refers to an assay designed to visualize, quantitate or identify cleavage of a nucleic acid. In some instances, the cleavage activity may be cis cleavage activity.

The terms, “cleave,” “cleaving” and “cleavage,” as used herein, in the context of a nucleic acid molecule or nuclease activity of an effector protein, refer to the hydrolysis of a phosphodiester bond of a nucleic acid molecule that results in breakage of that bond. The result of this breakage can be a nick (hydrolysis of a single phosphodiester bond on one side of a double-stranded molecule), single strand break (hydrolysis of a single phosphodiester bond on a single-stranded molecule) or double strand break (hydrolysis of two phosphodiester bonds on both sides of a double-stranded molecule) depending upon whether the nucleic acid molecule is single-stranded (e.g., ssDNA or ssRNA) or double-stranded (e.g., dsDNA) and the type of nuclease activity being catalyzed by the effector protein.

The term, “clustered regularly interspaced short palindromic repeats (CRISPR),” as used herein, refers to a segment of DNA found in the genomes of certain prokaryotic organisms, including some bacteria and archaea, that includes repeated short sequences of nucleotides interspersed at regular intervals between unique sequences of nucleotides derived from another organism.

The term, “conservative substitution,” as used herein, refers to the replacement of one amino acid for another such that the replacement takes place within a family of amino acids that are related in their side chains. Conversely, the term “non-conservative substitution” as used herein refers to the replacement of one amino acid residue for another that does not have a related side chain. Genetically encoded amino acids can be divided into four families having related side chains: (1) acidic (negatively charged): Asp (D), Glu (E); (2) basic (positively charged): Lys (K), Arg (R), His (H); (3) non-polar (hydrophobic): Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic: Ala (A), Val (V), Leu (L), Ile (I), Met (M), Phe (F); and (ii) moderately hydrophobic: Gly (G), Pro (P), Cys (C), Tyr (Y), Trp (W); and (4) uncharged polar: Asn (N), Gln (Q), Ser(S), Thr (T). Amino acids may be related by aliphatic side chains: Gly (G), Ala (A), Val (V), Leu (L), Ile (I), Ser(S), Thr (T), with Ser(S) and Thr (T) optionally being grouped separately as aliphatic-hydroxyl; Amino acids may be related by aromatic side chains: Phe (F), Tyr (Y), Trp (W). Amino acids may be related by amide side chains: Asn (N), Gln (Q). Amino acids may be related by sulfur-containing side chains: Cys (C) and Met (M).

The terms, “CRISPR RNA” and “crRNA,” as used herein, refer to a type of guide nucleic acid that is RNA comprising a first nucleotide sequence that is capable of hybridizing to a target sequence of a target nucleic acid and a second nucleotide sequence that is capable of interacting with an effector protein either directly (by being bound by an effector protein) or indirectly (e.g., by hybridization with a second nucleic acid molecule that can be bound by an effector). The first nucleotide sequence and the second nucleotide sequence are directly connected to each other or by a linker.

The term, “detectable product,” as used herein, refers to a unit produced after the cleavage of a reporter that is capable of being discovered, identified, perceived or noticed. A detectable product can comprise a detectable label and/or moiety that emits a detectable signal. A detectable product may include other components that are not capable of being readily discovered, identified, perceived or noticed at the same time as the detectable signal. For example, a detectable product may comprise remnants of the reporter. Accordingly, in some instances, the detectable product comprises RNA and/or DNA.

The term, “detectable signal,” as used herein, refers to an act, event, physical quantity or impulse that can be detected using optical, fluorescent, chemiluminescent, electrochemical or other detection methods known in the art.

The term, “diseased cell,” as used herein, refers to a cell comprising pathway conditions or pathway systems that are not conducive to cell survival, tissue survival, systemic survival, or organism survival.

The term, “edited target nucleic acid,” as used herein, refers to a target nucleic acid, wherein the target nucleic acid has undergone an editing, for example, after contact with an effector protein. In some instances, the editing is an alteration in the nucleotide sequence of the target nucleic acid. In some instances, the edited target nucleic acid comprises an insertion, deletion, or substitution of one or more nucleotides compared to the unedited target nucleic acid.

The term, “effector protein,” as used herein, refers to a protein, polypeptide, or peptide that is capable of interacting with a nucleic acid, such as a guide nucleic acid, to form a complex (e.g., a RNP complex), wherein the complex interacts with a target nucleic acid.

The term, “effector partner,” as used herein, refers to a protein, polypeptide or peptide that can, in combination with an effector protein, impart some function or activity that can be used to effectuate modification(s) of a target nucleic acid described herein and/or change expression of the target nucleic acid or other nucleic acids associated with the target nucleic acid, when used in connection with compositions, systems and methods described herein.

The term, “engineered modification,” as used herein, refers to a structural change of one or more nucleic acid residues of a nucleotide sequence or one or more amino acid residue of an amino acid sequence. The engineered modifications of a nucleotide sequence can include chemical modification of one or more nucleobases, or a chemical change to the phosphate backbone, a nucleotide, a nucleobase or a nucleoside. The engineered modifications can be made to an effector protein amino acid sequence or guide nucleic acid nucleotide sequence, or any sequence disclosed herein (e.g., a nucleic acid encoding an effector protein or a nucleic acid that encodes a guide nucleic acid). Methods of modifying a nucleic acid or amino acid sequence are known. One of ordinary skill in the art will appreciate that the engineered modification(s) may be located at any position(s) of a nucleic acid such that the function of the nucleic acid, protein, composition or system is not substantially decreased. Nucleic acids provided herein can be prepared according to any available technique including, but not limited to chemical synthesis, enzymatic synthesis, which is generally termed in vitro-transcription, cloning, enzymatic, or chemical cleavage, etc. In some instances, the nucleic acids provided herein are not uniformly modified along the entire length of the molecule. Different nucleotide modifications and/or backbone structures can exist at various positions within the nucleic acid.

The term, “functional domain,” as used herein, refers to a region of one or more amino acids in a protein that is required for an activity of the protein, or the full extent of that activity, as measured in an in vitro assay. Activities include, but are not limited to nucleic acid binding, nucleic acid editing, nucleic acid modifying, nucleic acid cleaving, protein binding. The absence of the functional domain, including mutations of the functional domain, would abolish or reduce activity.

The term, “functional fragment,” as used herein, refers to a fragment of a protein that retains some function relative to the entire protein. Non-limiting examples of functions are nucleic acid binding, nucleic acid editing, protein binding, nuclease activity, nickase activity, deaminase activity, demethylase activity, or acetylation activity. A functional fragment may be a recognized functional domain, e.g., a catalytic domain. In some instances, the catalytic domain comprises a RuvC domain.

The term, “functional protein,” as used herein, refers to protein that retains at least some if not all activity relative to the wildtype protein. A functional protein can also include a protein having enhanced activity relative to the wildtype protein. Assays are known and available for detecting and quantifying protein activity, e.g., colorimetric and fluorescent assays. In some instances, a functional protein is a wildtype protein. In some instances, a functional protein is a functional portion of a wildtype protein.

The term, “fused,” as used herein, refers to at least two sequences that are connected together, such as by a covalent bond (e.g., an amide bond or a phosphodiester bond) or by a linker. The covalent bond can be formed by conjugation (e.g., chemical conjugation or enzymatic conjugation) reaction.

The term, “fusion protein,” as used herein, refers to a protein comprising at least two polypeptides. The fusion protein may comprise one or more effector proteins and effector partners. In some instances, an effector protein and effector partner are not found connected to one another as a native protein or complex that occurs together in nature.

The term, “genetic disease,” as used herein, refers to a disease, disorder, condition, or syndrome associated with or caused by one or more mutations in the DNA of an organism having the genetic disease.

The term, “guide nucleic acid,” as used herein, refers to a nucleic acid that, when in a complex with one or more polypeptides described herein (e.g., an RNP complex) can impart sequence selectivity to the complex when the complex interacts with a target nucleic acid. A guide nucleic acid may be referred to interchangeably as a guide RNA, however it is understood that guide nucleic acids may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

The term, “heterologous,” as used herein, refers to at least two different polypeptide sequences that are not found similarly connected to one another in a native nucleic acid or protein. A protein that is heterologous to the effector protein is a protein that is not covalently linked by an amide bond to the effector protein in nature. In some instances, a protein is heterologous when the protein is not encoded by a species that encodes the effector protein. A guide nucleic acid may comprise “heterologous” sequences, which means that it includes a first nucleotide sequence and a second nucleotide sequence, wherein the first nucleotide sequence and the second nucleotide sequence are not found covalently linked by a phosphodiester bond in nature. Thus, the first nucleotide sequence is considered to be heterologous with the second nucleotide sequence, and the guide nucleic acid may be referred to as a heterologous guide nucleic acid. A heterologous system comprises at least one component that is not naturally occurring together with remaining components of the heterologous system.

The terms, “hybridize,” “hybridizable” and grammatical equivalents thereof, refer to a nucleotide sequence that is able to noncovalently interact, i.e. form Watson-Crick base pairs and/or G/U base pairs, or anneal, to another nucleotide sequence in a sequence-specific, antiparallel, manner (i.e., a nucleotide sequence specifically interacts to a complementary nucleotide sequence) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) for both DNA and RNA. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, a guanine (G) can be considered complementary to both an uracil (U) and to an adenine (A). Accordingly, when a G/U base-pair can be made at a given nucleotide position, the position is not considered to be non-complementary, but is instead considered to be complementary. While hybridization typically occurs between two nucleotide sequences that are complementary, mismatches between bases are possible. It is understood that two nucleotide sequences need not be 100% complementary to be specifically hybridizable, hybridizable, partially hybridizable, or for hybridization to occur. Moreover, a nucleotide sequence may hybridize over one or more segments such that intervening or adjacent segments are not involved in the hybridization event (e.g., a bulge, a loop structure or hairpin structure, etc.). The conditions appropriate for hybridization between two nucleotide sequences depend on the length of the nucleotide sequence and the degree of complementarity, variables which are well known in the art. For hybridizations between nucleic acids with short stretches of complementarity (e.g. complementarity over 35 or less, 30 or less, 25 or less, 22 or less, 20 or less, or 18 or less nucleotides) the position of mismatches may become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). Any suitable in vitro assay may be utilized to assess whether two sequences “hybridize”. One such assay is a melting point analysis where the greater the degree of complementarity between two nucleotide sequences, the greater the value of the melting temperature (Tm) for hybrids of nucleic acids having those sequences. The conditions of temperature and ionic strength determine the “stringency” of the hybridization. Temperature, wash solution salt concentration, and other conditions may be adjusted as necessary according to factors such as length of the region of complementation and the degree of complementation. Hybridization and washing conditions are well known and exemplified in Sambrook, J. and Russell, W., Molecular Cloning: A Laboratory Manual, Third Edition, Cold Spring Harbor Laboratory Press, Cold Spring Harbor (2001); and in Green, M. and Sambrook, J., Molecular Cloning: A Laboratory Manual, Fourth Edition, Cold Spring Harbor Laboratory Press, Cold Spring Harbor (2012).

The term, “indel,” as used herein, refers to an insertion-deletion or indel mutation, which is a type of genetic mutation that results from the insertion and/or deletion of one or more nucleotide in a target nucleic acid. An indel can vary in length (e.g., 1 to 1,000 nucleotides in length) and be detected by any suitable method, including sequencing.

The term, “indel percentage (% indel)” as used herein, refers to a percentage of sequencing reads that show at least one nucleotide has been edited from the insertion and/or deletion of nucleotides regardless of the size of insertion or deletion, or number of nucleotides edited. For example, if there is at least one nucleotide deletion detected in a given target nucleic acid, it counts towards the percent indel value. As another example, if one copy of the target nucleic acid has one nucleotide deleted, and another copy of the target nucleic acid has 10 nucleotides deleted, they are counted the same. This number reflects the percentage of target nucleic acids that are edited by a given effector protein. In some embodiments, % indel is represented as “Mod %.”

The term, “in vitro,” as used herein, refers to describing something outside an organism. An in vitro system, composition or method may take place in a container for holding laboratory reagents such that it is separated from the biological source from which a material in the container is obtained. In vitro assays can encompass cell-based assays in which living or dead cells are employed. In vitro assays can also encompass a cell-free assay in which no intact cells are employed. The term “in vivo” is used to describe an event that takes place within an organism. The term “ex vivo” is used to describe an event that takes place in a cell that has been obtained from an organism. An ex vivo assay is not performed on a subject. Rather, it is performed upon a sample separate from a subject.

The terms, “length” and “linked” as used herein, are used to characterize the number of nucleotides forming a polynucleotide or the number of amino acids forming a polypeptide, which may be expressed as “kilobases” (kb) or “base pairs” (bp) for a polynucleotide or “amino acids” (aa) for a polypeptide. Thus, a length of 1 kb refers to a length of 1000 linked nucleotides, and a length of 500 bp refers to a length of 500 linked nucleotides. Similarly, a polypeptide having a length of 500 linked amino acids may also be simply described as having a length of 500 amino acids.

The term, “linker,” as used herein, refers to a molecule that links a first polypeptide to a second polypeptide (e.g., by an amide bond) or a first nucleic acid to a second nucleic acid (e.g., by a phosphodiester bond).

The term, “mutation,” as used herein, refers to an alteration that changes an amino acid residue or a nucleotide as described herein. Such an alteration can include, for example, deletions, insertions and/or substitutions. The mutation can refer to a change in structure of an amino acid residue or nucleotide relative to the starting or reference residue or nucleotide. A mutation of an amino acid residue includes, for example, deletions, insertions and substituting one amino acid residue for a structurally different amino acid residue. Such substitutions can be a conservative substitution, a non-conservative substitution, a substitution to a specific sub-class of amino acids, or a combination thereof as described herein. A mutation of a nucleotide includes, for example, changing one naturally occurring base for a different naturally occurring base, such as changing an adenine to a thymine or a guanine to a cytosine or an adenine to a cytosine or a guanine to a thymine. A mutation of a nucleotide base may result in a structural and/or functional alteration of the encoding peptide, polypeptide or protein by changing the encoded amino acid residue of the peptide, polypeptide or protein. A mutation of a nucleotide base may not result in an alteration of the amino acid sequence or function of encoded peptide, polypeptide or protein, also known as a silent mutation. Methods of mutating an amino acid residue or a nucleotide are well known.

The terms, “mutation associated with a disease” and “mutation associated with a genetic disorder,” as used herein, refer to the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation.

The term, “nickase,” as used herein, refers to an enzyme that possess catalytic activity for single stranded nucleic acid cleavage of a double stranded nucleic acid.

The term, “nickase activity,” as used herein, refers to catalytic activity that results in single stranded nucleic acid cleavage of a double stranded nucleic acid.

The terms, “non-naturally occurring” and “engineered,” as used herein, refer to indicate involvement of the hand of man. The terms, when referring to a nucleic acid, nucleotide, protein, polypeptide, peptide or amino acid, refer to a molecule, such as but not limited to, a nucleic acid, nucleotide, protein, polypeptide, peptide or amino acid refers to a modification of that molecule (e.g., chemical modification, nucleotide sequence, or amino acid sequence) that is not present in the naturally molecule. The terms, when referring to a composition or system described herein, refer to a composition or system having at least one component that is not naturally associated with the other components of the composition or system. By way of a non-limiting example, a composition may include an effector protein and a guide nucleic acid that do not naturally occur together. Conversely, and as a non-limiting further clarifying example, an effector protein or guide nucleic acid that is “natural,” “naturally-occurring,” or “found in nature” includes an effector protein and a guide nucleic acid from a cell or organism that have not been genetically modified by the hand of man.

The term, “NUC lobe,” as used herein, refers to the nuclease lobe which typically houses the RuvC domains. The NUC lob is connected to the REC lobe by a bridge helix.

The terms, “nuclease” and “endonuclease” as used herein, refer to an enzyme which possesses catalytic activity for nucleic acid cleavage.

The term, “nuclease activity,” as used herein, refers to catalytic activity that results in nucleic acid cleavage (e.g., ribonuclease activity (ribonucleic acid cleavage), or deoxyribonuclease activity (deoxyribonucleic acid cleavage), etc.).

The term, “nucleic acid,” as used herein, refers to a polymer of nucleotides. A nucleic acid may comprise ribonucleotides, deoxyribonucleotides, combinations thereof and modified versions of the same. A nucleic acid may be single-stranded or double-stranded, unless specified. Non-limiting examples of nucleic acids are double stranded DNA (dsDNA), single stranded (ssDNA), messenger RNA, genomic DNA, cDNA, DNA-RNA hybrids, and a polymer comprising purine and pyrimidine bases or other natural, chemically or biochemically modified, non-natural, or derivatized nucleotide bases. Accordingly, nucleic acids as described herein may comprise one or more mutations, one or more engineered modifications, or both.

The term, “nucleic acid expression vector,” as used herein, refers to a plasmid that can be used to express a nucleic acid of interest.

The term, “nuclear localization signal (NLS),” as used herein, refers to an entity (e.g., peptide) that facilitates localization of a nucleic acid, protein, or small molecule to the nucleus, when present in a cell that contains a nuclear compartment.

The terms, “nucleotide(s)” and “nucleoside(s)”, as used herein, in the context of a nucleic acid molecule having multiple residues, refer to describing the sugar and base of the residue contained in the nucleic acid molecule. Similarly, a skilled artisan could understand that linked nucleotides and/or linked nucleosides, as used in the context of a nucleic acid having multiple linked residues, are interchangeable and describe linked sugars and bases of residues contained in a nucleic acid molecule. When referring to a “nucleobase(s)”, or linked nucleobase, as used in the context of a nucleic acid molecule, it can be understood as describing the base of the residue contained in the nucleic acid molecule, for example, the base of a nucleotide, nucleosides, or linked nucleotides or linked nucleosides. A person of ordinary skill in the art when referring to nucleotides, nucleosides and/or nucleobases would also understand the differences between RNA and DNA (generally the exchange of uridine for thymidine or vice versa) and the presence of nucleoside analogs, such as modified uridines, do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, NI-methyl pseudouridine, or 5-methoxyuridine, is considered 100% identical to AUG in that both are perfectly complementary to the same sequence (5′-CAU).

The term, “pharmaceutically acceptable excipient, carrier or diluent,” as used herein, refers to any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by suitable methods (see, e.g., Remington, The Science and Practice of Pharmacy 23^{rd} Ed. Academic Press, 2021).

The terms, “polypeptide” and “protein,” as used herein, refer to a polymeric form of amino acids. A polypeptide may include coded and non-coded amino acids, chemically or biochemically modified or derivatized amino acids, and polypeptides having modified peptide backbones. Accordingly, polypeptides as described herein may comprise one or more mutations, one or more engineered modifications, or both. It is understood that when describing coding sequences of polypeptides described herein, said coding sequences do not necessarily require a codon encoding an N-terminal Methionine (M) or a Valine (V) as described for the effector proteins described herein. One skilled in the art would understand that a start codon could be replaced or substituted with a start codon that encodes for an amino acid residue sufficient for initiating translation in a host cell. In some instances, when a heterologous peptide, such as an effector partner, protein tag or NLS, is located at the N terminus of the effector protein, a start codon for the heterologous peptide serves as a start codon for the effector protein as well. Thus, the natural start codon encoding an amino acid residue sufficient for initiating translation (e.g., Methionine (M) or a Valine (V)) of the effector protein may be removed or absent.

The term, “prime editing enzyme”, as used herein, refers to a protein, polypeptide, or fragment thereof that is capable of catalyzing the editing (insertion, deletion, or base-to-base conversion) of a target nucleotide or nucleotide sequence in a nucleic acid.

The terms, “promoter” and “promoter sequence,” as used herein, refer to a DNA regulatory region capable of binding RNA polymerase and initiating transcription of a downstream (3′ direction) coding or non-coding sequence. A transcription initiation site, as well as protein binding domains responsible for the binding of RNA polymerase, can also be found in a promoter region. Eukaryotic promoters will often, but not always, contain “TATA” boxes and “CAT” boxes. Various promoters, including inducible promoters, may be used to drive expression by the various vectors of the present disclosure.

The terms, “protospacer adjacent motif” and “PAM,” as used herein, refer to a nucleotide sequence found in a target nucleic acid that directs an effector protein to edit the target nucleic acid at a specific location. In some instances, a PAM is required for a complex of an effector protein and a guide nucleic acid (e.g., an RNP complex) to hybridize to and edit the target nucleic acid. In some instances, the complex does not require a PAM to edit the target nucleic acid.

The term, “REC domain,” as used herein, refers to domain in an α-helical recognition region or lobe. An effector protein may contain at least one REC domain (e.g., REC1, REC2) which generally helps to accommodate and stabilize the guide nucleic acid and target nucleic acid hybrid.

The term, “recombinant,” as used herein, in the context of proteins, polypeptides, peptides and nucleic acids, refers to proteins, polypeptides, peptides and nucleic acids that are products of various combinations of cloning, restriction and/or ligation steps resulting in a construct having a structural coding or non-coding sequence distinguishable from endogenous nucleic acids found in natural systems.

The term, “regulatory element,” used herein, refers to transcriptional and translational control sequences, such as promoters, enhancers, polyadenylation signals, terminators, protein degradation signals and the like, that provide for and/or regulate transcription of a non-coding sequence (e.g., a guide nucleic acid) or a coding sequence (e.g., effector proteins, fusion proteins and the like) and/or regulate translation of an encoded polypeptide.

The term, “repeat sequence,” as used herein, refers to a sequence of nucleotides in a guide nucleic acid that is capable of, at least partially, interacting with an effector protein.

The terms, “reporter,” “reporter nucleic acid,” and “reporter molecule,” as used herein, are used interchangeably and refer to a non-target nucleic acid molecule that can provide a detectable signal upon cleavage by an effector protein. Examples of detectable signals and detectable moieties that generate detectable signals are provided herein.

The terms, “ribonucleotide protein complex” and “RNP” as used herein, refer to a complex of one or more nucleic acids and one or more polypeptides described herein. While the term utilizes “ribonucleotides” it is understood that the one or more nucleic acid may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

The term, “R-Loop” as used herein, refers to a three-stranded nucleic acid structure comprising a DNA: RNA hybrid and a displaced strand of DNA. For example, an R-Loop can be formed upon hybridization of a guide nucleic acid as described herein to a target sequence of a target nucleic acid.

The terms, “RuvC” and “RuvC domain,” as used herein, refer to a region of an effector protein that is capable of cleaving a target nucleic acid and, in certain instances, of processing a pre-crRNA. In some instances, the RuvC domain is located near the C-terminus of the effector protein. A single RuvC domain may comprise RuvC subdomains, for example a RuvCI subdomain, a RuvCII subdomain and a RuvCIII subdomain. The term “RuvC” domain can also refer to a “RuvC-like” domain. Various RuvC-like domains are known in the art and are easily identified using online tools such as InterPro (https://www.ebi.ac.uk/interpro/). For example, a RuvC-like domain may be a domain which shares homology with a region of TnpB proteins of the IS605 and other related families of transposons.

The term, “sample,” as used herein, refers to something comprising a target nucleic acid. In some instances, the sample is a biological sample, such as a biological fluid or tissue sample. In some instances, the sample is an environmental sample. The sample may be a biological sample or environmental sample that is modified or manipulated. By way of non-limiting example, samples may be modified or manipulated with purification techniques, heat, nucleic acid amplification, salts and buffers.

The term, “single nucleic acid system,” as used herein, refers to a system that uses a guide nucleic acid complexed with one or more polypeptides described herein, wherein the complex is capable of interacting with a target nucleic acid in a sequence specific manner, and wherein the guide nucleic acid is capable of non-covalently interacting with the one or more polypeptides described herein, and wherein the guide nucleic acid is capable of hybridizing with a target sequence of the target nucleic acid. A single nucleic acid system lacks a duplex of a guide nucleic acid as hybridized to a second nucleic acid, wherein in such a duplex the second nucleic acid, and not the guide nucleic acid, is capable of interacting with the effector protein.

The term, “spacer sequence,” as used herein, refers to a nucleotide sequence in a guide nucleic acid that is capable of, at least partially, hybridizing to an equal length portion of a sequence (e.g., a target sequence) of a target nucleic acid.

The term, “subject,” as used herein, refers to an animal. The subject may be a mammal. The subject may be a human. The subject may be diagnosed or at risk for a disease.

The term, “sufficiently complementary,” as used herein, refers to a first nucleotide sequence that is partially complementarity to a second nucleotide sequence while still allowing the first nucleotide sequence to hybridize to the second nucleotide sequence with enough affinity to permit a biological activity to occur. Depending on the context, a biological activity may be the formation of a complex between two or more components described herein, such as an effector protein and a guide nucleic acid. A biological activity may also be bringing one or more components described herein into proximity of another component, such as bringing an effector protein-guide nucleic acid complex into proximity of a target nucleic acid. A biological activity may additionally be permitting a component described herein to act on another component described herein, such as permitting an effector protein to cleave a target nucleic acid. In some instances, sequences are said to be sufficiently complementary when at least 85% of the residues of a nucleotide sequence are complementary to residues in a reference nucleotide sequence.

The term, “syndrome,” as used herein, refers to a group of symptoms which, taken together, characterize a condition.

The term, “target nucleic acid,” as used herein, refers to a nucleic acid that is selected as the nucleic acid for editing, binding, hybridization or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA).

The term, “target sequence,” as used herein, in the context of a target nucleic acid, refers to a nucleotide sequence found within a target strand of a target nucleic acid. Such a nucleotide sequence can, for example, hybridize to a respective length portion of a guide nucleic acid.

The terms, “target strand” and “TS,” as used herein, in the context of a target nucleic acid being either a single stranded target nucleic acid or a double stranded target nucleic acid, refer to the nucleotide strand that comprises a target sequence to which at least a portion of a guide nucleic acid (e.g., a spacer sequence) is capable of, at least partially, hybridizing to an equal length portion of the target sequence. The terms, “non-target strand” and “NTS,” as used herein, in the context of a target nucleic acid being a double stranded target nucleic acid, refer to the nucleotide strand to which a guide nucleic acid is not capable of hybridizing to. The terms target strand and non-target strand differentiate between the strands of a double stranded target nucleic acid to which a guide nucleic acid is capable of or not capable of hybridizing. Reference may be made to a target sequence present in the target strand or the non-target strand of a double stranded target nucleic acid.

The term, “transcriptional activator,” as used herein, refers to a polypeptide or a fragment thereof that can activate or increase transcription of a target nucleic acid molecule.

The term, “transcriptional repressor,” as used herein, refers to a polypeptide or a fragment thereof that is capable of arresting, preventing, or reducing transcription of a target nucleic acid.

The term, “transgene,” as used herein, refers to a nucleotide sequence that is inserted into a cell for expression of said nucleotide sequence in the cell. A transgene is meant to include (1) a nucleotide sequence that is not naturally found in the cell (e.g., a heterologous nucleotide sequence); (2) a nucleotide sequence that is a mutant form of a nucleotide sequence naturally found in the cell into which it has been introduced; (3) a nucleotide sequence that serves to add additional copies of the same (e.g., exogenous or homologous) or a similar nucleotide sequence naturally occurring in the cell into which it has been introduced; or (4) a silent naturally occurring or homologous nucleotide sequence whose expression is induced in the cell into which it has been introduced. The cell in which transgene expression occurs can be a target cell, such as a host cell.

The terms, “treatment” and “treating,” as used herein, refer to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term, “variant,” as used herein, refers to a form or version of a protein that differs from the wild-type protein. A variant may have a different function or activity relative to the wild-type protein.

The term, “viral vector,” as used herein, refers to a nucleic acid to be delivered into a host cell by a recombinantly produced virus or viral particle.

### Introduction

Disclosed herein are compositions, systems and methods comprising at least one of: (1) a polypeptide or a nucleic acid encoding the polypeptide; and (2) a guide nucleic acid or a nucleic acid encoding the guide nucleic acid.

Polypeptides described herein may bind and cleave (e.g., nick) nucleic acids in a sequence-specific manner. Polypeptides described herein may also cleave (e.g., nick) the target nucleic acid within a target sequence or at a position adjacent to the target sequence. A polypeptide may be an effector protein, such as a CRISPR-associated (Cas) protein, which may bind a guide nucleic acid that imparts activity or sequence selectivity to the polypeptide. An effector protein may also be referred to as a programmable nuclease because the nickase activity of the protein may be directed to different target nucleic acids by way of revising the guide nucleic acid that the protein binds.

In some embodiments, compositions, systems and methods comprising guide nucleic acids comprise a first region or sequence, at least a portion of which interacts with a polypeptide. In some embodiments, the first sequence comprises a sequence that is similar or identical to a repeat sequence. In some embodiments, compositions, systems and methods comprising guide nucleic acids comprise a second sequence that is at least partially complementary to a target nucleic acid, and which may be referred to as a spacer sequence.

Effector proteins disclosed herein may bind and cleave (e.g., nick) nucleic acids, including double stranded RNA (dsRNA), single-stranded RNA (ssRNA), double stranded DNA (dsDNA) and single-stranded DNA (ssDNA). Polypeptides disclosed herein may provide cis cleavage activity, binding activity, nickase activity, or a combination thereof.

The compositions, systems and methods described herein are non-naturally occurring. In some embodiments, compositions, systems and methods comprise an engineered guide nucleic acid (also referred to herein as a guide nucleic acid) or a use thereof. In some embodiments, compositions, systems and methods comprise an engineered protein or a use thereof. In some embodiments, compositions, systems and methods comprise an isolated polypeptide or a use thereof. In general, compositions, methods and systems described herein are not found in nature. In some embodiments, compositions, methods and systems described herein comprise at least one non-naturally occurring component. For example, disclosed compositions, methods and systems comprise a guide nucleic acid, wherein the nucleotide sequence of the guide nucleic acid is different or modified from that of a naturally-occurring guide nucleic acid.

In some embodiments, compositions, systems and methods comprise at least two components that do not naturally occur together. For example, disclosed compositions, systems and methods comprise a guide nucleic acid comprising a first region, at least a portion of which, interacts with a polypeptide, and a second region that is at least partially complementary to a target sequence in a target nucleic acid, wherein the first region and second region do not naturally occur together and/or are heterologous to each other. Also, by way of non-limiting example, disclosed compositions, systems and methods comprise a guide nucleic acid and an effector protein that do not naturally occur together. Likewise, by way of non-limiting example, disclosed compositions, systems and methods comprise a ribonucleotide-protein (RNP) complex comprising an effector protein and a guide nucleic acid that do not occur together in nature. Conversely and for clarity, an effector protein or guide nucleic acid that is “natural,” “naturally-occurring,” or “found in nature” includes effector proteins and guide nucleic acids from cells or organisms that have not been genetically modified by a human or machine.

In some embodiments, the guide nucleic acid comprises a non-natural nucleotide sequence. In some embodiments, the non-natural nucleotide sequence is a nucleotide sequence that is not found in nature. The non-natural nucleotide sequence may comprise a portion of a naturally-occurring nucleotide sequence, wherein the portion of the naturally-occurring nucleotide sequence is not present in nature absent the remainder of the naturally-occurring nucleotide sequence. In some embodiments, the guide nucleic acid comprises two naturally-occurring nucleotide sequences arranged in an order or proximity that is not observed in nature. In some embodiments, compositions and systems comprise a ribonucleotide complex comprising an effector protein and a guide nucleic acid that do not occur together in nature. In some embodiments, compositions and systems comprise at least two components that do not occur together in nature, wherein the at least two components comprise at least one of an effector protein, an effector partner and a guide nucleic acid. Guide nucleic acids may comprise a first nucleotide sequence and a second nucleotide sequence that do not occur naturally together. For example, a guide nucleic acid comprises a naturally-occurring repeat sequence and a spacer sequence that is complementary to a naturally-occurring eukaryotic nucleotide sequence. The guide nucleic acid may comprise a repeat sequence that occurs naturally in an organism and a spacer sequence that does not occur naturally in that organism. A guide nucleic acid may comprise a first nucleotide sequence that occurs in a first organism and a second nucleotide sequence that occurs in a second organism, wherein the first organism and the second organism are different. The guide nucleic acid may comprise a third nucleotide sequence disposed at a 3′ or 5′ end of the guide nucleic acid, or between the first and second nucleotide sequences of the guide nucleic acid. In some embodiments, the guide nucleic acid comprises two heterologous nucleotide sequences arranged in an order or proximity that is not observed in nature. Therefore, compositions and systems described herein are not naturally occurring.

In some embodiments, compositions, systems and methods described herein comprise a polypeptide (e.g., an effector protein, an effector partner, a fusion protein, or a combination thereof) that is similar to a naturally occurring polypeptide. The polypeptide may lack a portion of the naturally occurring polypeptide. The polypeptide may comprise a mutation relative to the naturally-occurring polypeptide, wherein the mutation is not found in nature. The polypeptide may also comprise at least one additional amino acid relative to the naturally-occurring polypeptide. In some embodiments, the polypeptide comprises a heterologous peptide. For example, the polypeptide comprises an addition of a nuclear localization signal relative to the natural occurring polypeptide. In some embodiments, a nucleotide sequence encoding the polypeptide is codon optimized (e.g., for expression in a eukaryotic cell) relative to the naturally occurring sequence.

### I. Polypeptide Systems

Provided herein are compositions, systems and methods comprising a polypeptide, a nucleic acid encoding the polypeptide or polypeptide system, wherein the polypeptide, the nucleic acid encoding the polypeptide or polypeptide system described herein comprises one or more effector proteins or variants thereof, one or more effector partners or variants thereof, one or more linkers for peptides, or combinations thereof.

In some embodiments, the polypeptides described herein comprise modification activities. In some embodiments, the modification activity of the polypeptide described herein is cleavage activity for a single stranded nucleic acid, nickase activity for a double stranded nucleic acid, binding activity, insertion activity, substitution activity, chemical modification activity and the like. In some embodiments, the modification activity of the polypeptide results in: cleavage of at least one strand of a target nucleic acid, deletion of one or more nucleotides of a target nucleic acid, insertion of one or more nucleotides into a target nucleic acid, substitution of one or more nucleotides of a target nucleic acid with an alternative nucleotide, chemical modification of one or more nucleotides of a target nucleic acid to an alternative nucleotide, or combinations thereof. In some embodiments, the cleavage activity is a nicking activity.

### Effector Proteins

Provided herein are compositions, systems and methods comprising an effector protein or a use thereof, wherein the effector protein modifies a target nucleic acid, wherein the target nucleic acid is a single stranded target nucleic acid or a double stranded target nucleic acid, and wherein a modification of the target nucleic acid comprises cleaving of the single stranded target nucleic acid or nicking of the double stranded target nucleic acid. In some embodiments, the effector protein is not capable of cleaving both strands of the double stranded target nucleic acid. In some embodiments, such an effector protein is referred to as a nickase.

An effector protein provided herein interacts with a guide nucleic acid to form a complex. In some embodiments, the complex interacts with a target nucleic acid, a non-target nucleic acid, or both. In some embodiments, an interaction between the complex and a target nucleic acid, a non-target nucleic acid, or both comprises one or more of: recognition of a protospacer adjacent motif (PAM) sequence within the target nucleic acid by the effector protein, hybridization of the guide nucleic acid to the target nucleic acid, modification of the target nucleic acid and/or the non-target nucleic acid by the effector protein, or combinations thereof. In some embodiments, recognition of a PAM sequence within a target nucleic acid directs the modification activity of an effector protein. In some embodiments, recognition of a PAM sequence adjacent to a target sequence of a target nucleic acid directs the modification activity of an effector protein.

In some embodiments, effector proteins disclosed herein provides cleavage activity (e.g., nickase activity), such as cis cleavage activity. In some embodiments, effector proteins described herein edit a target nucleic acid, wherein the target nucleic acid comprises a target strand and a non-target strand. In some embodiments, the effector proteins edit the target nucleic acid by cis cleavage activity on the target strand. Effector proteins disclosed herein may nick double stranded RNA (dsRNA) and/or double stranded DNA (dsDNA). Effector proteins disclosed herein may cleave single stranded RNA (ssRNA) and/or single-stranded DNA (ssDNA). In some embodiments, effector proteins disclosed herein provides catalytic activity (e.g., cleavage activity for a single stranded nucleic acid or nickase activity for a double stranded nucleic acid). In some embodiments, the catalytic activity of the effector protein is similar to that of a naturally-occurring effector protein, such as, for example, a naturally-occurring effector protein with reduced cleavage activity including cis cleavage activity.

In some embodiments, effector proteins described herein comprise one or more functional domains. Effector protein functional domains can include a protospacer adjacent motif (PAM)-interacting domain, an oligonucleotide-interacting domain, one or more recognition domains, a non-target strand interacting domain and a RuvC domain. A PAM interacting domain can be a target strand PAM interacting domain (TPID) or a non-target strand PAM interacting domain (NTPID). In some embodiments, a PAM interacting domain, such as a TPID or a NTPID, on an effector protein describes a region of an effector protein that interacts with target nucleic acid. In some embodiments, the effector proteins comprise a RuvC domain. In some embodiments, a RuvC domain comprises substrate binding activity, catalytic activity, or both. In some embodiments, the RuvC domain is defined by a single, contiguous sequence, or a set of RuvC subdomains that are not contiguous with respect to the primary amino acid sequence of the protein. An effector protein of the present disclosure includes multiple RuvC subdomains, which may combine to generate a RuvC domain with substrate binding or catalytic activity. For example, an effector protein includes three RuvC subdomains (RuvC-I, RuvC-II and RuvC-III) that are not contiguous with respect to the primary amino acid sequence of the effector protein, but form a RuvC domain once the protein is produced and folds. In some embodiments, the RuvC domain described herein comprises variants thereof (e.g., one or more mutations including substitutions, additions, deletions (e.g., truncation), or combinations thereof. In some embodiments, effector proteins comprise one or more recognition domain (REC domain) with a binding affinity for a guide nucleic acid or for a guide nucleic acid-target nucleic acid heteroduplex. An effector protein may comprise a zinc finger domain. In some embodiments, the effector protein does not comprise an HNH domain.

An effector protein may be a CRISPR-associated (“Cas”) protein. An effector protein may be a modified effector protein having increased modification activity and/or increased substrate binding activity (e.g., substrate selectivity, specificity and/or affinity). In some embodiments, the substrate can be a double-stranded RNA (dsRNA), single stranded RNA (ssRNA), double stranded DNA (dsDNA), or single-stranded DNA (ssDNA). An effector protein may function as a single protein, including a single protein that binds to a guide nucleic acid and editing a target nucleic acid. Alternatively, an effector protein may function as part of a multiprotein complex.

TABLE 1 provides illustrative amino acid sequences of an effector protein that is modified for use in the compositions, systems and methods described herein. In general, the effector protein described herein does not comprise an amino acid sequence that is identical to any one of the amino acid sequences recited in TABLE 1.

In some embodiments, compositions, systems and methods described herein comprise an effector protein, or a nucleic acid encoding the effector protein, wherein the amino acid sequence of the effector protein comprises at least 200 contiguous amino acids or more of the amino acid sequence recited in TABLE 1, wherein the amino acid sequence of the effector protein is not identical to any one of the amino acid sequences recited in TABLE 1. In some embodiments, the amino acid sequence of an effector protein provided herein comprises at least 200, at least 220, at least 240, at least 260, at least 280, at least 300, at least 320, at least 340, at least 360, at least 380, at least 400 contiguous amino acids, at least 420 contiguous amino acids, at least 440 contiguous amino acids, at least 460 contiguous amino acids, at least 480 contiguous amino acids, at least 500 contiguous amino acids, at least 520 contiguous amino acids, at least 540 contiguous amino acids, at least 560 contiguous amino acids, at least 580 contiguous amino acids, at least 600 contiguous amino acids, at least 620 contiguous amino acids, at least 640 contiguous amino acids, at least 660 contiguous amino acids, at least 680 contiguous amino acids, at least 700 contiguous amino acids, at least 720 contiguous amino acids, at least 760 contiguous amino acids, or more of the amino acid sequence of TABLE 1, wherein the amino acid sequence of the effector protein is not identical to any one of the amino acid sequences recited in TABLE 1.

In some embodiments, compositions, systems and methods described herein comprise an effector protein or a nucleic acid encoding the effector protein, wherein the effector protein comprises a portion of the amino acid sequence recited in TABLE 1. In some embodiments, the effector protein comprises a portion of the amino acid sequence recited in TABLE 1, wherein the portion does not comprise at least the first 10 amino acids, at least the first 20 amino acids, at least the first 40 amino acids, at least the first 60 amino acids, at least the first 80 amino acids, at least the first 100 amino acids, at least the first 120 amino acids, at least the first 140 amino acids, at least the first 160 amino acids, at least the first 180 amino acids, or at least the first 200 amino acids of the amino acid sequence recited in TABLE 1. In some embodiments, the effector protein comprises a portion of the amino acid sequence recited in TABLE 1, wherein the portion does not comprise the last 10 amino acids, the last 20 amino acids, the last 40 amino acids, the last 60 amino acids, the last 80 amino acids, the last 100 amino acids, the last 120 amino acids, the last 140 amino acids, the last 160 amino acids, the last 180 amino acids, or the last 200 amino acids of the amino acid sequence recited in TABLE 1.

In some embodiments, compositions, systems and methods described herein comprise an effector protein, or a nucleic acid encoding the effector protein, wherein the effector protein comprises an amino acid sequence that is at least 65%, at least 70%, at least 75%, at least 80%, at least 85%, at least 90%, at least 95%, at least 97%, at least 98%, at least 99%, but less than 100% identical to the amino acid sequence as recited in TABLE 1. In some embodiments, an effector protein provided herein comprises an amino acid sequence that is at least 65% but less than 100% identical to the amino acid sequence as recited in TABLE 1. In some embodiments, an effector protein provided herein comprises an amino acid sequence that is at least 70% but less than 100% identical to the amino acid sequence as recited in TABLE 1. In some embodiments, an effector protein provided herein comprises an amino acid sequence that is at least 75% but less than 100% identical to the amino acid sequence as recited in TABLE 1. In some embodiments, an effector protein provided herein comprises an amino acid sequence that is at least 80% but less than 100% identical to the amino acid sequence as recited in TABLE 1. In some embodiments, an effector protein provided herein comprises an amino acid sequence that is at least 85% but less than 100% identical to the amino acid sequence as recited in TABLE 1. In some embodiments, an effector protein provided herein comprises an amino acid sequence that is at least 90% but less than 100% identical to the amino acid sequence as recited in TABLE 1. In some embodiments, an effector protein provided herein comprises an amino acid sequence that is at least 95% but less than 100% identical to the amino acid sequence as recited in TABLE 1. In some embodiments, an effector protein provided herein comprises an amino acid sequence that is at least 97% but less than 100% identical to the amino acid sequence as recited in TABLE 1. In some embodiments, an effector protein provided herein comprises an amino acid sequence that is at least 98% but less than 100% identical to the amino acid sequence as recited in TABLE 1. In some embodiments, an effector protein provided herein comprises an amino acid sequence that is at least 99% but less than 100% identical to the amino acid sequence as recited in TABLE 1. In some embodiments, an effector protein provided herein comprises an amino acid sequence that is less than 100% identical to the amino acid sequence as recited in TABLE 1.

In some embodiments, compositions, systems and methods described herein comprise an effector protein, or a nucleic acid encoding the effector protein, wherein the effector protein comprises an amino acid sequence that is at least 80%, at least 85%, at least 90%, at least 95%, at least 97%, at least 98%, at least 99%, or 100% similar, but not the same, to the amino acid sequence as recited in TABLE 1. In some embodiments, an effector protein provided herein comprises an amino acid sequence that is at least 80% similar, but not the same, to the amino acid sequence as recited in TABLE 1. In some embodiments, an effector protein provided herein comprises an amino acid sequence that is at least 85% similar, but not the same, to the amino acid sequence as recited in TABLE 1. In some embodiments, an effector protein provided herein comprises an amino acid sequence that is at least 90% similar, but not the same, to the amino acid sequence as recited in TABLE 1. In some embodiments, an effector protein provided herein comprises an amino acid sequence that is at least 95% similar, but not the same, to the amino acid sequence as recited in TABLE 1. In some embodiments, an effector protein provided herein comprises an amino acid sequence that is at least 97% similar, but not the same, to the amino acid sequence as recited in TABLE 1. In some embodiments, an effector protein provided herein comprises an amino acid sequence that is at least 98% similar, but not the same, to the amino acid sequence as recited in TABLE 1. In some embodiments, an effector protein provided herein comprises an amino acid sequence that is at least 99% similar, but not the same, to the amino acid sequence as recited in TABLE 1. In some embodiments, an effector protein provided herein comprises an amino acid sequence that is 100% similar, but not the same, to the amino acid sequence as recited in TABLE 1.

In some embodiments, compositions, systems and methods described herein comprise an effector protein, or a nucleic acid encoding the effector protein, wherein the effector protein comprises one or more amino acid alterations relative to the amino acid sequence recited in TABLE 1. In some embodiments, the one or more alterations comprises at least one, at least two, at least three, at least four, at least five, at least six, at least seven, at least eight, at least nine, at least ten, at least twelve, at least sixteen, at least twenty, or more amino acid alterations relative to the amino acid sequence recited in TABLE 1. In some embodiments, the one or more alterations comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, sixteen to twenty, or more amino acid alterations relative to the amino acid sequence recited in TABLE 1. In some embodiments, the one or more alterations comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 or more amino acid alterations relative to the amino acid sequence recited in TABLE 1. In some embodiments, the one or more alterations comprises one, two, three, four, five, six, seven, eight, nine, ten, or more amino acid alterations relative to the amino acid sequence recited in TABLE 1. In some embodiments, the one or more amino acid alterations comprises substitutions (e.g., conservative substitutions, non-conservative substitutions), deletions, or combinations thereof. In some embodiments, an effector protein or a nucleic acid encoding the effector protein comprises 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations or more relative to the amino acid sequence recited in TABLE 1. In some embodiments, the effector protein comprises one or more alterations independently selected from positions D369, E567 and D658 relative to the amino acid sequence recited in SEQ ID NO: 1. In some embodiments, the effector protein comprises one or more alterations independently selected at positions 13, 14, 15, 16, 17, 18, 19, 20, 21, 22, 23, 24, 25, 26, 28, 29, 30, 31, 32, 33, 34, 35, 36, 37, 38, 39, 40, 41, 42, 43, 44, 51, 52, 53, 54, 55, 56, 57, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 157, 164, 166, 170, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 316, 489, 490, 491, 495, 496, 498, 500, 501, 502, 504, 505, 506, 511, 512, 513, 514, 515, 516, 517, 540, 541, 542, 543, 544, 545, 546, 590, 591, 592, 593, 594, 595, 596, 602, 603, 604, 605, 606, 607 and 608 relative to the amino acid sequence of SEQ ID NO: 1. In some embodiments, the effector protein comprises one or more alterations independently selected at positions 26, 157, 164, 166, 170, 489, 490, 491, 495, 496, 498, 500, 501, 502, 504, 505 and 506 relative to the amino acid sequence of SEQ ID NO: 1. In some embodiments, the effector protein comprises one or more alterations independently selected at positions 26, 38, 108, 109, 114, 182, 183, 184, 198 and 208 relative to the amino acid sequence of SEQ ID NO: 1. In some embodiments, the effector protein comprises one or more alterations independently selected at positions 369, 567 and 658 relative to the amino acid sequence recited in SEQ ID NO: 1.

In some embodiments, compositions, systems and methods described herein comprise an effector protein, or a nucleic acid encoding the effector protein, wherein the effector protein comprises one or more substitutions relative to the amino acid sequence recited in TABLE 1. In some embodiments, the one or more substitutions comprises at least one, at least two, at least three, at least four, at least five, at least six, at least seven, at least eight, at least nine, at least ten, at least twelve, at least sixteen, at least twenty, or more substitutions relative to the amino acid sequence recited in TABLE 1. In some embodiments, the one or more substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, sixteen to twenty, or more substitutions relative to the amino acid sequence recited in TABLE 1. In some embodiments, the one or more substitutions comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 or more amino acid substitutions relative to the amino acid sequence recited in TABLE 1. In some embodiments, the one or more amino acid substitutions comprise one, two, three, four, five, six, seven, eight, nine, ten or more substitutions relative to the amino acid sequence recited in TABLE 1. In some embodiments, the one or more amino acid substitutions comprise one or more substitutions with a positively charged amino acid residues. In some embodiments, the positively charged amino acid residue is independently selected from Lys (K), Arg (R), or His (H). In some embodiments, the one or more substitutions comprise one or more conservative substitutions, one or more non-conservative substitutions, or combinations thereof.

TABLE 2 recites exemplary amino acid substitutions for effector protein having an amino acid sequence of SEQ ID NO: 1. In some embodiments, the effector protein described herein comprises one or more substitutions independently selected from substitutions at positions T11, S21, G25, L26, Q54R, G55, G56, 159, K65, E68, Q76, S77, S78, L79, T87, P89, K92, E100, E109, H110, P116, E119, N129, N147, L149, E157, E164, E166, E170, S186, P187, K189, H208, N209, Y220, S223, V228, S229, Y231, I240, E258, R261, C279, D283, C285, R294, K299, N340, K347, K364, A366, T367, G371, D403, C405, N406, K435Q, N449, I471, K480, I489, Y490, F491, D495, K496, K498, K500, D501, V502, M503, K504, S505, D506, K508, K516, V521, S526, W530, R531, D549, N568, G577, N601, R617, L620, P622, A623, R625, T629, K634, S638, 1653, T668, D703, D704, and A706 relative to the amino acid sequence of SEQ ID NO: 1. In some embodiments, the effector protein comprises one or more substitutions independently selected from T11R, S21W, S21F, S21Y, G25I, G25L, G25F, G25W, G25V, G25Y, L26R, Q54R, G55P, G56P, I59K, K65L, E68P, Q76R, S77V, S78F, S78M, S78I, L79R, T87G, P89T, K92E, E100K, E109K, H110T, P116G, E119S, N129I, N147K, L149R, E157A, E157R, E164A, E164L, E166A, E166I, E170A, S186G, P187K, K189P, H208R, N209F, N209Y, Y220S, S223P, V228R, V228K, S229L, Y231K, I240K, E258K, R261W, R261M, R261L, C279W, C279F, C279I, C279Y, D283L, C285I, C285V, R294L, K299W, N340L, N340M, K347A, K364I, A366V, T367I, T367V, G371F, G371Y, D403W, C405L, N406K, K435Q, N449W, I471T, K480L, 1489A, 1489S, Y490S, Y490A, F491A, F491S, F491G, D495G, D495R, D495K, K496A, K496S, K498A, K498S, K500A, K500S, D501R, D501G, D501K, V502A, V502S, M503K, K504A, K504S, S505R, D506A, K508R, K516R, V521T, S526N, W530K, W530R, R531E, D549W, D549I, D549Y, D549L, N568D, G577H, N601Y, N601F, R617W, R617Y, L620E, P622N, A623P, R625F, R625W, R625Y, T629V, K634G, S638K, I653A, T668W, D703G, D704G, and A706G relative to the amino acid sequence of SEQ ID NO: 1. In some embodiments, the effector protein described herein comprises one or more substitutions independently selected from substitutions at positions L26, E157, E164, E166, E170, 1489, Y490, F491, D495, K496, K498, K500, K501, V502, K504, S505 and D506 relative to the amino acid sequence of SEQ ID NO: 1. In some embodiments, the effector protein comprises one or more substitutions independently selected from L26R, E157A, E164A, E166A, E170A, 1489A, 1489S, Y490S, Y490A, F491A, F491S, F491G, D495G, D495R, D495K, K496A, K496S, K498A, K498S, K500A, K500S, D501R, D501G, D501K, V502A, V502S, K504A, K504S, S505R and D506A relative to the amino acid sequence of SEQ ID NO: 1.

In some embodiments, the effector protein described herein comprises one or more substitutions independently selected from substitutions at positions S21, L26, Q76, T87, A121, N147, L149, S186, H208, Y220, S223, Y251, E258, C279, C405, 1471, M503, D523, S526, D549, Q552, S638, and D703 relative to the amino acid sequence of SEQ ID NO: 1. In some embodiments, the effector protein comprises one or more substitutions independently selected from S21L, L26R, Q76R, T87G, A121Q, N147K, L149R, S186G, H208R, Y220S, S223P, Y251R, E258K, C279R, C405L, I471T, M503K, D523K, S526N, D549L, Q552R, S638K, and D703G relative to the amino acid sequence of SEQ ID NO: 1.

Also, TABLE 2 recites exemplary combinations of amino acid substitutions for effector protein having an amino acid sequence of SEQ ID NO: 1. In some embodiments, an effector protein or nucleic acids encoding the effector protein, wherein the effector protein is at least 70%, at least 75%, at least 80%, at least 85%, at least 90%, at least 95%, at least 98% or at least 99% identical to SEQ ID NO: 1, and wherein the effector protein comprises any combination of amino acid substitutions described in TABLE 2. In some embodiments, an effector protein or nucleic acids encoding the effector protein, wherein the effector protein comprises any combination of amino acid substitutions described in TABLE 2, and wherein the effector protein comprises an amino acid sequence, other than the amino acid substitutions described in TABLE 2, that is at least 70%, at least 75%, at least 80%, at least 85%, at least 90%, at least 95%, at least 98% or at least 99% identical to SEQ ID NO: 1. In some embodiments, the combination of amino acid substitutions is selected from: (a) L26R, I471T, S223P and D703G; (b) L26R, I471T, S223P, D703G and H208R; (c) L26R, I471T, S223P, D703G and L149R; (d) L26R, I471T, S223P, D703G, L149R and H208R; (e) L26R, I471T, S223P, D703G, D704G and A706G; (f) L26R, I471T, S223P, D703G, L149R, H208R, D704G and A706G; (g) I471T, S223P and D703G; (h) I471T, S223P, D703G and H208R; (i) I471T, S223P, D703G and L149R; (j) I471T, S223P, D703G, L149R and H208R; (k) I471T, S223P, D703G, D704G and A706G; (1) I471T, S223P, D703G, L149R, H208R, D704G and A706G;
(m) I471T and E157R; (n) I471T, E157R, S223P and D703G; (o) L26R, I471T, E157R, S223P and D703G (p) L26R, T87G, S186G, H208R, S223P, C405L, I471T, S526N and D703G; (q) L26R, A121Q, S223P, E258K, I471T, D523K, S526N and D703G; (r) L26R, N147K, H208R, S223P, E258K, I471T, M503K and D703G; (s) L26R, N147K, S186G, S223P, E258K, I471T, S526N, D549L, S638K and D703G; (t) S21L, L26R, S186G, Y220S, S223P, I471T and D703G; (u) L26R, T87G, A121Q, S186G, H208R, Y220S, S223P, C405L, I471T, D523K and D703G; (v) S21L, L26R, A121Q, N147K, S186G, Y220S, S223P, I471T, S526N, D549L and D703G; (w) S21L, L26R, Q76R, N147K, L149R, Y220S, S223P, Y251R, E258K, I471T, M503K, Q552R and D703G; (x) L26R, A121Q, Y220S, S223P, C405L, I471T, D523K, Q552R and D703G; (y) S21L, L26R, A121Q, N147K, Y220S, S223P, Y251R, C405L, I471T and D703G; (z) L26R, Q76R, T87G, S223P, E258K, C279R, I471T, M503K, D523K and D703G; (aa) L26R, N147K, S186G, S223P, I471T, M503K, S526N and D703G; (bb) S21L, L26R, T87G, N147K, H208R, Y220S, S223P, I471T and D703G; (cc) S21L, L26R, A121Q, N147K, S186G, S223P, E258K, I471T, D523K, Q552R and D703G; (dd) L26R, A121Q, L149R, S186G, Y220S, S223P, I471T and D703G; (ee) L26R, A121Q, N147K, Y220S, S223P, I471T, M503K, S526N, D549L and D703G; (ff) L26R, T87G, A121Q, Y220S, S223P, E258K, C405L, I471T and D703G; (gg) L26R, T87G, S186G, Y220S, S223P, I471T, M503K and D703G; (hh) S21L, L26R, Q76R, T87G, N147K, S186G, S223P, I471T, S526N, S638K and D703G; (ii) S21L, L26R, A121Q, Y220S, S223P, C405L, I471T, M503K and D703G; (jj) L26R, S223P, I471T and D703G; (kk) L26R, T87G, S223P, I471T, S526N and D703G; (11) L26R, T87G, N147K, S223P, I471T, S526N and D703G; (mm) L26R, T87G, N147K, S223P, E258K, I471T, S526N and D703G; (nn) L26R, T87G, Y220S, S223P, I471T, S526N and D703G; (oo) L26R, T87G, N147K, Y220S, S223P, E258K, I471T, S526N and D703G; (pp) L26R, Q76R, T87G, N147K, Y220S, S223P, E258K, C279R, I471T, M503K, D523K and D703G; (qq) L26R, Q76R, T87G, N147K, Y220S, S223P, E258K, I471T, M503K, D523K and D703G; (rr) L26R, Q76R, T87G, N147K, Y220S, S223P, E258K, I471T, M503K and D703G; (ss) S21L, L26R, Q76R, T87G, S223P, E258K, C279R, C405L, I471T, M503K, D523K and D703G; (tt) L26R, Q76R, T87G, N147K, S186G, Y220S, S223P, E258K, C405L, I471T, M503K and D703G; (uu) L26R, T87G, Y220S, S223P, I471T and D703G; (vv) L26R, T87G, N147K, Y220S, S223P, E258K, I471T and D703G; (ww) S21L, L26R, T87G, N147K, Y220S, S223P, E258K, I471T and D703G; (xx) S21L, L26R, T87G, N147K, Y220S, S223P, E258K, C405L, I471T, M503K and D703G; (yy) S21L, L26R, T87G, A121Q, N147K, Y220S, S223P, E258K, C405L, I471T, M503K, S638K and D703G; (zz) S21L, L26R, T87G, A121Q, N147K, S186G, Y220S, S223P, E258K, C405L, I471T, M503K, S638K and D703G; (aaa) L26R, T87G, S223P, I471T and D703G; (bbb) L26R, T87G, N147K, S223P, I471T and D703G; (ccc) L26R, T87G, N147K, S223P, E258K, I471T and D703G; (ddd) L26R, T87G, S186G, H208R, S223P, C405L, I471T and D703G; (eee) L26R, N147K, S186G, S223P, I471T, M503K and D703G; and (fff) L26R, S223P, E258K, I471T and D703G.

In some embodiments, the effector protein comprises an amino acid sequence that is at least 65%, at least 70%, at least 75%, at least 80%, at least 85%, at least 90%, at least 95%, at least 97%, at least 98%, at least 99%, but less than 100% identical to SEQ ID NO: 1, wherein the effector protein comprises at least one substitution selected from any one of the substitutions described in TABLE 2. In some embodiments, the effector protein comprises at least one substitution selected from any one of the substitutions described in TABLE 2, wherein the effector protein, other than the at least one substitution, comprises an amino acid sequence that is at least 65%, at least 70%, at least 75%, at least 80%, at least 85%, at least 90%, at least 95%, at least 97%, at least 98%, at least 99%, but less than 100% identical to SEQ ID NO: 1. In some embodiments, the effector protein comprises an amino acid sequence that is at least 65%, at least 70%, at least 75%, at least 80%, at least 85%, at least 90%, at least 95%, at least 97%, at least 98%, at least 99%, or 100% similar to SEQ ID NO: 1, wherein the effector protein comprises at least one substitution selected from any one of the substitutions described in TABLE 2. In some embodiments, the effector protein comprises at least one substitution selected from any one of the substitutions described in TABLE 2, wherein the effector protein, other than the at least one substitution, comprises an amino acid sequence that is at least 65%, at least 70%, at least 75%, at least 80%, at least 85%, at least 90%, at least 95%, at least 97%, at least 98%, at least 99%, but less than 100% similar to SEQ ID NO: 1. In some embodiments, the effector protein comprises an amino acid sequence that is at least 65%, at least 70%, at least 75%, at least 80%, at least 85%, at least 90%, at least 95%, at least 97%, at least 98%, at least 99%, but less than 100% identical to SEQ ID NO: 1, wherein the effector protein comprises a combination of substitutions selected from any one of the combinations of substitutions described in TABLE 2. In some embodiments, the effector protein comprises an amino acid sequence that is at least 65%, at least 70%, at least 75%, at least 80%, at least 85%, at least 90%, at least 95%, at least 97%, at least 98%, at least 99%, or 100% similar to SEQ ID NO: 1, wherein the effector protein comprises a combination of substitutions selected from any one of the combinations of substitutions described in TABLE 2.

In some embodiments, the effector protein described herein comprises one or more substitutions independently selected from substitutions at positions 13, 14, 15, 16, 17, 18, 19, 20, 21, 22, 23, 24, 25, 26, 28, 29, 30, 31, 32, 33, 34, 35, 36, 37, 38, 39, 40, 41, 42, 43, 44, 51, 52, 53, 54, 55, 56, 57, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 157, 164, 166, 170, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 316, 489, 490, 491, 495, 496, 498, 500, 501, 502, 504, 505, 506, 511, 512, 513, 514, 515, 516, 517, 540, 541, 542, 543, 544, 545, 546, 590, 591, 592, 593, 594, 595, 596, 602, 603, 604, 605, 606, 607 and 608 relative to the amino acid sequence of SEQ ID NO: 1. In some embodiments, the one or more substitutions are independently selected from substitutions by arginine (R), histidine (H), or lysine (K).

In some embodiments, the effector protein described herein comprises one or more substitutions independently selected from substitutions at positions E109, H208, K184, K38, L182, Q183, S108, S198, T114, or a combination thereof relative to the amino acid sequence of SEQ ID NO: 1. In some embodiments, the effector protein described herein comprises one or more substitutions independently selected from substitutions at positions L26, K38, S10, E109, T114, L182, Q183, K184, S198, H208, or a combination thereof relative to the amino acid sequence of SEQ ID NO: 1. In some embodiments, the effector protein comprises one or more amino acid substitutions independently selected from L26R, K38R, S108R, E109R, T114R, L182R, Q183R, K184R, S198R and H208R relative to the amino acid sequence of SEQ ID NO: 1. In some embodiments, the effector protein comprises a substitution of I471T relative to the amino acid sequence of SEQ ID NO: 1. In some embodiments, the effector protein comprises substitutions of L26R and I471T relative to the amino acid sequence of SEQ ID NO: 1. In some embodiments, the effector protein comprises substitutions of L26K, H208R and I471T relative to the amino acid sequence of SEQ ID NO: 1. In some embodiments, the effector protein comprises substitutions of L26K, L149R and I471T relative to the amino acid sequence of SEQ ID NO: 1. In some embodiments, the effector protein comprises substitutions of L26K, I471T and D703G relative to the amino acid sequence of SEQ ID NO: 1.

In some embodiments, the effector protein described herein comprises one or more substitutions independently selected from substitutions at positions L26, 1471, S186, S223, D703, or a combination thereof relative to the amino acid sequence of SEQ ID NO: 1. In some embodiments, the effector protein comprises one or more amino acid substitutions independently selected from L26R, I471T, S186G, S223P and D703G relative to the amino acid sequence of SEQ ID NO: 1. In some embodiments, the combination of amino acid substitutions is L26R, I471T, S223P and D703G relative to the amino acid sequence of SEQ ID NO: 1. In some embodiments, the combination of amino acid substitutions is L26R, I471T, S186G, S223P and D703G relative to the amino acid sequence of SEQ ID NO: 1.

In some embodiments, compositions, systems and methods described herein comprise an effector protein, or a nucleic acid encoding the effector protein, wherein the effector protein comprises one or more conservative substitutions relative to the amino acid sequence recited in TABLE 1. In some embodiments, the one or more conservative substitutions comprises at least one, at least two, at least three, at least four, at least five, at least six, at least seven, at least eight, at least nine, at least ten, at least twelve, at least sixteen, at least twenty, or more conservative substitutions relative to the amino acid sequence recited in TABLE 1. In some embodiments, the one or more conservative substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, sixteen to twenty, or more conservative substitutions relative to the amino acid sequence recited in TABLE 1. In some embodiments, the one or more conservative substitutions comprise one, two, three, four, five, six, seven, eight, nine, ten or more conservative substitutions relative to the amino acid sequence recited in TABLE 1. In some embodiments, compositions, systems and methods described herein comprise an effector protein, or a nucleic acid encoding the effector protein, wherein the effector protein comprises one or more alterations relative to the amino acid sequence recited in TABLE 1 with the exception of 1, 2, 3, 4, 5, 6, 7, 8, 9 or 10 conservative amino acid alterations.

In some embodiments, compositions, systems and methods described herein comprise an effector protein, or a nucleic acid encoding the effector protein, wherein the effector protein comprises one or more non-conservative substitutions relative to the amino acid sequence recited in TABLE 1. In some embodiments, the one or more non-conservative substitutions comprises at least one, at least two, at least three, at least four, at least five, at least six, at least seven, at least eight, at least nine, at least ten, at least twelve, at least sixteen, at least twenty, or more non-conservative substitutions relative to the amino acid sequence recited in TABLE 1. In some embodiments, the one or more non-conservative substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, sixteen to twenty, or more non-conservative substitutions relative to the amino acid sequence recited in TABLE 1. In some embodiments, the one or more non-conservative substitutions comprise one, two, three, four, five, six, seven, eight, nine, ten or more non-conservative substitutions relative to the amino acid sequence recited in TABLE 1. In some embodiments, compositions, systems and methods described herein comprise an effector protein, or a nucleic acid encoding the effector protein, wherein the effector protein comprises one or more alterations relative to the amino acid sequence recited in TABLE 1 with the exception of 1, 2, 3, 4, 5, 6, 7, 8, 9 or 10 non-conservative amino acid alterations.

In some embodiments, compositions, systems and methods described herein comprise an effector protein, or a nucleic acid encoding the effector protein, wherein the effector protein comprises a portion of its amino acid sequence (or domain) deleted relative to any one of the amino acid sequences recited in TABLE 1. In some embodiments, compositions, systems and methods described herein comprise an effector protein, or a nucleic acid encoding the effector protein, wherein the effector protein comprises a portion of its amino acid sequence (or domain) that is substituted with a different amino acid sequence relative to the amino acid sequence recited in TABLE 1.

TABLE 3 provides exemplary amino acid sequences (or domains) that can be deleted and/or substituted from corresponding wildtype amino acid sequences recited in TABLE 1. TABLE 3 also provides amino acid sequences that can be substituted in place of the portion of the amino acid sequence that has been deleted from the corresponding wildtype amino acid sequences recited TABLE 1. In some embodiments, the domain comprises an amino acid sequence that is at least 65%, at least 70%, at least 75%, at least 80%, at least 85%, at least 90%, at least 95%, at least 97%, at least 98%, at least 99%, or 100% identical to any one of SEQ ID NOS: 6-34 and 349-355. In some embodiments, the domain is substituted with a different amino acid sequence. In some embodiments, the different amino acid sequence comprises an amino acid sequence that is at least 65%, at least 70%, at least 75%, at least 80%, at least 85%, at least 90%, at least 95%, at least 97%, at least 98%, at least 99%, or 100% identical to any one of SEQ ID NOS: 18, 41-104 and 356-368. In some embodiments, the effector protein comprising an amino acid sequence that is at least 70% identical to SEQ ID NO: 1 comprises at least one substitution selected from any one of the substitutions or combinations thereof described in TABLE 2, wherein the effector protein further comprises a deletion and/or substitution of a domain with a different amino acid sequence as identified in TABLE 3. In some embodiments, the at least one substitution is selected from L26R, I471T, or a combination thereof relative to SEQ ID NO: 1. In some embodiments, the at least one substitution is selected from L26R, I471T, S223P, D703G, L149R, E157R, H208R, D704G, A706G, or a combination thereof relative to SEQ ID NO: 1. In some embodiments, the at least one substitution comprises a combination of L26R, I471T, S223P and D703G substitutions relative to SEQ ID NO: 1. In some embodiments, the at least one substitution comprises a combination of L26R, I471T, S223P D703G and H208R substitutions relative to SEQ ID NO: 1. In some embodiments, the at least one substitution comprises a combination of L26R, I471T, S223P D703G, L149R and H208R substitutions relative to SEQ ID NO: 1. In some embodiments, the at least one substitution comprises a combination of of L26R, I471T, S223P D703G, D704G and A706G substitutions relative to SEQ ID NO: 1.

In some embodiments, the different amino acid sequence comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 18, wherein the amino acid sequence comprises at least one of amino acid residues at positions F9 and K14 remain unchanged. In some embodiments, the different amino acid sequence comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 18, wherein the amino acid sequence comprises a substitution selected from V1G, V1H, V1K and V1N. In some embodiments, the different amino acid sequence comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 18, wherein the amino acid sequence comprises a substitution selected from N3D, N3S, N3G and N3E. In some embodiments, the different amino acid sequence comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 18, wherein the amino acid sequence comprises a substitution selected from F9S, F9V, F9Q, F9L, F9Y, F9I and F9D. In some embodiments, the different amino acid sequence comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 18, wherein the amino acid sequence comprises a substitution selected from K14P, K14R, K14G, K14D and K14S. In some embodiments, the different amino acid sequence comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 18, wherein the amino acid sequence comprises a substitution selected from M21K, M21L, M21R, M21F and M21D. In some embodiments, the different amino acid sequence comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 18, wherein the amino acid sequence comprises a substitution selected from K22R, K22I, K22L, K22F, K22P and K22W. In some embodiments, the different amino acid sequence comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 18, wherein the amino acid sequence comprises one or more substitutions selected from V1G, N3D, M21K and K22R, and wherein the amino acid sequence comprises at least one of amino acid residues at positions F9 and K14 remain unchanged.

In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 6, and the different amino acid sequence comprises the amino acid sequence that is at least 65%, at least 70%, at least 75%, at least 80%, at least 85%, at least 90%, at least 95%, at least 97%, at least 98%, at least 99%, or 100% identical to any one of SEQ ID NO: 41-47. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to any one of SEQ ID NO: 7-10, 13, 17-18 and 20, and the different amino acid sequence comprises the amino acid sequence of SEQ ID NO: 48. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to any one of SEQ ID NO: 11 and 12, and the different amino acid sequence comprises the amino acid sequence of SEQ ID NO: 49. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 14, and the different amino acid sequence comprises the amino acid sequence that is at least 65%, at least 70%, at least 75%, at least 80%, at least 85%, at least 90%, at least 95%, at least 97%, at least 98%, at least 99%, or 100% identical to any one of SEQ ID NO: 50-65. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 15, and the different amino acid sequence comprises the amino acid sequence that is at least 65%, at least 70%, at least 75%, at least 80%, at least 85%, at least 90%, at least 95%, at least 97%, at least 98%, at least 99%, or 100% identical to any one of SEQ ID NO: 66-100. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 16, and the different amino acid sequence comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 101. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 19, and the different amino acid sequence comprises the amino acid sequence of SEQ ID NO: 102. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 21, and the different amino acid sequence comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 103. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 22, and the different amino acid sequence comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 104. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to any one of SEQ ID NO: 23-25, and the different amino acid sequence comprises the amino acid sequence of SEQ ID NO: 48. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 24, and the different amino acid sequence comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 18. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to any one of SEQ ID NO: 26-28, and the different amino acid sequence comprises the amino acid sequence of SEQ ID NO: 48. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 27, and the different amino acid sequence comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 18. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to any one of SEQ ID NO: 29-31, and the different amino acid sequence comprises the amino acid sequence of SEQ ID NO: 48. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 30, and the different amino acid sequence comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 18. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to any one of SEQ ID NO: 32-34, and the different amino acid sequence comprises the amino acid sequence of SEQ ID NO: 48. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 33, and the different amino acid sequence comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 18. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 349, and the different amino acid sequence comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 356. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 350, and the different amino acid sequence comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 357. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 351, and the different amino acid sequence comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 358. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 352, and the different amino acid sequence comprises the amino acid sequence that is at least 65%, at least 70%, at least 75%, at least 80%, at least 85%, at least 90%, at least 95%, at least 97%, at least 98%, at least 99%, or 100% identical to any one of SEQ ID NO: 359-365. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 353, and the different amino acid sequence comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 366. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 354, and the different amino acid sequence comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 367. In some embodiments, the domain comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 355, and the different amino acid sequence comprises the amino acid sequence that is at least 65%, at least 70%, at least 75%, at least 80%, at least 85%, at least 90%, at least 95%, at least 97%, at least 98%, at least 99%, or 100% identical to SEQ ID NO: 368.

In some embodiments, effector proteins described herein comprises an amino acid sequence that is at least 70%, at least 75%, at least 80%, at least 85%, at least 90%, at least 95%, at least 98% or at least 99% identical to any one of the sequences set forth in TABLE 1, wherein the effector protein further comprises a deletion of one or more domains, a substitution of one or more domains for a different amino acid sequence, or a combination thereof, wherein the one or more domains independently comprise an amino acid sequence that is at least 90% identical to any one of the domains identified in TABLE 3. In some embodiments, (a) the effector protein comprises an amino acid sequence that is at least 70% identical to SEQ ID NO: 1, (b) the domain comprises an amino acid sequence that is at least 90% identical to any one of SEQ ID NO: 6-22 and 349-355, and (c) the different amino acid sequence comprises an amino acid sequence that is at least 90% identical to any one of SEQ ID NO: 41-104 and 356-368. In some embodiments, (a) the effector protein comprises an amino acid sequence that is at least 70% identical to SEQ ID NO: 2, (b) the domain comprises an amino acid sequence that is at least 90% identical to any one of SEQ ID NO: 23-25, and (c) the different amino acid sequence comprises an amino acid sequence of SEQ ID NO: 18 or 48. In some embodiments, (a) the effector protein comprises an amino acid sequence that is at least 70% identical to SEQ ID NO: 3, (b) the domain comprises an amino acid sequence that is at least 90% identical to any one of SEQ ID NO: 26-28, and (c) the different amino acid sequence comprises an amino acid sequence of SEQ ID NO: 18 or 48. In some embodiments, (a) the effector protein comprises an amino acid sequence that is at least 70% identical to SEQ ID NO: 4, (b) the domain comprises an amino acid sequence that is at least 90% identical to any one of SEQ ID NO: 29-31, and (c) the different amino acid sequence comprises an amino acid sequence of SEQ ID NO: 18 or 48. In some embodiments, (a) the effector protein comprises an amino acid sequence that is at least 70% identical to SEQ ID NO: 5, (b) the domain comprises an amino acid sequence that is at least 90% identical to any one of SEQ ID NO: 32-34, and (c) the different amino acid sequence comprises an amino acid sequence of SEQ ID NO: 18 or 48. In some embodiments, the effector protein or the nucleic acid encoding the effector protein comprises one or more portion of its amino acid sequence that is deleted, one or more portion of its amino acid sequence that is substituted, one or more amino acid substitutions, or combinations thereof relative to any one of the sequences recited in TABLE 1.

In some embodiments, compositions, systems and methods described herein comprise an effector protein comprising an amino acid sequence that is at least 70%, at least 75%, at least 80%, at least 85%, at least 90%, at least 95%, at least 98% or at least 99% identical to any one of SEQ ID NOS: 1-5 further comprises a deletion of one or more domains, a substitution of one or more domains for a different amino acid sequence, or a combination thereof, wherein the one or more domains independently comprise an amino acid sequence that is at least 90% identical to any one of the domains identified in TABLE 3. In some embodiments, the other In some embodiments, the effector protein comprises an amino acid sequence that is at least 70%, at least 75%, at least 80%, at least 85%, at least 90%, at least 95%, at least 98% or at least 99% identical to SEQ ID NO: 1, the domain comprises an amino acid sequence that is at least 70%, at least 75%, at least 80%, at least 85%, at least 90%, at least 95%, at least 98% or at least 99% identical to any one of SEQ ID NO: 6-22 and 349-355, and the different amino acid sequence comprises an amino acid sequence that is at least 70%, at least 75%, at least 80%, at least 85%, at least 90%, at least 95%, at least 98% or at least 99% identical to any one of SEQ ID NO: 41-104 and 356-368. In some embodiments, the effector protein further comprises one or more substitutions relative to SEQ ID NO: 1. In some embodiments, the one or more substitutions comprises any one of individual amino acid substitutions or combination of amino acid substitutions recited in TABLE 2. In some embodiments, the effector protein comprises an amino acid sequence that is at least 70%, at least 75%, at least 80%, at least 85%, at least 90%, at least 95%, at least 98% or at least 99% identical to SEQ ID NO: 2, the domain comprises an amino acid sequence that is at least 70%, at least 75%, at least 80%, at least 85%, at least 90%, at least 95%, at least 98% or at least 99% identical to any one of SEQ ID NO: 23-25, and the different amino acid sequence comprises an amino acid sequence that is at least 70%, at least 75%, at least 80%, at least 85%, at least 90%, at least 95%, at least 98% or at least 99% identical to SEQ ID NO: 18 or 48. In some embodiments, the effector protein comprises an amino acid sequence that is at least 70%, at least 75%, at least 80%, at least 85%, at least 90%, at least 95%, at least 98% or at least 99% identical to SEQ ID NO: 3, the domain comprises an amino acid sequence that is at least 70%, at least 75%, at least 80%, at least 85%, at least 90%, at least 95%, at least 98% or at least 99% identical to any one of SEQ ID NO: 26-28, and the different amino acid sequence comprises an amino acid sequence that is at least 70%, at least 75%, at least 80%, at least 85%, at least 90%, at least 95%, at least 98% or at least 99% identical to SEQ ID NO: 18 or 48. In some embodiments, the effector protein comprises an amino acid sequence that is at least 70%, at least 75%, at least 80%, at least 85%, at least 90%, at least 95%, at least 98% or at least 99% identical to SEQ ID NO: 4, the domain comprises an amino acid sequence that is at least 70%, at least 75%, at least 80%, at least 85%, at least 90%, at least 95%, at least 98% or at least 99% identical to any one of SEQ ID NO: 29-31, and the different amino acid sequence comprises an amino acid sequence that is at least 70%, at least 75%, at least 80%, at least 85%, at least 90%, at least 95%, at least 98% or at least 99% identical to SEQ ID NO: 18 or 48. In some embodiments, the effector protein comprises an amino acid sequence that is at least 70%, at least 75%, at least 80%, at least 85%, at least 90%, at least 95%, at least 98% or at least 99% identical to SEQ ID NO: 5, the domain comprises an amino acid sequence that is at least 70%, at least 75%, at least 80%, at least 85%, at least 90%, at least 95%, at least 98% or at least 99% identical to any one of SEQ ID NO: 32-34, and the different amino acid sequence comprises an amino acid sequence that is at least 70%, at least 75%, at least 80%, at least 85%, at least 90%, at least 95%, at least 98% or at least 99% identical to SEQ ID NO: 18 or 48.

In some embodiments, compositions, systems and methods described herein comprise an effector protein comprising an amino acid sequence that is at least 70%, at least 75%, at least 80%, at least 85%, at least 90%, at least 95%, at least 98% or at least 99% identical to SEQ ID NO: 1 further comprises (a) one or more substitutions relative to SEQ ID NO: 1; and (b) a deletion of one or more domains, a substitution of one or more domains for a different amino acid sequence, or a combination thereof. In some embodiments, the one or more substitutions comprises any one of individual amino acid substitutions or combination of amino acid substitutions recited in TABLE 2. In some embodiments, the domain comprises an amino acid sequence that is at least 70%, at least 75%, at least 80%, at least 85%, at least 90%, at least 95%, at least 98% or at least 99% identical to any one of SEQ ID NO: 6-22 and 349-355. In some embodiments, the different amino acid sequence comprises an amino acid sequence that is at least 70%, at least 75%, at least 80%, at least 85%, at least 90%, at least 95%, at least 98% or at least 99% identical to any one of SEQ ID NO: 41-104 and 356-368.

In some embodiments, compositions, systems and methods described herein comprise an effector protein, or a nucleic acid encoding the effector protein, wherein the amino acid sequence of the effector protein comprises at least 200 contiguous amino acids or more of the amino acid sequence recited in TABLE 4, wherein the amino acid sequence of the effector protein is not identical to any one of the amino acid sequences recited in TABLE 1. In some embodiments, the amino acid sequence of an effector protein provided herein comprises at least 200, at least 220, at least 240, at least 260, at least 280, at least 300, at least 320, at least 340, at least 360, at least 380, at least 400 contiguous amino acids, at least 420 contiguous amino acids, at least 440 contiguous amino acids, at least 460 contiguous amino acids, at least 480 contiguous amino acids, at least 500 contiguous amino acids, at least 520 contiguous amino acids, at least 540 contiguous amino acids, at least 560 contiguous amino acids, at least 580 contiguous amino acids, at least 600 contiguous amino acids, at least 620 contiguous amino acids, at least 640 contiguous amino acids, at least 660 contiguous amino acids, at least 680 contiguous amino acids, at least 700 contiguous amino acids, at least 720 contiguous amino acids, at least 760 contiguous amino acids, or more of the amino acid sequence of TABLE 4, wherein the amino acid sequence of the effector protein is not identical to any one of the amino acid sequences recited in TABLE 1.

In some embodiments, compositions, systems and methods described herein comprise an effector protein or a nucleic acid encoding the effector protein, wherein the effector protein comprises a portion of the amino acid sequence recited in TABLE 4. In some embodiments, the effector protein comprises a portion of the amino acid sequence recited in TABLE 4, wherein the portion does not comprise at least the first 10 amino acids, at least the first 20 amino acids, at least the first 40 amino acids, at least the first 60 amino acids, at least the first 80 amino acids, at least the first 100 amino acids, at least the first 120 amino acids, at least the first 140 amino acids, at least the first 160 amino acids, at least the first 180 amino acids, or at least the first 200 amino acids of the amino acid sequence recited in TABLE 4. In some embodiments, the effector protein comprises a portion of the amino acid sequence recited in TABLE 4, wherein the portion does not comprise the last 10 amino acids, the last 20 amino acids, the last 40 amino acids, the last 60 amino acids, the last 80 amino acids, the last 100 amino acids, the last 120 amino acids, the last 140 amino acids, the last 160 amino acids, the last 180 amino acids, or the last 200 amino acids of the amino acid sequence recited in TABLE 4. In some embodiments, the effector protein comprises at least 600, at least 620, at least 640, at least 660, at least 680, or at least 700 contiguous amino acids of any one of the sequences recited in TABLE 4.

In some embodiments, compositions, systems and methods described herein comprise an effector protein, or a nucleic acid encoding the effector protein, wherein the effector protein comprises an amino acid sequence that is at least 65%, at least 70%, at least 75%, at least 80%, at least 85%, at least 90%, at least 95%, at least 97%, at least 98%, at least 99%, or 100% identical to any one of the amino acid sequences as recited in TABLE 4. In some embodiments, an effector protein provided herein comprises an amino acid sequence that is at least 65% identical to any one of the amino acid sequences as recited in TABLE 4. In some embodiments, an effector protein provided herein comprises an amino acid sequence that is at least 70% identical to any one of the amino acid sequences as recited in TABLE 4. In some embodiments, an effector protein provided herein comprises an amino acid sequence that is at least 75% identical to any one of the amino acid sequences as recited in TABLE 4. In some embodiments, an effector protein provided herein comprises an amino acid sequence that is at least 80% identical to any one of the amino acid sequences as recited in TABLE 4. In some embodiments, an effector protein provided herein comprises an amino acid sequence that is at least 85% identical to any one of the amino acid sequences as recited in TABLE 4. In some embodiments, an effector protein provided herein comprises an amino acid sequence that is at least 90% identical to any one of the amino acid sequences as recited in TABLE 4. In some embodiments, an effector protein provided herein comprises an amino acid sequence that is at least 95% identical to any one of the amino acid sequences as recited in TABLE 4. In some embodiments, an effector protein provided herein comprises an amino acid sequence that is at least 97% identical to any one of the amino acid sequences as recited in TABLE 4. In some embodiments, an effector protein provided herein comprises an amino acid sequence that is at least 98% identical to any one of the amino acid sequences as recited in TABLE 4. In some embodiments, an effector protein provided herein comprises an amino acid sequence that is at least 99% identical to any one of the amino acid sequences as recited in TABLE 4. In some embodiments, an effector protein provided herein comprises an amino acid sequence that is 100% identical to any one of the amino acid sequences as recited in TABLE 4. In some embodiments, the effector protein comprises or consists of any one of the amino acid sequences selected from TABLE 4. In some embodiments, the effector protein comprises an amino acid sequence that is at least 90%, at least 95%, at least 97%, at least 98%, or at least 99% identical to any one of the amino acid sequences recited in TABLE 4, and wherein the amino acid sequence comprises all amino acid differences between an amino acid sequence recited in TABLE 4 and SEQ ID NO: 1. In some embodiments, the amino acid sequence that is at least 90%, at least 95%, at least 97%, at least 98%, or at least 99% identical to any one of the amino acid sequences recited in TABLE 4, other than all the amino acid differences between the amino acid sequence recited in TABLE 4 and SEQ ID NO: 1, is comprised of conservative amino acid substitutions relative to the amino acid sequence recited in TABLE 4. In some embodiments, the one or more amino acid alterations are conservative amino acid substitutions.

In some embodiments, compositions, systems and methods described herein comprise an effector protein, or a nucleic acid encoding the effector protein, wherein the effector protein comprises an amino acid sequence that is at least 65%, at least 70%, at least 75%, at least 80%, at least 85%, at least 90%, at least 95%, at least 97%, at least 98%, at least 99%, or 100% identical to any one of the amino acid sequence of SEQ ID NOS: 105, 106, 271, 281-293, 324-332 and 334. In some embodiments, an effector protein provided herein comprises an amino acid sequence that is at least 65% identical to any one of the amino acid sequences of SEQ ID NOS: 105, 106, 271, 281-293, 324-332 and 334. In some embodiments, an effector protein provided herein comprises an amino acid sequence that is at least 70% identical to any one of the amino acid sequences of SEQ ID NOS: 105, 106, 271, 281-293, 324-332 and 334. In some embodiments, an effector protein provided herein comprises an amino acid sequence that is at least 75% identical to any one of the amino acid sequences of SEQ ID NOS: 105, 106, 271, 281-293, 324-332 and 334. In some embodiments, an effector protein provided herein comprises an amino acid sequence that is at least 80% identical to any one of the amino acid sequences of SEQ ID NOS: 105, 106, 271, 281-293, 324-332 and 334. In some embodiments, an effector protein provided herein comprises an amino acid sequence that is at least 85% identical to any one of the amino acid sequences of SEQ ID NOS: 105, 106, 271, 281-293, 324-332 and 334. In some embodiments, an effector protein provided herein comprises an amino acid sequence that is at least 90% identical to any one of the amino acid sequences of SEQ ID NOS: 105, 106, 271, 281-293, 324-332 and 334. In some embodiments, an effector protein provided herein comprises an amino acid sequence that is at least 95% identical to any one of the amino acid sequences of SEQ ID NOS: 105, 106, 271, 281-293, 324-332 and 334. In some embodiments, an effector protein provided herein comprises an amino acid sequence that is at least 97% identical to any one of the amino acid sequences of SEQ ID NOS: 105, 106, 271, 281-293, 324-332 and 334. In some embodiments, an effector protein provided herein comprises an amino acid sequence that is at least 98% identical to any one of the amino acid sequences of SEQ ID NOS: 105, 106, 271, 281-293, 324-332 and 334. In some embodiments, an effector protein provided herein comprises an amino acid sequence that is at least 99% identical to any one of the amino acid sequences of SEQ ID NOS: 105, 106, 271, 281-293, 324-332 and 334. In some embodiments, an effector protein provided herein comprises an amino acid sequence that is 100% identical to any one of the amino acid sequences of SEQ ID NOS: 105, 106, 271, 281-293, 324-332 and 334.

In some embodiments, compositions, systems and methods described herein comprise an effector protein, or a nucleic acid encoding the effector protein, wherein the effector protein comprises an amino acid sequence that is at least 65%, at least 70%, at least 75%, at least 80%, at least 85%, at least 90%, at least 95%, at least 97%, at least 98%, at least 99%, or 100% identical to any one of the amino acid sequences of SEQ ID NOS: 271, 379-394 and 396-398. In some embodiments, an effector protein provided herein comprises an amino acid sequence that is at least 65% identical to any one of the amino acid sequences of SEQ ID NOS: 271, 379-394 and 396-398. In some embodiments, an effector protein provided herein comprises an amino acid sequence that is at least 70% identical to any one of the amino acid sequences of SEQ ID NOS: 271, 379-394 and 396-398. In some embodiments, an effector protein provided herein comprises an amino acid sequence that is at least 75% identical to any one of the amino acid sequences of SEQ ID NOS: 271, 379-394 and 396-398. In some embodiments, an effector protein provided herein comprises an amino acid sequence that is at least 80% identical to any one of the amino acid sequences of SEQ ID NOS: 271, 379-394 and 396-398. In some embodiments, an effector protein provided herein comprises an amino acid sequence that is at least 85% identical to any one of the amino acid sequences of SEQ ID NOS: 271, 379-394 and 396-398. In some embodiments, an effector protein provided herein comprises an amino acid sequence that is at least 90% identical to any one of the amino acid sequences of SEQ ID NOS: 271, 379-394 and 396-398. In some embodiments, an effector protein provided herein comprises an amino acid sequence that is at least 95% identical to any one of the amino acid sequences of SEQ ID NOS: 271, 379-394 and 396-398. In some embodiments, an effector protein provided herein comprises an amino acid sequence that is at least 97% identical to any one of the amino acid sequences of SEQ ID NOS: 271, 379-394 and 396-398. In some embodiments, an effector protein provided herein comprises an amino acid sequence that is at least 98% identical to any one of the amino acid sequences of SEQ ID NOS: 271, 379-394 and 396-398. In some embodiments, an effector protein provided herein comprises an amino acid sequence that is at least 99% identical to any one of the amino acid sequences of SEQ ID NOS: 271, 379-394 and 396-398. In some embodiments, an effector protein provided herein comprises an amino acid sequence that is 100% identical to any one of the amino acid sequences of SEQ ID NOS: 271, 379-394 and 396-398.

In some embodiments, compositions, systems and methods described herein comprise an effector protein, or a nucleic acid encoding the effector protein, wherein the effector protein comprises an amino acid sequence that is at least 80%, at least 85%, at least 90%, at least 95%, at least 97%, at least 98%, at least 99%, or 100% similar to any one of the amino acid sequences as recited in TABLE 4. In some embodiments, an effector protein provided herein comprises an amino acid sequence that is at least 80% similar to any one of the amino acid sequences as recited in TABLE 4. In some embodiments, an effector protein provided herein comprises an amino acid sequence that is at least 85% similar to any one of the amino acid sequences as recited in TABLE 4. In some embodiments, an effector protein provided herein comprises an amino acid sequence that is at least 90% similar to any one of the amino acid sequences as recited in TABLE 4. In some embodiments, an effector protein provided herein comprises an amino acid sequence that is at least 95% similar to any one of the amino acid sequences as recited in TABLE 4. In some embodiments, an effector protein provided herein comprises an amino acid sequence that is at least 97% similar to any one of the amino acid sequences as recited in TABLE 4. In some embodiments, an effector protein provided herein comprises an amino acid sequence that is at least 98% similar to any one of the amino acid sequences as recited in TABLE 4. In some embodiments, an effector protein provided herein comprises an amino acid sequence that is at least 99% similar to any one of the amino acid sequences as recited in TABLE 4. In some embodiments, an effector protein provided herein comprises an amino acid sequence that is 100% similar to any one of the amino acid sequences as recited in TABLE 4.

In some embodiments, compositions, systems and methods described herein comprise an effector protein, or a nucleic acid encoding the effector protein, wherein the effector protein comprises an amino acid sequence that is at least 80%, at least 85%, at least 90%, at least 95%, at least 97%, at least 98%, at least 99%, or 100% similar to any one of the amino acid sequence of SEQ ID NOS: 105, 106, 271, 281-293, 324-332 and 334. In some embodiments, an effector protein provided herein comprises an amino acid sequence that is at least 80% similar to any one of the amino acid sequences of SEQ ID NOS: 105, 106, 271, 281-293, 324-332 and 334. In some embodiments, an effector protein provided herein comprises an amino acid sequence that is at least 85% similar to any one of the amino acid sequences of SEQ ID NOS: 105, 106, 271, 281-293, 324-332 and 334. In some embodiments, an effector protein provided herein comprises an amino acid sequence that is at least 90% similar to any one of the amino acid sequences of SEQ ID NOS: 105, 106, 271, 281-293, 324-332 and 334. In some embodiments, an effector protein provided herein comprises an amino acid sequence that is at least 95% similar to any one of the amino acid sequences of SEQ ID NOS: 105, 106, 271, 281-293, 324-332 and 334. In some embodiments, an effector protein provided herein comprises an amino acid sequence that is at least 97% similar to any one of the amino acid sequences of SEQ ID NOS: 105, 106, 271, 281-293, 324-332 and 334. In some embodiments, an effector protein provided herein comprises an amino acid sequence that is at least 98% similar to any one of the amino acid sequences of SEQ ID NOS: 105, 106, 271, 281-293, 324-332 and 334. In some embodiments, an effector protein provided herein comprises an amino acid sequence that is at least 99% similar to any one of the amino acid sequences of SEQ ID NOS: 105, 106, 271, 281-293, 324-332 and 334. In some embodiments, an effector protein provided herein comprises an amino acid sequence that is 100% similar to any one of the amino acid sequences of SEQ ID NOS: 105, 106, 271, 281-293, 324-332 and 334.

In some embodiments, compositions, systems and methods described herein comprise an effector protein, or a nucleic acid encoding the effector protein, wherein the effector protein comprises an amino acid sequence that is at least 80%, at least 85%, at least 90%, at least 95%, at least 97%, at least 98%, at least 99%, or 100% similar to any one of the amino acid sequence of SEQ ID NOS: 271, 379-394 and 396-398. In some embodiments, an effector protein provided herein comprises an amino acid sequence that is at least 80% similar to any one of the amino acid sequences of SEQ ID NOS: 271, 379-394 and 396-398. In some embodiments, an effector protein provided herein comprises an amino acid sequence that is at least 85% similar to any one of the amino acid sequences of SEQ ID NOS: 271, 379-394 and 396-398. In some embodiments, an effector protein provided herein comprises an amino acid sequence that is at least 90% similar to any one of the amino acid sequences of SEQ ID NOS: 271, 379-394 and 396-398. In some embodiments, an effector protein provided herein comprises an amino acid sequence that is at least 95% similar to any one of the amino acid sequences of SEQ ID NOS: 271, 379-394 and 396-398. In some embodiments, an effector protein provided herein comprises an amino acid sequence that is at least 97% similar to any one of the amino acid sequences of SEQ ID NOS: 271, 379-394 and 396-398. In some embodiments, an effector protein provided herein comprises an amino acid sequence that is at least 98% similar to any one of the amino acid sequences of SEQ ID NOS: 271, 379-394 and 396-398. In some embodiments, an effector protein provided herein comprises an amino acid sequence that is at least 99% similar to any one of the amino acid sequences of SEQ ID NOS: 271, 379-394 and 396-398. In some embodiments, an effector protein provided herein comprises an amino acid sequence that is 100% similar to any one of the amino acid sequences of SEQ ID NOS: 271, 379-394 and 396-398.

In some embodiments, compositions, systems and methods described herein comprise an effector protein, or a nucleic acid encoding the effector protein, wherein the effector protein comprises one or more amino acid alterations relative to the amino acid sequence recited in TABLE 4. In some embodiments, the one or more alterations comprises at least one, at least two, at least three, at least four, at least five, at least six, at least seven, at least eight, at least nine, at least ten, at least twelve, at least sixteen, at least twenty, or more amino acid alterations relative to the amino acid sequence recited in TABLE 4. In some embodiments, the one or more alterations comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, sixteen to twenty, or more amino acid alterations relative to the amino acid sequence recited in TABLE 4. In some embodiments, the one or more alterations comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 or more amino acid alterations relative to the amino acid sequence recited in TABLE 4. In some embodiments, the one or more alterations comprises one, two, three, four, five, six, seven, eight, nine, ten, or more amino acid alterations relative to the amino acid sequence recited in TABLE 4. In some embodiments, the one or more amino acid alterations comprises substitutions (e.g., conservative substitutions, non-conservative substitutions), deletions, or combinations thereof. In some embodiments, an effector protein or a nucleic acid encoding the effector protein comprises 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations or more relative to the amino acid sequence recited in TABLE 4.

In some embodiments, compositions, systems and methods described herein comprise an effector protein, or a nucleic acid encoding the effector protein, wherein the effector protein comprises one or more substitutions relative to the amino acid sequence recited in TABLE 4. In some embodiments, the one or more substitutions comprises at least one, at least two, at least three, at least four, at least five, at least six, at least seven, at least eight, at least nine, at least ten, at least twelve, at least sixteen, at least twenty, or more substitutions relative to the amino acid sequence recited in TABLE 4. In some embodiments, the one or more substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, sixteen to twenty, or more substitutions relative to the amino acid sequence recited in TABLE 4. In some embodiments, the one or more substitutions comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 or more amino acid substitutions relative to the amino acid sequence recited in TABLE 4. In some embodiments, the one or more amino acid substitutions comprise one, two, three, four, five, six, seven, eight, nine, ten or more substitutions relative to the amino acid sequence recited in TABLE 4. In some embodiments, the one or more amino acid substitutions comprise one or more substitutions with a positively charged amino acid residues. In some embodiments, the positively charged amino acid residue is independently selected from Lys (K), Arg (R), or His (H). In some embodiments, the one or more substitutions comprise one or more conservative substitutions, one or more non-conservative substitutions, or combinations thereof.

### Effector Partners

Provided herein are compositions, systems and methods comprising one or more effector partners or uses thereof. In some embodiments, the effector partner is a heterologous protein. In some embodiments, the effector partner is fused or linked to any one of the effector proteins described herein. In some embodiments, the amino terminus of the effector partner is linked to the carboxy terminus of the effector protein directly or by a linker. In some embodiments, the carboxy terminus of the effector partner is linked to the amino terminus of the effector protein directly or by a linker. In some embodiments, the effector partner is functional when the effector protein is coupled to a guide nucleic acid. In some embodiments, the effector partner is functional when the effector protein is coupled to a target nucleic acid. In some embodiments, the guide nucleic acid imparts sequence specific activity to the effector partner. In some embodiments, the effector partner described herein does not comprise an effector protein. In some embodiments, the effector partner imparts some function or activity that is not provided by an effector protein. In some embodiments, the effector partner is capable of forming a multimeric protein with another effector partner. In some embodiments, the multimeric protein is a heteromeric protein. In some embodiments, the multimeric protein is a homomeric protein.

In some embodiments, an effector partner imparts a function or activity to a fusion protein comprising an effector protein that is not provided by the effector protein, including but not limited to nuclease activity, methyltransferase activity, demethylase activity, DNA repair activity, DNA damage activity, deamination activity, dismutase activity, alkylation activity, depurination activity, oxidation activity, dimer forming activity (e.g., pyrimidine dimer forming activity), integrase activity, transposase activity, recombinase activity, polymerase activity, ligase activity, helicase activity, photolyase activity, glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity, modification of a polypeptide associated with target nucleic acid (e.g., a histone) and/or signaling activity.

In some embodiments, the effector partner directly or indirectly modifies a target nucleic acid. Modifications can be of a nucleobase, nucleotide, or nucleotide sequence of a target nucleic acid. In some embodiments, the effector partner interacts with additional proteins, or functional fragments thereof, to make modifications to a target nucleic acid. In some embodiments, modification of a target nucleic acid comprises introducing or removing epigenetic modification(s). In other embodiments, the effector partner modifies proteins associated with a target nucleic acid. In some embodiments, an effector partner modulates transcription (e.g., inhibits transcription, increases transcription) of a target nucleic acid. In yet another example, an effector partner directly or indirectly inhibits, reduces, activates or increases expression of a target nucleic acid.

In some embodiments, the effector partner described herein comprises modification activities. In some embodiments, the modification activities comprise a nuclease activity, nickase activity, binding activity, insertion activity, substitution activity and the like. Modification activity of an effector partner may result in: nicking of a double-stranded target nucleic acid, breaking of a double-stranded target nucleic acid, chemical modification of one or more nucleotides of a target nucleic acid into an alternate nucleotide, substitution of one or more nucleotides of a target nucleic acid with an alternative nucleotide, more than one of the foregoing, or any combination thereof. A target nucleic acid comprises a target strand and a non-target strand. In some embodiments, the target strand comprises a target sequence. In some embodiments, the target sequence is hybridized to the guide nucleic acid-effector protein complex. In some embodiments, the non-target strand does not comprise the target sequence. Accordingly, in some embodiments, the effector partner edits a target strand and/or a non-target strand of a target nucleic acid. In some embodiments, an ability of an effector partner to modify a target nucleic acid depends upon the effector protein being complexed with a guide nucleic acid, the guide nucleic acid being hybridized to a target sequence of the target nucleic acid, the distance between the target sequence and a PAM sequence, concentration of the effector partner near to the target nucleic acid, distance between the effector protein and the effector partner, or combinations thereof.

In some embodiments, proteins (e.g., effector protein or effector partner) described herein have been modified (also referred to as an engineered protein). In some embodiments, a modification of the proteins includes addition of one or more amino acids, deletion of one or more amino acids, substitution of one or more amino acids, or combinations thereof. In some embodiments, the proteins disclosed herein are engineered proteins. Unless otherwise indicated, reference to the proteins throughout the present disclosure include engineered proteins thereof.

### Reverse Transcriptase (RT) Editing System

In some embodiments, systems and methods comprise components or uses of an RT editing system to modify a target nucleic acid. RT editing may also be referred to as prime editing or precise nucleobase editing. In some embodiments, an RT editing system comprises an effector protein and an effector partner comprising an RT editing enzyme. In some embodiments, the effector protein that is linked to the RT editing enzyme. In some embodiments, an RT editing enzyme comprises a polymerase. In some embodiments, an RT editing enzyme comprises a reverse transcriptase. A non-limiting example of a reverse transcriptase is an M-MLV RT enzyme and variants thereof having polymerase activity. In some embodiments, the M-MLV RT enzyme comprises at least one mutation selected from D200N, L603W, T330P, T306K and W313F relative to wildtype M-MLV RT enzyme. In some embodiments, systems and methods comprise an RT editing enzyme, wherein the RT editing enzyme is not fused or linked to the effector protein. In some embodiments, the RT editing enzyme comprises a recruiting moiety that recruits the RT editing enzyme to the target nucleic acid. By way of non-limiting example, the RT editing enzyme comprises a peptide that binds an aptamer, wherein the aptamer is located on a guide RNA, template RNA, or combination thereof. Also, by way of non-limiting example, the RT editing enzyme is linked to a protein that binds to (or is bound by) the effector protein or a protein linked/fused to the effector protein. In some embodiments, an RT editing enzyme requires an RT editing guide RNA (pegRNA) to catalyze editing. Such a pegRNA may be capable of identifying a target nucleotide or target sequence in a target nucleic acid to be edited and encoding a new genetic information that replaces the target nucleotide or target sequence in the target nucleic acid. An RT editing enzyme may require a pegRNA and a guide RNA, such as a single guide RNA, to catalyze the editing. In some embodiments, the RT editing system comprises a template RNA comprising a primer binding sequence that hybridizes to a primer sequence of the dsDNA molecule that is formed when target nucleic acid is nicked, and a template sequence that is complementary to at least a portion of the target sequence of the dsDNA molecule except for at least one nucleotide. In some embodiments, the template RNA is covalently linked to a guide RNA. In some embodiments, the template RNA is not covalently linked to a guide RNA. In some embodiments, at least a portion of the template RNA hybridizes to the target nucleic acid. In some embodiments, the target nucleic acid is a dsDNA molecule. In some embodiments, at least a portion of the template RNA hybridizes to a first strand of the target nucleic acid and at least a portion of the guide RNA hybridizes to a second strand of the target nucleic acid. In some embodiments, the pegRNA comprises: a guide RNA comprising a second region that is bound by the effector protein, and a first region comprising a spacer sequence that is complementary to a target sequence of the dsDNA molecule; and a template RNA comprising a primer binding sequence that hybridizes to a primer sequence of the dsDNA molecule that is formed when target nucleic acid is nicked, and a template sequence that is complementary to at least a portion of the target sequence of the dsDNA molecule with the exception of at least one nucleotide. In some embodiments, the at least one nucleotide is incorporated into the target nucleic acid by activity of the RT editing enzyme, thereby modifying the target nucleic acid. In some embodiments, the spacer sequence is complementary to the target sequence on a target strand of the dsDNA molecule. In some embodiments, the spacer sequence is complementary to the target sequence on a non-target strand of the dsDNA molecule. In some embodiments, the primer binding sequence hybridizes to a terminal portion of the non-target strand of the target nucleic acid (e.g., dsDNA) that is nicked. In some embodiments, the primer binding sequence hybridizes to a terminal portion of the target strand of the target nucleic acid (e.g., dsDNA) that is nicked. In some embodiments, the target strand is nicked. In some embodiments, the non-target strand is nicked.

### Nucleic Acid Modification Activity

In some embodiments, effector partners have enzymatic activity that modifies a nucleic acid, such as a target nucleic acid. In some embodiments, the target nucleic acid comprises or consist of a ssRNA, dsRNA, ssDNA, or a dsDNA. Examples of enzymatic activity that modifies the target nucleic acid include, but are not limited to: nuclease activity, which comprises the enzymatic activity of an enzyme which allows the enzyme to cleave the phosphodiester bonds between the nucleotide subunits of nucleic acids, such as that provided by a restriction enzyme, or a nuclease (e.g., FokI nuclease); methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants)); demethylase activity such as that provided by a demethylase (e.g., Ten-Eleven Translocation (TET) dioxygenase 1 (TET1CD), TET1, DME, DML1, DML2, ROS1); DNA repair activity; DNA damage (e.g., oxygenation) activity; deamination activity such as that provided by a deaminase (e.g., a cytosine deaminase enzyme such as rat APOBEC1); dismutase activity; alkylation activity; depurination activity; oxidation activity; pyrimidine dimer forming activity; integrase activity such as that provided by an integrase and/or resolvase (e.g., Gin invertase such as the hyperactive mutant of the Gin invertase, GinH106Y, human immunodeficiency virus type 1 integrase (IN), Tn3 resolvase); transposase activity; recombinase activity such as that provided by a recombinase (e.g., catalytic domain of Gin recombinase); polymerase activity; ligase activity; helicase activity; photolyase activity; and glycosylase activity.

In some embodiments, effector partners target a ssRNA, dsRNA, ssDNA, or a dsDNA. In some embodiments, effector partners target ssRNA. Non-limiting examples of effector partners for targeting ssRNA include, but are not limited to, splicing factors (e.g., RS domains); protein translation components (e.g., translation initiation, elongation and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; and RNA-binding proteins.

It is understood that an effector partner may include an entire protein, or in some embodiments, may include a fragment of the protein (e.g., a functional domain). In some embodiments, the functional domain binds or interacts with a nucleic acid, such as ssRNA, including intramolecular and/or intermolecular secondary structures thereof (e.g., hairpins, stem-loops, etc.). The functional domain may interact transiently or irreversibly, directly, or indirectly. In some embodiments, a functional domain comprises a region of one or more amino acids in a protein that is required for an activity of the protein, or the full extent of that activity, as measured in an in vitro assay. Activities include but are not limited to nucleic acid binding, nucleic acid editing, nucleic acid mutating, nucleic acid modifying, nucleic acid, cleaving, protein binding or combinations thereof. The absence of the functional domain, including mutations of the functional domain, would abolish or reduce activity.

Accordingly, effector partners may comprise a protein or domain thereof selected from: endonucleases (e.g., RNase III, the CRR22 DYW domain, Dicer and PIN (PilT N-terminus); exonucleases such as XRN-1 or Exonuclease T; SMG5 and SMG6; domains responsible for stimulating RNA cleavage (e.g., CPSF, CstF, CFIm and CFIIm); deadenylases such as HNT3; protein domains responsible for nonsense mediated RNA decay (e.g., UPF1, UPF2, UPF3, UPF3b, RNP S1, Y14, DEK, REF2 and SRm160); protein domains responsible for stabilizing RNA (e.g., PABP); proteins and protein domains responsible for polyadenylation of RNA (e.g., PAP1, GLD-2 and Star-PAP); proteins and protein domains responsible for polyuridinylation of RNA (e.g., CID1 and terminal uridylate transferase); and other suitable domains that affect nucleic acid modifications.

In some embodiments, effector partner comprises a chromatin-modifying enzyme. In some embodiments, the effector partner chemically modifies a target nucleic acid, for example by methylating, demethylating, or acetylating the target nucleic acid in a sequence specific or non-specific manner.

### Base Editing Enzymes

In some embodiments, effector partners edit a nucleobase of a target nucleic acid. Such effector partner may be referred to as a base editing enzyme. In some embodiments, a base editing enzyme variant that differs from a naturally occurring base editing enzyme, but it is understood that any reference to a base editing enzyme herein also refers to a base editing enzyme variant. In some embodiments, the base editing enzyme edits a base on a target strand of the target nucleic acid. In some embodiments, the base editing enzyme edits a base on a non-target strand of the target nucleic acid.

In some embodiments, a base editor is a system comprising an effector protein and a base editing enzyme. In some embodiments, the base editor comprises a base editing enzyme and an effector protein as independent components. In some embodiments, the base editor comprises a fusion protein comprising a base editing enzyme fused or linked to an effector protein. In some embodiments, the amino terminus of the effector partner is linked to the carboxy terminus of the effector protein by the linker. In some embodiments, the carboxy terminus of the effector partner is linked to the amino terminus of the effector protein by the linker. The base editor may be functional when the effector protein is coupled to a guide nucleic acid. The base editor may be functional when the effector protein is coupled to a target nucleic acid. The guide nucleic acid imparts sequence specific activity to the base editor. By way of non-limiting example, the effector protein comprises a catalytically inactive effector protein (e.g., a catalytically inactive variant of an effector protein described herein). Also, by way of non-limiting example, the base editing enzyme comprises deaminase activity. Additional base editors are described herein.

In some embodiments, base editing enzymes catalyzes editing (e.g., a chemical modification) of a nucleobase of a nucleic acid molecule, such as DNA or RNA (single stranded or double stranded). In some embodiments, a base editing enzyme and, therefore, a base editor is capable of converting an existing nucleobase to a different nucleobase, such as: an adenine (A) to guanine (G); cytosine (C) to thymine (T); cytosine (C) to guanine (G); uracil (U) to cytosine (C); guanine (G) to adenine (A); hydrolytic deamination of an adenine or adenosine, or methylation of cytosine (e.g., CpG, CpA, CpT or CpC). In some embodiments, base editing enzymes edit a nucleobase on a ssDNA. In some embodiments, base editing enzymes edit a nucleobase on both strands of dsDNA. In some embodiments, base editing enzymes edit a nucleobase of an RNA.

A base editing enzyme itself may or may not bind to the nucleic acid molecule containing the nucleobase. In some embodiments, upon binding to its target locus in the target nucleic acid (e.g., a DNA molecule), base pairing between the guide nucleic acid and target strand leads to displacement of a small segment of ssDNA in an “R-loop”. In some embodiments, DNA bases within the R-loop are edited by the base editing enzyme having the deaminase enzyme activity. In some embodiments, base editing systems for improved efficiency in eukaryotic cells comprise a base editing enzyme, and a catalytically inactive effector protein that may generate a nick in the non-edited strand and induce repair of the non-edited strand using the edited strand as a template.

In some embodiments, a base editing enzyme comprises a deaminase enzyme. Exemplary deaminases are described in US20210198330, WO2021041945, WO2021050571A1 and WO2020123887, all of which are incorporated herein by reference in their entirety. Exemplary deaminase domains are described WO 2018027078 and WO2017070632, and each are hereby incorporated in its entirety by reference. Also, additional exemplary deaminase domains are described in Komor et al., Nature, 533, 420-424 (2016); Gaudelli et al., Nature, 551, 464-471 (2017); Komor et al., Science Advances, 3: eaao4774 (2017) and Rees et al., Nat Rev Genet. 2018 December; 19(12):770-788. doi: 10.1038/s41576-018-0059-1, which are hereby incorporated by reference in their entirety. In some embodiments, the deaminase functions as a monomer. In some embodiments, the deaminase functions as heterodimer with an additional protein. In some embodiments, base editing enzymes comprise a DNA glycosylase inhibitor (e.g., an uracil glycosylase inhibitor (UGI) or uracil N-glycosylase (UNG)). In some embodiments, the effector partner is a deaminase, e.g., ADAR1/2, ADAR-2, AID, or any functional variant thereof.

In some embodiments, the base editor is a cytosine base editor (CBE), wherein the base editing enzyme is a cytosine base editing enzyme. In some embodiments, the cytosine base editing enzyme and, therefore, CBE convert a cytosine to a thymine. In some embodiments, a cytosine base editing enzyme accept ssDNA as a substrate but is not capable of cleaving dsDNA, wherein the CBE comprises a catalytically inactive effector protein. In some embodiments, when bound to its cognate DNA, the catalytically inactive effector protein of the CBE performs local denaturation of the DNA duplex to generate an R-loop in which the DNA strand not paired with a guide nucleic acid exists as a disordered single-stranded bubble. In some embodiments, the catalytically inactive effector protein generated ssDNA R-loop enables the CBE to perform efficient and localized cytosine deamination in vitro. In some embodiments, deamination activity is exhibited in a window of 4 to 10 base pairs. In some embodiments, the catalytically inactive effector protein presents a target site to the cytosine base editing enzyme in high effective molarity, which may enable the CBE to deaminate cytosines located in a variety of different sequence motifs, with differing efficacies. In some embodiments, the CBE mediates RNA-programmed deamination of target cytosines in vitro or in vivo. In some embodiments, the cytosine base editing enzyme is a cytidine deaminase. In some embodiments, the cytosine base editing enzyme is a cytosine base editing enzyme described by Koblan et al. (2018) Nature Biotechnology 36:848-846; Komor et al. (2016) Nature 533:420-424; Koblan et al. (2021) “Efficient C·G-to-G·C base editors developed using CRISPRi screens, target-library analysis and machine learning,” Nature Biotechnology; Kurt et al. (2021) Nature Biotechnology 39:41-46; Zhao et al. (2021) Nature Biotechnology 39:35-40; and Chen et al. (2021) Nature Communications 12:1384, all incorporated herein by reference.

In some embodiments, the effector partner comprises a uracil glycosylase inhibitor (UGI) . . . . In some embodiments, the CBEs described herein further comprises a UGI. Base excision repair (BER) of U·G in DNA is initiated by a uracil N-glycosylase (UNG), which recognizes a U·G mismatch generated by a CBE and cleaves the glyosidic bond between a uracil and a deoxyribose backbone of DNA. BER results in the reversion of the U·G intermediate created by the cytosine base editing enzyme back to a C·G base pair. Accordingly, the UNG may be inhibited by fusion of a UGI to the effector protein. In some embodiments, the UGI is a small protein from bacteriophage PBS. In some embodiments, the UGI is a DNA mimic that potently inhibits both human and bacterial UNG. In some embodiments, the UGI inhibitor is any protein or polypeptide that inhibits UNG.

In some embodiments, the CBEs described herein mediates efficient base editing in bacterial cells and moderately efficient editing in mammalian cells, enabling conversion of a C·G base pair to a T·A base pair through a U·G intermediate. In some embodiments, the CBE is modified to increase base editing efficiency while editing more than one strand of DNA.

In some embodiments, the CBEs described herein nicks a non-edited DNA strand. In some embodiments, the non-edited DNA strand nicked by the CBE biases cellular repair of a U·G mismatch to favor a U·A outcome, elevating base editing efficiency.

In some embodiments, a base editor described herein comprising one or more base editing enzymes (e.g., APOBEC1, nickase and UGI) efficiently edits in mammalian cells, while minimizing frequency of non-target indels. In some embodiments, base editors do not comprise a functional fragment of the base editing enzyme. In some embodiments, base editors do not comprise a function fragment of a UGI, where such a fragment excises a uracil residue from DNA by cleaving an N-glycosidic bond.

In some embodiments, the effector partner comprises a non-protein uracil-DNA glycosylase inhibitor (npUGI). In some embodiments, the npUGI is selected from a group of small molecule inhibitors of uracil-DNA glycosylase (UDG), or a nucleic acid inhibitor of UDG. In some embodiments, the npUGI is a small molecule derived from uracil. Examples of small molecule non-protein uracil-DNA glycosylase inhibitors, fusion proteins and Cas-CRISPR systems comprising base editing activity are described in WO2021087246, which is incorporated by reference in its entirety.

In some embodiments, the base editor is a cytosine base editor, wherein the based editing enzyme is a cytosine base editing enzyme. In some embodiments, the cytosine base editing enzyme is a cytidine deaminase. In some embodiments, the base editor comprising the cytidine deaminase is generated by ancestral sequence reconstruction as described in WO2019226953, which is hereby incorporated by reference in its entirety. Non-limiting exemplary cytidine deaminases suitable for use with effector proteins described herein include: APOBEC1, APOBEC2, APOBEC3C, APOBEC3D, APOBEC3F, APOBEC3G, APOBEC3H, APOBEC4, APOBEC3A, BEI (APOBEC1-XTEN-dCas9), BE2 (APOBEC1-XTEN-dCas9-UGI), BE3 (APOBEC1-XTEN-dCas9 (A840H)-UGI), BE3-Gam, saBE3, saBE4-Gam, BE4, BE4-Gam, saBE4 and saBE4-Gam as described in WO2021163587, WO2021087246, WO2021062227 and WO2020123887, which are incorporated herein by reference in their entirety.

In some embodiments, a base editor is a cytosine to guanine base editor (CGBE), wherein the base editing enzyme is a cytosine to guanine base editing enzyme. In some embodiments, the cytosine to guanine base editing enzyme and, therefore, the CGBE convert a cytosine to a guanine.

In some embodiments, a base editor is an adenine base editor (ABE), wherein the base editing enzyme is an adenine base editing enzyme. In some embodiments, the adenine base editing enzyme and, therefore, the ABE convert an adenine to a guanine. In some embodiments, the adenine base editing enzyme converts an A·T base pair to a G·C base pair. In some embodiments, the adenine base editing enzyme converts a target A·T base pair to G·C in vivo or in vitro. In some embodiments, the adenine base editing enzymes provided herein reverse spontaneous cytosine deamination, which has been linked to pathogenic point mutations. In some embodiments, the adenine base editing enzymes provided herein enable correction of pathogenic SNPs (˜47% of disease-associated point mutations). In some embodiments, the adenine comprises exocyclic amine that has been deaminated (e.g., resulting in altering its base pairing preferences). In some embodiments, deamination of adenosine yields inosine. In some embodiments, inosine exhibits the base-pairing preference of guanine in the context of a polymerase active site, although inosine in the third position of a tRNA anticodon pairs with A, U, or C in mRNA during translation. Non-limiting exemplary adenine base editing enzymes suitable for use with effector proteins described herein include: ABE8e, ABE8.20m, APOBEC3A, Anc APOBEC (a.k.a. AncBE4Max) and BtAPOBEC2. Non-limiting exemplary ABEs suitable for use herein include: ABE7, ABE8.1m, ABE8.2m, ABE8.3m, ABE8.4m, ABE8.5m, ABE8.6m, ABE8.7m, ABE8.8m, ABE8.9m, ABE8.10m, ABE8.11m, ABE8.12m, ABE8.13m, ABE8.14m, ABE8.15m, ABE8.16m, ABE8.17m, ABE8.18m, ABE8.19m, ABE8.20m, ABE8.21m, ABE8.22m, ABE8.23m, ABE8.24m, ABE8.1d, ABE8.2d, ABE8.3d, ABE8.4d, ABE8.5d, ABE8.6d, ABE8.7d, ABE8.8d, ABE8.9d, ABE8.10d, ABE8.11d, ABE8.12d, ABE8.13d, ABE8.14d, ABE8.15d, ABE8.16d, ABE8.17d, ABE8.18d, ABE8.19d, ABE8.20d, ABE8.21d, ABE8.22d, ABE8.23d and ABE8.24d. In some embodiments, the adenine base editing enzyme is an adenine base editing enzyme described in Chu et al., (2021) The CRISPR Journal 4:2:169-177, incorporated herein by reference. In some embodiments, the adenine deaminase is an adenine deaminase described by Koblan et al. (2018) Nature Biotechnology 36:848-846, incorporated herein by reference. In some embodiments, the adenine base editing enzyme is an adenine base editing enzyme described by Tran et al. (2020) Nature Communications 11:4871.

In some embodiments, the ABE described herein is targets polyA signals, splice site acceptors and start codons. In some embodiments, the ABE cannot create stop codons for knock-down.

In some embodiments, an adenine base editing enzyme is an adenosine deaminase.

Non-limiting exemplary adenosine base editors suitable for use herein include ABE9. In some embodiments, the ABE comprises an engineered adenosine deaminase enzyme acts on ssDNA. The engineered adenosine deaminase enzyme may be an adenosine deaminase variant that differs from a naturally occurring deaminase. Relative to the naturally occurring deaminase, the adenosine deaminase variant may comprise one or more amino acid alteration, including a V82S alteration, a T166R alteration, a Y147T alteration, a Y147R alteration, a Q154S alteration, a Y123H alteration, a Q154R alteration, or a combination thereof.

In some embodiments, the base editor comprises an adenine deaminase (e.g., TadA). In some embodiments, the adenosine deaminase is a TadA monomer (e.g., Tad*7.10, TadA*8 or TadA*9). In some embodiments, the adenosine deaminase is a TadA*8 variant (e.g., any one of TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24 as described in WO2021163587 and WO2021050571, which are each hereby incorporated by reference in its entirety). In some embodiments, the base editor comprises TadA.

In some embodiments, a base editing enzyme is a deaminase dimer. In some embodiments, the ABE comprises the effector protein, the adenine base editing enzyme and the deaminase dimer. In some embodiments, the deaminase dimer comprises an adenosine deaminase. In some embodiments, the deaminase dimer comprises TadA and a suitable adenine base editing enzyme including an: ABE8e, ABE8.20m, APOBEC3A, Anc APOBEC (a.k.a. AncBE4Max), BtAPOBEC2 and variants thereof. In some embodiments, the adenine base editing enzyme is fused to amino-terminus or the carboxy-terminus of TadA.

In some embodiments, a base editor is an RNA base editor, wherein the base editing enzyme is an RNA base editing enzyme. In some embodiments, the RNA base editing enzyme comprises an adenosine deaminase. In some embodiments, ADAR proteins bind to RNAs and alter their sequence by changing an adenosine into an inosine. In some embodiments, RNA base editors comprise an effector protein that is activated by or binds RNA.

In some embodiments, base editing enzymes and, therefore, base editors are used for treating a subject having or a subject suspected of having a disease related to a gene of interest. In some embodiments, base editing enzymes and, therefore, base editors are useful for treating a disease or a disorder caused by a point mutation in a gene of interest. In some embodiments, compositions, systems and methods described herein comprise a base editor and a guide nucleic acid, wherein the base editor comprises an effector protein and a base editing enzyme, and wherein the guide nucleic acid directs the base editor to a sequence in a target gene.

### Protein Modification Activity

In some embodiments, an effector partner provides enzymatic activity that modifies a protein associated with a target nucleic acid. The protein may be a histone, an RNA binding protein, or a DNA binding protein. Examples of such protein modification activities include: methyltransferase activity, such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, SET1A, SET1B, MLL1 to 5, ASH1, SYMD2, NSD1, DOT1L, Pr-SET7/8, SUV4-20H1, EZH2, RIZ1); demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3); acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK); deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11); kinase activity; phosphatase activity; ubiquitin ligase activity; deubiquitinating activity; adenylation activity; deadenylation activity; SUMOylating activity; deSUMOylating activity; ribosylation activity; deribosylation activity; myristoylation activity; and demyristoylation activity.

### CRISPRa Fusions and CRISPRi Fusions

In some embodiments, effector partners include, but are not limited to, a protein that directly and/or indirectly provides for increased or decreased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). In some embodiments, effector partners that increase or decrease transcription include a transcription activator domain or a transcription repressor domain, respectively.

In some embodiments, effector partners activate or increase expression of a target nucleic acid. In some embodiments, effector partners increase expression of the target nucleic acid relative to its expression in the absence of the effector partners. Relative expression, including transcription and RNA levels, may be assessed, quantified and compared, e.g., by RT-qPCR. In some embodiments, effector partners comprise a transcriptional activator. In some embodiments, the transcriptional activators promote transcription by: recruitment of other transcription factor proteins; modification of target DNA such as demethylation; recruitment of a DNA modifier; modulation of histones associated with target DNA; recruitment of a histone modifier such as those that modify acetylation and/or methylation of histones; or a combination thereof.

Non-limiting examples of effector partners that promote or increase transcription include: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SETIA, SET1B, MLL1 to 5, ASH1, SYMD2, NSD1; histone lysine demethylases such as JHDM2a/b, UTX, JMJD3; histone acetyltransferases such as GCN5, PCAF, CBP, p300, TAF1, TIP60/PLIP, MOZ/MYST3, MORF/MYST4, SRC1, ACTR, P160, CLOCK; and DNA demethylases such as Ten-Eleven Translocation (TET) dioxygenase 1 (TET1CD), TET1, DME, DML1, DML2 and ROS1; and functional domains thereof. Other non-limiting examples of suitable effector partners include: proteins and protein domains responsible for stimulating translation (e.g., Staufen); proteins and protein domains responsible for (e.g., capable of) modulating translation (e.g., translation factors such as initiation factors, elongation factors, release factors, etc., e.g., eIF4G); proteins and protein domains responsible for stimulation of RNA splicing (e.g., Serine/Arginine-rich (SR) domains); and proteins and protein domains responsible for stimulating transcription (e.g., CDK7 and HIV Tat).

In some embodiments, effector partners inhibit or reduce expression of a target nucleic acid. In some embodiments, effector partners reduce expression of the target nucleic acid relative to its expression in the absence of the effector partners. Relative expression, including transcription and RNA levels, may be assessed, quantified and compared, e.g., by RT-qPCR. In some embodiments, effector partners comprise a transcriptional repressor. In some embodiments, the transcriptional repressors inhibit transcription by: recruitment of other transcription factor proteins; modification of target DNA such as methylation; recruitment of a DNA modifier; modulation of histones associated with target DNA; recruitment of a histone modifier such as those that modify acetylation and/or methylation of histones; or a combination thereof.

Non-limiting examples of effector partners that decrease or inhibit transcription include: transcriptional repressors such as the Krüppel associated box (KRAB or SKD); KOX1 repression domain; the Mad mSIN3 interaction domain (SID); the ERF repressor domain (ERD), the SRDX repression domain (e.g., for repression in plants); histone lysine methyltransferases such as Pr-SET7/8, SUV4-20H1, RIZ1 and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY; histone lysine deacetylases such as HDAC1, HDAC2, HDAC3, HDAC8, HDAC4, HDAC5, HDAC7, HDAC9, SIRT1, SIRT2, HDAC11; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants); and periphery recruitment elements such as Lamin A and Lamin B; and functional domains thereof. Other non-limiting examples of suitable effector partners include: proteins and protein domains responsible for repressing translation (e.g., Ago2 and Ago4); proteins and protein domains responsible for repression of RNA splicing (e.g., PTB, Sam68 and hnRNP A1); proteins and protein domains responsible for reducing the efficiency of transcription (e.g., FUS (TLS)).

In some embodiments, fusion proteins comprising the described effector partners and an effector protein are referred to as CRISPRa fusions, wherein the effector partners activate or increase expression of a target nucleic acid. In some embodiments, fusion proteins comprising the described effector partners and an effector protein are referred to as CRISPRi fusions, wherein the effector partners inhibit or reduce expression of a target nucleic acid. In some embodiments, fusion proteins are targeted by a guide nucleic acid (e.g., guide RNA) to a specific location in a target nucleic acid and exert locus-specific regulation such as blocking RNA polymerase binding to a promoter (which selectively inhibits transcription activator function) and/or changes a local chromatin status (e.g., when a fusion sequence is used that edits the target nucleic acid or modifies a protein associated with the target nucleic acid). In some embodiments, the modifications are transient (e.g., transcription repression or activation). In some embodiments, the modifications are inheritable. For example, epigenetic modifications made to a target nucleic acid, or to proteins associated with the target nucleic acid, e.g., nucleosomal histones, in a cell, can be observed in a successive generation.

In some embodiments, effector partner comprises an RNA splicing factor. The RNA splicing factor may be used (in whole or as fragments thereof) for modular organization, with separate sequence-specific RNA binding modules and splicing effector domains. In some embodiments, the RNA splicing factors comprise members of the Serine/Arginine-rich (SR) protein family containing N-terminal RNA recognition motifs (RRMs) that bind to exonic splicing enhancers (ESEs) in pre-mRNAs and C-terminal RS domains that promote exon inclusion. In some embodiments, a hnRNP Al binds to exonic splicing silencers (ESSs) through its RRM domains and inhibits exon inclusion through a C-terminal Glycine-rich domain. In some embodiments, the RNA splicing factors regulate alternative use of splice site (ss) by binding to regulatory sequences between two alternative sites. For example, in some embodiments, ASF/SF2 recognize ESEs and promote the use of intron proximal sites, whereas hnRNP Al binds to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. Long splicing isoform Bcl-xL is a potent apoptosis inhibitor expressed in long-lived postmitotic cells and is up-regulated in many cancer cells, protecting cells against apoptotic signals. Short isoform Bcl-xS is a pro-apoptotic isoform and expressed at high levels in cells with a high turnover rate (e.g., developing lymphocytes). A ratio of the two Bcl-x splicing isoforms is regulated by multiple c\acute{ω}-elements that are located in either core exon region or exon extension region (i.e., between the two alternative 5′ splice sites). For more examples, see WO2010075303, which is hereby incorporated by reference in its entirety.

### Recombinases

In some embodiments, effector partners comprise a recombinase. In some embodiments, provided herein is a recombinase system comprising effector proteins described herein and the recombinase. In some embodiments, the effector proteins have reduced nuclease activity or no nuclease activity. In some embodiments, the recombinase is a site-specific recombinase.

In some embodiments, the recombinase system comprises a catalytically inactive effector protein, wherein the recombinase can be a site-specific recombinase. Such systems can be used for site-directed transgene insertion. Non-limiting examples of site-specific recombinases include a tyrosine recombinase (e.g., Cre, Flp or lambda integrase), a serine recombinase (e.g., gamma-delta resolvase, Tn3 resolvase, Sin resolvase, Gin invertase, Hin invertase, Tn5044 resolvase, IS607 transposase and integrase), or mutants or variants thereof. In some embodiments, the recombinase is a serine recombinase. Non-limiting examples of serine recombinases include gamma-delta resolvase, Tn3 resolvase, Sin resolvase, Gin invertase, Hin invertase, Tn5044 resolvase, IS607 transposase and IS607 integrase. In some embodiments, the site-specific recombinase is an integrase. Non-limiting examples of integrases include: Bxb1, wBeta, BL3, phiR4, A118, TG1, MR11, phi370, SPBc, TP901-1, phiRV, FC1, K38, phiBT1 and phiC31. Further discussion and examples of suitable recombinase effector partners are described in U.S. Pat. No. 10,975,392, which is incorporated herein by reference in its entirety. In some embodiments, the fusion protein comprises a linker that links the recombinase to the Cas-CRISPR domain of the effector protein. In some embodiments, the linker is The-Ser.

### Linkers for Peptides

In some embodiments, a linker comprises a bond or molecule that links a first polypeptide to a second polypeptide. Accordingly, in some embodiments, effector proteins, effector partners, or combinations thereof are connected by linkers. The linker may comprise or consist of a covalent bond. The linker may comprise or consist of a chemical group. In some embodiments, the linker comprises an amino acid. In some embodiments, a peptide linker comprises at least two amino acids linked by an amide bond. In general, the linker connects a terminus of the effector protein to a terminus of the effector partner. In some embodiments, carboxy terminus of the effector protein is linked to the amino terminus of the fusion effector. In some embodiments, carboxy terminus of the effector partner is linked to the amino terminus of the effector protein. In some embodiments, the effector protein and the effector partner are directly linked by a covalent bond.

In some embodiments, linkers comprise one or more amino acids. In some embodiments, linker is a protein. In some embodiments, a terminus of the effector protein is linked to a terminus of the effector partner through an amide bond. In some embodiments, a terminus of the effector protein is linked to a terminus of the effector partner through a peptide bond. In some embodiments, linkers comprise an amino acid. In some embodiments, linkers comprise a peptide. In some embodiments, an effector protein is coupled to an effector partner by a linker protein. In some embodiments, the linkers have any of a variety of amino acid sequences. In some embodiments, the linkers comprise a region of rigidity (e.g., beta sheet, alpha helix), a region of flexibility, or any combination thereof. In some embodiments, the linker comprises small amino acids, such as glycine and alanine, that impart high degrees of flexibility. The ordinarily skilled artisan will recognize that design of a peptide conjugated to any desired element may include linkers that are all or partially flexible, such that the linker may include a flexible linker as well as one or more portions that confer less flexible structure. Suitable linkers include proteins of 4 linked amino acids to 40 linked amino acids in length, or between 4 linked amino acids and 25 linked amino acids in length. In some embodiments, linked amino acids described herein comprise at least two amino acids linked by an amide bond.

Linkers may be produced by using synthetic, linker-encoding oligonucleotides to couple proteins, or may be encoded by a nucleic acid sequence encoding a fusion protein (e.g., an effector protein coupled to an effector partner). In some embodiments, the linker is from 1 to 300, from 1 to 250, from 1 to 200, from 1 to 150, from 1 to 100, from 1 to 50, from 1 to 25, from 1 to 10, from 10 to 300, from 10 to 250, from 10 to 200, from 10 to 150, from 10 to 100, from 10 to 50, from 10 to 25, from 25 to 300, from 25 to 250, from 25 to 200, from 25 to 150, from 25 to 100, from 25 to 50, from 50 to 300, from 50 to 250, from 50 to 200, from 50 to 150, from 50 to 100, from 100 to 300, from 100 to 250, from 100 to 200, from 100 to 150, from 150 to 300, from 150 to 250, from 150 to 200, from 200 to 300, from 200 to 250, or from 250 to 300 amino acids in length. In some embodiments, the linker is from 1 to 100 amino acids in length. In some embodiments, the linker is more 100 amino acids in length. In some embodiments, the linker is from 10 to 27 amino acids in length. In some embodiments, linker proteins include glycine polymers (G)ₙ, glycine-serine polymers (including, for example,

(SEQ ID NO: 369)
(GS)ₙ, GSGGSₙ,
(SEQ ID NO: 370)
GGSGGSₙ
and
(SEQ ID NO: 371)
GGGSₙ,

where n is an integer of at least one), glycine-alanine polymers and alanine-serine polymers. In some embodiments, linkers comprise amino acid sequences including, but not limited to, GGSG (SEQ ID NO: 378), GGSGG (SEQ ID NO: 372), GSGSG (SEQ ID NO: 373), GSGGG (SEQ ID NO: 374), GGGSG (SEQ ID NO: 375) and GSSSG (SEQ ID NO: 376). In some embodiments, the linker comprises one or more repeats a tri-peptide GGS. In some embodiments, the linker is an XTEN linker. In some embodiments, the XTEN linker is an XTEN80 linker. In some embodiments, the XTEN linker is an XTEN20 linker. In some embodiments, the XTEN20 linker has an amino acid sequence of

(SEQ ID NO: 209)
GSGGSPAGSPTSTEEGTSESATPGSG.

In some embodiments, linkers do not comprise an amino acid. In some embodiments, linkers do not comprise a peptide. In some embodiments, linkers comprise a nucleotide, a polynucleotide, a polymer, or a lipid. In some embodiments, linker is a polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacrylamide, polyacrylate, polycyanoacrylates, lipid polymers, chitins, hyaluronic acid, heparin, or an alkyl linker.

In some embodiments, a linker is recognized and cleaved by a protein described herein. In some embodiments, a linker comprises a recognition sequence that is recognized and cleaved by the protein. In some embodiments, a guide nucleic acid comprises an aptamer, which serves a similar function as a linker, bringing an effector protein and an effector partner protein into proximity. The aptamer can functionally connect two proteins (e.g., effector protein, effector partner) by interacting non-covalently with both, thereby bringing both proteins into proximity of the guide nucleic acid. In some embodiments, the first protein and/or the second protein comprise or is covalently linked to an aptamer binding moiety. In some embodiments, the aptamer is a short single stranded DNA (ssDNA) or RNA (ssRNA) molecule capable of being bound by the aptamer binding moiety. In some embodiments, the aptamer is a molecule that mimics antibody binding activity and is classified as a chemical antibody. In some instances, the aptamer described herein refers to artificial oligonucleotides that bind one or more specific molecules. In some embodiments, aptamers exhibit a range of affinities (K_{D} in the pM to μM range) with little or no off-target binding.

### Fusion Proteins

In some embodiments, compositions, systems and methods comprise a fusion protein or uses thereof. A fusion protein generally comprises at least one effector protein, at least one effector partner, or a combination thereof. In some embodiments, the effector partner is fused or linked to the effector protein. In some embodiments, the effector partner is fused to the N-terminus of the effector protein. In some embodiments, the effector partner is fused to the C-terminus of the effector protein.

In some embodiments, the fusion protein comprising the effector partner is an effector protein. Accordingly, in such embodiments, the fusion protein can comprise at least two effector proteins that are same. In some embodiments, the fusion protein comprises at least two effector proteins that are different. Unless otherwise indicated, reference to effector proteins throughout the present disclosure include fusion proteins described herein.

In some embodiments, the fusion protein complexes with a guide nucleic acid and the complex interacts with the target nucleic acid, a non-target nucleic acid, or both. In some embodiments, the interaction comprises one or more of: recognition of a protospacer adjacent motif (PAM) sequence within the target nucleic acid by the effector protein, hybridization of the guide nucleic acid to the target nucleic acid, nicking of the target nucleic acid, modification of the target nucleic acid and/or the non-target nucleic acid by the fusion protein, or combinations thereof. In some embodiments, recognition of a PAM sequence within a target nucleic acid directs the modification activity of a fusion protein.

Modification activity of a fusion protein described herein may be nicking activity, binding activity, substitution activity and the like. Modification activity of a fusion protein may result in: nicking of a target nucleic acid (target or non-target strand), chemical modification of one or more nucleotides of a target nucleic acid (target or non-target strand) into an alternative nucleotide, substitution of one or more nucleotides of a target nucleic acid (target or non-target strand) with an alternative nucleotide, more than one of the foregoing, or any combination thereof. In some embodiments, an ability of a fusion protein to edit a target nucleic acid depends upon the effector protein being complexed with a guide nucleic acid, the guide nucleic acid being hybridized to a target sequence of the target nucleic acid, or combinations thereof. A target nucleic acid comprises a target strand and a non-target strand. Accordingly, in some embodiments, the fusion protein edits a target strand and/or a non-target strand of a target nucleic acid.

In some embodiments, the fusion proteins described herein comprises the effector protein described herein and the base editing enzyme described herein. In some embodiments, the fusion proteins edit a base on a non-target strand of the target nucleic acid. In some embodiments, the fusion proteins edit a base on a target strand of the target nucleic acid. In some embodiments, the fusion proteins are provided with an additional effector partner comprising a ssDNA binding protein. In some embodiments, the ssDNA binding protein prevents non-target strand editing by the fusion protein.

### Heterologous Peptides

In some embodiments, proteins (e.g., effector proteins, effector partners, fusion proteins or combinations thereof) described herein can be modified with the addition of one or more heterologous peptides. In some embodiments, the effector protein further comprises one or more heterologous peptides that are heterologous to the effector protein.

In some embodiments, a heterologous peptide comprises a subcellular localization signal. In some embodiments, a subcellular localization signal can be a nuclear localization signal (NLS). In some embodiments, the NLS facilitates localization of a nucleic acid, protein, or small molecule to the nucleus, when present in a cell that contains a nuclear compartment. TABLE 5 lists exemplary NLS sequences. In some embodiments, the subcellular localization signal is a nuclear export signal (NES), a sequence to keep the protein retained in the cytoplasm, a mitochondrial localization signal for targeting to the mitochondria, a chloroplast localization signal for targeting to a chloroplast, an ER retention signal and the like. In some embodiments, the protein described herein is not modified with a subcellular localization signal so that the protein is not targeted to the nucleus, which can be advantageous depending on the circumstance (e.g., when the target nucleic acid is an RNA that is present in the cytosol).

In some embodiments, a heterologous peptide comprises a chloroplast transit peptide (CTP), also referred to as a chloroplast localization signal or a plastid transit peptide, which targets the protein to a chloroplast. Chromosomal transgenes from bacterial sources may require a sequence encoding a CTP sequence fused to a sequence encoding an expressed protein (e.g., effector protein, effector partner) if the expressed protein is to be compartmentalized in the plant plastid (e.g., chloroplast). The CTP may be removed in a processing step during translocation into the plastid. Accordingly, localization of the protein to a chloroplast is often accomplished by means of operably linking a polynucleotide sequence encoding a CTP sequence to the 5′ region of a polynucleotide encoding the exogenous protein.

In some embodiments, the heterologous peptide is an endosomal escape peptide (EEP). An EEP is an agent that quickly disrupts the endosome in order to minimize the amount of time that a delivered molecule, such protein, spends in the endosome-like environment, and to avoid getting trapped in the endosomal vesicles and degraded in the lysosomal compartment. An exemplary EEP is recited in TABLE 5.

In some embodiments, the heterologous peptide is a cell penetrating peptide (CPP), also known as a Protein Transduction Domain (PTD). A CPP or PTD is a polypeptide, polynucleotide, carbohydrate, or organic or inorganic compound that facilitates traversing a lipid bilayer, micelle, cell membrane, organelle membrane, or vesicle membrane.

Further suitable heterologous peptide includes, but are not limited to, proteins (or fragments/domains thereof) that are boundary elements (e.g., CTCF), proteins and fragments thereof that provide periphery recruitment (e.g., Lamin A, Lamin B, etc.) and protein docking elements (e.g., FKBP/FRB, Pil1/Aby1, etc.).

In some embodiments, a heterologous peptide comprises a protein tag. In some embodiments, the protein tag is referred to as purification tag or a fluorescent protein. The protein tag may be detectable for use in detection of the protein and/or purification of the protein. Accordingly, in some embodiments, compositions, systems and methods comprise a protein tag or use thereof. Any suitable protein tag may be used depending on the purpose of its use. Non-limiting examples of protein tags include a fluorescent protein, a histidine tag, e.g., a 6XHis tag (SEQ ID NO: 377); a hemagglutinin (HA) tag; a FLAG tag; a Myc tag; and maltose binding protein (MBP). In some embodiments, the protein tag is a portion of MBP that can be detected and/or purified. Non-limiting examples of fluorescent proteins include green fluorescent protein (GFP), yellow fluorescent protein (YFP), red fluorescent protein (RFP), cyan fluorescent protein (CFP), mCherry and tdTomato.

A heterologous peptide may be located at or near the amino terminus (N-terminus) of the protein (e.g., effector protein, effector partner) disclosed herein. A heterologous peptide may be located at or near the carboxy terminus (C-terminus) of the proteins disclosed herein. In some embodiments, a heterologous peptide is located internally in the protein described herein (i.e., is not at the N- or C-terminus of the protein described herein) at a suitable insertion site.

In some embodiments, protein (e.g., an effector protein, an effector partner, or a fusion protein) described herein comprises 1, 2, 3, 4, 5, 6, 7, 8, 9, 10, or more heterologous peptide at or near the N-terminus, 1, 2, 3, 4, 5, 6, 7, 8, 9, 10, or more heterologous peptide at or near the C-terminus, or a combination of these (e.g., one or more heterologous peptide at the amino-terminus and one or more heterologous peptide at the carboxy terminus). When more than one heterologous peptide is present, each may be selected independently of the others, such that a single heterologous peptide may be present in more than one copy and/or in combination with one or more other heterologous peptide present in one or more copies. In some embodiments, a heterologous peptide is considered near the N- or C-terminus when the nearest amino acid of the heterologous peptide is within 1, 2, 3, 4, 5, 10, 15, 20, 25, 30, 40, 50, or more amino acids along the polypeptide chain from the N- or C-terminus.

In some embodiments, a heterologous peptide described herein comprises a heterologous peptide sequence recited in TABLE 5. In some embodiments, proteins described herein comprise any one of the proteins (e.g., effector proteins, effector partners, fusion proteins, or combinations thereof) described herein fused to one or more of the amino acid sequences recited in TABLE 5. In some embodiments, a heterologous peptide described herein is an effector partner as described en supra. For example, in some embodiments, effector proteins or fusion proteins thereof are covalently linked to a heterologous peptide or protein. In some embodiments, effector proteins or fusion proteins thereof are covalently linked to a heterologous peptide or protein via a linker molecule. In some embodiments, the effector protein is covalently linked to a heterologous peptide or protein, optionally via a linker molecule.

In some embodiments, proteins (e.g., effector protein, effector partner, or fusion protein) described herein are encoded by a codon optimized nucleic acid. In some embodiments, a nucleic acid sequence encoding the protein described herein, is codon optimized. In some embodiments, the proteins described herein is codon optimized for expression in a specific cell, for example, a bacterial cell, a plant cell, a eukaryotic cell, an animal cell, a mammalian cell, or a human cell. In some embodiments, the effector protein is codon optimized for a human cell. In some embodiments, the effector partner is codon optimized for a human cell.

### Multimeric Complexes

Compositions, systems and methods of the present disclosure may comprise a multimeric complex or uses thereof, wherein the multimeric complex comprises one or more polypeptides (e.g., effector proteins, effector partners, fusion proteins, or combinations thereof) that non-covalently interact with one another. In some embodiments, the polypeptide functions as part of a multiprotein complex, including, for example, a complex having two or more polypeptides, including two or more of the same polypeptides (e.g., dimer or multimer). The polypeptide, when functioning in a multiprotein complex, may have only one functional activity (e.g., binding to a guide nucleic acid), while other polypeptides present in the multiprotein complex comprise (are capable of) the other functional activity (e.g., editing a target nucleic acid). In some embodiments, the polypeptide, when functioning in a multiprotein complex, have differing and/or complementary functional activity to other polypeptides in the multiprotein complex. In some embodiments, the polypeptide is modified to have increased substrate binding activity (e.g., substrate selectivity, specificity and/or affinity) relative to an unmodified counterpart wildtype polypeptide. In some embodiments, the substrate can be a double-stranded RNA (dsRNA), single stranded RNA (ssRNA), double stranded DNA (dsDNA), or single-stranded DNA (ssDNA).

A multimeric complex may comprise enhanced modification activity relative to the modification activity of a monomeric form thereof. For example, a multimeric complex comprising two polypeptides (e.g., in dimeric form) comprises greater nucleic acid binding affinity than that of either of the polypeptides provided in monomeric form. A multimeric complex may comprise one or more polypeptides fused to form a fusion protein, wherein the fusion protein comprises (is capable of) different activity than that of the one or more polypeptides. In another example, a multimeric complex comprises at least two polypeptides, wherein the multimeric complex may comprise greater nucleic acid binding affinity and/or modification activity than that of either of the polypeptide provided in monomeric form. A multimeric complex may have an affinity for a target sequence of a target nucleic acid and comprises (is capable of) catalytic activity (e.g., nicking, substituting or otherwise editing the nucleic acid) at or near the target sequence. Multimeric complexes may be activated when complexed with a guide nucleic acid. Multimeric complexes may be activated when complexed with a target nucleic acid. Multimeric complexes may be activated when complexed with a guide nucleic acid, a target nucleic acid, or a combination thereof. In some embodiments, the multimeric complex nicks the target nucleic acid.

Various aspects of the present disclosure include compositions and methods comprising multiple polypeptides (e.g., effector proteins, effector partners, fusion proteins, or combinations thereof) and uses thereof, respectively. For example, in some embodiments, two polypeptides are provided each targeting different nucleic acid sequences. Two polypeptides may target different types of nucleic acids (e.g., a first polypeptide may target double- and single-stranded nucleic acids and a second polypeptide may only target single-stranded nucleic acids). Two polypeptides may provide different types of activities (e.g., nucleic acid modification activity, nucleic acid expression modification activity). It is understood that when discussing the use of more than one polypeptide in compositions, systems and methods provided herein, the multimeric complex form is also described.

In some embodiments, multimeric complexes comprise at least one polypeptide (e.g., effector protein, effector partner, or fusion protein) as described herein. In some embodiments, the multimeric complex is a dimer comprising a first polypeptide and a second polypeptide. In some embodiments, the first polypeptide and the second polypeptide comprise identical amino acid sequences. In some embodiments, the first polypeptide and the second polypeptide comprise amino acid sequences that are at least 90%, at least 92%, at least 94%, at least 96%, at least 98% identical, at least 99%, or 100% identical to each other. In some embodiments, the first polypeptide and the second polypeptide comprise amino acid sequences that are at least 90%, at least 92%, at least 94%, at least 96%, at least 98% identical, at least 99%, or 100% similar to each other.

In some embodiments, the multimeric complex is a heterodimeric complex comprising at least two polypeptides (e.g., effector proteins, effector partners, fusion proteins, or combinations thereof) of different amino acid sequences. In some embodiments, the at least two polypeptides comprise two, three, four, five, six, seven, eight, nine, or ten polypeptides. In some embodiments, the multimeric complex is a heterodimeric complex comprising a first polypeptide and a second polypeptide, wherein the amino acid sequence of the first polypeptide is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% identical to the amino acid sequence of the second polypeptide.

In some embodiments, at least one effector protein of the multimeric complex comprises an amino acid sequence with at least 65%, at least 70%, at least 75%, at least 80%, at least 85%, at least 90%, at least 95% or 100% identity to any one of the sequences of TABLE 4. In some embodiments, each effector protein of the multimeric complex independently comprises an amino acid sequence with at least 65%, at least 70%, at least 75%, at least 80%, at least 85%, at least 90%, at least 95% or 100% identity to any one of the sequences of TABLE 4.

In some embodiments, the multimeric complex described herein targets polyA signals, splice site acceptors and start codons. In some embodiments, the multimeric complex cannot create stop codons for knock-down. In some embodiments, the multimeric complex is a dimer comprising two monomers, each independently selected from an effector protein, an effector partner and a fusion protein. In some embodiments, the dimer is formed due to non-covalent interactions between the monomers. In some embodiments, N- and C-termini of “formerly active” monomer is closer to 5′ region of non-target strand, while the termini of the “other” monomer is closer to 3′ region, which results in a larger editing window of the multimeric complex having a larger editing window on the non-target strand. In some embodiments, the multimeric complex has a lower editing window for a target strand due to inaccessibility for the effector partner or the fusion protein.

### Multimeric Complex Formation Modification Activity

In some embodiments, an effector partner inhibits the formation of a multimeric complex of an effector protein. Alternatively, the effector partner promotes the formation of a multimeric complex of the effector protein. In some embodiments, two of more effector partners forms a multimeric complex of the effector protein. In some embodiments, two of more effector partners forms a multimeric complex of the effector partner. In some embodiments, the effector partner comprises a Calcineurin A tag, which promotes formation of a multimeric complex (e.g., dimer) in the presence of Tacrolimus (FK506). In some embodiments, the effector partner comprises a SpyTag configured to dimerize or associate with another protein in a multimeric complex.

In some embodiments, the effector partner described herein comprises an effector protein. Accordingly, in some embodiments, the effector partner forms a multimeric protein with an effector protein, wherein the effector partner is an effector protein described herein. In some embodiments, the multimeric protein is a heteromeric protein. In such embodiments, the effector protein has decreased catalytic activity, also referred to as catalytically or enzymatically inactive, catalytically or enzymatically dead, as a dead protein or a dCas protein, whereas the effector partner is an effector protein described herein having catalytic activity as described herein. In some embodiments, such a multimeric protein comprises an effector protein having an enzymatically inactive domain (e.g., inactive nuclease domain). For example, a nuclease domain (e.g., RuvC domain) of the effector protein is deleted or mutated relative to a counterpart wildtype so that it is no longer functional or comprises reduced nuclease activity. In some embodiments, the catalytically inactive effector protein binds to a guide nucleic acid and/or a target nucleic acid but does not cleave the target nucleic acid by itself. In some embodiments, the catalytically inactive effector protein is associate with a guide nucleic acid to selectively target the multimeric protein to a target nucleic acid.

### Synthesis, Isolation and Assaying

Polypeptides (e.g., effector proteins, effector partners and fusion proteins) of the present disclosure may be synthesized, using any suitable method. In some embodiments, the polypeptides are produced in vitro or by eukaryotic cells or by prokaryotic cells. In some embodiments, the polypeptides are further processed by unfolding (e.g. heat denaturation, dithiothreitol reduction, etc.) and are further refolded, using any suitable method. In some embodiments, the nucleic acid(s) encoding the polypeptides described herein, the recombinant nucleic acid(s) described herein, the vectors described herein are produced in vitro or in vivo by eukaryotic cells or by prokaryotic cells.

Any suitable method of generating and assaying the polypeptides (e.g., effector proteins, effector partners and fusion proteins) described herein may be used. Such methods include, but are not limited to, site-directed mutagenesis, random mutagenesis, combinatorial libraries and other mutagenesis methods described herein (see, e.g., Sambrook et al., Molecular Cloning: A Laboratory Manual, Fourth Ed., Cold Spring Harbor Laboratory, Cold Spring Harbor (2012); Ausubel et al., Current Protocols in Molecular Biology, John Wiley and Sons, Baltimore, MD (1999); Gillman et al., Directed Evolution Library Creation: Methods and Protocols (Methods in Molecular Biology) Springer, 2nd ed (2014)). One non-limiting example of a method for preparing the polypeptide is to express recombinant nucleic acids encoding the polypeptide in a suitable microbial organism, such as a bacterial cell, a yeast cell, or other suitable cell, using methods well known in the art. Exemplary methods are also described in the Examples provided herein.

In some embodiments, a polypeptide provided herein is an isolated polypeptide (e.g., effector protein, effector partner and fusion protein). In some embodiments, the polypeptide is isolated and purified for use in compositions, systems and/or methods described herein. In some embodiments, methods described here include the step of isolating polypeptides described herein. Any suitable method to provide isolated polypeptides described herein may be used in the present disclosure, for example, recombinant expression systems, precipitation, gel filtration, ion-exchange, reverse-phase and affinity chromatography and the like. Other well-known methods are described in Deutscher et al., Guide to Protein Purification: Methods in Enzymology, 2nd Edition, Vol. 463, (Academic Press, (2009)). Alternatively, the isolated polypeptides of the present disclosure can be obtained using well-known recombinant methods (see, e.g., Sambrook et al., Molecular Cloning: A Laboratory Manual, Fourth Ed., Cold Spring Harbor Laboratory, Cold Spring Harbor (2012); and Ausubel et al., Current Protocols in Molecular Biology, John Wiley and Sons, Baltimore, MD (1999)). The methods and conditions for biochemical purification of a polypeptide described herein can be chosen by those skilled in the art and purification monitored, for example, by a functional assay.

In some embodiments, compositions, systems and methods described herein further comprise a purification tag that can be attached to a polypeptide (e.g., effector protein, effector partner and fusion protein), or a nucleic acid encoding the purification tag that can be attached to a nucleic acid encoding the polypeptide as described herein. In some embodiments, the purification tag is an amino acid sequence which can attach or bind with high affinity to a separation substrate and assist in isolating the polypeptide of interest from its environment, which is its biological source, such as a cell lysate. Attachment of the purification tag may be at the N or C terminus of the polypeptide. Furthermore, an amino acid sequence recognized by a protease or a nucleic acid encoding for an amino acid sequence recognized by a protease, such as TEV protease or the HRV3C protease may be inserted between the purification tag and the polypeptide, such that biochemical cleavage of the sequence with the protease after initial purification liberates the purification tag. Purification and/or isolation may be performed through high performance liquid chromatography (HPLC), exclusion chromatography, gel electrophoresis, affinity chromatography, or other purification technique. Non-limiting examples of purification tags are as described herein.

In some embodiments, polypeptides (e.g., effector proteins, effector partners and fusion proteins) described herein are isolated from cell lysate. In some embodiments, the compositions described herein comprise 20% or more by weight, 75% or more by weight, 95% or more by weight, or 99.5% or more by weight of the polypeptide, related to the method of preparation of compositions described herein and its purification thereof, wherein percentages are upon total polypeptide content in relation to contaminants. Thus, in some embodiments, the polypeptide is at least 80% pure, at least 85% pure, at least 90% pure, at least 95% pure, at least 98% pure, or at least 99% pure (e.g., free of contaminants, non-engineered proteins or other macromolecules, etc.).

### Protospacer Adjacent Motif (PAM) Sequences

Polypeptide (e.g., effector protein, effector partner and fusion protein) of the present disclosure may bind, nick and/or modify a target nucleic acid within or near a protospacer adjacent motif (PAM) sequence of the target nucleic acid. In some embodiments, the target nucleic acid is a double stranded nucleic acid comprising a target strand and a non-target strand. In some embodiments, binding, nicking and/or modifying occurs within 1, 2, 3, 4, 5, 6, 7, 8, 9, 10, 15, 20, 25, 30, 35, 40, 45, or 50 nucleotides of a 5′ or 3′ terminus of a PAM sequence. In some embodiments, effector protein described herein recognize a PAM sequence. In some embodiments, recognizing a PAM sequence comprises interacting with a sequence adjacent to the PAM. In some embodiments, a target nucleic acid comprises a target sequence that is adjacent to a PAM sequence. In some embodiments, the polypeptide does not require a PAM to bind, nick and/or modify a target nucleic acid.

In some embodiments, a target nucleic acid is a single stranded target nucleic acid comprising a target sequence. Accordingly, in some embodiments, the single stranded target nucleic acid comprises a PAM sequence described herein that is adjacent (e.g., within 1, 2, 3, 4, 5, 6, 7, 8, 9, 10, 15, 20, 25, 30, 35, 40, 45, or 50 nucleotides) or directly adjacent to the target sequence. In some embodiments, an RNP binds, cleaves and/or modifies the single stranded target nucleic acid.

In some embodiments, a target nucleic acid is a double stranded nucleic acid comprising a target strand and a non-target strand, wherein the target strand comprises a target sequence. In some embodiments, the PAM sequence is located on the target strand. In some embodiments, the PAM sequence is located on the non-target strand. In some embodiments, the PAM sequence described herein is adjacent (e.g., within 1, 2, 3, 4, 5, 6, 7, 8, 9, 10, 15, 20, 25, 30, 35, 40, 45, or 50 nucleotides) to the target sequence on the target strand or the non-target strand. In some embodiments, the PAM sequence is located 5′ of a reverse complement of the target sequence on the non-target strand. In some embodiments, such a PAM described herein is directly adjacent to the target sequence on the target strand or the non-target strand. In some embodiments, an RNP binds, nicks and/or modifies the target strand or the non-target strand. In some embodiments, an RNP recognizes the PAM sequence, hybridizes to a target sequence of the target nucleic acid and, optionally, modifies the target nucleic acid. In some embodiments, the RNP binds, nicks and/or modifies the target nucleic acid, wherein the RNP has recognized the PAM sequence, is hybridized to the target sequence of the target nucleic acid and, optionally, modifies the target nucleic acid.

In some embodiments, a polypeptide (e.g., an effector protein described herein, an effector partner described herein) or a multimeric complex thereof, recognizes a PAM on a target nucleic acid. In some embodiments, multiple polypeptides of the multimeric complex recognize a PAM on a target nucleic acid. In some embodiments, at least two of the multiple polypeptides recognize the same PAM sequence. In some embodiments, at least two of the multiple polypeptides recognize different PAM sequences. In some embodiments, only one polypeptide of the multimeric complex recognizes a PAM on a target nucleic acid.

An effector protein of the present disclosure, or a multimeric complex thereof, may bind, cleave, nick, or modify a target nucleic acid within or near a protospacer adjacent motif (PAM) sequence of the target nucleic acid. In some embodiments, binding, cleavage, nicking and/or modification occurs within 1, 2, 3, 4, 5, 6, 7, 8, 9 or 10 nucleotides of a 5′ or 3′ terminus of a PAM sequence.

In some embodiments, a PAM sequence provided herein comprises any one of the nucleotide sequences recited in TABLE 6. PAMs used in compositions, systems and methods herein are further described throughout the application.

In some embodiments, compositions, methods and systems described herein do not comprise a PAM sequence. In some embodiments, polypeptides (e.g., effector protein, effector partner and fusion protein) do not recognize a PAM sequence. In some embodiments, compositions, methods and systems described herein comprise a protospacer-flanking site (PFS) sequence. A PFS sequence may be useful for the detection and/or modification of RNA.

### II. Nucleic Acid Systems

### Guide Nucleic Acids

The compositions, systems and methods of the present disclosure may comprise a guide nucleic acid or a use thereof. Unless otherwise indicated, compositions, systems and methods comprising guide nucleic acids or uses thereof, as described herein and throughout, include DNA molecules, such as expression vectors, that encode a guide nucleic acid. Accordingly, compositions, systems and methods of the present disclosure comprise a guide nucleic acid or a nucleotide sequence encoding the guide nucleic acid. Guide nucleic acids are also referred to herein as “guide RNA.” A guide nucleic acid, as well as any components thereof (e.g., spacer sequence, repeat sequence, linker nucleotide sequence, handle sequence, intermediary sequence etc.) may comprise one or more deoxyribonucleotides, ribonucleotides, biochemically or chemically modified nucleotides (e.g., one or more engineered modifications as described herein), or any combinations thereof. Such nucleotide sequences described herein may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the sequence that encodes a guide nucleic acid, such as a nucleotide sequence described herein for a vector. Similarly, disclosure of the nucleotide sequences described herein also discloses the complementary nucleotide sequence, the reverse nucleotide sequence and the reverse complement nucleotide sequence, any one of which can be a nucleotide sequence for use in a guide nucleic acid as described herein. In some embodiments, a guide nucleic acid sequence(s) comprises one or more nucleotide alterations at one or more positions in any one of the sequences described herein. Alternative nucleotides can be any one or more of A, C, G, T or U, or a deletion, or an insertion. In some embodiments, a nucleotide “U” is a uracil or a 1N-Methyl-Pseudouridine.

A guide nucleic acid may comprise a naturally occurring sequence. A guide nucleic acid may comprise a non-naturally occurring sequence, wherein the nucleotide sequence of the guide nucleic acid, or any portion thereof, may be different from the sequence of a naturally occurring guide nucleic acid. A guide nucleic acid of the present disclosure comprises one or more of the following: a) a single nucleic acid molecule; b) a DNA base; c) an RNA base; d) a modified base; e) a modified sugar; f) a modified backbone; and the like. Modifications are described herein and throughout the present disclosure (e.g., in the section entitled “Engineered Modifications”). A guide nucleic acid may be chemically synthesized or recombinantly produced by any suitable methods. Guide nucleic acids and portions thereof may be found in or identified from a CRISPR array present in the genome of a host organism or cell.

In general, the guide nucleic acid comprises a nucleotide sequence that is at least 80%, at least 85%, at least 90%, at least 95%, at least 98%, at least 99%, or 100% complementary to the target sequence. In some embodiments, the guide nucleic acid comprises at least 10 contiguous nucleotides that are complementary to the target sequence in the target nucleic acid. In some embodiments, guide nucleic acid comprises a spacer sequence that is at least 80%, at least 85%, at least 90%, at least 95%, at least 98%, at least 99%, or 100% complementary to the target sequence.

In general, a guide nucleic acid comprises a first region that is not complementary to a target nucleic acid (FR) and a second region is complementary to the target nucleic acid (SR), wherein the FR and the SR are heterologous to each other. In some embodiments, FR is located 5′ to SR (FR-SR). In some embodiments, SR is located 5′ to FR (SR-FR). In some embodiments, the FR comprises one or more repeat sequence, handle sequence, intermediary sequence, or combinations thereof. In some embodiments, at least a portion of the FR interacts or binds to an effector protein. In some embodiments, the SR comprises a spacer sequence, wherein the spacer sequence can interact in a sequence-specific manner with (e.g., has complementarity with, or can hybridize to a target sequence in) a target nucleic acid.

In some embodiments, the first region, the second region, or both are 8 nucleic acids, nucleic acids, 12 nucleic acids, 14 nucleic acids, 16 nucleic acids, 18 nucleic acids, 20 nucleic acids, 22 nucleic acids, 24 nucleic acids, 26 nucleic acids, 28 nucleic acids, 30 nucleic acids, 32 nucleic acids, 34 nucleic acids, 36 nucleic acids, 38 nucleic acids, 40 nucleic acids, 42 nucleic acids, 44 nucleic acids, 46 nucleic acids, 48 nucleic acids, or 50 nucleic acids long.

In some embodiments, the first region, the second region, or both are from about 8 to about 12, from about 8 to about 16, from about 8 to about 20, from about 8 to about 24, from about 8 to about 28, from about 8 to about 30, from about 8 to about 32, from about 8 to about 34, from about 8 to about 36, from about 8 to about 38, from about 8 to about 40, from about 8 to about 42, from about 8 to about 44, from about 8 to about 48, or from about 8 to about 50 nucleic acids long.

In some embodiments, the first region, the second region, or both comprise a GC content of about 1%, about 5%, about 10%, about 15%, about 20%, about 25%, about 30%, about 40%, about 50%, about 60%, about 70%, about 80%, about 90%, about 95%, or about 99%. In some embodiments, the first region, the second region, or both comprise a GC content of from about 1% to about 95%, from about 5% to about 90%, from about 10% to about 80%, from about 15% to about 70%, from about 20% to about 60%, from about 25% to about 50%, or from about 30% to about 40%.

In some embodiments, the first region, the second region, or both have a melting temperature of about 38° C., about 40° C., about 42° C., about 44° C., about 46° C., about 48° C., about 50° C., about 52° C., about 54° C., about 56° C., about 58° C., about 60° C., about 62° C., about 64° C., about 66° C., about 68° C., about 70° C., about 72° C., about 74° C., about 76° C., about 78° C., about 80° C., about 82° C., about 84° C., about 86° C., about 88° C., about 90° C., or about 92° C. In some embodiments, the first region, the second region, or both have a melting temperature of from about 35° C. to about 40° C., from about 35° C. to about 45° C., from about 35° C. to about 50° C., from about 35° C. to about 55° C., from about 35° C. to about 60° C., from about 35° C. to about 65° C., from about 35° C. to about 70° C., from about 35° C. to about 75° C., from about 35° C. to about 80° C., or from about 35° C. to about 85° C.

In some embodiments, the compositions, systems and methods of the present disclosure further comprise an additional nucleic acid, wherein a portion of the additional nucleic acid at least partially hybridizes to the first region of the guide nucleic acid. In some embodiments, the additional nucleic acid is at least partially hybridized to the 5′ end of the second region of the guide nucleic acid. In some embodiments, an unhybridized portion of the additional nucleic acid, at least partially, interacts with an effector protein or polypeptide. In some embodiments, the compositions, systems and methods of the present disclosure comprise a dual nucleic acid system comprising the guide nucleic acid and the additional nucleic acid as described herein.

The guide nucleic acid may also form complexes as described through herein. For example, a guide nucleic acid hybridizes to another nucleic acid, such as target nucleic acid, or a portion thereof. In another example, a guide nucleic acid complexes with an effector protein. In such embodiments, a guide nucleic acid-effector protein complex is described herein as an RNP. In some embodiments, when in a complex, at least a portion of the complex binds, recognizes and/or hybridizes to a target nucleic acid. For example, when a guide nucleic acid and an effector protein are complexed to form an RNP, at least a portion of the guide nucleic acid hybridizes to a target sequence in a target nucleic acid. Those skilled in the art in reading the below specific examples of guide nucleic acids as used in RNPs described herein, will understand that. in some embodiments, a RNP hybridizes to one or more target sequences in a target nucleic acid, thereby allowing the RNP to modify and/or recognize a target nucleic acid or sequence contained therein (e.g., PAM) or to modify and/or recognize non-target sequences depending on the guide nucleic acid and, in some embodiments, the effector protein, used.

In some embodiments, a guide nucleic acid comprises or forms intramolecular secondary structure (e.g., hairpins, stem-loops, etc.). In some embodiments, a guide nucleic acid comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the guide nucleic acid comprises a pseudoknot (e.g., a secondary structure comprising a stem, at least partially, hybridized to a second stem or half-stem secondary structure). An effector protein may recognize a guide nucleic acid comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the guide nucleic acid comprises at least 2, at least 3, at least 4, or at least 5 stem regions.

In some embodiments, the compositions, systems and methods of the present disclosure comprise two or more guide nucleic acids (e.g., 2, 3, 4, 5, 6, 7, 9, 10 or more guide nucleic acids) and/or uses thereof. Multiple guide nucleic acids may target an effector protein to different locations in the target nucleic acid by hybridizing to different target sequences. In some embodiments, a first guide nucleic acid hybridizes within a location of the target nucleic acid that is different from where a second guide nucleic acid may hybridize the target nucleic acid. In some embodiments, the first loci and the second loci of the target nucleic acid are located at least 1, at least 10, at least 20, at least 30, at least 40, at least 50, at least 60, at least 70, at least 80, at least 90 or at least 100 nucleotides apart. In some embodiments, the first loci and the second loci of the target nucleic acid are located between 100 and 200, 200 and 300, 300 and 400, 400 and 500, 500 and 600, 600 and 700, 700 and 800, 800 and 900 or 900 and 1000 nucleotides apart. In some embodiments, the first loci and/or the second loci of the target nucleic acid are located in an intron of a gene. In some embodiments, the first loci and/or the second loci of the target nucleic acid are located in an exon of a gene. In some embodiments, the first loci and/or the second loci of the target nucleic acid span an exon-intron junction of a gene. In some embodiments, the first portion and/or the second portion of the target nucleic acid are located on either side of an exon and cutting at both sites results in deletion of the exon. In some embodiments, composition, systems and methods comprise a donor nucleic acid that is inserted in replacement of a deleted or cleaved sequence of the target nucleic acid. In some embodiments, compositions, systems and methods comprising multiple guide nucleic acids or uses thereof comprise multiple effector proteins, wherein the effector proteins are identical, non-identical, or combinations thereof.

In some embodiments, a guide nucleic acid comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In general, a guide nucleic acid comprises at least: 10, 15, 20, 25, 30, 35, 40, 45, 50, 55, 60 linked nucleotides. In some embodiments, the guide nucleic acid has about 10 to about 60, about 20 to about 50, or about 30 to about 40 linked nucleotides.

In some embodiments, a guide nucleic acid comprises at least 10, 11, 12, 13, 14, 15, 16, 17, 18, 19, 20, 21, 22, 23, 24, 25, 26, 27, 28, 29, or 30 contiguous nucleotides that are complementary to a eukaryotic sequence. Such a eukaryotic sequence is a nucleotide sequence that is present in a host eukaryotic cell. Such a nucleotide sequence is distinguished from nucleotide sequences present in other host cells, such as prokaryotic cells, or viruses. Said sequences present in a eukaryotic cell can be located in a gene, an exon, an intron, a non-coding (e.g., promoter or enhancer) region, a selectable marker, tag, signal and the like. In some embodiments, a target sequence is a eukaryotic sequence.

In some embodiments, a length of a guide nucleic acid is about 30 to about 120 linked nucleotides. In some embodiments, the length of a guide nucleic acid is about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a guide nucleic acid is about 40, about 45, about 50, about 55, about 60, about 65, about 70 or about 75 linked nucleotides. In some embodiments, the length of a guide nucleic acid is greater than about 15, about 20, about 25, about 30, about 35, about 40, about 45, about 50, about 55, about 60, about 65, about 70 or about 75 linked nucleotides. In some embodiments, the length of a guide nucleic acid is not greater than about 40, about 45, about 50, about 55, about 60, about 65, about 70, about 75, about 80, about 85, about 90, about 95, about 100, about 105, about 110, about 115, about 120, or about 125 linked nucleotides.

In some embodiments, guide nucleic acids comprise additional elements that contribute additional functionality (e.g., stability, heat resistance, etc.) to the guide nucleic acid. Such elements may be one or more nucleotide alterations, nucleotide sequences, intermolecular secondary structures, or intramolecular secondary structures (e.g., one or more hair pin regions, one or more bulges, etc.).

In some embodiments, guide nucleic acids comprise one or more linkers connecting different nucleotide sequences as described herein. A linker may comprise 1, 2, 3, 4, 5, 6, 7, 8, 9, 10, or more nucleotides. A linker may be any suitable linker, examples of which are described herein.

In some embodiments, guide nucleic acids comprise one or more nucleotide sequences as described herein (e.g., TABLE 7). Such nucleotide sequences described herein (e.g., TABLE 7) may be described as a nucleotide sequence of either DNA or RNA, however, no matter the form of the sequence described, it is readily understood that such nucleotide sequences may be revised to be RNA or DNA, as needed, for describing a sequence within a guide nucleic acid itself or the sequence that encodes a guide nucleic acid, such as a nucleotide sequence described herein for a vector. Similarly, disclosure of the nucleotide sequences described herein (e.g., TABLE 7) also discloses the complementary nucleotide sequence, the reverse nucleotide sequence and the reverse complement nucleotide sequence, any one of which may be a nucleotide sequence for use in a guide nucleic acid as described herein. In some embodiments, guide nucleic acid sequence(s) comprises one or more nucleotide alterations at one or more positions in any one of the sequences described herein. Alternative nucleotides may be any one or more of A, C, G, T or U, or a deletion, or an insertion.

In some embodiments, the guide nucleic acid comprises a nucleotide sequence that hybridizes to a target sequence in a target nucleic acid, wherein the target nucleic acid is any one of: a naturally occurring eukaryotic sequence, a naturally occurring prokaryotic sequence, a naturally occurring viral sequence, a naturally occurring bacterial sequence, a naturally occurring fungal sequence, an engineered eukaryotic sequence, an engineered prokaryotic sequence, an engineered viral sequence, an engineered bacterial sequence, an engineered fungal sequence, a fragment of a naturally occurring sequence, a fragment of an engineered sequence and combinations thereof.

In some embodiments, the guide nucleic acid is isolated from any one of: a naturally occurring cell, a eukaryotic cell, a prokaryotic cell, a plant cell, a fungal cell, an animal cell, cell of an invertebrate, a fly cell, a cell of a vertebrate, a mammalian cell, a primate cell, a non-human primate cell, a human cell, a living cell, a non-living cell, a modified cell, a derived cell and a non-naturally occurring cell.

### Repeat Sequence

Guide nucleic acids described herein may comprise one or more repeat sequences. In some embodiments, a repeat sequence comprises a nucleotide sequence that is not complementary to a target sequence of a target nucleic acid. In some embodiments, a repeat sequence comprises a nucleotide sequence that interacts with an effector protein. In some embodiments, a repeat sequence is connected to another sequence of a guide nucleic acid, such as an intermediary sequence, that non-covalently interacts with an effector protein. In some embodiments, a repeat sequence includes a nucleotide sequence that forms a guide nucleic acid-effector protein complex (e.g., a RNP complex).

In some embodiments, the repeat sequence is between 10 and 50, 12 and 48, 14 and 46, 16 and 44 and 18 and 42 nucleotides in length.

In some embodiments, a repeat sequence is adjacent to a spacer sequence. In some embodiments, a repeat sequence is followed by a spacer sequence in the 5′ to 3′ direction. In some embodiments, a repeat sequence is preceded by a spacer sequence in the 5′ to 3′ direction. In some embodiments, a repeat sequence is adjacent to an intermediary sequence. In some embodiments, a repeat sequence is 3′ to an intermediary sequence. In some embodiments, an intermediary sequence is followed by a repeat sequence, which is followed by a spacer sequence in the 5′ to 3′ direction. In some embodiments, a repeat sequence is linked to a spacer sequence and/or an intermediary sequence. In some embodiments, a guide nucleic acid comprises a repeat sequence linked to a spacer sequence and/or to an intermediary sequence, which is a direct link or by any suitable linker, examples of which are described herein.

In some embodiments, guide nucleic acids comprise more than one repeat sequence (e.g., two or more, three or more, or four or more repeat sequences). In some embodiments, a guide nucleic acid comprises more than one repeat sequence separated by another sequence of the guide nucleic acid. For example, in some embodiments, a guide nucleic acid comprises two repeat sequences, wherein the first repeat sequence is followed by a spacer sequence and the spacer sequence is followed by a second repeat sequence in the 5′ to 3′ direction. In some embodiments, the more than one repeat sequences are identical. In some embodiments, the more than one repeat sequences are not identical.

In some embodiments, the repeat sequence comprises two sequences that are complementary to each other and hybridize to form a double stranded RNA duplex (dsRNA duplex). In some embodiments, the two sequences are not directly linked and hybridize to form a stem loop structure. In some embodiments, the dsRNA duplex comprises 5, 10, 15, 20 or 25 base pairs (bp). In some embodiments, not all nucleotides of the dsRNA duplex are paired and, therefore, the duplex forming sequence includes a bulge. In some embodiments, the repeat sequence comprises a hairpin or stem-loop structure, optionally at the 5′ portion of the repeat sequence. In some embodiments, a strand of the stem portion comprises a sequence and the other strand of the stem portion comprises a sequence that is, at least partially, complementary. In some embodiments, such sequences have 65% to 100% complementarity (e.g., 65%, 70%, 75%, 80%, 85%, 90%, 95%, or 100% complementarity). In some embodiments, a guide nucleic acid comprises nucleotide sequence that when involved in hybridization events hybridizes over one or more segments such that intervening or adjacent segments are not involved in the hybridization event (e.g., a bulge, a loop structure or hairpin structure, etc.).

In some embodiments, a repeat sequence comprises a nucleotide sequence that is at least 65%, at least 70%, at least 75%, at least 80%, at least 85%, at least 90%, at least 95%, at least 97%, or at least 98%, at least 99%, or 100% identical to an equal length portion of any one of the repeat sequences in TABLE 7. In some embodiments, the repeat sequence is at least 85% identical to any one of nucleotide sequences recited in TABLE 7. In some embodiments, a repeat sequence comprises at least 5, at least 6, at least 7, at least 8, at least 9, at least 10, at least 11, at least 12, at least 13, at least 14, at least 15, at least 16, at least 17, at least 18, at least 19, at least 20, or at least 21 contiguous nucleotides of any one of the nucleotide sequences recited in TABLE 7.

In some embodiments, a repeat sequence comprises one or more nucleotide alterations at one or more positions in the nucleotide sequence recited in TABLE 7. Alternative nucleotides can be any one or more of A, C, G, T or U, or a deletion, or an insertion.

### Spacer Sequence

Guide nucleic acids described herein may comprise one or more spacer sequences. In some embodiments, a spacer sequence hybridizes to a target sequence of a target nucleic acid. In some embodiments, a spacer sequence comprises a nucleotide sequence that is, at least partially, hybridizable to an equal length of a sequence (e.g., a target sequence) of a target nucleic acid. Exemplary hybridization conditions are described herein. In some embodiments, the spacer sequence functions to direct an RNP complex comprising the guide nucleic acid to the target nucleic acid for detection and/or modification. The spacer sequence may function to direct a RNP to the target nucleic acid for detection and/or modification. A spacer sequence may be complementary to a target sequence that is adjacent to a PAM that is recognizable by an effector protein described herein.

In some embodiments, a spacer sequence comprises at least 5 to about 50 contiguous nucleotides that are complementary to a target sequence in a target nucleic acid. In some embodiments, a spacer sequence comprises at least 5 to about 50 linked nucleotides. In some embodiments, a spacer sequence comprises at least 5 to about 50, at least 5 to about 25, at least 10 to about 25, or at least 15 to about 25 linked nucleotides. In some embodiments, the spacer sequence comprises 15-28 linked nucleotides. In some embodiments, a spacer sequence comprises 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleotides. In some embodiments, the spacer sequence comprises 10, 11, 12, 13, 14, 15, 16, 17, 18, 19, 20, 21, 22, 23, 24, 25, 26, 27, 28, 29, 30, or more nucleotides. In some embodiments, the spacer sequence comprises a nucleotide sequence of 13 to 15 linked nucleotides. In some embodiments, the spacer sequence comprises a nucleotide sequence of 14 linked nucleotides.

In some embodiments, a spacer sequence is adjacent to a repeat sequence. In some embodiments, a spacer sequence follows a repeat sequence in a 5′ to 3′ direction. In some embodiments, a spacer sequence precedes a repeat sequence in a 5′ to 3′ direction. In some embodiments, the spacer sequence(s) and the repeat sequence(s) of the guide nucleic acid are present within the same molecule. In some embodiments, the spacer(s) and repeat sequence(s) are linked directly to one another. In some embodiments, a linker is present between the spacer(s) and repeat sequences. Linkers may be any suitable linker. In some embodiments, the spacer sequence(s) and the repeat sequence(s) of the guide nucleic acid are present in separate molecules, which are joined to one another by base pairing interactions.

In some embodiments, a spacer sequence comprises a nucleotide sequence that is at least 65%, at least 70%, at least 75%, at least 80%, at least 85%, at least 90%, at least 95% or 100% complementary to a target sequence of a target nucleic acid. A spacer sequence hybridizes to an equal length portion of a target nucleic acid (e.g., a target sequence). In some embodiments, a target nucleic acid, such as DNA or RNA, is a cancer gene or gene associated with a genetic disorder, or an amplicon thereof, as described herein. In some embodiments, a target nucleic acid is a gene selected from TABLE 8. In some embodiments, a spacer sequence comprises a nucleotide sequence that is at least 65%, at least 70%, at least 75%, at least 80%, at least 85%, at least 90%, at least 95% or 100% complementary to a target sequence of a target nucleic acid selected from TABLE 8. In some embodiments, a target nucleic acid is a nucleic acid associated with a disease or syndrome set forth in TABLE 9. In some embodiments, a spacer sequence comprises a nucleotide sequence that is at least 65%, at least 70%, at least 75%, at least 80%, at least 85%, at least 90%, at least 95% or 100% complementary to a target sequence of a target nucleic acid associated with a disease or syndrome set forth in TABLE 9. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that hybridize to the target sequence. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that are complementary to the target sequence.

It is understood that the spacer sequence of a spacer sequence need not be 100% complementary to that of a target sequence of a target nucleic acid to hybridize or hybridize specifically to the target sequence. For example, the spacer sequence comprises at least one alteration, such as a substituted or modified nucleotide, that is not complementary to the corresponding nucleotide of the target sequence.

### Linker for Nucleic Acids

In some embodiments, a guide nucleic acid for use with compositions, systems and methods described herein comprises one or more linkers, or a nucleic acid encoding one or more linkers. In some embodiments, the guide nucleic acid comprises at least one, at least two, at least three, at least four, at least five, at least six, at least seven, at least eight, at least nine, or at least ten linkers. In some embodiments, the guide nucleic acid comprises one, two, three, four, five, six, seven, eight, nine, or ten linkers. In some embodiments, the guide nucleic acid comprises more than one linker. In some embodiments, at least two of the more than one linker are the same. In some embodiments, at least two of the more than one linker are not same.

In some embodiments, a linker comprises one to ten, one to seven, one to five, one to three, two to ten, two to eight, two to six, two to four, three to ten, three to seven, three to five, four to ten, four to eight, four to six, five to ten, five to seven, six to ten, six to eight, seven to ten, or eight to ten linked nucleotides. In some embodiments, the linker comprises one, two, three, four, five, six, seven, eight, nine, or ten linked nucleotides. In some embodiments, a linker comprises a nucleotide sequence of 5′-GAAA-3′.

In some embodiments, a guide nucleic acid comprises one or more linkers connecting one or more repeat sequences. In some embodiments, the guide nucleic acid comprises one or more linkers connecting one or more repeat sequences and one or more spacer sequences. In some embodiments, the guide nucleic acid comprises at least two repeat sequences connected by a linker.

### A Single Nucleic Acid System

In some embodiments, compositions, systems and methods described herein comprise a single nucleic acid system comprising a guide nucleic acid or a nucleotide sequence encoding the guide nucleic acid, and one or more effector proteins or a nucleotide sequence encoding the one or more effector proteins. In some embodiments, a first region (FR) of the guide nucleic acid non-covalently interacts with the one or more polypeptides described herein. In some embodiments, a second region (SR) of the guide nucleic acid hybridizes with a target sequence of the target nucleic acid. In the single nucleic acid system having a complex of the guide nucleic acid and the effector protein, the effector protein is not transactivated by the guide nucleic acid. In other words, activity of effector protein does not require binding to a second non-target nucleic acid molecule. An exemplary guide nucleic acid for a single nucleic acid system is a crRNA.

crRNA

In some embodiments, a guide nucleic acid comprises a crRNA. In some embodiments, the guide nucleic acid is the crRNA. In general, a crRNA comprises a first region (FR) and a second region (SR), wherein the FR of the crRNA comprises a repeat sequence, and the SR of the crRNA comprises a spacer sequence. In some embodiments, the repeat sequence and the spacer sequences are directly connected to each other (e.g., covalent bond (phosphodiester bond)). In some embodiments, the repeat sequence and the spacer sequence are connected by a linker.

In some embodiments, a crRNA is useful as a single nucleic acid system for compositions, methods and systems described herein or as part of a single nucleic acid system for compositions, methods and systems described herein. In some embodiments, a crRNA is useful as part of a single nucleic acid system for compositions, methods and systems described herein. In such embodiments, a single nucleic acid system comprises a guide nucleic acid comprising a crRNA wherein, a repeat sequence of a crRNA connects the crRNA to an effector protein.

A crRNA may include deoxyribonucleosides, ribonucleosides, chemically modified nucleosides, or any combination thereof. In some embodiments, a crRNA comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In some embodiments, a crRNA comprises at least: 10, 15, 20, 25, 30, 35, 40, 45, 50, 55, 60 linked nucleotides. In some embodiments, the length of the crRNA is about 20 to about 120 linked nucleotides. In some embodiments, the length of a crRNA is about 20 to about 100, about 30 to about 100, about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a crRNA is about 20, about 25, about 30, about 35, about 40, about 45, about 50, about 55, about 60, about 65, about 70 or about 75 linked nucleotides.

### III. Engineered Modifications

Polypeptides (e.g., effector proteins) and nucleic acids (e.g., engineered guide nucleic acids) can be further modified as described herein. Examples are modifications that do not alter the primary sequence of the polypeptides or nucleic acids, such as chemical derivatization of polypeptides (e.g., acylation, acetylation, carboxylation, amidation, etc.), or modifications that do alter the primary sequence of the polypeptide or nucleic acid. Also included are polypeptides that have a modified glycosylation pattern (e.g., those made by: modifying the glycosylation patterns of a polypeptide during its synthesis and processing or in further processing steps; by exposing the polypeptide to enzymes which affect glycosylation, such as mammalian glycosylating or deglycosylating enzymes). Also embraced are polypeptides that have phosphorylated amino acid residues (e.g., phosphotyrosine, phosphoserine, or phosphothreonine).

Modifications disclosed herein can also include modification of described polypeptides and/or guide nucleic acids through any suitable method, such as molecular biological techniques and/or synthetic chemistry, to improve their resistance to proteolytic degradation, to change the target sequence specificity, to optimize solubility properties, to alter protein activity (e.g., transcription modulatory activity, enzymatic activity, etc.) or to render them more suitable for their intended purpose (e.g., in vivo administration, in vitro methods, or ex vivo applications). Analogs of such polypeptides include those containing residues other than naturally occurring L-amino acids, e.g. D-amino acids or non-naturally occurring synthetic amino acids. D-amino acids may be substituted for some or all of the amino acid residues. Modifications can also include modifications with non-naturally occurring unnatural amino acids. The particular sequence and the manner of preparation will be determined by convenience, economics, purity required and the like.

Modifications can further include the introduction of various groups to polypeptides and/or guide nucleic acids described herein. For example, groups can be introduced during synthesis or during expression of a polypeptide (e.g., an effector protein), which allow for linking to other molecules or to a surface. Thus, e.g., cysteines may be used to make thioethers, histidines for linking to a metal ion complex, carboxyl groups for forming amides or esters, amino groups for forming amides and the like.

Modifications can further include changing of nucleic acids described herein (e.g., engineered guide nucleic acids) to provide the nucleic acid with a new or enhanced feature, such as improved stability. Such modifications of a nucleic acid include a base editing, a base modification, a backbone modification, a sugar modification, or combinations thereof. In some embodiments, the modifications can be of one or more nucleotides, nucleosides, or nucleobases in a nucleic acid.

In some embodiments, nucleic acids (e.g., nucleic acids encoding effector proteins, engineered guide nucleic acids, or nucleic acids encoding engineered guide nucleic acids) described herein comprise one or more modifications comprising: 2′O-methyl modified nucleotides (e.g., 2′-O-Methyl(2′OMe) sugar modifications); 2′ fluoro modified nucleotides (e.g., 2′-fluoro (2′-F) sugar modifications); locked nucleic acid (LNA) modified nucleotides; peptide nucleic acid (PNA) modified nucleotides; nucleotides with phosphorothioate linkages; a 5′ cap (e.g., a 7-methylguanylate cap (m7G)), phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkyl phosphoramidates, phosphorodiamidates, thionophosphor amidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage; phosphorothioate and/or heteroatom internucleoside linkages, such as —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—); morpholino linkages (formed in part from the sugar portion of a nucleoside); morpholino backbones; phosphorodiamidate or other non-phosphodiester internucleoside linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; other backbone modifications having mixed N, O, S and CH₂ component parts; and combinations thereof.

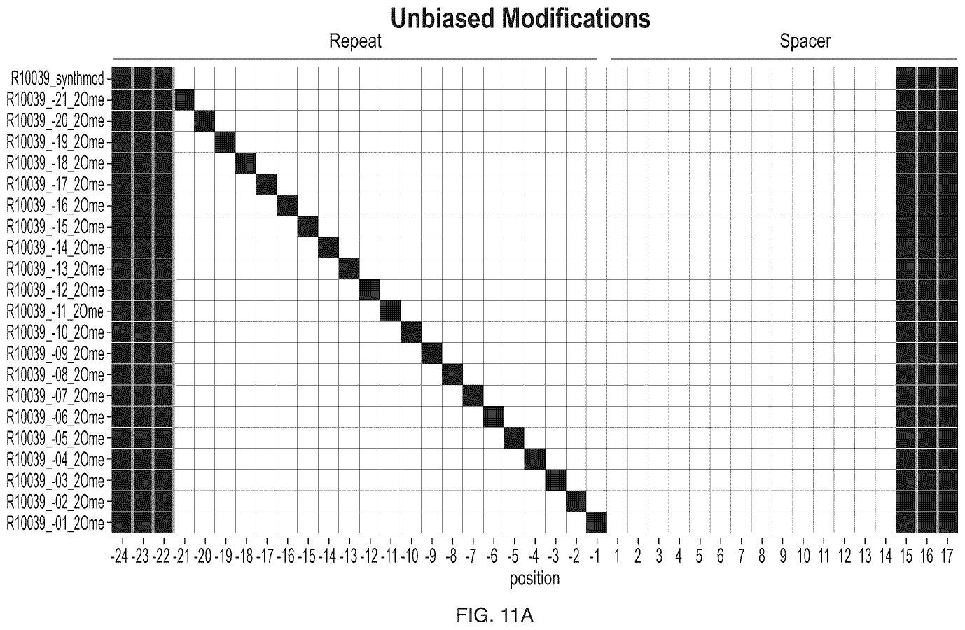

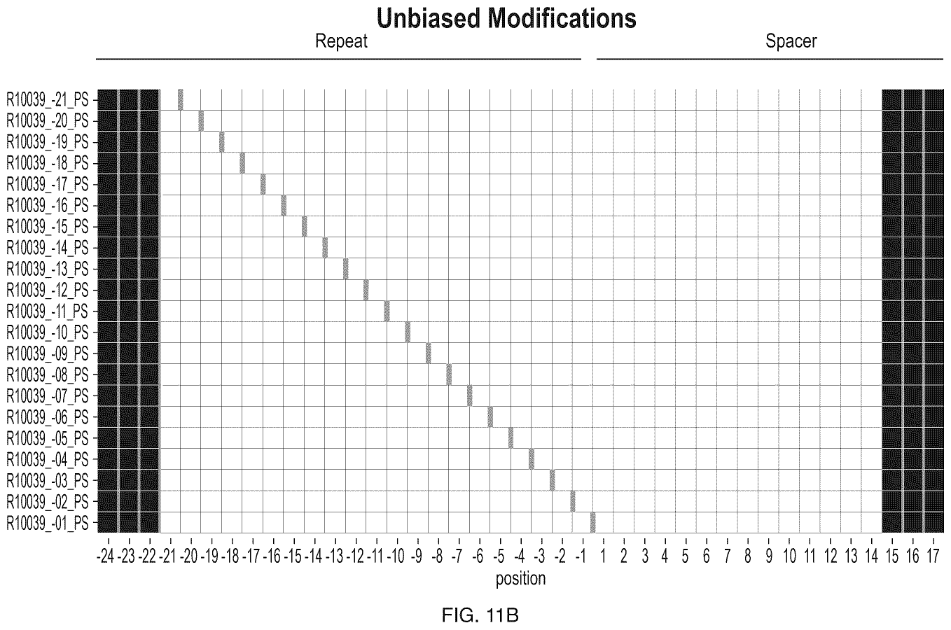

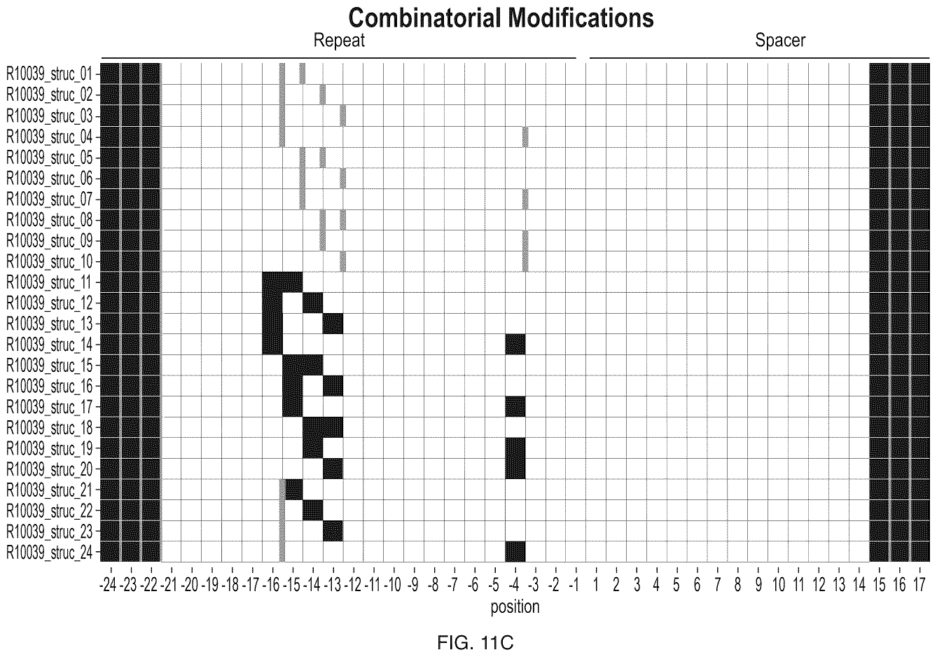

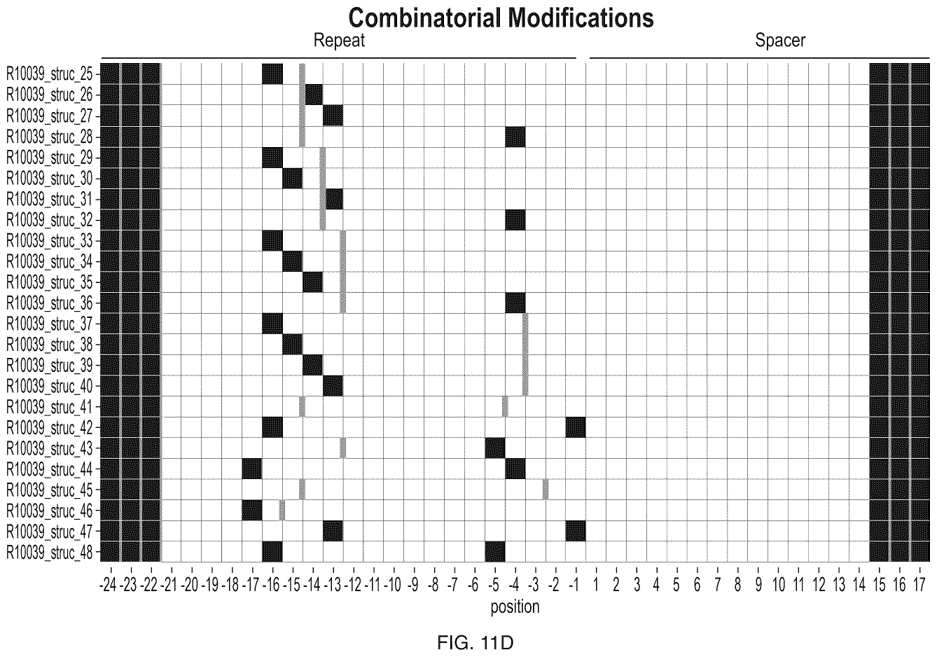

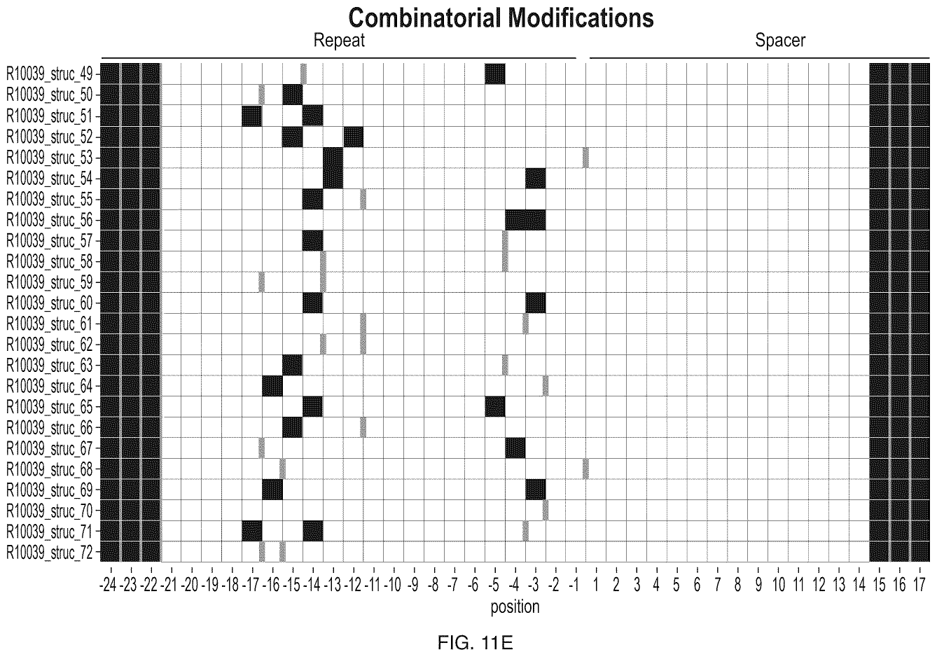

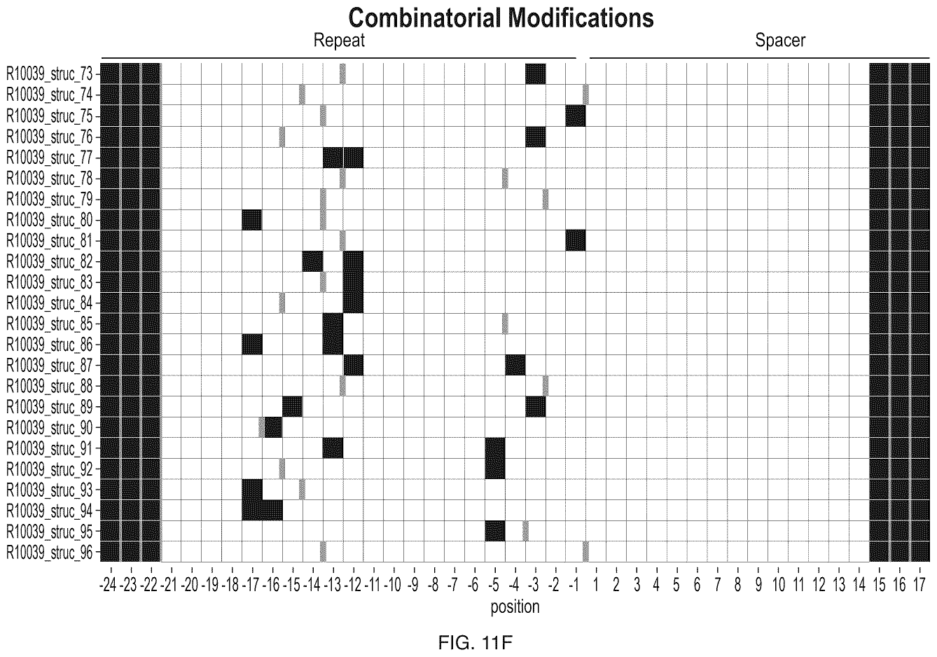

In some embodiments, guide nucleic acids described herein comprise one or more 2′-O-Methyl(2′OMe) sugar modifications, one or more phosphorothioate (PS) backbone modifications, or combinations thereof. In some embodiments, the one or more 2′OMe sugar modification, PS backbone modification, or combinations thereof are contained within a portion of a guide nucleic acid (e.g., repeat sequence) that at least partially interacts with an effector protein described herein. For example, in some embodiments, the guide nucleic acids comprise PS backbone modification between −3 and −2 positions of a repeat sequence present in the guide nucleic acid, wherein the repeat sequence comprises a nucleotide length described herein, such as at least 24 nucleotides. In some embodiments, the guide nucleic acids comprise PS backbone modification between −3 and −2 positions of a repeat sequence present in the guide nucleic acid in combination with at least one modification between −16 and −12 positions of the repeat sequence present in the guide nucleic acid, wherein the repeat sequence comprises a nucleotide length described herein, such as at least 24 nucleotides. In some embodiments, the guide nucleic acids comprise a 2′OMe sugar modification at −14 position of a repeat sequence present in the guide nucleic acid, and a PS backbone modification between −3 and −2 positions of the repeat sequence present in the guide nucleic acid, wherein the repeat sequence comprises a nucleotide length described herein, such as at least 24 nucleotides. In some embodiments, the guide nucleic acids comprise a 2′OMe sugar modification at −16 position, and a PS backbone modification between −3 and −2 positions of a repeat sequence present in the guide nucleic acid, wherein the repeat sequence comprises at least 24 nucleotides. In some embodiments, the guide nucleic acids comprise PS backbone modifications between −3 and −2 positions of a repeat sequence present in the guide nucleic acid, and −13 and −12 positions of the repeat sequence, wherein the repeat sequence comprises a nucleotide length described herein, such as at least 24 nucleotides. In some embodiments, the guide nucleic acids comprise PS backbone modifications between −3 and −2 positions of a repeat sequence present in the guide nucleic acid, and −14 and −13 positions of the repeat sequence, wherein the repeat sequence comprises a nucleotide length described herein, such as at least 24 nucleotides. In some embodiments, the guide nucleic acids comprise PS backbone modifications between −3 and −2 positions of a repeat sequence present in the guide nucleic acid, and −15 and −14 positions of the repeat sequence, wherein the repeat sequence comprises a nucleotide length described herein, such as at least 24 nucleotides. It is understood that the position of such modifications is described herein relative to the 3′ of the repeat sequence contained within the guide nucleic acid, as exemplified in FIGS. 11A-11F.

### IV. Vectors and Multiplexed Expression Vectors

Compositions, systems and methods described herein comprise a vector or a use thereof. A vector can comprise a nucleic acid of interest. In some embodiments, the nucleic acid of interest comprises one or more components of a composition or system described herein. In some embodiments, the nucleic acid of interest comprises a nucleotide sequence that encodes one or more components of the composition or system described herein. In some embodiments, one or more components comprises a polypeptide(s) (e.g., effector protein(s), effector partner(s), fusion protein(s), or combinations thereof), guide nucleic acid(s) and target nucleic acid(s). In some embodiments, a “u” of the nucleotide sequence is a uracil or a IN-Methyl-Pseudouridine. In some embodiments, the component comprises a nucleic acid encoding the polypeptide, and a guide nucleic acid or a nucleic acid encoding the guide nucleic acid. In some embodiments, a vector is part of a vector system. The vector system may comprise a library of vectors each encoding one or more component of a composition or system described herein. In some embodiments, components described herein (e.g., an effector protein, a guide nucleic acid and/or a target nucleic acid) are encoded by the same vector. In some embodiments, components described herein (e.g., a polypeptide (e.g., an effector protein, an effector partner, a fusion protein, or a combination thereof) a guide nucleic acid and/or a target nucleic acid) are each encoded by different vectors of the system.

In some embodiments, a vector comprises a nucleotide sequence encoding one or more polypeptides (e.g., effector proteins, effector partners, fusion proteins, or combinations thereof as described herein. In some embodiments, the one or more polypeptides comprise at least two polypeptides. In some embodiments, the at least two polypeptides are the same. In some embodiments, the at least two polypeptides are different from each other. In some embodiments, the nucleotide sequence is operably linked to a promoter that is operable in a target cell, such as a eukaryotic cell. In some embodiments, the vector comprises the nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more polypeptides.

In some embodiments, a vector encodes one or more of any system components, including but not limited to polypeptides (e.g., effector proteins, effector partners, fusion proteins, or combinations thereof), guide nucleic acids and target nucleic acids as described herein. In some embodiments, a system component encoding sequence is operably linked to a promoter that is operable in a target cell, such as a eukaryotic cell. In some embodiments, a vector encodes 1, 2, 3, 4 or more of any system components. For example, a vector encodes two or more guide nucleic acids, wherein each guide nucleic acid comprises a different sequence. A vector may encode the polypeptide and the guide nucleic acid.

In some embodiments, a vector comprises one or more guide nucleic acids, or a nucleotide sequence encoding the one or more guide nucleic acids as described herein. In some embodiments, the one or more guide nucleic acids comprise at least two guide nucleic acids. In some embodiments, the at least two guide nucleic acids are the same. In some embodiments, the at least two guide nucleic acids are different from each other. In some embodiments, the guide nucleic acid or the nucleotide sequence encoding the guide nucleic acid is operably linked to a promoter that is operable in a target cell, such as a eukaryotic cell. In some embodiments, the vector comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids. In some embodiments, the vector comprises a nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids.

In some embodiments, a vector comprises or encode one or more regulatory elements. Regulatory elements may refer to transcriptional and translational control sequences, such as promoters, enhancers, polyadenylation signals, terminators, protein degradation signals and the like, that provide for and/or regulate transcription of a non-coding sequence or a coding sequence and/or regulate translation of an encoded polypeptide. In some embodiments, a vector comprises or encodes for one or more additional elements, such as, for example, replication origins, antibiotic resistance (or a nucleic acid encoding the same), a tag (or a nucleic acid encoding the same), selectable markers and the like. In some embodiments, a vector comprises or encodes for one or more elements, such as, for example, ribosome binding sites and RNA splice sites.

Vectors described herein can encode a promoter-a regulatory region on a nucleic acid, such as a DNA sequence, initiates transcription of a downstream (3′ direction) coding or non-coding sequence. A promoter can be linked at its 3′ terminus to a nucleic acid, the expression or transcription of which is desired, and extends upstream (5′ direction) to include bases or elements necessary to initiate transcription or induce expression, which could be measured at a detectable level. A promoter can comprise a nucleotide sequence, referred to herein as a “promoter sequence”. The promoter sequence can include a transcription initiation site, and one or more protein binding domains responsible for the binding of transcription machinery, such as RNA polymerase. When eukaryotic promoters are used, such promoters can contain “TATA” boxes and “CAT” boxes. Various promoters, including inducible promoters, may be used to drive expression, i.e., transcriptional activation, of the nucleic acid of interest.

Accordingly, in some embodiments, the nucleic acid of interest can be operably linked to a promoter.

Promotors may be any suitable type of promoter envisioned for the compositions, systems and methods described herein. Examples include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc. Suitable promoters include, but are not limited to: SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter and a human Hl promoter (Hl). By transcriptional activation, it is intended that transcription will be increased above basal levels in the target cell by 2 fold, 5 fold, 10 fold, 50 fold, by 100 fold, 500 fold, or by 1000 fold, or more. In addition, vectors used for providing a nucleic acid that, when transcribed, produces a guide nucleic acid and/or a nucleic acid that encodes a polypeptide (e.g., an effector protein, an effector partner, a fusion protein, or a combination thereof) to a cell may include nucleic acid sequences that encode for selectable markers in the target cells, so as to identify cells that have taken up the guide nucleic acid and/or the polypeptide.

In general, vectors provided herein comprise at least one promotor or a combination of promoters driving expression or transcription of one or more genome editing tools described herein. In some embodiments, the vector comprises a nucleotide sequence of a promoter. In some embodiments, the vector comprises two promoters. In some embodiments, the vector comprises three promoters. In some embodiments, a length of the promoter is less than about 500, less than about 400, less than about 300, or less than about 200 linked nucleotides. In some embodiments, a length of the promoter is at least 100, at least 200, at least 300, at least 400, or at least 500 linked nucleotides. Non-limiting examples of promoters include CMV, 7SK, EF1a, RPBSA, hPGK, EFS, SV40, PGK1, Ubc, human beta actin, TRE, UAS, Ac5, Polyhedrin, CaMKIIa, GAL1-10, H1, TEF1, GDS, ADH1, CaMV35S, HSV TK, Ubi, U6, MNDU3, MSCV, MND and CAG.

In some embodiments, some promoters (e.g., U6, enhanced U6, Hl and 7SK) prefers the nucleic acid being transcribed having “g” nucleotide at the 5′ end of the coding sequence. Accordingly, when such coding sequence is expressed, it comprises an additional “g” nucleotide at 5′ end. In some embodiments, vectors provided herein comprise a promotor driving expression or transcription of any one of the guide nucleic acids described herein further comprises “g” nucleotide at 5′ end of the guide nucleic acid, wherein the promotor is selected from U6, enhanced U6, Hl and 7SK.

In some embodiments, the promoter is a constitutive promoter. In some embodiments, the promoter is an inducible promoter. In some embodiments, the inducible promoter only drives expression of its corresponding coding sequence (e.g., polypeptide or guide nucleic acid) when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter and an estrogen receptor-regulated promoter. In some embodiments, the promoter is an activation-inducible promoter, such as a CD69 promoter. In some embodiments, the promoter for expressing a polypeptide (e.g., an effector protein, an effector partner, a fusion protein, or a combination thereof) is a ubiquitous promoter. In some embodiments, the ubiquitous promoter comprises MND or CAG promoter sequence.

In some embodiments, the promoters are prokaryotic promoters (e.g., drive expression of a gene in a prokaryotic cell). In some embodiments, the promoters are eukaryotic promoters, (e.g., drive expression of a gene in a eukaryotic cell). In some embodiments, the promoter is EF1a. In some embodiments, the promoter is ubiquitin. In some embodiments, vectors are bicistronic or polycistronic vector (e.g., having or involving two or more loci responsible for generating a protein) having an internal ribosome entry site (IRES) is for translation initiation in a cap-independent manner.

In some embodiments, a vector described herein is a nucleic acid expression vector. In some embodiments, a vector described herein is a recombinant expression vector. In some embodiments, a vector described herein is a messenger RNA. For example, in some embodiments, the nucleic acids encoding effector proteins, effector partners, fusion proteins thereof, or combinations thereof are messenger RNAs. In some embodiments, the nucleic acid encoding the effector protein comprises a messenger RNA. In some embodiments, a vector comprising the recombinant nucleic acid as described herein, wherein the vector is a viral vector, an adeno associated viral (AAV) vector, a retroviral vector, or a lentiviral vector. In some embodiments, a vector described herein or a recombinant nucleic acid described herein is comprised in a cell. In some embodiments, a recombinant nucleic acid integrated into a genomic DNA sequence of the cell, wherein the cell is a eukaryotic cell or a prokaryotic cell.

In some embodiments, a vector described herein is a delivery vector. In some embodiments, the delivery vector is a eukaryotic vector, a prokaryotic vector (e.g., a bacterial vector) a viral vector, or any combination thereof. In some embodiments, the delivery vehicle is a non-viral vector. In some embodiments, the delivery vector is a plasmid. In some embodiments, the plasmid comprises DNA. In some embodiments, the plasmid comprises RNA. In some embodiments, the plasmid comprises circular double-stranded DNA. In some embodiments, the plasmid is linear. In some embodiments, the plasmid comprises one or more coding sequences of interest and one or more regulatory elements. In some embodiments, the plasmid comprises a bacterial backbone containing an origin of replication and an antibiotic resistance gene or other selectable marker for plasmid amplification in bacteria. In some embodiments, the plasmid is a minicircle plasmid. In some embodiments, the plasmid contains one or more genes that provide a selective marker to induce a target cell to retain the plasmid. In some examples, the plasmids are engineered through synthetic or other suitable means known in the art. For example, in some embodiments, the genetic elements are assembled by restriction digest of the desired genetic sequence from a donor plasmid or organism to produce ends of the DNA which is then be readily ligated to another genetic sequence.

In some embodiments, vectors comprise an enhancer. Enhancers are nucleotide sequences that have the effect of enhancing promoter activity. In some embodiments, enhancers augment transcription regardless of the orientation of their sequence. In some embodiments, enhancers activate transcription from a distance of several kilo basepairs. Furthermore, enhancers are located optionally upstream or downstream of a gene region to be transcribed and/or located within the gene, to activate the transcription. Exemplary enhancers include, but are not limited to, WPRE; CMV enhancers; the R-U5′ segment in LTR of HTLV-I.

### Administration of a Non-Viral Vector

In some embodiments, an administration of a non-viral vector comprises contacting a cell, such as a host cell, with the non-viral vector. In some embodiments, a physical method or a chemical method is employed for delivering the vector into the cell. Exemplary physical methods include electroporation, gene gun, sonoporation, magnetofection, or hydrodynamic delivery. Exemplary chemical methods include delivery of the recombinant polynucleotide by liposomes such as, cationic lipids or neutral lipids; lipofection; dendrimers; lipid nanoparticle (LNP); or cell-penetrating peptides.

In some embodiments, a vector is administered as part of a method of nucleic acid detection, editing and/or treatment as described herein. In some embodiments, a vector is administered in a single vehicle, such as a single expression vector. In some embodiments, at least two of the three components, a nucleic acid encoding one or more polypeptides (e.g., effector proteins, effector partners, fusion proteins, or combinations thereof), and one or more guide nucleic acids or a nucleic acid encoding the one or more guide nucleic acid, are provided in the single expression vector. In some embodiments, components, such as the guide nucleic acid and the polypeptide, are encoded by the same vector. In some embodiments, an effector protein (or a nucleic acid encoding same) and/or an engineered guide nucleic acid (or a nucleic acid that, when transcribed, produces same) are not co-administered with an effector partner (or a nucleic acid encoding the same) and/or a fusion protein (or a nucleic acid encoding the same) in a single vehicle. In some embodiments, an effector protein (or a nucleic acid encoding same), an engineered guide nucleic acid (or a nucleic acid that, when transcribed, produces same), an effector partner (or a nucleic acid encoding the same), and/or a fusion protein (or a nucleic acid encoding the same) are administered in one or more or two or more vehicles, such as one or more, or two or more expression vectors.

In some embodiments, a vector system is administered as part of a method of nucleic acid detection, editing and/or treatment as described herein, wherein at least two vectors are co-administered. In some embodiments, the at least two vectors comprise different components. In some embodiments, the at least two vectors comprise the same component having different sequences. In some embodiments, at least one of the three components, a nucleic acid encoding one or more polypeptides (e.g., effector proteins, effector partners, fusion proteins, or combinations thereof), and one or more guide nucleic acids or a nucleic acid encoding the one or more guide nucleic acids, or a variant thereof is provided in a different vector. In some embodiments, the nucleic acid encoding the polypeptide, and a guide nucleic acid or a nucleic acid encoding the guide nucleic acid are provided in different vectors. In some embodiments, the effector partner is encoded by a different vector than the vector encoding the effector protein and the guide nucleic acid.

### Lipid Particles and Non-Viral Vectors

In some embodiments, compositions and systems provided herein comprise a lipid particle. In some embodiments, a lipid particle is a lipid nanoparticle (LNP). In some embodiments, a lipid or a lipid nanoparticle can encapsulate an expression vector as described herein. LNPs are a non-viral delivery system for delivery of the composition and/or system components described herein. LNPs are particularly effective for delivery of nucleic acids. Beneficial properties of LNP include ease of manufacture, low cytotoxicity and immunogenicity, high efficiency of nucleic acid encapsulation and cell transfection, multi-dosing capabilities and flexibility of design (Kulkarni et al., (2018) Nucleic Acid Therapeutics, 28(3):146-157). In some embodiments, compositions and methods comprise a lipid, polymer, nanoparticle, or a combination thereof, or use thereof, to introduce one or more effector proteins, one or more guide nucleic acids, one or more effector partners, one or more fusion proteins or any combinations thereof to a cell. Non-limiting examples of lipids and polymers are cationic polymers, cationic lipids, ionizable lipids, or bio-responsive polymers. In some embodiments, the ionizable lipids exploits chemical-physical properties of the endosomal environment (e.g., pH) offering improved delivery of nucleic acids. In some embodiments, the ionizable lipids are neutral at physiological pH. In some embodiments, the ionizable lipids are protonated under acidic pH. In some embodiments, the bio-responsive polymer exploits chemical-physical properties of the endosomal environment (e.g., pH) to preferentially release the genetic material in the intracellular space.

In some embodiments, a LNP comprises an outer shell and an inner core. In some embodiments, the outer shell comprises lipids. In some embodiments, the lipids comprise modified lipids. In some embodiments, the modified lipids comprise pegylated lipids. In some embodiments, the lipids comprise one or more of cationic lipids, anionic lipids, ionizable lipids and non-ionic lipids. In some embodiments, the LNP comprises one or more of N1,N3,N5-tris(3-(didodecylamino)propyl)benzene-1,3,5-tricarboxamide (TT3), 1,2-Dioleoyl-sn-glycero-3-phosphocholine (DOPC), 2-dioleoyl-sn-glycero-3-phosphoethanolamine (DOPE), 1-palmitoyl-2-oleoyl-sn-glycero-3-phosphoethanolamine (POPE), 1,2-distearoyl-sn-glycero-3-phosphocholine (DSPC), cholesterol (Chol), 1,2-dimyristoyl-sn-glycerol, methoxypolyethylene glycol, 1,2-dimyristoyl-rac-glycero-3-methoxypolyethylene glycol-2000 (DMG-PEG 2000), derivatives, analogs, variants thereof or any combination thereof.

In some embodiments, the LNP comprises one or more ionizable lipid. Such ionizable lipids include, but are not limited to: 4-(dimethylamino)-butanoic acid, (10Z,13Z)-1-(9Z,12Z)-9,12-octadecadien-1-yl-10,13-nonadecadien-1-yl ester (DLin-MC3-DMA, CAS No. 1224606-06-7); N,N-dimethyl-2,2-di-(9Z,12Z)-9,12-octadecadien-1-yl-1,3-dioxolane-4-ethanamine (DLin-KC2-DMA, CAS No. 1190197-97-7); 8-[(2-hydroxyethyl) [6-oxo-6-(undecyloxy) hexyl]amino]-octanoic acid, 1-octylnonyl ester (SM-102, CAS No. 2089251-47-6); 8-[(2-hydroxyethyl) [8-(nonyloxy)-8-oxooctyl]amino]-octanoic acid, 1-octylnonyl ester (Lipid 5, CAS No. 2089251-33-0); 1,1′-[[2-[4-[2-[2-[bis(2-hydroxydodecyl)amino]ethyl](2-hydroxydodecyl)amino]ethyl]-1-piperazinyl]ethyl]imino]bis-2-dodecanol (C12-200, CAS No. 1220890-25-4); 2-hexyl-decanoic acid, 1,1′-[[(4-hydroxybutyl)imino]di-6,1-hexanediyl]ester (ALC-0315, CAS No. 2036272-55-4); 9,12-octadecadienoic acid, (9Z,12Z)-1,1′,1″,1″-[(3,6-dioxo-2,5-piperazinediyl)bis(4,1-butanediylnitrilodi-4,1-butanediyl)] ester (OF-C4-Deg-Lin, CAS No. 1853203-01-6); bis(2-(dodecyldisulfaneyl)ethyl) 3,3′-((3-methyl-9-oxo-10-oxa-13,14-dithia-3,6-diazahexacosyl)azanediyl)dipropionate (BAMEA-O16B, CAS No. 2490668-30-7); 3,6-bis[4-[bis[(9Z,12Z)-2-hydroxy-9,12-octadecadien-1-yl]amino]butyl]-2,5-piperazinedione (OF-02, CAS No. 1883431-67-1); tetrakis(8-methylnonyl) 3,3′,3″,3′-(((methylazanediyl)bis(propane-3,1-diyl))bis(azanetriyl))tetrapropionate (306Oi10, CAS No. 2322290-93-5); tetrakis(2-(octyldisulfaneyl)ethyl) 3,3′,3″,3′-(((methylazanediyl)bis(propane-3,1-diyl))bis(azanetriyl))tetrapropionate (306-O12B, CAS No. 2566523-06-4); bis(2-butyloctyl) 10-(N-(3-(dimethylamino)propyl)nonanamido)nonadecanedioate (Lipid A9, CAS No. 2036272-50-9); Arcturus Lipid 2,2 (8,8) 4C CH₃ (ATX-0114, CAS No. 2230647-28-4)); di((Z)-non-2-en-1-yl) 8,8′-((2-((2-(dimethylamino)ethyl)thio)acetyl)azanediyl)dioctanoate (ATX-001, CAS No. 1777792-33-2); di((Z)-non-2-en-1-yl) 8,8′-((((2-(dimethylamino)ethyl)thio)carbonyl)azanediyl)dioctanoate (ATX-002, CAS No. 1777792-34-3); Genevant CL1 (CAS No. 1450888-71-7); LP01; hexa(octan-3-yl) 9,9′,9″,9′″,9″″,9″″″-((((benzene-1,3,5-tricarbonyl)yris(azanediyl)) tris (propane-3,1-diyl)) tris(azanetriyl))hexanonanoate (FTT5); 5A2-SC8 (CAS No. 1857341-90-2); COATSOME® SS-OP; cKK-E12 (3,6-bis({4-[bis(2-hydroxydodecyl)amino]butyl}piperazine-2,5-dione); derivatives; analogs; or variants thereof. In some embodiments, the LNP comprise a combination of two, three, four, five or more of the foregoing ionizable lipids.

In some embodiments, the LNP has a negative net overall charge prior to complexation with one or more of a guide nucleic acid, a nucleic acid encoding the one or more guide nucleic acid, a nucleic acid encoding a polypeptide (e.g., an effector protein, an effector partner, a fusion protein, or a combination thereof). In some embodiments, the inner core is a hydrophobic core. In some embodiments, the one or more of a guide nucleic acid, the nucleic acid encoding the one or more guide nucleic acid, the nucleic acid encoding the polypeptide forms a complex with one or more of the cationic lipids and the ionizable lipids. In some embodiments, the nucleic acid encoding the polypeptide or the nucleic acid encoding the guide nucleic acid is self-replicating.

In some embodiments, a LNP comprises one or more of cationic lipids, ionizable lipids and modified versions thereof. In some embodiments, the ionizable lipid comprises TT3 or a derivative thereof. Accordingly, in some embodiments, the LNP comprises one or more of TT3 and pegylated TT3. The publication WO2016187531 is hereby incorporated by reference in its entirety, which describes representative LNP formulations in Table 2 and Table 3, and representative methods of delivering LNP formulations in Example 7.

In some embodiments, a LNP comprises a lipid composition targeting to a specific organ. In some embodiments, the lipid composition comprises lipids having a specific alkyl chain length that controls accumulation of the LNP in the specific organ (e.g., liver or spleen). In some embodiments, the lipid composition comprises a biomimetic lipid that controls accumulation of the LNP in the specific organ (e.g., brain). In some embodiments, the lipid composition comprises lipid derivatives (e.g., cholesterol derivatives) that controls accumulation of the LNP in a specific cell (e.g., liver endothelial cells, Kupffer cells, hepatocytes).

In some embodiments, the LNP described herein comprises nucleic acids (e.g., DNA or RNA) encoding an effector protein described herein, an effector partner described herein, a fusion protein described herein, a guide nucleic acid described herein, or combinations thereof. In some embodiments, the LNP comprises an mRNA that produces an effector protein described herein, an effector partner described herein, or a fusion protein described herein when translated. In some embodiments, the LNP comprises chemically modified guide nucleic acids. In some embodiments, the LNP contains the nucleic acid encoding the effector protein and the guide nucleic acid, wherein the nucleic acid encoding the effector protein comprises a messenger RNA.

### Delivery of Viral Vectors

In some embodiments, a vector described herein comprises a viral vector. In some embodiments, the viral vector comprises a nucleic acid to be delivered into a host cell by a recombinantly produced virus or viral particle. The nucleic acid may be single-stranded or double stranded, linear or circular, segmented or non-segmented. The nucleic acid may comprise DNA, RNA, or a combination thereof. In some embodiments, the vector is an adeno-associated viral vector. There are a variety of viral vectors that are associated with various types of viruses, including but not limited to retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. In some embodiments, the vector is an adeno-associated viral (AAV) vector. In some embodiments, the viral vector is a recombinant viral vector. In some embodiments, the vector is a retroviral vector. In some embodiments, the retroviral vector is a lentiviral vector. In some embodiments, the retroviral vector comprises gamma-retroviral vector. A viral vector provided herein may be derived from or based on any such virus. For example, in some embodiments, the gamma-retroviral vector is derived from a Moloney Murine Leukemia Virus (MoMLV, MMLV, MuLV, or MLV) or a Murine Stem cell Virus (MSCV) genome. In some embodiments, the lentiviral vector is derived from the human immunodeficiency virus (HIV) genome. In some embodiments, the viral vector is a chimeric viral vector. In some embodiments, the chimeric viral vector comprises viral portions from two or more viruses. In some embodiments, the viral vector corresponds to a virus of a specific serotype.

In some embodiments, a viral vector is an adeno-associated viral vector (AAV vector). In some embodiments, a viral particle that delivers a viral vector described herein is an AAV. In some embodiments, the AAV comprises any AAV known in the art. In some embodiments, the viral vector corresponds to a virus of a specific AAV serotype. In some embodiments, the AAV serotype is selected from an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, an AAV12 serotype, an AAV-rh10 serotype, and any combination, derivative, or variant thereof. In some embodiments, the AAV vector is a recombinant vector, a hybrid AAV vector, a chimeric AAV vector, a self-complementary AAV (scAAV) vector, a single-stranded AAV, or any combination thereof. scAAV genomes are generally known in the art and contain both DNA strands which can anneal together to form double-stranded DNA.

In some embodiments, an AAV vector described herein is a chimeric AAV vector. In some embodiments, the chimeric AAV vector comprises an exogenous amino acid or an amino acid substitution, or capsid proteins from two or more serotypes. In some examples, a chimeric AAV vector is genetically engineered to increase transduction efficiency, selectivity, or a combination thereof.

In some embodiments, AAV vector described herein comprises two inverted terminal repeats (ITRs). According, in some embodiments, the viral vector provided herein comprises two inverted terminal repeats of AAV. A nucleotide sequence between the ITRs of an AAV vector provided herein comprises a sequence encoding genome editing tools. In some embodiments, the genome editing tools comprise a nucleic acid encoding one or more polypeptides (e.g., effector proteins, effector partners, fusion proteins, or combinations thereof), a nucleic acid encoding the one or more polypeptides comprising a heterologous peptide (e.g., a nuclear localization signal (NLS), polyA tail), one or more guide nucleic acids, a nucleic acid encoding the one or more guide nucleic acids, respective promoter(s), or any combinations thereof. In some embodiments, viral vectors provided herein comprise at least one promotor or a combination of promoters driving expression or transcription of one or more genome editing tools described herein. In some embodiments, a coding region of the AAV vector forms an intramolecular double-stranded DNA template thereby generating the AAV vector that is a self-complementary AAV (scAAV) vector. In some embodiments, the scAAV vector comprises the sequence encoding genome editing tools that has a length of about 2 kb to about 3 kb. In some embodiments, the AAV vector provided herein is a self-inactivating AAV vector. In some embodiments, the AAV vector provided herein comprises a modification, such as an insertion, deletion, chemical alteration, or synthetic modification, relative to a wild-type AAV vector.

### Producing AAV Delivery Vectors

In some embodiments, methods of producing AAV delivery vectors herein comprise packaging a nucleic acid encoding a polypeptide (e.g., an effector protein, an effector partner, a fusion protein, or a combination thereof) and a guide nucleic acid, or a combination thereof, into an AAV vector. In some embodiments, methods of producing the delivery vector comprises, (a) contacting a cell with at least one nucleic acid encoding: (i) a guide nucleic acid; (ii) a Replication (Rep) gene; and (iii) a Capsid (Cap) gene that encodes an AAV capsid protein; (b) expressing the AAV capsid protein in the cell; (c) assembling an AAV particle; and (d) packaging the polypeptide encoding nucleic acid into the AAV particle, thereby generating an AAV delivery vector. In some embodiments, promoters, stuffer sequences and any combination thereof are packaged in the AAV vector. In some examples, the AAV vector is package 1, 2, 3, 4, or 5 guide nucleic acids or copies thereof. In some embodiments, the AAV vector comprises inverted terminal repeats, e.g., a 5′ inverted terminal repeat and a 3′ inverted terminal repeat. In some embodiments, the AAV vector comprises a mutated inverted terminal repeat that lacks a terminal resolution site. In some embodiments, an AAV vector comprises a donor nucleic acid.

In some embodiments, a hybrid AAV vector is produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) are used in a capsid from a second AAV serotype (e.g., AAV9), wherein the first and second AAV serotypes are not the same. As a non-limiting example, a hybrid AAV serotype comprising the AAV2 ITRs and AAV9 capsid protein is indicated AAV2/9. In some examples, the hybrid AAV delivery vector comprises an AAV2/1, AAV2/2, AAV 2/4, AAV2/5, AAV2/8, or AAV2/9 vector.

### Producing AAV Particles

In some embodiments, AAV particles described herein are recombinant AAV (rAAV). In some embodiments, rAAV particles are generated by transfecting AAV producing cells with an AAV-containing plasmid carrying the nucleotide sequence encoding the genome editing tools, a plasmid that carries viral encoding regions, i.e., Rep and Cap gene regions; and a plasmid that provides the helper genes such as E1A, E1B, E2A, E4ORF6 and VA. In some embodiments, the AAV producing cells are mammalian cells. In some embodiments, host cells for rAAV viral particle production are mammalian cells. In some embodiments, a mammalian cell for rAAV viral particle production is a COS cell, a HEK293T cell, a HeLa cell, a KB cell, a variant thereof, or a combination thereof. In some embodiments, rAAV virus particles can be produced in the mammalian cell culture system by providing the rAAV plasmid to the mammalian cell. In some embodiments, producing rAAV virus particles in a mammalian cell comprises transfecting vectors that express the rep protein, the capsid protein, and the gene-of-interest expression construct flanked by the ITR sequence on the 5′ and 3′ ends. Methods of such processes are provided in, for example, Naso et al., BioDrugs, 2017 August; 31(4):317-334 and Benskey et al., (2019), Methods Mol Biol., 1937:3-26, each of which is incorporated by reference in their entireties.

In some embodiments, rAAV is produced in a non-mammalian cell. In some embodiments, rAAV is produced in an insect cell. In some embodiments, the insect cell for producing rAAV viral particles comprises a Sf9 cell. In some embodiments, production of rAAV virus particles in insect cells comprise baculovirus. In some embodiments, production of rAAV virus particles in insect cells comprise infecting the insect cells with three recombinant baculoviruses, one carrying the cap gene, one carrying the rep gene and one carrying the gene-of-interest expression construct enclosed by an ITR on both the 5′ and 3′ end. In some embodiments, rAAV virus particles are produced by the One Bac system. In some embodiments, rAAV virus particles can be produced by the Two Bac system. In some embodiments, in the Two Bac system, the rep gene and the cap gene of the AAV is integrated into one baculovirus virus genome, and the ITR sequence and the gene-of-interest expression construct is integrated into another baculovirus virus genome. In some embodiments, in the One Bac system, an insect cell line that expresses both the rep protein and the capsid protein is established and infected with a baculovirus virus integrated with the ITR sequence and the gene-of-interest expression construct. Details of such processes are provided in, for example, Smith et. al., (1983), Mol. Cell. Biol., 3(12):2156-65; Urabe et al., (2002), Hum. Gene. Ther., 1; 13(16):1935-43; and Benskey et al., (2019), Methods Mol Biol., 1937:3-26, each of which is incorporated by reference in its entirety.

### V. Target Nucleic Acids

Disclosed herein are compositions, systems and methods for editing a target nucleic acid. In some embodiments, the target nucleic acid is a double stranded nucleic acid. In some embodiments, the target nucleic acid is a single stranded nucleic acid. Alternatively, or in combination, the target nucleic acid is a double stranded nucleic acid and is prepared into single stranded nucleic acids before or upon contacting an RNP. In some embodiments, the single stranded nucleic acid comprises a RNA, wherein the RNA comprises a mRNA, a rRNA, a tRNA, a non-coding RNA, a long non-coding RNA, a microRNA (miRNA) and a single-stranded RNA (ssRNA). In some embodiments, the target nucleic acid is complementary DNA (cDNA) synthesized from a single-stranded RNA template in a reaction catalyzed by a reverse transcriptase. In some embodiments, the target nucleic acid comprises an RNA, a DNA, or combination thereof. In some embodiments, guide nucleic acids described herein hybridize to a portion of the target nucleic acid. In some embodiments, the target nucleic acid is from a virus, a parasite, or a bacterium described herein.

In some embodiments, a target nucleic acid comprising a target sequence comprises a PAM sequence. In some embodiments, the PAM sequence is adjacent to the target sequence. In some embodiments, the PAM sequence is 3′ to the target sequence. In some embodiments, the PAM sequence is directly 3′ to the target sequence. In some embodiments, the PAM sequence 5′ to the target sequence. In some embodiments, the PAM sequence is directly 5′ to the target sequence. In some embodiments, the target nucleic acid as described in the methods herein does not initially comprise a PAM sequence. However, any target nucleic acid of interest may be generated using the methods described herein to comprise a PAM sequence, and thus be a PAM target nucleic acid. A PAM target nucleic acid, as used herein, refers to a target nucleic acid that has been amplified to insert a PAM sequence that is recognized by a polypeptide system.

In some embodiments, a target nucleic acid comprises 5 to 100, 5 to 90, 5 to 80, 5 to 70, 5 to 60, 5 to 50, 5 to 40, 5 to 30, 5 to 25, 5 to 20, 5 to 15, or 5 to 10 linked nucleotides. In some embodiments, the target nucleic acid comprises 10 to 90, 20 to 80, 30 to 70, or 40 to 60 linked nucleotides. In some embodiments, the target nucleic acid comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 linked nucleotides. In some embodiments, the target nucleic acid comprises at least 10, at least 20, at least 30, at least 40, at least 50, at least 60, at least 70, at least 80, at least 90, or at least 100 linked nucleotides. In some embodiments, the target sequence in the target nucleic acid comprises at least 10 contiguous nucleotides that are complementary to the guide nucleic acid or engineered guide nucleic acid.

In some embodiments, compositions, systems and methods described herein comprise a target nucleic acid that is responsible for a disease, contain a mutation (e.g., single strand polymorphism, point mutation, insertion, or deletion), be contained in an amplicon, or be uniquely identifiable from the surrounding nucleic acids (e.g., contain a unique sequence of nucleotides). In some embodiments, the target nucleic acid has undergone a modification (e.g., an editing) after contacting with an RNP. In some embodiments, the editing is a change in the nucleotide sequence of the target nucleic acid. In some embodiments, the change comprises an insertion, deletion, or substitution of one or more nucleotides compared to the target nucleic acid that has not undergone any modification.

In some embodiments, the target nucleic acid comprises a nucleic acid sequence from a pathogen responsible for a disease. Non-limiting examples of pathogens are bacteria, a virus and a fungus. In some embodiments, the target sequence is a portion of a nucleic acid from a virus or a bacterium or other agents responsible for a disease in the sample. The target sequence, in some embodiments, is a portion of a nucleic acid from a sexually transmitted infection or a contagious disease, in the sample. The target sequence, in some embodiments, is a portion of a nucleic acid from an upper respiratory tract infection, a lower respiratory tract infection, or a contagious disease, in the sample. The target sequence, in some embodiments, is a portion of a nucleic acid from a hospital acquired infection or a contagious disease, in the sample. The target sequence, in some embodiments, is a portion of a nucleic acid from sepsis, in the sample. In some embodiments, the target nucleic acid is a portion of a nucleic acid from a genomic locus, or any DNA amplicon, such as a reverse transcribed mRNA or a cDNA from a gene locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya and leishmaniasis. Pathogens include viruses, fungi, helminths, protozoa, malarial parasites, Plasmodium parasites, Toxoplasma parasites and Schistosoma parasites. Helminths include roundworms, heartworms, phytophagous nematodes, flukes, Acanthocephala and tapeworms. Protozoan infections include infections from Giardia spp., Trichomonas spp., African trypanosomiasis, amoebic dysentery, babesiosis, balantidial dysentery, Chagas disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis and Candida albicans. A pathogenic virus can be a DNA virus or an RNA virus. Pathogenic viruses include but are not limited to respiratory viruses, adenoviruses, parainfluenza viruses, severe acute respiratory syndrome (SARS), coronavirus (e.g., SARS-COV), MERS, gastrointestinal viruses (e.g., noroviruses, rotaviruses, some adenoviruses, astroviruses), exanthematous viruses (e.g., the virus that causes measles, the virus that causes rubella, the virus that causes chickenpox/shingles, the virus that causes roseola, the virus that causes smallpox, the virus that causes fifth disease, chikungunya virus infection), hepatic viral diseases (e.g., hepatitis A, B, C, D, E), cutaneous viral diseases (e.g., warts (including genital, anal), herpes (including oral, genital, anal), molluscum contagiosum), hemmorhagic viral diseases (e.g., Ebola, Lassa fever, dengue fever, yellow fever, Marburg hemorrhagic fever, Crimean-Congo hemorrhagic fever), neurologic viruses (e.g., polio, viral meningitis, viral encephalitis, rabies), sexually transmitted viruses (e.g., HIV, HPV and the like), Adenovirus, coronavirus (i.e., a virus that causes COVID-19), Coronavirus HKU1, Coronavirus NL63, Coronavirus 229E, Coronavirus OC43, Severe Acute Respiratory Syndrome Coronavirus 2 (SARS-COV-2), Human Metapneumovirus (hMPV), Human Rhinovirus/Enterovirus, influenza virus, Influenza A, Influenza A/H1, Influenza A/H3, Influenza A/H1-2009, Influenza B, Influenza C, Parainfluenza Virus 1, Parainfluenza Virus 2, Parainfluenza Virus 3, Parainfluenza Virus 4, Respiratory Syncytial Virus), human immunodeficiency virus (e.g., HIV), human papillomavirus (e.g., HPV), chlamydia, gonorrhea, syphilis, trichomoniasis, sexually transmitted infection, malaria, Dengue fever, Ebola, chikungunya, leishmaniasis, Orthopoxvirus (e.g., monkeypox virus, cowpox virus, camelpox virus, horsepox virus, vaccinia virus and variola virus), West Nile virus, herpes virus, yellow fever virus, Hepatitis Virus C, Hepatitis Virus A, Hepatitis Virus B, papillomavirus and the like. Pathogens include, e.g., HIV virus, Bordetella parapertussis, Bordetella pertussis, Chlamydia pneumoniae, Mycoplasma pneumoniae, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium, M. pneumoniae, Enterobacter cloacae, Kiebsiella aerogenes, Proteus vulgaris, Serratia macesens, Enterococcus faecalis, Enterococcus faecium, Streptococcus intermdius, Streptococcus pneumoniae and Streptococcus pyogenes. In some embodiments, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment.

In some embodiments, the target sequence is comprised in a sample. In some embodiments, the sample used for genetic disorder testing, cancer testing, or cancer risk testing can comprise at least one target sequence or target nucleic acid segment that can bind to a guide nucleic acid of the reagents described herein. In some embodiments, the sample used comprises a target sequence or target nucleic acid of a gene recited in TABLE 8.

In some embodiments, the sample used for phenotyping testing can comprise at least one target nucleic acid segment that can bind to a guide nucleic acid of the reagents described herein. The target nucleic acid segment, in some cases, is a portion of a nucleic acid from a gene associated with a phenotypic trait.

In some embodiments, the sample used for genotyping testing can comprise at least one target nucleic acid segment that can bind to a guide nucleic acid of the reagents described herein. The target nucleic acid segment, in some cases, is a portion of a nucleic acid from a gene associated with a genotype.

In some embodiments, the target nucleic acid comprises a nucleic acid sequence of a virus, a bacterium, or other pathogen responsible for a disease in a plant (e.g., a crop). Methods and compositions of the disclosure may be used to treat or detect a disease in a plant. For example, the methods of the disclosure are used for targeting a viral nucleic acid sequence in a plant. A polypeptide (e.g., an effector protein, an effector partner, a fusion protein, or a combination thereof) of the disclosure may cleave the viral nucleic acid. In some embodiments, the target nucleic acid comprises a nucleic acid sequence of a virus or a bacterium or other agents (e.g., any pathogen) responsible for a disease in the plant (e.g., a crop). In some embodiments, the target nucleic acid comprises RNA. The target nucleic acid, in some embodiments, is a portion of a nucleic acid from a virus or a bacterium or other agents responsible for a disease in the plant (e.g., a crop). In some embodiments, the target nucleic acid is a portion of a nucleic acid from a genomic locus, or any NA amplicon, such as a reverse transcribed mRNA or a cDNA from a gene locus, a transcribed mRNA, or a reverse transcribed cDNA from a gene locus in at a virus or a bacterium or other agents (e.g., any pathogen) responsible for a disease in the plant (e.g., a crop). A virus infecting the plant may be an RNA virus. A virus infecting the plant may be a DNA virus. Non-limiting examples of viruses that are targeted with the disclosure include Tobacco mosaic virus (TMV), Tomato spotted wilt virus (TSWV), Cucumber mosaic virus (CMV), Potato virus Y (PVY), Cauliflower mosaic virus (CaMV) (RT virus), Plum pox virus (PPV), Brome mosaic virus (BMV) and Potato virus X (PVX).

In some embodiments, a target nucleic acid comprises a portion or a specific region of a nucleic acid from a genomic locus, any DNA amplicon of, a reverse transcribed mRNA, or a cDNA from a gene described herein. In some embodiments, the target nucleic acid is an amplicon of at least a portion of a gene. Non-limiting examples of genes are recited in TABLE 8. Nucleic acid sequences of target nucleic acids and/or corresponding genes are readily available in public databases as known and used in the art. In some embodiments, the target nucleic acid is selected from TABLE 8. In some embodiments, the target nucleic acid comprises one or more target sequences. In some embodiments, the one or more target sequence is within any one of the target nucleic acids set forth in TABLE 8.

In some embodiments, the target nucleic acid is any one of: a naturally occurring eukaryotic sequence, a naturally occurring prokaryotic sequence, a naturally occurring viral sequence, a naturally occurring bacterial sequence, a naturally occurring fungal sequence, an engineered eukaryotic sequence, an engineered prokaryotic sequence, an engineered viral sequence, an engineered bacterial sequence, an engineered fungal sequence, a fragment of a naturally occurring sequence, a fragment of an engineered sequence and combinations thereof.

In some embodiments, the target nucleic acid is isolated from any one of: a naturally occurring cell, a eukaryotic cell, a prokaryotic cell, a plant cell, a fungal cell, an animal cell, cell of an invertebrate, a fly cell, a cell of a vertebrate, a mammalian cell, a primate cell, a non-human primate cell, a human cell, a living cell, a non-living cell, a modified cell, a derived cell and a non-naturally occurring cell. In some embodiments, the target nucleic acid is isolated from a population of cells.

Nucleic acids, such as DNA and pre-mRNA, described herein can contain at least one intron and at least one exon, wherein as read in the 5′ to the 3′ direction of a nucleic acid strand, the 3′ end of an intron can be adjacent to the 5′ end of an exon, and wherein said intron and exon correspond for transcription purposes. If a nucleic acid strand contains more than one intron and exon, the 5′ end of the second intron is adjacent to the 3′ end of the first exon, and 5′ end of the second exon is adjacent to the 3′ end of the second intron. The junction between an intron and an exon can be referred to herein as a splice junction, wherein a 5′ splice site (SS) can refer to the +1/+2 position at the 5′ end of intron and a 3′SS can refer to the last two positions at the 3′ end of an intron. Alternatively, a 5′ SS can refer to the 5′ end of an exon and a 3′SS can refer to the 3′ end of an exon. In some embodiments, nucleic acids can contain one or more elements that act as a signal during transcription, splicing and/or translation. In some embodiments, signaling elements include a 5′SS, a 3′SS, a premature stop codon, U1 and/or U2 binding sequences, and cis acting elements such as branch site (BS), polypyridine tract (PYT), exonic and intronic splicing enhancers (ESEs and ISEs) or silencers (ESSs and ISSs). In some embodiments, nucleic acids also comprise an untranslated region (UTR), such as a 5′ UTR or a 3′ UTR. In some embodiments, the start of an exon or intron is referred to interchangeably herein as the 5′ end of an exon or intron, respectively. Likewise, in some embodiments, the end of an exon or intron is referred to interchangeably herein as the 3′ end of an exon or intron, respectively.

In some embodiments, at least a portion of at least one target sequence is within 1, about 5 or more, about 10 or more, about 15 or more, about 20 or more, about 25 or more, about 30 or more, about 35 or more, about 40 or more, about 45 or more, about 50 or more, about 55 or more, about 60 or more, about 65 or more, about 70 or more, about 75 or more, about 80 or more, about 85 or more, about 90 or more, about 95 or more, about 100 or more, about 105 or more, about 110 or more, about 115 or more, about 120 or more, about 125 or more, about 130 or more, about 135 or more, about 140 or more, about 145 or more, or about 150 to about 300 nucleotides adjacent to: the 5′ end of an exon; the 3′ end of an exon; the 5′ end of an intron; the 3′ end of an intron; one or more signaling element comprising a 5′SS, a 3′SS, a premature stop codon, U1 binding sequence, U2 binding sequence, a BS, a PYT, ESE, an ISE, an ESS, an ISS; a 5′ UTR; a 3′ UTR; more than one of the foregoing, or any combination thereof. In some embodiments, the target nucleic acid comprises a target locus. In some embodiments, the target nucleic acid comprises more than one target loci. In some embodiments, the target nucleic acid comprises two target loci. Accordingly, in some embodiments, the target nucleic acid can comprise one or more target sequences.

In some embodiments, compositions, systems and methods described herein comprise an edited target nucleic acid which can describe a target nucleic acid wherein the target nucleic acid has undergone a change, for example, after contact with a polypeptide (e.g., an effector protein, an effector partner, a fusion protein, or a combination thereof). In some embodiments, the editing is an alteration in the nucleotide sequence of the target nucleic acid. In some embodiments, the edited target nucleic acid comprises a nicked target strand or a nicked non-target strand. In some embodiments, the edited target nucleic acid comprises an insertion, deletion, or replacement of one or more nucleotides compared to the unedited target nucleic acid. In some embodiments, the editing is a mutation.

### Mutations

In some embodiments, target nucleic acids described herein comprise a mutation. In some embodiments, a composition, system or method described herein can be used to edit a target nucleic acid comprising a mutation such that the mutation is edited to be the wild-type nucleotide or nucleotide sequence. In some embodiments, a composition, system or method described herein can be used to detect a target nucleic acid comprising a mutation. A mutation may result in the insertion of at least one amino acid in a protein encoded by the target nucleic acid. A mutation may result in the deletion of at least one amino acid in a protein encoded by the target nucleic acid. A mutation may result in the substitution of at least one amino acid in a protein encoded by the target nucleic acid. A mutation that results in the deletion, insertion, or substitution of one or more amino acids of a protein encoded by the target nucleic acid may result in misfolding of a protein encoded by the target nucleic acid. A mutation may result in a premature stop codon, thereby resulting in a truncation of the encoded protein.

Non-limiting examples of mutations are insertion-deletion (indel), a point mutation, single nucleotide polymorphism (SNP), a chromosomal mutation, a copy number mutation or variation, and frameshift mutations. In some embodiments, an indel mutation is an insertion or deletion of one or more nucleotides. In some embodiments, a point mutation comprises a substitution, insertion, or deletion. In some embodiments, a frameshift mutation occurs when the number of nucleotides in the insertion/deletion is not divisible by three, and it occurs in a protein coding region. In some embodiments, a chromosomal mutation can comprise an inversion, a deletion, a duplication, or a translocation of one or more nucleotides. In some embodiments, a copy number variation can comprise a gene amplification or an expanding trinucleotide repeat. In some embodiments, an SNP is associated with a phenotype of the sample or a phenotype of the organism from which the sample was taken. In some embodiments, an SNP is associated with altered phenotype from wild type phenotype. In some embodiments, the SNP is a synonymous substitution or a nonsynonymous substitution. In some embodiments, the nonsynonymous substitution is a missense substitution or a nonsense point mutation. In some embodiments, the synonymous substitution is a silent substitution.

In some embodiments, a target nucleic acid described herein comprises a mutation of one or more nucleotides. In some embodiments, the one or more nucleotides comprises 1, 2, 3, 4, 5, 6, 7, 8, 9, 10, 11, 12, 13, 14, 15, 16, 17, 18, 19, 20 or more nucleotides. In some embodiments, the mutation comprises a deletion, insertion, and/or substitution of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. In some embodiments, the mutation comprises a deletion, insertion and/or substitution of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides. The mutation may be located in a non-coding region or a coding region of a gene, wherein the gene is a target nucleic acid. A mutation may be in an open reading frame of a target nucleic acid. In some embodiments, guide nucleic acids described herein hybridize to a portion of the target nucleic acid comprising or adjacent to the mutation.

In some embodiments, the target nucleic acid comprises one or more mutations. In some embodiments, the target nucleic acid comprises one or more, two or more, three or more, four or more, five or more, six or more, seven or more, eight or more, nine or more, or ten or more mutations as compared to the unmutated target nucleic acid. In some embodiments, the target nucleic acid comprises a sequence comprising one or more, two or more, three or more, four or more, five or more, six or more, seven or more, eight or more, nine or more, or ten or more mutations as compared to the wildtype sequence. In some embodiments, the target nucleic acid comprises a mutation associated with a disease or disorder.

In some embodiments, target nucleic acids comprise a mutation, wherein the mutation is a SNP. In some embodiments, the single nucleotide mutation or SNP is associated with a phenotype of the sample or a phenotype of the organism from which the sample was taken. In some embodiments, the SNP is associated with altered phenotype from wild type phenotype. In some embodiments, a single nucleotide mutation, SNP, or deletion described herein is associated with a disease, such as a genetic disease. In some embodiments, the SNP is a synonymous substitution or a nonsynonymous substitution. In some embodiments, the nonsynonymous substitution is a missense substitution or a nonsense point mutation. In some embodiments, the synonymous substitution is a silent substitution. In some embodiments, the mutation is a deletion of one or more nucleotides. In some embodiments, the single nucleotide mutation, SNP, or deletion is associated with a disease such as a genetic disorder. In some embodiments, the mutation, such as a single nucleotide mutation, a SNP or a deletion, is encoded in the nucleotide sequence of a target nucleic acid from the germline of an organism or is encoded in a target nucleic acid from a diseased cell.

In some embodiments, the mutation is associated with a disease, such as a genetic disorder. In some embodiments, the mutation is encoded in the nucleotide sequence of a target nucleic acid from the germline of an organism or is encoded in a target nucleic acid from a diseased cell. In some embodiments, a target nucleic acid described herein comprises a mutation associated with a disease. In some examples, a mutation associated with a disease refers to a mutation whose presence in a subject indicates that the subject is susceptible to or suffers from, a disease, disorder, condition, or syndrome. In some examples, a mutation associated with a disease refers to a mutation which causes, contributes to the development of, or indicates the existence of the disease, disorder, condition, or syndrome. A mutation associated with a disease may also refer to any mutation which generates transcription or translation products at an abnormal level, or in an abnormal form, in cells affected by a disease relative to a control without the disease. In some examples, a mutation associated with a disease refers to a mutation whose presence in a subject indicates that the subject is susceptible to, or suffers from, a disease, disorder, or pathological state. In some embodiments, a mutation associated with a disease, comprises the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation. In some embodiments, a target nucleic acid described herein comprises a mutation associated with a disease, wherein the target nucleic acid is any one of the target nucleic acids set forth in TABLE 8. In some embodiments, a target nucleic acid described herein comprises a mutation associated with a disease, wherein the disease is any one of the diseases set forth in TABLE 9.

### VI. Compositions

Disclosed herein are compositions comprising one or more polypeptides (e.g., effector proteins, effector partners, fusion proteins, or combinations thereof) described herein or nucleic acids encoding the one or more polypeptides, one or more guide nucleic acids described herein or nucleic acids encoding the one or more guide nucleic acids described herein, or combinations thereof. In some embodiments, repeat sequences of the one or more guide nucleic acids interact with the one or more of the effector proteins. In some embodiments, spacer sequences of the one or more guide nucleic acids hybridizes with a target sequence of a target nucleic acid. In some embodiments, the compositions cleave (e.g., nick) a target strand or a non-target strand of a target nucleic acid. In some embodiments, the compositions are not capable of cleaving a target strand or a non-target strand of a target nucleic acid. In some embodiments, the compositions modify a target strand or a non-target strand of a target nucleic acid. In some embodiments, the compositions modify expression of the target nucleic acids, proteins associated with the expression of the target nucleic acids, other nucleic acids associated with the target nucleic acids, or combinations thereof. In some embodiments, the compositions edit a target nucleic acid in a cell or a subject. In some embodiments, the compositions edit a target nucleic acid or the expression thereof in a cell, in a tissue, in an organ, in vitro, in vivo, or ex vivo. In some embodiments, the compositions edit a target nucleic acid in a sample comprising the target nucleic.

In some embodiments, compositions described herein comprise plasmids described herein, viral vectors described herein, non-viral vectors described herein, or combinations thereof. In some embodiments, compositions described herein comprise the viral vectors. In some embodiments, compositions described herein comprise an AAV. In some embodiments, compositions described herein comprise liposomes (e.g., cationic lipids or neutral lipids), dendrimers, lipid nanoparticle (LNP), or cell-penetrating peptides. In some embodiments, compositions described herein comprise an LNP.

### Pharmaceutical Compositions

Described herein are formulations of introducing compositions or components of a system described herein to a host.

In some embodiments, compositions described herein are pharmaceutical compositions. In some embodiments, the pharmaceutical compositions comprise compositions described herein or systems described herein. In some embodiments, the pharmaceutical composition comprises a pharmaceutically acceptable salt, one or more of a vehicle, adjuvant, excipient, or carrier, such as a filler, disintegrant, a surfactant, a binder, a lubricant, or combinations thereof. Remington: The Science and Practice of Pharmacy, 21st edition, 2005, ed. D. B. Troy, Lippincott Williams & Wilkins, Philadelphia; Encyclopedia of Pharmaceutical Technology, eds. J. Swarbrick and J. C. Boylan, 1988-1999, Marcel Dekker, New York; and Encyclopedia of Pharmaceutical Technology, eds. J. Swarbrick, 2015, CRC Press, Boca Raton disclose various carriers used in formulating pharmaceutically acceptably compositions and known techniques for the preparation thereof. Non-limiting examples of pharmaceutically acceptable carriers and diluents suitable for the pharmaceutical compositions disclosed herein include buffers (e.g., neutral buffered saline, phosphate buffered saline); carbohydrates (e.g., glucose, mannose, sucrose, dextran, mannitol); polypeptides or amino acids (e.g., glycine); antioxidants; chelating agents (e.g., EDTA, glutathione); adjuvants (e.g., aluminum hydroxide); surfactants (Polysorbate 80, Polysorbate 20, or Pluronic F68); glycerol; sorbitol; mannitol; polyethyleneglycol; and preservatives. In some embodiments, the vector is formulated for delivery through injection by a needle carrying syringe. In some embodiments, the composition is formulated for delivery by electroporation. In some embodiments, the composition is formulated for delivery by chemical method. In some embodiments, the pharmaceutical compositions comprise a virus vector or a non-viral vector.

Pharmaceutical compositions described herein comprise a salt. In some embodiments, the salt is a sodium salt. In some embodiments, the salt is a potassium salt. In some embodiments, the salt is a magnesium salt. In some embodiments, the salt is NaCl. In some embodiments, the salt is KNO₃. In some embodiments, the salt is Mg²⁺SO₄²⁻.

Pharmaceutical compositions described herein are in the form of a solution (e.g., a liquid). In some embodiments, the solution is formulated for injection, e.g., intravenous or subcutaneous injection. In some embodiments, the pH of the solution is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some cases, the pH of the solution is less than 7. In some cases, the pH is greater than 7.

### VII. Methods and Formulations for Introducing System Components and Compositions into a Target Cell

Disclosed herein, in some aspects, are systems and methods for introducing systems and components of such systems into a target cell. Such systems may comprise, as described herein, one or more components having any one of the polypeptides (e.g., effector proteins, effector partners, fusion proteins, or combination thereof) or a nucleic acid comprising a nucleotide sequence encoding same. In some embodiments, such systems comprise, as described herein, one or more components having a guide nucleic acid or a nucleic acid comprising a nucleotide sequence encoding same. In some embodiments, systems comprise one or more components having a guide nucleic acid and an additional nucleic acid. Systems and components thereof may be used to introduce the polypeptides, guide nucleic acids, or combinations thereof into a target cell. Such methods may be used to modify or edit a target nucleic acid. In some embodiments, systems comprise the polypeptide, one or more guide nucleic acids, and a reagent for facilitating the introduction of the polypeptide and the one or more guide nucleic acids. In some embodiments, system components for the methods comprise a solution, a buffer, a reagent for facilitating the introduction of the polypeptide and the one or more guide nucleic acids, or combinations thereof. A guide nucleic acid (or a nucleic acid comprising a nucleotide sequence encoding same) and/or a polypeptide (e.g., effector protein, effector partner, fusion protein, or combination thereof) (or a nucleic acid comprising a nucleotide sequence encoding same) described herein may be introduced into a host cell by any of a variety of well-known methods. As a non-limiting example, the guide nucleic acid and/or polypeptide are combined with a lipid. As another non-limiting example, the guide nucleic acid and/or polypeptide are combined with a particle or formulated into a particle.

### Methods for Introducing System Components and Compositions to a Host

Described herein are methods of introducing various components described herein to a host. A host may be any suitable host, such as a host cell. When described herein, a host cell may be an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells may be, or have been, used as recipients for methods of introduction described herein, and include the progeny of the original cell which has been transformed by the methods of introduction described herein. It is understood that the progeny of a single cell may not necessarily be completely identical in morphology or in genomic or total DNA complement as the original parent, due to natural, accidental, or deliberate mutation. A host cell may be a recombinant host cell or a genetically modified host cell, if a heterologous nucleic acid, e.g., an expression vector, has been introduced into the cell.

Methods of introducing a nucleic acid and/or protein into a host cell are known in the art, and any convenient method may be used to introduce a subject nucleic acid (e.g., an expression construct/vector) into a target cell (e.g., a human cell and the like). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al. Adv Drug Deliv Rev. 2012 Sep. 13. pii: S0169-409X(12)00283-9. doi: 10.1016/j.addr.2012.09.023) and the like. In some embodiments, the nucleic acid and/or protein(s) are introduced into a disease cell comprised in a pharmaceutical composition comprising the guide nucleic acid, the polypeptide, a pharmaceutically acceptable excipient, or combinations thereof.

In some embodiments, molecules of interest, such as nucleic acids of interest, are introduced to a host. In some embodiments, polypeptides are introduced to a host. In some embodiments, vectors, such as lipid particles and/or viral vectors may be introduced to a host. Introduction may be for contact with a host or for assimilation into the host, for example, introduction into a host cell.

In some embodiments, described herein are methods of introducing one or more nucleic acids, such as a nucleic acid encoding a polypeptide, a nucleic acid that, when transcribed, produces an engineered guide nucleic acid, or combinations thereof, into a host cell. Any suitable method may be used to introduce a nucleic acid into a cell. Suitable methods include, for example, viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery and the like. Further methods are described throughout.

Introducing one or more nucleic acids into a host cell may occur in any culture media and under any culture conditions that promote the survival of the cells. Introducing one or more nucleic acids into a host cell may be carried out in vivo or ex vivo. Introducing one or more nucleic acids into a host cell may be carried out in vitro.

In some embodiments, polypeptides (e.g., effector proteins, effector partners, fusion proteins, or combination thereof) are provided as RNA. The RNA may be provided by direct chemical synthesis or may be transcribed in vitro from a DNA (e.g., encoding the polypeptide). Once synthesized, the RNA may be introduced into a cell by way of any suitable technique for introducing nucleic acids into cells (e.g., microinjection, electroporation, transfection, etc.). In some embodiments, introduction of one or more nucleic acid is through the use of a vector and/or a vector system, accordingly, in some embodiments, compositions and system described herein comprise a vector and/or a vector system.

Vectors may be introduced directly to a host. In some embodiments, host cells are contacted with one or more vectors as described herein and, in some embodiments, said vectors are taken up by the cells. Methods for contacting cells with vectors include but are not limited to electroporation, calcium chloride transfection, microinjection, lipofection, micro-injection, contact with the cell or particle that comprises a molecule of interest, or a package of cells or particles that comprise molecules of interest.

Components described herein may also be introduced directly to a host. For example, an engineered guide nucleic acid is introduced to a host, specifically introduced into a host cell. Methods of introducing nucleic acids, such as RNA into cells include, but are not limited to direct injection, transfection, or any other method used for the introduction of nucleic acids.

Polypeptides (e.g., effector proteins, effector partners, fusion proteins, or combination thereof) described herein may also be introduced directly to a host. In some embodiments, polypeptides described herein are modified to promote introduction to a host. For example, polypeptides described herein may be modified to increase the solubility of the polypeptide. Such a polypeptide may optionally be fused to a polypeptide domain that increases solubility. The domain may be linked to the polypeptide through a defined protease cleavage site, such as TEV sequence which is cleaved by TEV protease. The linker may also include one or more flexible sequences, e.g., from 1 to 10 glycine residues. In some embodiments, the cleavage of the polypeptide is performed in a buffer that maintains solubility of the product, e.g., in the presence of from 0.5 to 2 M urea, in the presence of polypeptides and/or polynucleotides that increase solubility and the like. Domains of interest include endosomolytic domains, e.g., influenza HA domain; and other polypeptides that aid in production, e.g., IF2 domain, GST domain, GRPE domain and the like. In another example, the polypeptide is modified to improve stability. For example, the polypeptides is PEGylated, where the polyethyleneoxy group provides for enhanced lifetime in the blood stream. Polypeptides may also be modified to promote uptake by a host, such as a host cell. For example, a polypeptide described herein is fused to a polypeptide permeant domain to promote uptake by a host cell. Any suitable permeant domains may be used in the non-integrating polypeptides of the present disclosure, including peptides, peptidomimetics and non-peptide carriers. Examples include penetratin, a permeant peptide may be derived from the third alpha helix of Drosophila melanogaster transcription factor Antennapaedia; the HIV-1 tat basic region amino acid sequence, e.g., amino acids 49-57 of a naturally-occurring tat protein; and poly-arginine motifs, for example, the region of amino acids 34-56 of HIV-1 rev protein, nonaarginine, octa-arginine and the like. The site at which the fusion is made may be selected in order to optimize the biological activity, secretion or binding characteristics of the polypeptide. The optimal site may be determined by suitable methods.

### Formulations for Introducing System Components and Compositions to a Host

Described herein are formulations of introducing compositions or components of a system described herein to a host. In some embodiments, such formulations, systems and compositions described herein comprise polypeptides (e.g., effector proteins, effector partners, fusion proteins, or combination thereof) and a carrier (e.g., excipient, diluent, vehicle, or filling agent). In some aspects of the present disclosure, the polypeptides are provided in a pharmaceutical composition comprising the polypeptides and any pharmaceutically acceptable excipient, carrier, or diluent.

### VIII. Methods of Modifying a Nucleic Acid

Provided herein are compositions, methods and systems for modifying (e.g., editing) target nucleic acids. In general, modifying refers to changing the physical composition of a target nucleic acid. However, compositions, methods and systems disclosed herein may also modify target nucleic acids, such as making epigenetic modifications of target nucleic acids, which does not change the nucleotide sequence of the target nucleic acids per se. Polypeptides (e.g., effector proteins, effector partners, fusion proteins, or combination thereof), compositions and systems described herein may be used for modifying a target nucleic acid, which includes editing a target nucleic acid sequence. Modifying a target nucleic acid may comprise one or more of: cleaving (e.g., nicking) the target nucleic acid, deleting one or more nucleotides of the target nucleic acid, inserting one or more nucleotides into the target nucleic acid, mutating one or more nucleotides of the target nucleic acid, or otherwise changing one or more nucleotides of the target nucleic acid. Modifying a target nucleic acid may comprise one or more of: methylating, demethylating, deaminating, or oxidizing one or more nucleotides of the target nucleic acid.

Compositions, methods and systems described herein may modify a coding portion of a gene, a non-coding portion of a gene, or a combination thereof. Modifying at least one gene using the compositions, methods or systems described herein may reduce or increase expression of one or more genes. In some embodiments, the compositions, methods or systems reduce expression of one or more genes by at least 10%, at least 20%, at least 30%, at least 40%, at least 50%, at least 60%, at least 70%, at least 80%, at least 90%, or at least 95%. In some embodiments, the compositions, methods or systems remove all expression of a gene, also referred to as genetic knock out. In some embodiments, the compositions, methods or systems increase expression of one or more genes by at least 10%, at least 20%, at least 30%, at least 40%, at least 50%, at least 60%, at least 70%, at least 80%, at least 90%, or at least 100%.

In some embodiments, the compositions, methods or systems comprise a nucleic acid expression vector, or use thereof, to introduce polypeptides (e.g., effector proteins, effector partners, fusion proteins, or combination thereof), guide nucleic acid, or any combination thereof to a cell. In some embodiments, the nucleic acid expression vector is a viral vector. Viral vectors include, but are not limited to, retroviruses, adenoviruses, adeno-associated viruses and herpes simplex viruses. In some embodiments, the viral vector is a replication-defective viral vector, comprising an insertion of a therapeutic gene inserted in genes essential to the lytic cycle, preventing the virus from replicating and exerting cytotoxic effects. In some embodiments, the viral vector is an adeno associated viral (AAV) vector. In some embodiments, the nucleic acid expression vector is a non-viral vector. In some embodiments, compositions and methods comprise a lipid, polymer, nanoparticle, or a combination thereof, or use thereof, to introduce the polypeptide, guide nucleic acid, or any combination thereof to a cell. Non-limiting examples of lipids and polymers are cationic polymers, cationic lipids, or bio-responsive polymers. In some embodiments, the bio-responsive polymer exploits chemical-physical properties of the endosomal environment (e.g., pH) to preferentially release the genetic material in the intracellular space.

Methods of modifying may comprise contacting a target nucleic acid with one or more components, compositions or systems described herein. In some embodiments, a method of modifying comprises contacting a target nucleic acid with at least one of: a) one or more polypeptides (e.g., effector proteins, effector partners, fusion proteins, or combination thereof), or one or more nucleic acids encoding the one or more polypeptides; or b) one or more guide nucleic acids, or one or more nucleic acids encoding one or more guide nucleic acids. In some embodiments, a method of modifying comprises contacting a target nucleic acid with a system described herein wherein the system comprises components comprising at least one of: a) one or more polypeptides (e.g., effector proteins, effector partners, fusion proteins, or combination thereof), or one or more nucleic acids encoding the one or more polypeptides; or b) one or more guide nucleic acids, or one or more nucleic acids encoding one or more guide nucleic acids. In some embodiments, a method of modifying comprises contacting a target nucleic acid with a composition described herein comprising at least one of: a) one or more polypeptides (e.g., effector proteins, effector partners, fusion proteins, or combination thereof), or one or more nucleic acids encoding the one or more polypeptides; or b) one or more guide nucleic acids, or one or more nucleic acids encoding one or more guide nucleic acids; in a composition. In some embodiments, a method of modifying as described herein produces a modified target nucleic acid.

Editing a target nucleic acid sequence may introduce a mutation (e.g., point mutations, deletions) in a target nucleic acid relative to a corresponding wildtype nucleotide sequence. Editing may remove or correct a disease-causing mutation in a nucleic acid sequence to produce a corresponding wildtype nucleotide sequence. Editing a target nucleic acid sequence may remove/correct point mutations, deletions, null mutations, or tissue-specific mutations in a target nucleic acid. Editing a target nucleic acid sequence may be used to generate gene knock-out, gene knock-in, gene editing, gene tagging, or a combination thereof. Methods of the disclosure may be targeted to any locus in a genome of a cell.

Modifying may comprise cleaving of a single stranded target nucleic acid, nicking of a double stranded target nucleic acid, epigenetic modification (e.g., methylation, demethylation, acetylation, or deacetylation), or a combination thereof. In some embodiments, cleaving of a single stranded target nucleic acid or nicking of a double stranded target nucleic acid is site-specific, meaning cleavage or nicking occurs at a specific site in the target nucleic acid, often within the region of the target nucleic acid that hybridizes with the guide nucleic acid spacer sequence. In some embodiments, polypeptides (e.g., effector proteins, effector partners, fusion proteins, or combination thereof) introduce a single-stranded break in a target nucleic acid to produce a cleaved nucleic acid. In some embodiments, the polypeptides introduce a break in a single stranded RNA (ssRNA). The polypeptides may be coupled to a guide nucleic acid that targets a particular region of interest in the ssRNA. In some embodiments, the target nucleic acid and the resulting cleaved nucleic acid is contacted with a nucleic acid for homologous recombination (e.g., homology directed repair (HDR)) or non-homologous end joining (NHEJ). In some embodiments, a double-stranded break in the target nucleic acid is repaired (e.g., by NHEJ or HDR) such that the repair results in an indel in the target nucleic acid at or near the site of the double-stranded break. In some embodiments, an indel, sometimes referred to as an insertion-deletion or indel mutation, is a type of genetic mutation that results from the insertion and/or deletion of one or more nucleotide in a target nucleic acid. An indel may vary in length (e.g., 1 to 1,000 nucleotides in length) and be detected using methods well known in the art, including sequencing. If the number of nucleotides in the insertion/deletion is not divisible by three, and it occurs in a protein coding region, it is also a frameshift mutation. Indel percentage is the percentage of sequencing reads that show at least one nucleotide has been mutation that results from the insertion and/or deletion of nucleotides regardless of the size of insertion or deletion, or number of nucleotides mutated. For example, if there is at least one nucleotide deletion detected in a given target nucleic acid, it counts towards the percent indel value. As another example, if one copy of the target nucleic acid has one nucleotide deleted, and another copy of the target nucleic acid has 10 nucleotides deleted, they are counted the same. This number reflects the percentage of target nucleic acids that are edited by a given polypeptide.

In some embodiments, methods of modifying described herein nick a target nucleic acid at one or more locations to generate a nicked target nucleic acid. In some embodiments, the nicked target nucleic acid undergoes recombination (e.g., NHEJ or HDR). In some embodiments, nicking in the target nucleic acid is repaired (e.g., by NHEJ or HDR), such that the repair results in an indel in the target nucleic acid at or near the site of the cleavage site.

In some embodiments, wherein the compositions, systems and methods of the present disclosure restore a wild-type reading frame. A wild-type reading frame may be a reading frame that produces at least a partially, or fully, functional protein. A non-wild-type reading frame may be a reading frame that produces a non-functional or partially non-functional protein.

Accordingly, in some embodiments, compositions, systems and methods described herein edit 1 to 1,000 nucleotides or any integer in between, in a target nucleic acid. In some embodiments, 1 to 1,000, 2 to 900, 3 to 800, 4 to 700, 5 to 600, 6 to 500, 7 to 400, 8 to 300, 9 to 200, or 10 to 100 nucleotides, or any integer in between, are edited by the compositions, systems and methods described herein. In some embodiments, 1, 2, 3, 4, 5, 6, 7, 8, 9, 10, or more nucleotides are edited by the compositions, systems and methods described herein. In some embodiments, 10, 20, 30, 40, 50, 60, 70, 80 90, 100 or more nucleotides, or any integer in between, are edited by the compositions, systems and methods described herein. In some embodiments, 100, 200, 300, 400, 500, 600, 700, 800, 900 or more nucleotides, or any integer in between, are edited by the compositions, systems and methods described herein.

Methods may comprise use of two or more polypeptides (e.g., effector proteins, effector partners, fusion proteins, or combination thereof). An illustrative method for introducing a nick in a target nucleic acid comprises contacting the target nucleic acid with: (a) a first engineered guide nucleic acid comprising a region that binds to a first polypeptide described herein; and (b) a second engineered guide nucleic acid comprising a region that binds to a second polypeptide described herein, wherein the first engineered guide nucleic acid comprises an additional region that hybridizes to the target nucleic acid and wherein the second engineered guide nucleic acid comprises an additional region that hybridizes to the target nucleic acid. In some embodiments, the first and second polypeptide are identical. In some embodiments, the first and second polypeptide are not identical.

In some embodiments, editing a target nucleic acid comprises genome editing. Genome editing may comprise editing a genome, chromosome, plasmid, or other genetic material of a cell or organism. In some embodiments, the genome, chromosome, plasmid, or other genetic material of the cell or organism is modified in vivo. In some embodiments, the genome, chromosome, plasmid, or other genetic material of the cell or organism is modified in a cell. In some embodiments, the genome, chromosome, plasmid, or other genetic material of the cell or organism is modified in vitro. For example, a plasmid is edited in vitro using a composition described herein and introduced into a cell or organism.

In some embodiments, editing a target nucleic acid comprises deleting a sequence from a target nucleic acid. For example, a mutated sequence or a sequence associated with a disease is removed from a target nucleic acid. In some embodiments, editing a target nucleic acid comprises replacing a sequence in a target nucleic acid with a second sequence. For example, a mutated sequence or a sequence associated with a disease is replaced with a second sequence lacking the mutation or that is not associated with the disease. In some embodiments, editing a target nucleic acid comprises deleting or replacing a sequence comprising markers associated with a disease or disorder.

In some embodiments, methods comprise editing a target nucleic acid with two or more polypeptides (e.g., effector proteins, effector partners, fusion proteins, or combination thereof). Editing a target nucleic acid may comprise introducing a single-stranded break in a target nucleic acid. In some embodiments, a break is introduced by contacting a target nucleic acid with the polypeptide and guide nucleic acid. The guide nucleic acid may bind to the polypeptide and hybridize to a region of the target nucleic acid, thereby recruiting the polypeptide to the region of the target nucleic acid. Binding of the polypeptide to the guide nucleic acid and the region of the target nucleic acid may activate the second polypeptide. In some embodiments, the second polypeptide introduces a base pair modification in the region of the target nucleic acid. In some embodiments, editing a target nucleic acid comprises introducing a break in a first region of the target nucleic acid and a base pair modification in a second region of the target nucleic acid. The first polypeptide may introduce a break in a first strand at the first region of the target nucleic acid, and the second polypeptide may introduce a base pair modification in a second strand at the second region of the target nucleic acid during base mismatch repair.

In some embodiments, methods comprise editing a target nucleic acid with two or more polypeptides (e.g., effector proteins, effector partners, fusion proteins, or combination thereof). Editing a target nucleic acid may comprise introducing a two or more single-stranded breaks in a target nucleic acid. In some embodiments, a break is introduced by contacting a target nucleic acid with a first polypeptide and a guide nucleic acid. The guide nucleic acid may bind to the first polypeptide and hybridize to a first region of the target nucleic acid, thereby recruiting the first polypeptide to the first region of the target nucleic acid. Binding of the effector protein to the guide nucleic acid and the region of the target nucleic acid may activate the second polypeptide, and the second polypeptide may introduce a base pair modification. The events lead to activation of a third polypeptide, and the third polypeptide may introduce a second break (e.g., a single stranded break) in a third region of the target nucleic acid. In some embodiments, a segment of the target nucleic acid between the first break and the second break is removed, thereby favoring a use of uncleaved strand of the target nucleic acid as a template sequence during base mismatch repair.

Methods, systems and compositions described herein may edit a target nucleic acid wherein such editing may result in one or more indels. In some embodiments, where compositions, systems and/or methods described herein effect one or more indels, the impact on the transcription and/or translation of the target nucleic acid is predicted depending on: 1) the amount of indels generated; and 2) the location of the indel on the target nucleic acid. For example, as described herein, in some embodiments, if the amount of indels is not divisible by three, and the indels occur within or along a protein coding region, then the edit or mutation is a frameshift mutation. In some embodiments, if the amount of indels is divisible by three, then a frameshift mutation isnot be effected, but a splicing disruption mutation and/or sequence skip mutation is effected, such as an exon skip mutation. In some embodiments, if the amount of indels is not evenly divisible by three, then a frameshift mutation is effected.

Methods, systems and compositions described herein may edit a target nucleic acid wherein such editing may be measured by indel activity. Indel activity measures the amount of change in a target nucleic acid (e.g., nucleotide deletion(s) and/or insertion(s)) compared to a target nucleic acid that has not been contacted by a polypeptide described in compositions, systems and methods described herein. For example, indel activity is detected by next generation sequencing of one or more target loci of a target nucleic acid where indel percentage is calculated as the fraction of sequencing reads containing insertions or deletions relative to an unedited reference sequence. In some embodiments, methods, systems and compositions comprising polypeptides (e.g., effector proteins, effector partners, fusion proteins, or combination thereof) and guide nucleic acid described herein exhibit about 0.0001% to about 65% or more indel activity upon contact to a target nucleic acid compared to a target nucleic acid non-contacted with compositions, systems, or by methods described herein. For example, methods, systems and compositions comprising polypeptides (e.g., effector proteins, effector partners, fusion proteins, or combination thereof) and guide nucleic acid described herein exhibit about 0.0001%, about 0.001%, about 0.01%, about 0.1%, about 1%, about 5%, about 10%, about 15%, about 20%, about 25%, about 30%, about 35%, about 40%, about 45%, about 50%, about 55%, about 60%, about 65% or more indel activity.

In some embodiments, editing of a target nucleic acid as described herein effects one or more mutations comprising splicing disruption mutations, frameshift mutations (e.g., 1+ or 2+ frameshift mutation), sequence deletion, sequence skipping, sequence reframing, sequence knock-in, or any combination thereof. In some embodiments, the splicing disruption can be an editing that disrupts a splicing of a target nucleic acid or a splicing of a sequence that is transcribed from a target nucleic acid relative to a target nucleic acid without the splicing disruption. In some embodiments, the frameshift mutation can be an editing that alters the reading frame of a target nucleic acid relative to a target nucleic acid without the frameshift mutation. In some embodiments, the frameshift mutation can be a +2 frameshift mutation, wherein a reading frame is edited by 2 bases. In some embodiments, the frameshift mutation can be a +1 frameshift mutation, wherein a reading frame is edited by 1 base. In some embodiments, the frameshift mutation is an editing that alters the number of bases in a target nucleic acid so that it is not divisible by three. In some embodiments, the frameshift mutation can be an editing that is not a splicing disruption. In some embodiments a sequence as described in reference to the sequence deletion, sequence skipping, sequence reframing, and sequence knock-in can be a DNA sequence, a RNA sequence, an edited DNA or RNA sequence, a mutated sequence, a wild-type sequence, a coding sequence, a non-coding sequence, an exonic sequence (exon), an intronic sequence (intron), or any combination thereof. In some embodiments, the sequence deletion is an editing where one or more sequences in a target nucleic acid are deleted relative to a target nucleic acid without the sequence deletion. In some embodiments, the sequence deletion can result in or effect a splicing disruption or a frameshift mutation. In some embodiments, the sequence deletion result in or effect a splicing disruption.

In some embodiments, the sequence skipping is an editing where one or more sequences in a target nucleic acid are skipped upon transcription or translation of the target nucleic acid relative to a target nucleic acid without the sequence skipping. In some embodiments, the sequence skipping can result in or effect a splicing disruption or a frameshift mutation. In some embodiments, the sequence skipping can result in or effect a splicing disruption. In some embodiments, the sequence reframing is an editing where one or more bases in a target are edited so that the reading frame of the sequence is reframed relative to a target nucleic acid without the sequence reframing. In some embodiments, the sequence reframing can result in or effect a splicing disruption or a frameshift mutation. In some embodiments, the sequence reframing can result in or effect a frameshift mutation. In some embodiments, the sequence knock-in is an editing where one or more sequences is inserted into a target nucleic acid relative to a target nucleic acid without the sequence knock-in. In some embodiments, the sequence knock-in can result in or effect a splicing disruption or a frameshift mutation. In some embodiments, the sequence knock-in can result in or effect a splicing disruption.

In some embodiments, editing of a target nucleic acid can be locus specific, wherein compositions, systems and methods described herein can edit a target nucleic acid at one or more specific loci to effect one or more specific mutations comprising splicing disruption mutations, frameshift mutations, sequence deletion, sequence skipping, sequence reframing, sequence knock-in, or any combination thereof. For example, editing of a specific locus can affect any one of a splicing disruption, frameshift (e.g., 1+ or 2+ frameshift), sequence deletion, sequence skipping, sequence reframing, sequence knock-in, or any combination thereof. In some embodiments, editing of a target nucleic acid can be locus specific, modification specific, or both. In some embodiments, editing of a target nucleic acid can be locus specific, modification specific, or both, wherein compositions, systems and methods described herein comprise polypeptides (e.g., effector proteins, effector partners, fusion proteins, or combination thereof) described herein and a guide nucleic acid described herein.

Methods of editing a target nucleic acid or modulating the expression of a target nucleic acid may be performed in vivo. Methods of editing a target nucleic acid or modulating the expression of a target nucleic acid may be performed in vitro. For example, a plasmid is edited in vitro using a composition described herein and introduced into a cell or organism. Methods of editing a target nucleic acid or modulating the expression of a target nucleic acid may be performed ex vivo. For example, methods comprise obtaining a cell from a subject, editing a target nucleic acid in the cell with methods described herein, and returning the cell to the subject.

In some embodiments, methods of modifying described herein comprise contacting a target nucleic acid with one or more components, compositions or systems described herein. In some embodiments, the one or more components, compositions or systems described herein comprise at least one of: a) one or more polypeptides (e.g., effector proteins, effector partners, fusion proteins, or combination thereof), or one or more nucleic acids encoding the one or more polypeptides; and b) one or more guide nucleic acids, or one or more nucleic acids encoding the one or more guide nucleic acids. In some embodiments, the one or more polypeptides introduce a break in a single-stranded break in a target nucleic acid comprising a target strand and a non-target strand. In some embodiments, the one or more polypeptides introduce the single-stranded break in the target strand. In some embodiments, the one or more polypeptides introduce the single-stranded break in the non-target strand. In some embodiments, the one or more polypeptides introduce a base pair modification in the target nucleic acid. In some embodiments, the one or more polypeptides introduce the base pair modification in the target strand. In some embodiments, the one or more polypeptides introduce the base pair modification in the non-target strand. In some embodiments, methods of modifying described herein produce a modified target nucleic acid comprising an engineered nucleic acid sequence that expresses polypeptide having new activity as compared to an unmodified target nucleic acid, or alters expression of an endogenous polypeptide as compared to an unmodified target nucleic acid.

In some embodiments, methods of modifying described herein comprise using one or more guide nucleic acids or uses thereof, wherein the methods modify a target nucleic acid at a single location. In some embodiments, the methods comprise contacting an RNP comprising polypeptides (e.g., effector proteins, effector partners, fusion proteins, or combination thereof) and a guide nucleic acid to the target nucleic acid. In some embodiments, the methods introduce a mutation (e.g., point mutations, deletions) in the target nucleic acid relative to a corresponding wildtype nucleotide sequence. In some embodiments, the methods remove or correct a disease-causing mutation in a nucleic acid sequence to produce a corresponding wildtype nucleotide sequence. In some embodiments, the methods remove/correct point mutations, deletions, null mutations, or tissue-specific mutations in a target nucleic acid. In some embodiments, the methods introduce a single stranded cleavage, a nick, a deletion of one or two nucleotides, an insertion of one or two nucleotides, a substitution of one or two nucleotides, an epigenetic modification (e.g., methylation, demethylation, acetylation, or deacetylation), or a combination thereof to the target nucleic acid.

### Genetically Modified Cells and Organisms

Methods of editing described herein may be employed to generate a genetically modified cell. In some embodiments, the cell is a eukaryotic cell (e.g., a mammalian cell) or a prokaryotic cell (e.g., an archaeal cell). In some embodiments, the cell is derived from a multicellular organism and cultured as a unicellular entity. In some embodiments, the cell comprises a heritable genetic modification, such that progeny cells derived therefrom comprise the heritable genetic mutation. In some embodiments, the cell is progeny of a genetically modified cell comprising a genetic modification of the genetically modified parent cell. In some embodiments, the genetically modified cell comprises a deletion, insertion, mutation, or non-native sequence relative to a wild-type version of the cell or the organism from which the cell was derived.

Methods of editing described herein may be performed in a cell. In some embodiments, the cell is in vivo. In some embodiments, the cell is ex vivo. In some embodiments, the cell is an isolated cell. In some embodiments, the cell is inside of an organism. In some embodiments, the cell is an organism. In some embodiments, the cell is in a cell culture. In some embodiments, the cell is one of a collection of cells. In some embodiments, the cell is a mammalian cell or derived there from. In some embodiments, the cell is a rodent cell or derived there from. In some embodiments, the cell is a human cell or derived there from. In some embodiments, the cell is a eukaryotic cell or derived there from. In some embodiments, the cell is a progenitor cell or derived there from. In some embodiments, the cell is a pluripotent stem cell or derived there from. In some embodiments, the cell is an animal cell or derived there from. In some embodiments, the cell is an invertebrate cell or derived there from. In some embodiments, the cell is a vertebrate cell or derived there from. In some embodiments, the cell is from a specific organ or tissue. In some embodiments, the cell is a hepatocyte. In some embodiments, the tissue is a subject's blood, bone marrow, or cord blood. In some embodiments, the tissue is a heterologous donor blood, cord blood, or bone marrow. In some embodiments, the tissue is an allogenic blood, cord blood, or bone marrow. In some embodiments, the tissue is muscle. In some embodiments, the muscle is a skeletal muscle. In some embodiments, skeletal muscles include the following: abductor digiti minimi (foot), abductor digiti minimi (hand), abductor hallucis, abductor pollicis brevis, abductor pollicis longus, adductor brevis, adductor hallucis, adductor longus, adductor magnus, adductor pollicis, anconeus, articularis cubiti, articularis genu, aryepiglotticus, auricularis, biceps brachii, biceps femoris, brachialis, brachioradialis, buccinator, bulbospongiosus, constrictor of pharynx-inferior, constrictor of pharynx-middle, constrictor of pharynx-superior, coracobrachialis, corrugator supercilii, cremaster, cricothyroid, dartos, deep transverse perinei, deltoid, depressor anguli oris, depressor labii inferioris, diaphragm, digastric, digastric (anterior view), erector spinae-spinalis, erector spinae-iliocostalis, erector spinae-longissimus, extensor carpi radialis brevis, extensor carpi radialis longus, extensor carpi ulnaris, extensor digiti minimi (hand), extensor digitorum (hand), extensor digitorum brevis (foot), extensor digitorum longus (foot), extensor hallucis brevis, extensor hallucis longus, extensor indicis, extensor pollicis brevis, extensor pollicis longus, external oblique abdominis, flexor carpi radialis, flexor carpi ulnaris, flexor digiti minimi brevis (foot), flexor digiti minimi brevis (hand), flexor digitorum brevis, flexor digitorum longus (foot), flexor digitorum profundus, flexor digitorum superficialis, flexor hallucis brevis, flexor hallucis longus, flexor pollicis brevis, flexor pollicis longus, frontalis, gastrocnemius, gemellus inferior, gemellus superior, genioglossus, geniohyoid, gluteus maximus, gluteus medius, gluteus minimus, gracilis, hyoglossus, iliacus, inferior oblique, inferior rectus, infraspinatus, intercostals external, intercostals innermost, intercostals internal, internal oblique abdominis, interossei-dorsal of hand, interossei-dorsal of foot, interossei-palmar of hand, interossei-plantar of foot, interspinales, intertransversarii, intrinsic muscles of tongue, ishiocavernosus, lateral cricoarytenoid, lateral pterygoid, lateral rectus, latissimus dorsi, levator anguli oris, levator ani-coccygeus, levator ani-iliococcygeus, levator ani-pubococcygeus, levator ani-puborectalis, levator ani-pubovaginalis, levator labii superioris, levator labii superioris, alaeque nasi, levator palpebrae superioris, levator scapulae, levator veli palatini, levatores costarum, longus capitis, longus colli, lumbricals of foot, lumbricals of hand, masseter, medial pterygoid, medial rectus, mentalis, m. uvulae, mylohyoid, nasalis, oblique arytenoid, obliquus capitis inferior, obliquus capitis superior, obturator externus, obturator internus (A), obturator internus (B), omohyoid, opponens digiti minimi (hand), opponens pollicis, orbicularis oculi, orbicularis oris, palatoglossus, palatopharyngeus, palmaris brevis, palmaris longus, pectineus, pectoralis major, pectoralis minor, peroneus brevis, peroneus longus, peroneus tertius, piriformis (A), piriformis (B), plantaris, platysma, popliteus, posterior cricoarytenoid, procerus, pronator quadratus, pronator teres, psoas major, psoas minor, pyramidalis, quadratus femoris, quadratus lumborum, quadratus plantae, rectus abdominis, rectus capitus anterior, rectus capitus lateralis, rectus capitus posterior major, rectus capitus posterior minor, rectus femoris, rhomboid major, rhomboid minor, risorius, salpingopharyngeus, sartorius, scalenus anterior, scalenus medius, scalenus minimus, scalenus posterior, semimembranosus, semitendinosus, serratus anterior, serratus posterior inferior, serratus posterior superior, soleus, sphincter ani, sphincter urethrae, splenius capitis, splenius cervicis, stapedius, sternocleidomastoideohyoid, sternothyroid, styloglossus, stylohyoid, stylohyoid (anterior view), stylopharyngeus, subclavius, subcostalis, subscapularis, superficial transverse perinei, superior oblique, superior rectus, supinator, supraspinatus, temporalis, temporoparietalis, tensor fasciae lata, tensor tympani, tensor veli palatini, teres major, teres minor, thyro-arytenoid & vocalis, thyro-epiglotticus, thyrohyoid, tibialis anterior, tibialis posterior, transverse arytenoid, transversospinalis-multifidus, transversospinalis-rotatores, transversospinalis-semispinalis, transversus abdominis, transversus thoracis, trapezius, triceps, vastus intermedius, vastus lateralis, vastus medialis, zygomaticus major, or zygomaticus minor. In some embodiments, the cell is a myocyte. In some embodiments, the cell is a muscle cell. In some embodiments, the muscle cell is a skeletal muscle cell. In some embodiments, the skeletal muscle cell is a red (slow) skeletal muscle cell, a white (fast) skeletal muscle cell or an intermediate skeletal muscle cell.

Methods of editing described herein may comprise contacting cells with compositions or systems described herein. In some embodiments, the contacting comprises electroporation, acoustic poration, optoporation, viral vector-based delivery, iTOP, nanoparticle delivery (e.g., lipid or gold nanoparticle delivery), cell-penetrating peptide (CPP) delivery, DNA nanostructure delivery, or any combination thereof.

Methods of editing described herein may be performed in a subject. In some embodiments, the methods comprise administering compositions described herein to the subject. In some embodiments, the subject is a human. In some embodiments, the subject is a mammal (e.g., rat, mouse, cow, dog, pig, sheep, horse). In some embodiments, the subject is a vertebrate or an invertebrate. In some embodiments, the subject is a laboratory animal. In some embodiments, the subject is a patient. In some embodiments, the subject is at risk of developing, suffering from, or displaying symptoms of a disease. In some embodiments, the subject has a mutation associated with a gene described herein. In some embodiments, the subject displays symptoms associated with a mutation of a gene described herein.

### IX. Methods of Treating a Disease or Disorder

Described herein are methods for treating a disease in a subject by contacting a target nucleic acid with a composition or system described herein, wherein the target nucleic acid is associated with a gene or expression of a gene related to the disease. In some embodiments, methods comprise treating, preventing, or inhibiting a disease or disorder associated with a mutation or aberrant expression of a gene. In some embodiments, methods for treating a disease or disorder comprise methods of editing a nucleic acid described herein.

Methods may comprise administration of a composition(s) or component(s) of a system described herein. In some embodiments, the composition(s) or component(s) of the system comprises use of a recombinant nucleic acid (DNA or RNA), administered for the purpose to edit a nucleic acid. In some embodiments, the composition or component of the system comprises use of a vector to introduce a functional gene or transgene. In some embodiments, vectors comprise nonviral vectors, including cationic polymers, cationic lipids, or bio-responsive polymers. In some embodiments, the bio-responsive polymer exploits chemical-physical properties of the endosomal environment (e.g., pH) to preferentially release the genetic material in the intracellular space. In some embodiments, vectors comprise viral vectors, including retroviruses, adenoviruses, adeno-associated viruses and herpes simplex viruses. In some embodiments, the vector comprises a replication-defective viral vector, comprising an insertion of a therapeutic gene inserted in genes essential to the lytic cycle, preventing the virus from replicating and exerting cytotoxic effects. By way of non-limiting example, the composition(s) comprises pharmaceutical compositions described herein. Methods of gene therapy that are applicable to the compositions and systems described herein are described in more detail in Ingusci et al., “Gene Therapy Tools for Brain Diseases”, Front. Pharmacol. 10:724 (2019), which is hereby incorporated by reference in its entirety.

In some embodiments, treating, preventing, or inhibiting disease or disorder in a subject comprises contacting a target nucleic acid associated with a particular ailment with a composition described herein. In some aspects, the methods of treating, preventing, or inhibiting a disease or disorder involves removing, editing, modifying, replacing, transposing, or affecting the regulation of a genomic sequence of a patient in need thereof. In some embodiments, the methods of treating, preventing, or inhibiting a disease or disorder involves modulating gene expression.

In some embodiments, the compositions and systems described herein are for use in therapy. In some embodiments, the compositions and systems described herein are for use in treating a disease or condition described herein. Also provided is the use of the compositions described herein in the manufacture of a medicament. Also provided is the use of the compositions described herein in the manufacture of a medicament for therapeutic and/or prophylactic treatment of a disease or condition described herein.

In some embodiments, the polypeptides (e.g., effector proteins, effector partners, fusion proteins, or combination thereof) described herein are for use in therapy. In some embodiments, the polypeptides described herein are for use in treating a disease or condition described herein. Also provided is the use of the polypeptides described herein in the manufacture of a medicament. Also provided is the use of the polypeptides described herein in the manufacture of a medicament for therapeutic and/or prophylactic treatment of a disease or condition described herein.

In some embodiments, the guide nucleic acids described herein are for use in therapy. In some embodiments, the guide nucleic acids described herein are for use in treating a disease or condition described herein. Also provided is the use of the guide nucleic acids described herein in the manufacture of a medicament. Also provided is the use of the guide nucleic acids described herein in the manufacture of a medicament for therapeutic and/or prophylactic treatment of a disease or condition described herein.

Described herein are compositions and methods for treating a disease in a subject by editing a target nucleic acid associated with a gene or expression of a gene related to the disease. In some embodiments, methods comprise administering a composition or cell described herein to a subject. By way of non-limiting example, the disease is a cancer, an ophthalmological disorder, a neurological disorder, a neurodegenerative disease, a blood disorder, or a metabolic disorder, or a combination thereof. The disease may be an inherited disorder, also referred to as a genetic disorder. The disease may be the result of an infection or associated with an infection. Also, by way of non-limiting example, the compositions are pharmaceutical compositions described herein.

The compositions and methods described herein may be used to treat, prevent, or inhibit a disease or syndrome in a subject. In some embodiments, the disease is a liver disease, a lung disease, an eye disease, or a muscle disease. Exemplary diseases and syndromes include but are not limited to the diseases and syndromes listed in TABLE 9.

In some embodiments, compositions and methods edit at least one gene associated with a disease described herein or the expression thereof. In some embodiments, the disease is Alzheimer's disease, and the gene is selected from APP, BACE-1, PSD95, MAPT, PSEN1, PSEN2 and APOE&4. In some embodiments, the disease is Parkinson's disease, and the gene is selected from SNCA, GDNF and LRRK2. In some embodiments, the disease is congenital muscular dystrophy 1A (MDC1A), and the gene is LAMA1 or LAMA2. In some embodiments, the disease is Ullrich Congenital Muscular Dystrophy (UCMD), and the gene is selected from COL6A1, COL6A2 and COL6A3. In some embodiments, the disease is Limb Girdle Muscular Dystrophies (LGMD1B, LGMD2A, LGMD2B), and the gene is selected from LMNA, DYSF and CAPN3. In some embodiments, the disease is Nemaline Myopathy, and the gene is selected from ACTA1, NEB, TPM2, TPM3, TNNT1, TNNT3, TNNI2 and LMOD3. In some embodiments, the disease comprises Centronuclear myopathy, and the gene is DNM2. In some embodiments, the disease is Huntington's disease, and the gene is HTT. In some embodiments, the disease is Alpha-1 antitrypsin deficiency (AATD), and the gene is SERPINA1. In some embodiments, the disease is amyotrophic lateral sclerosis (ALS), and the gene is selected from SOD1, FUS, C9ORF72, ATXN2, TARDBP and CHCHD10. In some embodiments, the disease comprises Alexander Disease, and the gene is GFAP. In some embodiments, the disease comprises anaplastic large cell lymphoma, and the gene is CD30. In some embodiments, the disease comprises Angelman Syndrome, and the gene is UBE3A. In some embodiments, the disease comprises calcific aortic stenosis, and the gene is Apo(a). In some embodiments, the disease comprises CD3Z-associated primary T-cell immunodeficiency, and the gene is CD3Z or CD247. In some embodiments, the disease comprises CD18 deficiency, and the gene is ITGB2. In some embodiments, the disease comprises CD40L deficiency, and the gene is CD40L. In some embodiments, the disease is congenital adrenal hyperplasia, and the gene is CAH1. In some embodiments, the disease comprises CNS trauma, and the gene is VEGF. In some embodiments, the disease comprises coronary heart disease, and the gene is selected from FGA, FGB and FGG. In some embodiments, the disease comprises MECP2 Duplication syndrome and Rett syndrome, and the gene is MECP2. In some embodiments, the disease comprises a bleeding disorder (coagulation), and the gene is FXI. In some embodiments, the disease comprises fragile X syndrome, and the gene is FMRI. In some embodiments, the disease comprises Fuchs corneal dystrophy, and the gene is selected from ZEBI, SLC4A11 and LOXHD1. In some embodiments, the disease comprises GM2-Gangliosidoses (e.g., Tay Sachs Disease, Sandhoff disease), and the gene is selected from HEXA and HEXB. In some embodiments, the disease comprises Hearing loss disorders, and the gene is DFNA36. In some embodiments, the disease is Pompe disease, including infantile onset Pompe disease (IOPD) and late onset Pompe disease (LOPD), and the gene is GAA. In some embodiments, the disease is Retinitis pigmentosa, and the gene is selected from PDE6B, RHO, RP1, RP2, RPGR, PRPH2, IMPDH1, PRPF31, CRB1, PRPF8, TULP1, CA4, HPRPF3, ABCA4, EYS, CERKL, FSCN2, TOPORS, SNRNP200, PRCD, NR2E3, MERTK, USH2A, PROM1, KLHL7, CNGB1, TTC8, ARL6, DHDDS, BEST1, LRAT, SPARA7, CRX, CLRN1, RPE65 and WDR19. In some embodiments, the disease comprises Leber Congenital Amaurosis Type 10, and the gene is CEP290. In some embodiments, the disease is cardiovascular disease and/or lipodystrophies, and the gene is selected from ABCG5, ABCG8, AGT, ANGPTL3, APOCIII, APOA1, APOL1, ARH, CDKN2B, CFB, CXCL12, FXI, FXII, GATA-4, MIA3, MKL2, MTHFD1L, MYH7, NKX2-5, NOTCH1, PKK, PCSK9, PSRC1, SMAD3 and TTR. In some embodiments, the disease is cardiovascular disease and/or lipodystrophies, and the gene is ANGPTL3. In some embodiments, the disease is cardiovascular disease and/or lipodystrophies, and the gene is PCSK9. In some embodiments, the disease is cardiovascular disease and/or lipodystrophies, and the gene is TTR. In some embodiments, the disease is severe hypertriglyceridemia (SHTG), and the gene is APOCIII or ANGPTL4. In some embodiments, the disease comprises acromegaly, and the gene is GHR. In some embodiments, the disease comprises acute myeloid leukemia, and the gene is CD22. In some embodiments, the disease is diabetes, and the gene is GCGR. In some embodiments, the disease is NAFLD/NASH, and the gene is selected from HSD17B13, PSD3, GPAM, CIDEB, DGAT2 and PNPLA3. In some embodiments, the disease is NASH/cirrhosis, and the gene is MARCI. In some embodiments, the disease is cancer, and the gene is selected from STAT3, YAP1, FOXP3, AR (Prostate cancer) and IRF4 (multiple myeloma). In some embodiments, the disease is cystic fibrosis, and the gene is CFTR. In some embodiments, the disease is Duchenne muscular dystrophy, and the gene is DMD. In some embodiments, the disease is ornithine transcarbamylase deficiency (OTCD), and the gene is OTC. In some embodiments, the disease is congenital adrenal hyperplasia (CAH), and the gene is CYP21A2. In some embodiments, the disease is atherosclerotic cardiovascular disease (ASCVD), and the gene is LPA. In some embodiments, the disease is hepatitis B virus infection (CHB), and the gene is HBV covalently closed circular DNA (cccDNA). In some embodiments, the disease is citrullinemia type I, and the gene is ASS1. In some embodiments, the disease is citrullinemia type I, and the gene is SLC25A13. In some embodiments, the disease is citrullinemia type I, and the gene is ASS1. In some embodiments, the disease is arginase-1 deficiency, and the gene is ARGI. In some embodiments, the disease is carbamoyl phosphate synthetase I deficiency, and the gene is CPSI. In some embodiments, the disease is argininosuccinic aciduria, and the gene is ASL. In some embodiments, the disease comprises angioedema, and the gene is PKK. In some embodiments, the disease comprises thalassemia, and the gene is TMPRSS6. In some embodiments, the disease comprises achondroplasia, and the gene is FGFR3. In some embodiments, the disease comprises Cri du chat syndrome, and the gene is selected from CTNND2. In some embodiments, the disease comprises sickle cell anemia, and the gene is Beta globin gene. In some embodiments, the disease comprises Alagille Syndrome, and the gene is selected from JAG1 and NOTCH2. In some embodiments, the disease comprises Charcot-Marie-Tooth disease, and the gene is selected from PMP22 and MFN2. In some embodiments, the disease comprises Crouzon syndrome, and the gene is selected from FGFR2, FGFR3 and FGFR3. In some embodiments, the disease comprises Dravet Syndrome, and the gene is selected from SCN1A and SCN2A. In some embodiments, the disease comprises Emery-Dreifuss syndrome, and the gene is selected from EMD, LMNA, SYNE1, SYNE2, FHL1 and TMEM43. In some embodiments, the disease comprises Factor V Leiden thrombophilia, and the gene is F5. In some embodiments, the disease is fabry disease, and the gene is GLA. In some embodiments, the disease is facioscapulohumeral muscular dystrophy, and the gene is FSHD1. In some embodiments, the disease comprises Fanconi anemia, and the gene is selected from FANCA, FANCB, FANCC, FANCD1, FANCD2, FANCE, FANCF, FANCG, FANCI, FANCJ, FANCL, FANCM, FANCN, FANCP, FANCS, RAD51C and XPF. In some embodiments, the disease comprises Familial Creutzfeld-Jakob disease, and the gene is PRNP. In some embodiments, the disease comprises Familial Mediterranean Fever, and the gene is MEFV. In some embodiments, the disease comprises Friedreich's ataxia, and the gene is FXN. In some embodiments, the disease comprises Gaucher disease, and the gene is GBA. In some embodiments, the disease comprises human papilloma virus (HPV) infection, and the gene is HPV E7. In some embodiments, the disease comprises hemochromatosis, and the gene is HFE, optionally comprising a C282Y mutation. In some embodiments, the disease comprises Hemophilia A, and the gene is FVIII. In some embodiments, the disease is hereditary angioedema, and the gene is SERPING1 or KLKB1. In some embodiments, the disease comprises histiocytosis, and the gene is CD1. In some embodiments, the disease comprises immunodeficiency 17, and the gene is CD3D. In some embodiments, the disease comprises immunodeficiency 13, and the gene is CD4. In some embodiments, the disease comprises Common Variable Immunodeficiency, and the gene is selected from CD19 and CD81. In some embodiments, the disease comprises Joubert syndrome, and the gene is selected from INPP5E, TMEM216, AHI1, NPHP1, CEP290, TMEM67, RPGRIP1L, ARL13B, CC2D2A, OFD1, TMEM138, TCTN3, ZNF423 and AMRC9. In some embodiments, the disease comprises leukocyte adhesion deficiency, and the gene is CD18. In some embodiments, the disease comprises Li-Fraumeni syndrome, and the gene is TP53. In some embodiments, the disease comprises lymphoproliferative syndrome, and the gene is CD27. In some embodiments, the disease comprises Lynch syndrome, and the gene is selected from MSH2, MLH1, MSH6, PMS2, PMS1, TGFBR2 and MLH3. In some embodiments, the disease comprises mantle cell lymphoma, and the gene is CD5. In some embodiments, the disease comprises Marfan syndrome, and the gene is FBN1. In some embodiments, the disease comprises mastocytosis, and the gene is CD2. In some embodiments, the disease comprises methylmalonic acidemia, and the gene is selected from MMAA, MMAB and MUT. In some embodiments, the disease comprises mycosis fungoides, and the gene is CD7. In some embodiments, the disease comprises myotonic dystrophy, and the gene is selected from CNBP and DMPK. In some embodiments, the disease comprises neurofibromatosis, and the gene is selected from NF1 and NF2. In some embodiments, the disease comprises osteogenesis imperfecta, and the gene is selected from COLIA1, COLIA2 and IFITM5. In some embodiments, the disease comprises non-small cell lung cancer, and the gene is selected from KRAS, EGFR, ALK, METex14, BRAF V600E, ROSI, RET and NTRK. In some embodiments, the disease comprises subependymal glioma and the gene is RPTOR. In some embodiments, the disease comprises Peutz-Jeghers syndrome, and the gene is STK11. In some embodiments, the disease comprises polycystic kidney disease, and the gene is selected from PKD1 and PKD2. In some embodiments, the disease comprises Pitt-Hopkins Syndrome and the gene is TCF4. In some embodiments, the disease comprises Severe Combined Immune Deficiency, and the gene is selected from IL7R, RAGI and JAK3. In some embodiments, the disease comprises PRKAG2 cardiac syndrome, and the gene is PRKAG2. In some embodiments, the disease comprises spinocerebellar ataxia, and the gene is selected from ATXN1, ATXN2, ATXN3, PLEKHG4, SPTBN2, CACNA1A, ATXN7, ATXN8OS, ATXN10, TTBK2, PPP2R2B, KCNC3, PRKCG, ITPR1, TBP, KCND3 and FGF14. In some embodiments, the disease comprises thrombophilia due to antithrombin III deficiency, and the gene is SERPINC1. In some embodiments the disease comprises spinal muscular atrophy, and the gene is SMN1. In some embodiments, the disease comprises Usher Syndrome, and the gene is selected from MYO7A, USH1C, CDH23, PCDH15, USH1G, USH2A, GPR98, DFNB31 and CLRN1. In some embodiments, the disease comprises von Willebrand disease, and the gene is VWF. In some embodiments, the disease comprises Waardenburg syndrome, and the gene is selected from PAX3, MITF, WS2B, WS2C, SNAI2, EDNRB, EDN3, and SOX10. In some embodiments, the disease comprises Wiskott-Aldrich Syndrome, and the gene is WAS. In some embodiments, the disease comprises von Hippel-Lindau disease, and the gene is VHL. In some embodiments, the disease comprises Wilson disease, and the gene is ATP7B. In some embodiments, the disease comprises Zellweger syndrome, and the gene is selected from PEX1, PEX2, PEX3, PEX5, PEX6, PEX10, PEX12, PEX13, PEX14, PEX16, PEX19, and PEX26. In some embodiments, the disease comprises infantile myofibromatosis, and the gene is CD34. In some embodiments, the disease comprises platelet glycoprotein IV deficiency, and the gene is CD36. In some embodiments, the disease comprises immunodeficiency with hyper-IgM type 3, and the gene is CD40. In some embodiments, the disease comprises hemolytic uremic syndrome, and the gene is CD46. In some embodiments, the disease comprises complement hyperactivation, angiopathic thrombosis, or protein-losing enteropathy, and the gene is CD55. In some embodiments, the disease comprises hemolytic anemia, and the gene is CD59. In some embodiments, the disease comprises calcification of joints and arteries, and the gene is CD73. In some embodiments, the disease comprises immunoglobulin alpha deficiency, and the gene is CD79A. In some embodiments, the disease comprises C syndrome, and the gene is CD96. In some embodiments, the disease comprises pain and the gene is NAV1.7. In some embodiments, the disease comprises hairy cell leukemia, and the gene is CD123. In some embodiments, the disease comprises histiocytic sarcoma, and the gene is CD163. In some embodiments, the disease comprises autosomal dominant deafness, and the gene is CD164. In some embodiments, the disease comprises immunodeficiency 25, and the gene is CD247. In some embodiments, the disease comprises methymalonic acidemia due to transcobalamin receptor defect, and the gene is CD320.

### Cancer

In some embodiments, the disease comprises a cancer. Non-limiting examples of cancers include: acute lymphoblastic leukemia; acute lymphoblastic lymphoma; acute lymphocytic leukemia; acute myelogenous leukemia; acute myeloid leukemia (adult/childhood); adrenocortical carcinoma; AIDS-related cancers; AIDS-related lymphoma; anal cancer; appendix cancer; astrocytoma; atypical teratoid/rhabdoid tumor; basal-cell carcinoma; bile duct extrahepatic (cholangiocarcinoma); bladder cancer; cancer; bone osteosarcoma/malignant fibrous histiocytoma; brain cancer (adult/childhood); brain tumor; cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; Ewing sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver cancer); Hodgkin's lymphoma; hypopharyngeal cancer; hypothalamic and visual pathway glioma; intraocular melanoma; islet cell carcinoma (endocrine pancreas); Kaposi Sarcoma; kidney cancer (renal cell cancer); Langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenström; male breast cancer; malignant fibrous histiocytoma of bone/osteosarcoma; medulloblastoma; medulloepithelioma; melanoma; melanoma, intraocular (eye); Merkel cell cancer; Merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-Hodgkin's lymphoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer; pituitary tumor, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituitary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; NUT midline carcinoma; retinoblastoma; rhabdomyosarcoma, childhood; salivary gland cancer; sarcoma, Ewing family of tumors; Sézary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; subependymal glioma; supratentorial primitive neuroectodermal tumor; T-cell lymphoma, cutaneous (Mycosis Fungoides and Sézary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor.

In some embodiments, mutations are associated with cancer or are causative of cancer. The target nucleic acid, in some embodiments, comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, a gene associated with cell cycle, or combinations thereof. Non-limiting examples of genes comprising a mutation associated with cancer are ABL, ACE, AF4/HRX, AKT-2, ALK, ALK/NPM, AML1, AML1/MTG8, APC, ATM, AXIN2, AXL, BAP1, BARD1, BCL-2, BCL-3, BCL-6, BCR/ABL, BLM, BMPR1A, BRCA1, BRCA2, BRIP1, c-MYC, CASR, CCR5, CDC73, CDH1, CDK4, CDKN1B, CDKN1C, CDKN2A, CEBPA, CHEK2, CREBBP, CTNNA1, DBL, DEK/CAN, DICER1, DIS3L2, E2A/PBX1, EGFR, ENL/HRX, EPCAM, ERG/TLS, ERBB, ERBB-2, ETS-1, EWS/FLI-1, FH, FKRP, FLCN, FMS, FOS, FPS, GATA2, GCG, GLI, GPC3, GPGSP, GREM1, HER2/neu, HOX11, HOXB13, HRAS, HST, IL-3, INT-2, JAK1, JUN, KIT, KS3, K-SAM, LBC, LCK, LMO1, LMO2, L-MYC, LYL-1, LYT-10, LYT-10/Cα1, MAS, MAX, MDM-2, MEN1, MET, MITF, MLH1, MLL, MOS, MSH1, MSH2, MSH3, MSH6, MTG8/AML1, MUTYH, MYB, MYH11/CBFB, NBN, NEU, NF1, NF2, N-MYC, NTHL1, OST, PALB2, PAX-5, PBX1/E2A, PCDC1, PDGFRA, PHOX2B, PIM-1, PMS2, POLD1, POLE, POT1, PPARG, PRAD-1, PRKAR1A, PTCH1, PTEN, RAD50, RAD51C, RAD51D, RAF, RAR/PML, RAS-H, RAS-K, RAS-N, RB1, RECQL4, REL/NRG, RET, RHOM1, RHOM2, ROS, RPTOR, RUNX1, SDHA, SDHAF, SDHAF2, SDHB, SDHC, SDHD, SET/CAN, SIS, SKI, SMAD4, SMARCA4, SMARCB1, SMARCE1, SRC, STK11, SUFU, TAL1, TAL2, TAN-1, TIAM1, TERC, TERT, TIMP 3, TMEM127, TNF, TP53, TRAC, TSC1, TSC2, TRK, VHL, WRN and WT1. Non-limiting examples of oncogenes are KRAS, NRAS, BRAF, MYC, CTNNB1 and EGFR. In some embodiments, the oncogene is a gene that encodes a cyclin dependent kinase (CDK). Non-limiting examples of CDKs are Cdk1, Cdk4, Cdk5, Cdk7, Cdk8, Cdk9, Cdk11 and Cdk20. Non-limiting examples of tumor suppressor genes are TP53, RBI and PTEN.

### Infections

Described herein are compositions, systems and methods for treating an infection in a subject. Infections may be caused by a pathogen (e.g., bacteria, viruses, fungi and parasites). Compositions, systems and methods may modify a target nucleic acid associated with the pathogen or parasite causing the infection. In some embodiments, the target nucleic acid is in the pathogen or parasite itself or in a cell, tissue or organ of the subject that the pathogen or parasite infects. In some embodiments, the methods described herein include treating an infection caused by one or more bacterial pathogens. Non-limiting examples of bacterial pathogens include Acholeplasma laidlawii, Brucella abortus, Chlamydia psittaci, Chlamydia trachomatis, Cryptococcus neoformans, Escherichia coli, Legionella pneumophila, Lyme disease spirochetes, methicillin-resistant Staphylococcus aureus, Mycobacterium leprae, Mycobacterium tuberculosis, Mycoplasma arginini, Mycoplasma arthritidis, Mycoplasma genitalium, Mycoplasma hyorhinis, Mycoplasma orale, Mycoplasma pneumoniae, Mycoplasma salivarium, Neisseria gonorrhoeae, Neisseria meningitidis, Pneumococcus, Pseudomonas aeruginosa, sexually transmitted infection, Streptococcus agalactiae, Streptococcus pyogenes and Treponema pallidum.

In some embodiments, compositions, systems or methods described herein treat an infection caused by one or more viral pathogens. Non-limiting examples of viral pathogens include adenovirus, blue tongue virus, chikungunya, coronavirus (e.g., SARS-COV-2), cytomegalovirus, Dengue virus, Ebola, Epstein-Barr virus, feline leukemia virus, Hemophilus influenzae B, Hepatitis Virus A, Hepatitis Virus B, Hepatitis Virus C, herpes simplex virus I, herpes simplex virus II, human papillomavirus (HPV) including HPV16 and HPV18, human serum parvo-like virus, human T-cell leukemia viruses, immunodeficiency virus (e.g., HIV), influenza virus, lymphocytic choriomeningitis virus, measles virus, mouse mammary tumor virus, mumps virus, murine leukemia virus, polio virus, rabies virus, Reovirus, respiratory syncytial virus (RSV), rubella virus, Sendai virus, simian virus 40, Sindbis virus, varicella-zoster virus, vesicular stomatitis virus, wart virus, West Nile virus, yellow fever virus, or any combination thereof.

In some embodiments, compositions, systems or methods described herein treat an infection caused by one or more parasites. Non-limiting examples of parasites include helminths, annelids, platyhelminthes, nematodes and thorny-headed worms. In some embodiments, parasitic pathogens comprise, without limitation, Babesia bovis, Echinococcus granulosus, Eimeria tenella, Leishmania tropica, Mesocestoides corti, Onchocerca volvulus, Plasmodium falciparum, Plasmodium vivax, Schistosoma japonicum, Schistosoma mansoni, Schistosoma spp., Taenia hydatigena, Taenia ovis, Taenia saginata, Theileria parva, Toxoplasma gondii, Toxoplasma spp., Trichinella spiralis, Trichomonas vaginalis, Trypanosoma brucei, Trypanosoma cruzi, Trypanosoma rangeli, Trypanosoma rhodesiense, Balantidium coli, Entamoeba histolytica, Giardia spp., Isospora spp., Trichomonas spp., or any combination thereof.

### X. Illustrative Embodiments

Embodiment 1. An effector protein or a nucleic acid encoding the effector protein, wherein the effector protein comprising an amino acid sequence that is at least 70%, at least 75%, at least 80%, at least 85%, at least 90%, at least 95%, at least 98% or at least 99% identical to any one of the sequences set forth in TABLE 1, wherein the effector protein comprises a deletion of one or more domains, a substitution of one or more domains for a different amino acid sequence, or a combination thereof, wherein the one or more domains independently comprise an amino acid sequence that is at least 90% identical to any one of the domains identified in TABLE 3.

Embodiment 2. The effector protein or the nucleic acid encoding the effector protein of Embodiment 1, wherein the different amino acid sequence comprises any one of SEQ ID NO: 18, 41-104 and 356-368.

Embodiment 3. The effector protein or the nucleic acid encoding the effector protein of Embodiment 1, wherein: (a) the effector protein comprises an amino acid sequence that is at least 70% identical to SEQ ID NO: 1, (b) the domain comprises an amino acid sequence that is at least 90% identical to SEQ ID NO: 6, and (c) the different amino acid sequence comprises an amino acid sequence that is at least 90% identical to any one of SEQ ID NO: 41-47.

Embodiment 4. The effector protein or the nucleic acid encoding the effector protein of Embodiment 1, wherein: (a) the effector protein comprises an amino acid sequence that is at least 70% identical to SEQ ID NO: 1, (b) the domain comprises an amino acid sequence that is at least 90% identical to any one of SEQ ID NO: 7-10, 13, 17-18 and 20, and (c) the different amino acid sequence comprises an amino acid sequence of SEQ ID NO: 48.

Embodiment 5. The effector protein or the nucleic acid encoding the effector protein of Embodiment 1, wherein: (a) the effector protein comprises an amino acid sequence that is at least 70% identical to SEQ ID NO: 1, (b) the domain comprises an amino acid sequence that is at least 90% identical to any one of SEQ ID NO: 11 and 12, and (c) the different amino acid sequence comprises an amino acid sequence of SEQ ID NO: 49.

Embodiment 6. The effector protein or the nucleic acid encoding the effector protein of Embodiment 1, wherein: (a) the effector protein comprises an amino acid sequence that is at least 70% identical to SEQ ID NO: 1, (b) the domain comprises an amino acid sequence that is at least 90% identical to SEQ ID NO: 14, and (c) the different amino acid sequence comprises an amino acid sequence that is at least 90% identical to any one of SEQ ID NO: 50-65.

Embodiment 7. The effector protein or the nucleic acid encoding the effector protein of Embodiment 1, wherein: (a) the effector protein comprises an amino acid sequence that is at least 70% identical to SEQ ID NO: 1, (b) the domain comprises an amino acid sequence that is at least 90% identical to SEQ ID NO: 15, and (c) the different amino acid sequence comprises an amino acid sequence that is at least 90% identical to any one of SEQ ID NO: 66-100.

Embodiment 8. The effector protein or the nucleic acid encoding the effector protein of Embodiment 1, wherein: (a) the effector protein comprises an amino acid sequence that is at least 70% identical to SEQ ID NO: 1, (b) the domain comprises an amino acid sequence that is at least 90% identical to SEQ ID NO: 16, and (c) the different amino acid sequence comprises an amino acid sequence that is at least 90% identical to SEQ ID NO: 101.

Embodiment 9. The effector protein or the nucleic acid encoding the effector protein of Embodiment 1, wherein: (a) the effector protein comprises an amino acid sequence that is at least 70% identical to SEQ ID NO: 1, (b) the domain comprises an amino acid sequence that is at least 90% identical to SEQ ID NO: 19, and (c) the different amino acid sequence comprises an amino acid sequence of SEQ ID NO: 102.

Embodiment 10. The effector protein or the nucleic acid encoding the effector protein of Embodiment 1, wherein: (a) the effector protein comprises an amino acid sequence that is at least 70% identical to SEQ ID NO: 1, (b) the domain comprises an amino acid sequence that is at least 90% identical to SEQ ID NO: 21, and (c) the different amino acid sequence comprises an amino acid sequence that is at least 90% identical to SEQ ID NO: 103.

Embodiment 11. The effector protein or the nucleic acid encoding the effector protein of Embodiment 1, wherein: (a) the effector protein comprises an amino acid sequence that is at least 70% identical to SEQ ID NO: 1, (b) the domain comprises an amino acid sequence that is at least 90% identical to SEQ ID NO: 22, and (c) the different amino acid sequence comprises an amino acid sequence that is at least 90% identical to SEQ ID NO: 104.

Embodiment 12. The effector protein or the nucleic acid encoding the effector protein of Embodiment 1, wherein: (a) the effector protein comprises an amino acid sequence that is at least 70% identical to SEQ ID NO: 1, (b) the domain comprises an amino acid sequence that is at least 90% identical to SEQ ID NO: 349, and (c) the different amino acid sequence comprises an amino acid sequence that is at least 90% identical to SEQ ID NO: 356.

Embodiment 13. The effector protein or the nucleic acid encoding the effector protein of Embodiment 1, wherein: (a) the effector protein comprises an amino acid sequence that is at least 70% identical to SEQ ID NO: 1, (b) the domain comprises an amino acid sequence that is at least 90% identical to SEQ ID NO: 350, and (c) the different amino acid sequence comprises an amino acid sequence that is at least 90% identical to SEQ ID NO: 357.

Embodiment 14. The effector protein or the nucleic acid encoding the effector protein of Embodiment 1, wherein: (a) the effector protein comprises an amino acid sequence that is at least 70% identical to SEQ ID NO: 1, (b) the domain comprises an amino acid sequence that is at least 90% identical to SEQ ID NO: 351, and (c) the different amino acid sequence comprises an amino acid sequence that is at least 90% identical to SEQ ID NO: 358.

Embodiment 15. The effector protein or the nucleic acid encoding the effector protein of Embodiment 1, wherein: (a) the effector protein comprises an amino acid sequence that is at least 70% identical to SEQ ID NO: 1, (b) the domain comprises an amino acid sequence that is at least 90% identical to SEQ ID NO: 352, and (c) the different amino acid sequence comprises an amino acid sequence that is at least 90% identical to any one of SEQ ID NOS: 359-365.

Embodiment 16. The effector protein or the nucleic acid encoding the effector protein of Embodiment 1, wherein: (a) the effector protein comprises an amino acid sequence that is at least 70% identical to SEQ ID NO: 1, (b) the domain comprises an amino acid sequence that is at least 90% identical to SEQ ID NO: 353, and (c) the different amino acid sequence comprises an amino acid sequence that is at least 90% identical to SEQ ID NO: 366.

Embodiment 17. The effector protein or the nucleic acid encoding the effector protein of Embodiment 1, wherein: (a) the effector protein comprises an amino acid sequence that is at least 70% identical to SEQ ID NO: 1, (b) the domain comprises an amino acid sequence that is at least 90% identical to SEQ ID NO: 354, and (c) the different amino acid sequence comprises an amino acid sequence that is at least 90% identical to SEQ ID NO: 367.

Embodiment 18. The effector protein or the nucleic acid encoding the effector protein of Embodiment 1, wherein: (a) the effector protein comprises an amino acid sequence that is at least 70% identical to SEQ ID NO: 1, (b) the domain comprises an amino acid sequence that is at least 90% identical to SEQ ID NO: 355, and (c) the different amino acid sequence comprises an amino acid sequence that is at least 90% identical to SEQ ID NO: 368.

Embodiment 19. The effector protein or the nucleic acid encoding the effector protein of Embodiment 1, wherein: (a) the effector protein comprises an amino acid sequence that is at least 70% identical to SEQ ID NO: 2, (b) the domain comprises an amino acid sequence that is at least 90% identical to any one of SEQ ID NO: 23-25, and (c) the different amino acid sequence comprises an amino acid sequence of SEQ ID NO: 48.

Embodiment 20. The effector protein or the nucleic acid encoding the effector protein of Embodiment 1, wherein: (a) the effector protein comprises an amino acid sequence that is at least 70% identical to SEQ ID NO: 2, (b) the domain comprises an amino acid sequence that is at least 90% identical to SEQ ID NO: 24, and (c) the different amino acid sequence comprises an amino acid sequence that is at least 90% identical to SEQ ID NO: 18.

Embodiment 21. The effector protein or the nucleic acid encoding the effector protein of Embodiment 1, wherein: (a) the effector protein comprises an amino acid sequence that is at least 70% identical to SEQ ID NO: 3, (b) the domain comprises an amino acid sequence that is at least 90% identical to any one of SEQ ID NO: 26-28, and (c) the different amino acid sequence comprises an amino acid sequence of SEQ ID NO: 48.

Embodiment 22. The effector protein or the nucleic acid encoding the effector protein, wherein the effector protein of Embodiment 1, wherein: (a) the effector protein comprises an amino acid sequence that is at least 70% identical to SEQ ID NO: 3, (b) the domain comprises an amino acid sequence that is at least 90% identical to SEQ ID NO: 27, and (c) the different amino acid sequence comprises an amino acid sequence that is at least 90% identical to SEQ ID NO: 18.

Embodiment 23. The effector protein or the nucleic acid encoding the effector protein of Embodiment 1, wherein: (a) the effector protein comprises an amino acid sequence that is at least 70% identical to SEQ ID NO: 4, (b) the domain comprises an amino acid sequence that is at least 90% identical to any one of SEQ ID NO: 29-31, and (c) the different amino acid sequence comprises an amino acid sequence of SEQ ID NO: 48.

Embodiment 24. The effector protein or the nucleic acid encoding the effector protein of Embodiment 1, wherein: (a) the effector protein comprises an amino acid sequence that is at least 70% identical to SEQ ID NO: 4, (b) the domain comprises an amino acid sequence that is at least 90% identical to SEQ ID NO: 30, and (c) the different amino acid sequence comprises an amino acid sequence that is at least 90% identical to SEQ ID NO: 18.

Embodiment 25. The effector protein or the nucleic acid encoding the effector protein of Embodiment 1, wherein: (a) the effector protein comprises an amino acid sequence that is at least 70% identical to SEQ ID NO: 5, (b) the domain comprises an amino acid sequence that is at least 90% identical to any one of SEQ ID NO: 32-34, and (c) the different amino acid sequence comprises an amino acid sequence of SEQ ID NO: 48.

Embodiment 26. The effector protein or the nucleic acid encoding the effector protein of Embodiment 1, wherein: (a) the effector protein comprises an amino acid sequence that is at least 70% identical to SEQ ID NO: 5, (b) the domain comprises an amino acid sequence that is at least 90% identical to SEQ ID NO: 33, and (c) the different amino acid sequence comprises an amino acid sequence that is at least 90% identical to SEQ ID NO: 18.

Embodiment 27. An effector protein or a nucleic acid encoding the effector protein, wherein the effector protein comprising an amino acid sequence that is at least 85%, at least 90%, at least 95%, at least 98%, at least 99% or 100% identical to any one of the amino acid sequences set forth in TABLE 4.

Embodiment 28. The effector protein or the nucleic acid encoding the effector protein of any one of Embodiments 1-18, wherein the effector protein further comprises any one of the amino acid substitutions recited in TABLE 2.

Embodiment 29. The effector protein or the nucleic acid encoding the effector protein of any one of Embodiments 1-18 and 28, wherein the effector protein comprises a substitution of I471T relative to an amino acid sequence of SEQ ID NO: 1.

Embodiment 30. The effector protein or the nucleic acid encoding the effector protein of any one of Embodiments 1-18 and 28-29, wherein the effector protein comprises a substitution of L26R relative to an amino acid sequence of SEQ ID NO: 1.

Embodiment 31. The effector protein or the nucleic acid encoding the effector protein of Embodiment 29, wherein the effector protein comprises substitutions of L26K and, H208R relative to an amino acid sequence of SEQ ID NO: 1.

Embodiment 32. The effector protein or the nucleic acid encoding the effector protein of Embodiment 29, wherein the effector protein comprises substitutions of L26K and D703G relative to an amino acid sequence of SEQ ID NO: 1.

Embodiment 33. The effector protein or the nucleic acid encoding the effector protein of any one of Embodiments 1-28, wherein the effector protein comprises substitutions of L26K, L149R and I471T relative to an amino acid sequence of SEQ ID NO: 1.

Embodiment 34. The effector protein or the nucleic acid encoding the effector protein, wherein the effector protein of any one of Embodiments 1-18 comprises a combination of substitutions relative to an amino acid sequence of SEQ ID NO: 1, wherein the combination is selected from: L26R, I471T, S223P and D703G; L26R, I471T, S223P, D703G and H208R; L26R, I471T, S223P, D703G and L149R; L26R, I471T, S223P, D703G, L149R and H208R; L26R, I471T, S223P, D703G, D704G and A706G; L26R, I471T, S223P, D703G, L149R, H208R, D704G and A706G; I471T, S223P and D703G; I471T, S223P, D703G and H208R; I471T, S223P, D703G and L149R; I471T, S223P, D703G, L149R and H208R; I471T, S223P, D703G, D704G and A706G; I471T, S223P, D703G, L149R, H208R, D704G and A706G; I471T and E157R; I471T, E157R, S223P and D703G; L26R, I471T, E157R, S223P and D703G; L26R, T87G, S186G, H208R, S223P, C405L, I471T, S526N and D703G; L26R, A121Q, S223P, E258K, I471T, D523K, S526N and D703G; L26R, N147K, H208R, S223P, E258K, I471T, M503K and D703G; L26R, N147K, S186G, S223P, E258K, I471T, S526N, D549L, S638K and D703G; S21L, L26R, S186G, Y220S, S223P, I471T and D703G; L26R, T87G, A121Q, S186G, H208R, Y220S, S223P, C405L, I471T, D523K and D703G; S21L, L26R, A121Q, N147K, S186G, Y220S, S223P, I471T, S526N, D549L and D703G; S21L, L26R, Q76R, N147K, L149R, Y220S, S223P, Y251R, E258K, I471T, M503K, Q552R and D703G; L26R, A121Q, Y220S, S223P, C405L, I471T, D523K, Q552R and D703G; S21L, L26R, A121Q, N147K, Y220S, S223P, Y251R, C405L, I471T and D703G; L26R, Q76R, T87G, S223P, E258K, C279R, I471T, M503K, D523K and D703G; L26R, N147K, S186G, S223P, I471T, M503K, S526N and D703G; S21L, L26R, T87G, N147K, H208R, Y220S, S223P, I471T and D703G; S21L, L26R, A121Q, N147K, S186G, S223P, E258K, I471T, D523K, Q552R and D703G; L26R, A121Q, L149R, S186G, Y220S, S223P, I471T and D703G; L26R, A121Q, N147K, Y220S, S223P, I471T, M503K, S526N, D549L and D703G; L26R, T87G, A121Q, Y220S, S223P, E258K, C405L, I471T and D703G; L26R, T87G, S186G, Y220S, S223P, I471T, M503K and D703G; S21L, L26R, Q76R, T87G, N147K, S186G, S223P, I471T, S526N, S638K and D703G; S21L, L26R, A121Q, Y220S, S223P, C405L, I471T, M503K and D703G; L26R, S223P, I471T and D703G; L26R, T87G, S223P, I471T, S526N and D703G; L26R, T87G, N147K, S223P, I471T, S526N and D703G; L26R, T87G, N147K, S223P, E258K, I471T, S526N and D703G; L26R, T87G, Y220S, S223P, I471T, S526N and D703G; L26R, T87G, N147K, Y220S, S223P, E258K, I471T, S526N and D703G; L26R, Q76R, T87G, N147K, Y220S, S223P, E258K, C279R, I471T, M503K, D523K and D703G; L26R, Q76R, T87G, N147K, Y220S, S223P, E258K, I471T, M503K, D523K and D703G; L26R, Q76R, T87G, N147K, Y220S, S223P, E258K, I471T, M503K and D703G; S21L, L26R, Q76R, T87G, S223P, E258K, C279R, C405L, I471T, M503K, D523K and D703G; L26R, Q76R, T87G, N147K, S186G, Y220S, S223P, E258K, C405L, I471T, M503K and D703G; L26R, T87G, Y220S, S223P, I471T and D703G; L26R, T87G, N147K, Y220S, S223P, E258K, I471T and D703G; S21L, L26R, T87G, N147K, Y220S, S223P, E258K, I471T and D703G; S21L, L26R, T87G, N147K, Y220S, S223P, E258K, C405L, I471T, M503K and D703G; S21L, L26R, T87G, A121Q, N147K, Y220S, S223P, E258K, C405L, I471T, M503K, S638K and D703G; S21L, L26R, T87G, A121Q, N147K, S186G, Y220S, S223P, E258K, C405L, I471T, M503K, S638K and D703G; L26R, T87G, S223P, I471T and D703G; L26R, T87G, N147K, S223P, I471T and D703G; L26R, T87G, N147K, S223P, E258K, I471T and D703G; L26R, T87G, S186G, H208R, S223P, C405L, I471T and D703G; L26R, N147K, S186G, S223P, I471T, M503K and D703G; and L26R, S223P, E258K, I471T and D703G.

Embodiment 35. The effector protein or the nucleic acid encoding the effector protein of any one of Embodiments 1-34, wherein the effector protein, when in complex with a guide nucleic acid and the guide nucleic acid is hybridized to a target sequence of a double stranded target nucleic acid, nicks the double stranded target nucleic acid.

Embodiment 36. The effector protein or the nucleic acid encoding the effector protein of Embodiment 35, wherein the effector protein nicks a target strand of the double stranded target nucleic acid.

Embodiment 37. The effector protein or the nucleic acid encoding the effector protein of Embodiment 35, wherein the effector protein nicks a non-target strand of the double stranded target nucleic acid.

Embodiment 38. The effector protein or the nucleic acid encoding the effector protein of any one of Embodiments 1-37, wherein the effector protein comprises cis cleavage activity.

Embodiment 39. The effector protein or the nucleic acid encoding the effector protein of any one of Embodiments 1-38, wherein the effector protein further comprises one or more heterologous peptides that are heterologous to the effector protein.

Embodiment 40. The effector protein or the nucleic acid encoding the effector protein of Embodiment 39, wherein the one or more heterologous peptides are located at N-terminus, C-terminus, or both of the effector protein.

Embodiment 41. The effector protein or the nucleic acid encoding the effector protein of Embodiment 39 or 40, wherein the one or more heterologous peptide independently comprises any one of the amino acid sequences recited in TABLE 5.

Embodiment 42. The effector protein or the nucleic acid encoding the effector protein of any one of Embodiments 1-41, wherein the effector protein recognizes any one of protospacer adjacent motif (PAM) sequences set forth in TABLE 6.

Embodiment 43. An effector protein comprising an amino acid sequence or a nucleic acid encoding the effector protein, wherein the effector protein comprises a deletion of one or more domains, a substitution of one or more domains for a different amino acid sequence, or a combination thereof, wherein the one or more domains independently comprise an amino acid sequence that is at least 90% identical to any one of the domains identified in TABLE 3, and wherein the effector protein, other than the deletion of one or more domains, the substitution of one or more domains for a different amino acid sequence, or the combination thereof, comprises an amino acid sequence that is at least 70%, at least 75%, at least 80%, at least 85%, at least 90%, at least 95%, at least 98% or at least 99% identical to any one of the sequences set forth in TABLE 1.

Embodiment 44. An effector protein comprising an amino acid sequence or a nucleic acid encoding the effector protein, wherein the effector protein comprises any combination of amino acid substitutions described in TABLE 2, and wherein the effector protein, other than the amino acid substitutions described in TABLE 2, is at least 70%, at least 75%, at least 80%, at least 85%, at least 90%, at least 95%, at least 98% or at least 99% identical to SEQ ID NO:

1.

Embodiment 45. A system comprising one or more components, wherein the one or more components individually comprise: (a) the effector protein or the nucleic acid encoding the effector protein of any one of Embodiments 1-44; and (b) a guide nucleic acid or a nucleic acid that encodes the guide nucleic acid, wherein the guide nucleic acid comprises a repeat sequence and a spacer sequence, wherein the repeat sequence, at least partially, interacts with the effector protein, wherein the spacer sequence comprises a nucleic acid sequence that hybridizes to a target sequence in a target nucleic acid.

Embodiment 46. The system of Embodiment 45, wherein the repeat sequence comprises a nucleotide sequence that is at least 75%, at least 80%, at least 85%, at least 90%, at least 95%, at least 97%, or at least 98%, at least 99%, or 100% identical to the nucleotide sequence recited in TABLE 7.

Embodiment 47. The system of any one of Embodiments 45 or 46, wherein the guide nucleic acid is a crRNA.

Embodiment 48. The system of any one of Embodiments 45-47, wherein the spacer sequence comprises a nucleotide sequence in a range of from 10 to 20 linked nucleotides.

Embodiment 49. The system of any one of Embodiments 45-48, wherein the spacer sequence comprises a nucleotide sequence of 14 linked nucleotides.

Embodiment 50. The system of any one of Embodiments 45-49, wherein the spacer sequence comprises a nucleotide sequence that is about 80% to about 95% complementary to the target sequence.

Embodiment 51. The system of any one of Embodiments 45-50, wherein the guide nucleic acid comprises one or more phosphorothioate (PS) backbone modifications, 2′-fluoro (2′-F) sugar modifications, or 2′-O-Methyl(2′OMe) sugar modifications.

Embodiment 52. The system of Embodiment 51, wherein the guide nucleic acid comprises PS backbone modification between −3 and −2 positions of a repeat sequence present in the guide nucleic acid, and wherein the repeat sequence comprises at least 24 nucleotides.

Embodiment 53. The system of Embodiment 52, wherein the guide nucleic acid further comprises at least one modification between −16 and −12 positions of the repeat sequence present in the guide nucleic acid.

Embodiment 54. The system of Embodiment 53, wherein the at least one modification comprises 2′OMe sugar modification at −14 position of the repeat sequence present in the guide nucleic acid.

Embodiment 55. The system of Embodiment 53, wherein the at least one modification comprises 2′OMe sugar modification at −16 position of the repeat sequence present in the guide nucleic acid.

Embodiment 56. The system of Embodiment 53, wherein the at least one modification comprises PS backbone modification between −13 and −12 positions of the repeat sequence present in the guide nucleic acid.

Embodiment 57. The system of Embodiment 53, wherein the at least one modification comprises PS backbone modification between −14 and −13 positions of the repeat sequence present in the guide nucleic acid.

Embodiment 58. The system of Embodiment 53, wherein the at least one modification comprises PS backbone modification between −15 and −14 positions of the repeat sequence present in the guide nucleic acid.

Embodiment 59. The system of any one of Embodiments 45-58, wherein the target sequence is within a human gene.

Embodiment 60. The system of any one of Embodiments 45-59, wherein the system further comprises the target nucleic acid, wherein the target nucleic acid is a double stranded DNA comprising a target strand and a non-target strand.

Embodiment 61. The system of any one of Embodiments 45-60, wherein the spacer sequence hybridizes to the target strand and the PAM is located on a non-target strand of the target nucleic acid.

Embodiment 62. The system of any one of Embodiments 45-61, wherein the PAM is located 5′ of a reverse complement of the target sequence on the non-target strand.

Embodiment 63. The system of any one of Embodiments 45-62, wherein the target nucleic acid is isolated from a human cell.

Embodiment 64. The system of any one of Embodiments 45-63, wherein the target nucleic acid is any one of the nucleic acids set forth in TABLE 8.

Embodiment 65. The system of any one of Embodiments 45-64, wherein the target nucleic acid is associated with any one of the diseases or disorders of TABLE 9.

Embodiment 66. The system of any one of Embodiments 45-65, further comprising an effector partner, or a nucleic acid encoding the effector partner.

Embodiment 67. The system of Embodiment 66, wherein the system comprises a fusion protein, or a nucleic acid encoding the fusion protein, wherein the fusion protein comprises the effector protein and the effector partner fused to each other.

Embodiment 68. The system of Embodiment 67, wherein N-terminus of the effector protein is linked to C-terminus of the effector partner.

Embodiment 69. The system of Embodiment 67, wherein C-terminus of the effector protein is linked to C-terminus of the effector partner.

Embodiment 70. The system of any one of Embodiments 67-69, wherein the effector protein and the effector partner are directly fused to each other.

Embodiment 71. The system of any one of Embodiments 67-70, wherein the effector protein and the effector partner are fused by a linker.

Embodiment 72. The system of any one of Embodiments 45-71, wherein the system comprises an expression vector, wherein the expression vector encodes the effector protein, the effector partner, the guide nucleic acid, or a combination thereof.

Embodiment 73. The system of Embodiment 72, wherein the nucleic acid expression vector is a viral vector or a non-viral vector.

Embodiment 74. The system of Embodiment 73, wherein the viral vector is an adeno associated viral (AAV) vector.

Embodiment 75. The system of any one of Embodiments 45-74, wherein the nucleic acids encoding the effector protein, the effector partner, or the combination thereof are messenger RNAs.

Embodiment 76. The system of any one of Embodiments 45-75 comprising a lipid or a lipid nanoparticle.

Embodiment 77. A library of nucleic acid expression vectors comprising at least one of the nucleic acid expression vectors of any one of the systems of Embodiments 72-76.

Embodiment 78. A composition comprising: (a) the effector protein or the nucleic acid encoding the effector protein of any one of Embodiments 1-44; or (b) one or more components of any one of the systems of Embodiments 45-76.

Embodiment 79. A pharmaceutical composition comprising: (a) the effector protein or the nucleic acid encoding the effector protein of any one of Embodiments 1-44, one or more components of any one of the system of Embodiments 45-76, or the composition of Embodiment 78; and (b) a pharmaceutically acceptable excipient.

Embodiment 80. A cell comprising: (a) the effector protein or the nucleic acid encoding the effector protein of any one of Embodiments 1-44; (b) one or more components of any one of the systems of Embodiments 45-76; (c) the library of nucleic acid expression vectors of Embodiment 77; (d) the composition of Embodiment 78; or (e) the pharmaceutical composition of Embodiment 79.

Embodiment 81. A method of nicking a target nucleic acid within a human gene or associated with expression of a human gene, the method comprising contacting the target nucleic acid with one or more of: (a) the effector protein or the nucleic acid encoding the effector protein of any one of Embodiments 1-44; (b) one or more components of any one of the systems of Embodiments 45-76; (c) the library of nucleic acid expression vectors of Embodiment 77; (d) the composition of Embodiment 78; or (e) the pharmaceutical composition of Embodiment 79, thereby nicking the target nucleic acid.

Embodiment 82. The method of Embodiment 81, wherein the method is performed in a cell.

Embodiment 83. The method of Embodiment 81, wherein the method is performed in vivo.

Embodiment 84. The method of Embodiment 81, wherein the method is performed in vitro.

Embodiment 85. The method of any one of Embodiments 81-84, wherein the target nucleic acid comprises a mutation associated with a disease.

Embodiment 86. The method of Embodiment 85, wherein the one or more mutations comprise a point mutation, a single nucleotide polymorphism (SNP), a chromosomal mutation, a copy number mutation, or any combination thereof.

Embodiment 87. The method of Embodiment 85 or 86, wherein the target nucleic acid is any one of the nucleic acids set forth in TABLE 8.

Embodiment 88. The method of any one of Embodiments 85-87, wherein the target nucleic acid is associated with any one of the diseases set forth in TABLE 9.

Embodiment 89. A cell contacted by: (a) the effector protein or the nucleic acid encoding the effector protein of any one of Embodiments 1-44; (b) one or more components of any one of the systems of Embodiments 45-76; (c) the library of nucleic acid expression vectors of Embodiment 77; (d) the composition of Embodiment 78; (e) the pharmaceutical composition of Embodiment 79; or (f) the method of any one of Embodiments 81-88.

Embodiment 90. A cell comprising a target nucleic acid modified by: (a) the effector protein or the nucleic acid encoding the effector protein of any one of Embodiments 1-44; (b) one or more components of any one of the systems of Embodiments 45-76; (c) the library of nucleic acid expression vectors of Embodiment 77; (d) the composition of Embodiment 78; (e) the pharmaceutical composition of Embodiment 79; or (f) the method of any one of Embodiments 81-88.

Embodiment 91. The cell of any one of Embodiments 80 and 89-90, wherein the cell is a eukaryotic cell.

Embodiment 92. The cell of any one of Embodiments 80 and 89-91, wherein the cell is a mammalian cell.

Embodiment 93. The cell of any one of Embodiments 80 and 89-92, wherein the cell is a human cell.

Embodiment 94. A population of cells that comprises at least one cell of any one of Embodiments 80 and 89-93.

Embodiment 95. An effector protein or a nucleic acid encoding the effector protein, wherein the effector protein is at least 70%, at least 75%, at least 80%, at least 85%, at least 90%, at least 95%, at least 98% or at least 99% identical to SEQ ID NO: 1, and wherein the effector protein comprises any combination of amino acid substitutions described in TABLE 2.

Embodiment 96. The effector protein or the nucleic acid encoding the effector protein of Embodiment 95, wherein the combination of amino acid substitutions is selected from: L26R, I471T, S223P and D703G; L26R, I471T, S223P, D703G and H208R; L26R, I471T, S223P, D703G and L149R; L26R, I471T, S223P, D703G, L149R and H208R; L26R, I471T, S223P, D703G, D704G and A706G; L26R, I471T, S223P, D703G, L149R, H208R, D704G and A706G; I471T, S223P and D703G; I471T, S223P, D703G and H208R; I471T, S223P, D703G and L149R; I471T, S223P, D703G, L149R and H208R; I471T, S223P, D703G, D704G and A706G; I471T, S223P, D703G, L149R, H208R, D704G and A706G; I471T and E157R; I471T, E157R, S223P and D703G; L26R, I471T, E157R, S223P and D703G; L26R, T87G, S186G, H208R, S223P, C405L, I471T, S526N and D703G; L26R, A121Q, S223P, E258K, I471T, D523K, S526N and D703G; L26R, N147K, H208R, S223P, E258K, I471T, M503K and D703G; L26R, N147K, S186G, S223P, E258K, I471T, S526N, D549L, S638K and D703G; S21L, L26R, S186G, Y220S, S223P, I471T and D703G; L26R, T87G, A121Q, S186G, H208R, Y220S, S223P, C405L, I471T, D523K and D703G; S21L, L26R, A121Q, N147K, S186G, Y220S, S223P, I471T, S526N, D549L and D703G; S21L, L26R, Q76R, N147K, L149R, Y220S, S223P, Y251R, E258K, I471T, M503K, Q552R and D703G; L26R, A121Q, Y220S, S223P, C405L, I471T, D523K, Q552R and D703G; S21L, L26R, A121Q, N147K, Y220S, S223P, Y251R, C405L, I471T and D703G; L26R, Q76R, T87G, S223P, E258K, C279R, I471T, M503K, D523K and D703G; L26R, N147K, S186G, S223P, I471T, M503K, S526N and D703G; S21L, L26R, T87G, N147K, H208R, Y220S, S223P, I471T and D703G; S21L, L26R, A121Q, N147K, S186G, S223P, E258K, I471T, D523K, Q552R and D703G; L26R, A121Q, L149R, S186G, Y220S, S223P, I471T and D703G; L26R, A121Q, N147K, Y220S, S223P, I471T, M503K, S526N, D549L and D703G; L26R, T87G, A121Q, Y220S, S223P, E258K, C405L, I471T and D703G; L26R, T87G, S186G, Y220S, S223P, I471T, M503K and D703G; S21L, L26R, Q76R, T87G, N147K, S186G, S223P, I471T, S526N, S638K and D703G; S21L, L26R, A121Q, Y220S, S223P, C405L, I471T, M503K and D703G; L26R, S223P, I471T and D703G; L26R, T87G, S223P, I471T, S526N and D703G; L26R, T87G, N147K, S223P, I471T, S526N and D703G; L26R, T87G, N147K, S223P, E258K, I471T, S526N and D703G; L26R, T87G, Y220S, S223P, I471T, S526N and D703G; L26R, T87G, N147K, Y220S, S223P, E258K, I471T, S526N and D703G; L26R, Q76R, T87G, N147K, Y220S, S223P, E258K, C279R, I471T, M503K, D523K and D703G; L26R, Q76R, T87G, N147K, Y220S, S223P, E258K, I471T, M503K, D523K and D703G; L26R, Q76R, T87G, N147K, Y220S, S223P, E258K, I471T, M503K and D703G; S21L, L26R, Q76R, T87G, S223P, E258K, C279R, C405L, I471T, M503K, D523K and D703G; L26R, Q76R, T87G, N147K, S186G, Y220S, S223P, E258K, C405L, I471T, M503K and D703G; L26R, T87G, Y220S, S223P, I471T and D703G; L26R, T87G, N147K, Y220S, S223P, E258K, I471T and D703G; S21L, L26R, T87G, N147K, Y220S, S223P, E258K, I471T and D703G; S21L, L26R, T87G, N147K, Y220S, S223P, E258K, C405L, I471T, M503K and D703G; S21L, L26R, T87G, A121Q, N147K, Y220S, S223P, E258K, C405L, I471T, M503K, S638K and D703G; S21L, L26R, T87G, A121Q, N147K, S186G, Y220S, S223P, E258K, C405L, I471T, M503K, S638K and D703G; L26R, T87G, S223P, I471T and D703G; L26R, T87G, N147K, S223P, I471T and D703G; L26R, T87G, N147K, S223P, E258K, I471T and D703G; L26R, T87G, S186G, H208R, S223P, C405L, I471T and D703G; L26R, N147K, S186G, S223P, I471T, M503K and D703G; and L26R, S223P, E258K, I471T and D703G.

Embodiment 97. The effector protein or the nucleic acid encoding the effector protein of Embodiment 95 or 96, wherein the effector protein further comprises a deletion of one or more domains, a substitution of one or more domains for a different amino acid sequence, or a combination thereof, wherein the one or more domains independently comprise an amino acid sequence that is at least 90% identical to any one of the domains identified in TABLE 3.

Embodiment 98. A composition comprising: the effector protein or the nucleic acid encoding the effector protein of any one of Embodiments 95-97; and a guide nucleic acid.

Embodiment 99. The composition of Embodiment 98, wherein the guide nucleic acid comprises a nucleotide sequence that is at least 70%, at least 75%, at least 80%, at least 85%, at least 90%, at least 95% or 100% identical to SEQ ID NO: 348.

### Sequences and Tables

TABLE 1 provides illustrative parental effector protein sequences that are useful in the compositions, systems and methods described herein.

TABLE 1
EXEMPLARY AMINO ACID SEQUENCE(S) OF WT EFFECTOR PROTEINS

| SEQ ID No: | Effector Sequence |
|---|---|
| 1 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 2 | MPKPAVESEFSKVLKKHFPGERFRSSYMKRGGKILAAQGEEAVVAYLQGKSEEE |
| | PPNFQPPAKCHVVTKSRDFAEWPIMKASEAIQRYIYALSTTERAACKPGKSSES |
| | HAAWFAATGVSNHGYSHVQGLNLIFDHTLGRYDGVLKKVQLRNEKARARLESIN |
| | ASRADEGLPEIKAEEEEVATNETGHLLQPPGINPSFYVYQTISPQAYRPRDEIV |
| | LPPEYAGYVRDPNAPIPLGVVRNRCDIQKGCPGYIPEWQREAGTAISPKTGKAV |
| | TVPGLSPKKNKRMRRYWRSEKEKAQDALLVTVRIGTDWVVIDVRGLLRNARWRT |
| | IAPKDISLNALLDLFTGDPVIDVRRNIVTFTYTLDACGTYARKWTLKGKQTKAT |
| | LDKLTATQTVALVAIDLGQTNPISAGISRVTQENGALQCEPLDRFTLPDDLLKD |
| | ISAYRIAWDRNEEELRARSVEALPEAQQAEVRALDGVSKETARTQLCADFGLDP |
| | KRLPWDKMSSNTTFISEALLSNSVSRDQVFFTPAPKKGAKKKAPVEVMRKDRTW |
| | ARAYKPRLSVEAQKLKNEALWALKRTSPEYLKLSRRKEELCRRSINYVIEKTRR |
| | RTQCQIVIPVIEDLNVRFFHGSGKRLPGWDNFFTAKKENRWFIQGLHKAFSDLR |
| | THRSFYVFEVRPERTSITCPKCGHCEVGNRDGEAFQCLSCGKTCNADLDVATHN |
| | LTQVALTGKTMPKREEPRDAQGTAPARKTKKASKSKAPPAEREDQTPAQEPSQT |
| | S |
| 3 | MEKEITELTKIRREFPNKKFSSTDMKKAGKLLKAEGPDAVRDELNSCQEIIGDF |
| | KPPVKTNIVSISRPFEEWPVSMVGRAIQEYYFSLTKEELESVHPGTSSEDHKSF |
| | FNITGLSNYNYTSVQGLNLIFKNAKAIYDGTLVKANNKNKKLEKKFNEINHKRS |
| | LEGLPIITPDFEEPFDENGHLNNPPGINRNIYGYQGCAAKVFVPSKHKMVSLPK |
| | EYEGYNRDPNLSLAGFRNRLEIPEGEPGHVPWFQRMDIPEGQIGHVNKIQRFNF |
| | VHGKNSGKVKFSDKTGRVKRYHHSKYKDATKPYKFLEESKKVSALDSILAIITI |
| | GDDWVVFDIRGLYRNVFYRELAQKGLTAVQLLDLFTGDPVIDPKKGVVTFSYKE |
| | GVVPVFSQKIVPRFKSRDTLEKLTSQGPVALLSVDLGQNEPVAARVCSLKNIND |
| | KITLDNSCRISFLDDYKKQIKDYRDSLDELEIKIRLEAINSLETNQQVEIRDLD |
| | VFSADRAKANTVDMFDIDPNLISWDSMSDARVSTQISDLYLKNGGDESRVYFEI |
| | NNKRIKRSDYNISQLVRPKLSDSTRKNLNDSIWKLKRTSEEYLKLSKRKLELSR |
| | AVVNYTIRQSKLLSGINDIVIILEDLDVKKKFNGRGIRDIGWDNFFSSRKENRW |
| | FIPAFHKTFSELSSNRGLCVIEVNPAWTSATCPDCGFCSKENRDGINFTCRKCG |
| | VSYHADIDVATLNIARVAVLGKPMSGPADRERLGDTKKPRVARSRKTMKRKDIS |
| | NSTVEAMVTA |
| 4 | MPKIKKPTEISLLRKEVFPDLHFAKDRMRAASLVLKNEGREAAIEYLRVNHEDK |
| | PPNFMPPAKTPYVALSRPLEQWPIAQASIAIQKYIFGLTKDEFSATKKLLYGDK |
| | STPNTESRKRWFEVTGVPNFGYMSAQGLNAIFSGALARYEGVVQKVENRNKKRF |
| | EKLSEKNQLLIEEGQPVKDYVPDTAYHTPETLQKLAENNHVRVEDLGDMIDRLV |
| | HPPGIHRSIYGYQQVPPFAYDPDNPKGIILPKAYAGYTRKPHDIIEAMPNRLNI |
| | PEGQAGYIPEHQRDKLKKGGRVKRLRTTRVRVDATETVRAKAEALNAEKARLRG |
| | KEAILAVFQIEEDWALIDMRGLLRNVYMRKLIAAGELTPTTLLGYFTETLTLDP |
| | RRTEATFCYHLRSEGALHAEYVRHGKNTRELLLDLTKDNEKIALVTIDLGQRNP |
| | LAAAIFRVGRDASGDLTENSLEPVSRMLLPQAYLDQIKAYRDAYDSFRQNIWDT |
| | ALASLTPEQQRQILAYEAYTPDDSKENVLRLLLGGNVMPDDLPWEDMTKNTHYI |
| | SDRYLADGGDPSKVWFVPGPRKRKKNAPPLKKPPKPRELVKRSDHNISHLSEFR |
| | PQLLKETRDAFEKAKIDTERGHVGYQKLSTRKDQLCKEILNWLEAEAVRLTRCK |
| | TMVLGLEDLNGPFFNQGKGKVRGWVSFFRQKQENRWIVNGFRKNALARAHDKGK |
| | YILELWPSWTSQTCPKCKHVHADNRHGDDFVCLQCGARLHADAEVATWNLAVVA |
| | IQGHSLPGPVREKSNDRKKSGSARKSKKANESGKVVGAWAAQATPKRATSKKET |
| | GTARNPVYNPLETQASCPAP |
| 5 | QAVIKYLSDKGAVDPPDFRPPAKCNIIAQSRPFDEWPICKASMAIQQHIYGLTK |
| | NEFDESSPGTSSASHEQWFAKTGVDTHGFTHVQGLNLIFQHAKKRYEGVIKKVE |
| | NYNEKERKKFEGINERRSKEGMPLLEPRLRTAFGDDGKFAEKPGVNPSIYLYQQ |
| | TSPRPYDKTKHPYVHAPFELKEITTIPTQDDRLKIPFGAPGHVPEKHRSQLSMA |
| | KHKRRRAWYALSQNKPRPPKDGSKGRRSVRDLADLKAASLADAIPLVSRVGFDW |
| | VVIDGRGLLRNLRWRKLAHEGMTVEEMLGFFSGDPVIDPRRNVATFIYKAEHAT |
| | VKSRKPIGGAKRAREELLKATASSDGVIRQVGLISVDLGQTNPVAYEISRMHQA |
| | NGELVAEHLEYGLLNDEQVNSIQRYRAAWDSMNESFRQKAIESLSMEAQDEIMQ |
| | ASTGAAKRTREAVLTMFGPNATLPWSRMSSNTTCISDALIEVGKEEETNFVTSN |
| | GPRKRTDAQWAAYLRPRVNPETRALLNQAVWDLMKRSDEYERLSKRKLEMARQC |
| | VNFVVARAEKLTQCNNIGIVLENLVVRNFHGSGRRESGWEGFFEPKRENRWFMQ |
| | VLHKAFSDLAQHRGVMVFEVHPAYSSQTCPACRYVDPKNRSSEDRERFKCLKCG |
| | RSFNADREVATFNIREIARTGVGLPKPDCERSRDVQTPGTARKSGRSLKSQDNL |
| | SEPKRVLQSKTRKKITSTETQNEPLATDLKT |

TABLE 2 provides illustrative mutations that are useful in the compositions, systems and methods described herein.

TABLE 2
EXEMPLARY VARIANTS RELATIVE TO SEQ ID NO: 1

| | Variants |
|---|---|
| Individual | T11R, S21L, S21W, S21F, S21Y, G25I, G25L, G25F, G25W, G25V, |
| Amino Acid | G25Y, L26R, Q54R, G55P, G56P, I59K, K65L, E68P, Q76R, S77V, |
| Substitutions | S78F, S78M, S78I, L79R, T87G, P89T, K92E, E100K, E109K, H110T, |
| | P116G, E119S, A121Q, N129I, N147K, L149R, E157A, E157R, E164A, |
| | E164L, E166A, E166I, E170A, S186G, P187K, K189P, H208R, N209F, |
| | N209Y, Y220S, S223P, V228R, V228K, S229L, Y231K, I240K, Y251R, |
| | E258K, R261W, R261M, R261L, C279W, C279F, C279R, C279I, |
| | C279Y, D283L, C285I, C285V, R294L, K299W, N340L, N340M, |
| | K347A, K364I, A366V, T367I, T367V, G371F, G371Y, D403W, |
| | C405L, N406K, K435Q, N449W, I471T, K480L, I489A, I489S, Y490S, |
| | Y490A, F491A, F491S, F491G, D495G, D495R, D495K, K496A, |
| | K496S, K498A, K498S, K500A, K500S, D501R, D501G, D501K, |
| | V502A, V502S, M503K, K504A, K504S, S505R, D506A, K508R, |
| | K516R, V521T, D523K, S526N, W530K, W530R, R531E, D549W, |
| | D549I, D549Y, D549L, Q552R, N568D, G577H, N601Y, N601F, |
| | R617W, R617Y, L620E, P622N, A623P, R625F, R625W, R625Y, |
| | T629V, K634G, S638K, I653A, T668W, D703G, D704G, A706G |
| Combination | K189P and I471T; S638K and I471T; Q54R and I471T; E258K and |
| of Amino | I471T; L79R and I471T; Y220S and I471T; N406K and I471T; E109K |
| Acid | I471T; E100K and I471T; E119S and I471T; K92E and I471T; K435Q |
| Substitutions | and I471T; I240K and I471T; N568D and I471T; V521T and I471T; |
| | H110T and I471T; C405L and I471T; N147K and I471T; P116G and |
| | I471T; S186G and I471T; G55P, G56P and I471T; Q76R and I471T; |
| | K347A and I471T; K516R and I471T; P622N and A623P I471T; K634G |
| | and I471T; T11R and I471T; T87G and I471T; P89T and I471T; Y231K |
| | and I471T; S77V and I471T; E68P and I471T; L620E and I471T; S526N |
| | and I471T; G577H and I471T; K480L and I471T; R617Y and I471T; |
| | I653A and I471T; R531E and I471T; M503K and I471T; A366V and |
| | I471T; I471T and W530K; I471T and W530R; I471T and K508R; I471T |
| | and V228R; I471T and V228K; I471T and P187K; I471T and I59K; |
| | L26R, I471T, S223P and D703G; L26R, I471T, S223P, D703G and |
| | H208R; L26R, I471T, S223P, D703G and L149R; L26R, I471T, S223P, |
| | D703G, L149R and H208R; L26R, I471T, S223P, D703G, D704G and |
| | A706G; L26R, I471T, S223P, D703G, L149R, H208R, D704G and |
| | A706G; I471T, S223P and D703G; I471T, S223P, D703G and H208R; |
| | I471T, S223P, D703G and L149R; I471T, S223P, D703G, L149R and |
| | H208R; I471T, S223P, D703G, D704G and A706G; I471T, S223P, |
| | D703G, L149R, H208R, D704G and A706G; I471T and E157R; I471T, |
| | E157R, S223P and D703G; L26R, I471T, E157R, S223P and D703G; |
| | R617W, G25W, C285I and I471T; R617W, N601Y, D549I and I471T; |
| | G25W, N601Y, G371Y and I471T; C285I, D549I, G371Y and I471T; |
| | G25Y, R617Y, K65L and I471T; G25Y, R625F, N209F and I471T; |
| | R625F, R617Y, G25F and I471T; N209F, K65L, G25F and I471T; |
| | T367I, S21W, R294L and I471T; S78M, R261W, C279F and I471T; |
| | T367I, D549W, R261M and I471T; S78M, D549Y, S21F and I471T; |
| | C279I, D549L, N601F and I471T; D549L, S78F, R261L and I471T; |
| | C279I, S78F, T668W and I471T; D549W, S21W, R625Y and I471T; |
| | N601F, T668W, R261L and I471T; T367V, G25L, R625W and I471T; |
| | D283L, G25L, N449W and I471T; R261W, D549Y, C285V and I471T; |
| | S78I, N340M, D403W and I471T; N209Y, K364I, S21Y and I471T; |
| | G371F, G25V, T629V and I471T; K364I, N129I, N340L and I471T; |
| | C279W, N449W, R625W and I471T; S78I, S229L, C279Y and I471T; |
| | N209Y, N129I, K299W and I471T; S229L, N340M, G25I and I471T; |
| | C279Y, D403W, G25I and I471T; S21Y, K299W, N340L and I471T; |
| | L26R, T87G, S186G, H208R, S223P, C405L, I471T, S526N and D703G; |
| | L26R, A121Q, S223P, E258K, I471T, D523K, S526N and D703G; |
| | L26R, N147K, H208R, S223P, E258K, I471T, M503K and D703G; |
| | L26R, N147K, S186G, S223P, E258K, I471T, S526N, D549L, S638K |
| | and D703G; S21L, L26R, S186G, Y220S, S223P, I471T and D703G; |
| | L26R, T87G, A121Q, S186G, H208R, Y220S, S223P, C405L, I471T, |
| | D523K and D703G; S21L, L26R, A121Q, N147K, S186G, Y220S, |
| | S223P, I471T, S526N, D549L and D703G; S21L, L26R, Q76R, N147K, |
| | L149R, Y220S, S223P, Y251R, E258K, I471T, M503K, Q552R and |
| | D703G; L26R, A121Q, Y220S, S223P, C405L, I471T, D523K, Q552R |
| | and D703G; S21L, L26R, A121Q, N147K, Y220S, S223P, Y251R, |
| | C405L, I471T and D703G; L26R, Q76R, T87G, S223P, E258K, C279R, |
| | I471T, M503K, D523K and D703G; L26R, N147K, S186G, S223P, |
| | I471T, M503K, S526N and D703G; S21L, L26R, T87G, N147K, H208R, |
| | Y220S, S223P, I471T and D703G; S21L, L26R, A121Q, N147K, S186G, |
| | S223P, E258K, I471T, D523K, Q552R and D703G; L26R, A121Q, |
| | L149R, S186G, Y220S, S223P, I471T and D703G; L26R, A121Q, |
| | N147K, Y220S, S223P, I471T, M503K, S526N, D549L and D703G; |
| | L26R, T87G, A121Q, Y220S, S223P, E258K, C405L, I471T and |
| | D703G; L26R, T87G, S186G, Y220S, S223P, I471T, M503K and |
| | D703G; S21L, L26R, Q76R, T87G, N147K, S186G, S223P, I471T, |
| | S526N, S638K and D703G; S21L, L26R, A121Q, Y220S, S223P, |
| | C405L, I471T, M503K and D703G; L26R, S223P, I471T and D703G; |
| | L26R, T87G, S223P, I471T, S526N and D703G; L26R, T87G, N147K, |
| | S223P, I471T, S526N and D703G; L26R, T87G, N147K, S223P, E258K, |
| | I471T, S526N and D703G; L26R, T87G, Y220S, S223P, I471T, S526N |
| | and D703G; L26R, T87G, N147K, Y220S, S223P, E258K, I471T, |
| | S526N and D703G; L26R, Q76R, T87G, N147K, Y220S, S223P, E258K, |
| | C279R, I471T, M503K, D523K and D703G; L26R, Q76R, T87G, |
| | N147K, Y220S, S223P, E258K, I471T, M503K, D523K and D703G; |
| | L26R, Q76R, T87G, N147K, Y220S, S223P, E258K, I471T, M503K and |
| | D703G; S21L, L26R, Q76R, T87G, S223P, E258K, C279R, C405L, |
| | I471T, M503K, D523K and D703G; L26R, Q76R, T87G, N147K, |
| | S186G, Y220S, S223P, E258K, C405L, I471T, M503K and D703G; |
| | L26R, T87G, Y220S, S223P, I471T and D703G; L26R, T87G, N147K, |
| | Y220S, S223P, E258K, I471T and D703G; S21L, L26R, T87G, N147K, |
| | Y220S, S223P, E258K, I471T and D703G; S21L, L26R, T87G, N147K, |
| | Y220S, S223P, E258K, C405L, I471T, M503K and D703G; S21L, |
| | L26R, T87G, A121Q, N147K, Y220S, S223P, E258K, C405L, I471T, |
| | M503K, S638K and D703G; S21L, L26R, T87G, A121Q, N147K, |
| | S186G, Y220S, S223P, E258K, C405L, I471T, M503K, S638K and |
| | D703G; L26R, T87G, S223P, I471T and D703G; L26R, T87G, N147K, |
| | S223P, I471T and D703G; L26R, T87G, N147K, S223P, E258K, I471T |
| | and D703G; L26R, T87G, S186G, H208R, S223P, C405L, I471T and |
| | D703G; L26R, N147K, S186G, S223P, I471T, M503K and D703G; |
| | L26R, S223P, E258K, I471T and D703G |

TABLE 3 provides illustrative mutations that are useful in the compositions, systems and methods described herein.

TABLE 3
EXEMPLARY AMINO ACID SEQUENCE(S) MUTATIONS RELATIVE
TO WT EFFECTOR PROTEIN AMINO ACID SEQUENCES

| | | Mutations | | |
|---|---|---|---|---|
| Effector | | Deletion | | |
| Protein | | of | Substitution of Domain | |
| SEQ ID NO: | Domain | Domain | SEQ Id NO: | Substituent Amino Acid Sequence |
| 1 | G55-159 | Yes | 356 | PPAK |
| | (GGPAI | | | |
| | (SEQ ID | | | |
| | NO: 349)) | | | |
| 1 | D113-S190 | Yes | 41 | CTDYFSVQGLNLIFQNARKRYIGVQTKV |
| | (DTVPYKE | | | TNRNEKRHKKLKRINAKRIAEGLPELTS |
| | AAGLNLII | | | DEPESALDETGHLIDPPGLNTN |
| | KNAVNTYK | | 42 | PDRGLPVQAINKIAKAAVNRAFGVVRKV |
| | GVQVKVDN | | | ENRNEKRRSRDNRIAEHNRENGLTEVVR |
| | KNKNNLAK | | | EAPEVATNADGFLLHPPGIDPS |
| | INRKNEIA | | 43 | PNFGYMSAQGLNAIFSGALARYEGVVQK |
| | KLNGEQEI | | | VENRNKKRFEKLSEKNQLLIEEGQPVKD |
| | SFEEIKAF | | | YVPDTAYHTPETLQKLAENNHVRVEDLG |
| | DDKGYLLQ | | | DMIDRLVHPPGIHRS |
| | KPSPNKS | | 44 | SEDDLMALEAQLLETIMGNAISLHGGVL |
| | (SEQ ID | | | KKIDNANVKAAKRLSGRNEARLNKGLQE |
| | NO: 6)) | | | LPPEQEGSAYGADGLLVNPPGLNLN |
| | | | 45 | SNYNYTSVQGLNLIFKNAKAIYDGTLVK |
| | | | | ANNKNKKLEKKFNEINHKRSLEGLPIIT |
| | | | | PDFEEPFDENGHLNNPPGINRN |
| | | | 46 | LTFPMNQVQMLNGIFNRAFSVYLGVEKK |
| | | | | VANRNAEAREKLKNRGQEDQFVEEFAYS |
| | | | | YCPVDFKDGMFVHDSAEEGETQGCLLHP |
| | | | | PGIDPK |
| | | | 47 | NIEYQNVAGLNLIFNNVKNTYNGVILKV |
| | | | | KNRNEKLKKKAIKNNYEFEEIKTFNDDG |
| | | | | CLINKPGINNV |
| 1 | A130-N148 | Yes | 48 | GSSG |
| | (AVNTYKG | | | |
| | VQVKVDNK | | | |
| | NKNN | | | |
| | (SEQ ID | | | |
| | NO: 7)) | | | |
| 1 | L149-F169 | Yes | 48 | GSSG |
| | (LAKINRK | | | |
| | NEIAKLNG | | | |
| | EQEISF | | | |
| | (SEQ ID | | | |
| | NO: 8)) | | | |
| 1 | L149-E170 | Yes | 48 | GSSG |
| | (LAKINRK | | | |
| | NEIAKLNG | | | |
| | EQEISFE | | | |
| | (SEQ ID | | | |
| | NO: 9)) | | | |
| 1 | I152-S168 | Yes | 48 | GSSG |
| | (INRKNEI | | | |
| | AKLNGEQE | | | |
| | IS (SEQ | | | |
| | ID NO: | | | |
| | 10)) | | | |
| 1 | N153-I167 | Yes | 49 | GS |
| | (NRKNEIA | | | |
| | KLNGEQEI | | | |
| | (SEQ ID | | | |
| | NO: 11)) | | | |
| 1 | N156-E166 | Yes | 49 | GS |
| | (NEIAKLN | | | |
| | GEQE | | | |
| | (SEQ ID | | | |
| | NO: 12)) | | | |
| 1 | N162-E170 | Yes | 48 | GSSG |
| | (NGEQEIS | | | |
| | FE (SEQ | | | |
| | ID NO: 13) | | | |
| 1 | P247-F287 | Yes | 50 | PEWQHPLLNRRKNRRRRDWYSASLNKPK |
| | (PKWQYTF | | | ATCSKRSGTPNRKNSRTDQIQSGRFKGA |
| | LSKKENKR | | | IPVLMRFQDEWVII |
| | RKLSKRIK | | 51 | PGHQRFADTGQNNSGKANPNKKGRMRKY |
| | NVSPILGI | | | YGHGTKYTQPGEYQEVFRKGHREGNKRR |
| | ICIKKDWC | | | YWEEDFRSEAHDCILYVIHIGDDWVVC |
| | VF (SEQ | | 52 | PEHQRDKLKKGGRVKRLRTTRVRVDATE |
| | ID NO: 14)) | | | TVRAKAEALNAEKARLRGKEAILAVFQI |
| | | | | EEDWALI |
| | | | 53 | PEPHREGLIGRKDRRMRRYYETERGTKL |
| | | | | KRPPLTAKGRADKANEALLVVVRIDSDW |
| | | | | VVM |
| | | | 54 | PEHQRKNLKKKGRVRLYRRTPPKTKALA |
| | | | | SILAVLQIGKDWVLF |
| | | | 55 | PEWQRPLLNRHKGRRHRSWYANSLNKPR |
| | | | | KSRTEEAKDRQNAGKRTALIEAERLKGV |
| | | | | LPVLMRFKEDWLII |
| | | | 56 | PAWQREQGLVKPGGRRRRLSGSESNMRQ |
| | | | | KVDPSTGPRRSTRSGTVNRSNQRTGRNG |
| | | | | DPLLVEIRMKEDWVLL |
| | | | 57 | DEYGKLISKRRKERINKDDAILCVSNFG |
| | | | | DDWIIF |
| | | | 58 | PPWDRENLSVKKHRRKRASWARSRGGAI |
| | | | | DDNMLLAVVRVADDWALL |
| | | | 59 | PLHDREKLTSNKHRRMKLPKSLRAQGAL |
| | | | | PVCFRVEDDWAVV |
| | | | 60 | PEWMRTAGEKTNPRTQKKEMHPGLSTRK |
| | | | | NKRMRLPRSVRSAPLGALLVTIHLGEDW |
| | | | | LVI |
| | | | 61 | NKIQRFNFVHGKNSGKVKFSDKTGRVKR |
| | | | | YHHSKYKDATKPYKFLEESKKVSALDSI |
| | | | | LAIITIGDDWVVF |
| | | | 62 | PWFQRMDIPEGQIGHVNKIQRFNFVHGK |
| | | | | NSGKVKFSDKTGRVKRYHHSKYKDATKP |
| | | | | YKFLEESKKVSALDSILAIITIGDDWVV |
| | | | | F |
| | | | 63 | PEKHRSQLSMAKHKRRRAWYALSQNKPR |
| | | | | PPKDGSKGRRSVRDLADLKAASLADAIP |
| | | | | LVSRVGFDWVVI |
| | | | 64 | PYWQRPFLSKRRNRRVRAGWGKQVSSIQ |
| | | | | AWLTGALLVIVRLGNEAFLA |
| | | | 65 | PGHQRKESTTEGPKINFRKGRIRRSYTA |
| | | | | LYAKRDSRRVRQGKLALPSYRHHMMRLN |
| | | | | SNAESAILAVIFFGKDWVVF |
| 1 | K248-N259 | Yes | 357 | EWQREAGTAISP |
| | (KWQYTFL | | | |
| | SKKEN | | | |
| | (SEQ ID | | | |
| | NO: 350)) | | | |
| 1 | L264-P273 | Yes | 358 | AVTVPGLSPKKNKRMRRYWRSEKEKAQD |
| | (LSKRIKN | | | A |
| | VSP (SEQ | | | |
| | ID NO: | | | |
| | 351)) | | | |
| 1 | K248-P273 | Yes | 359 | EKHRSQLSMAKHKRRRAWYALSQNKPRP |
| | (KWQYTFL | | | PKDGSKGRRSVRDLADLKAASLADA |
| | SKKENKRR | | 360 | EWMRTAGEKTNPRTQKKFMHPGLSTRKN |
| | KLSKRIKN | | | KRMRLPRSVRSAPLGA |
| | VSP (SEQ | | 361 | EWQRPHLSMKCKRVRMWYARANWRRKPG |
| | ID NO: 352)) | | | RRSVLNEARLKEASAKGA |
| | | | 362 | EWQREAGTAISPKTGKAVTVPGLSPKKN |
| | | | | KRMRRYWRSEKEKAQDA |
| | | | 363 | EWQRSQLTTQKHRRKRSWYSAQKWKPRT |
| | | | | GRTSTFDPDRLNCARAQGA |
| | | | 364 | EPHREGLTGRKDRRMRRYYETERGTKLK |
| | | | | RPPLTAKGRADKANEA |
| | | | 365 | EWQRLKCSTNKHRRMRQWSNQDYKPKAG |
| | | | | RRAKPLEFQAHLTRERAKGA |
| 1 | Y251-P273 | Yes | 366 | REAGTAISPKTGKAVTVPGLSPKKNKRM |
| | (YTFLSKK | | | RRYWRSEKEKAQDA |
| | ENKRRKLS | | | |
| | KRIKNVSP | | | |
| | (SEQ ID | | | |
| | NO: 353)) | | | |
| 1 | R261-P273 | Yes | 367 | TGKAVTVPGLSPKKNKRMRRYWRSEKEK |
| | (RRKLSKR | | | AQDA |
| | IKNVSP | | | |
| | (SEQ ID | | | |
| | NO: 354)) | | | |
| 1 | L264-P273 | Yes | 368 | AVTVPGLSPKKNKRMRRYWRSEKEKAQD |
| | (LSKRIKN | | | A |
| | VSP (SEQ | | | |
| | ID NO: | | | |
| | 355)) | | | |
| 1 | S478-S505 | Yes | 66 | TDLFLARGGDPKKCMFTSEPKKKKNSKQ |
| | (SEKAQVS | | | VLYKIR |
| | NKSEIYFT | | 67 | ATAYLEKGGDRKVATLKPKNRPEMLRR |
| | STDKGKTK | | 68 | SDDFLRRGGDPNIVHFDRQPKKGKVSKK |
| | DVMKS | | | SQRIKRS |
| | (SEQ ID | | 69 | KQFLKKNGGNKSLIEYIPYQKKKSKKTP |
| | NO: 15)) | | | KAVLRS |
| | | | 70 | SEALLSNSVSRDQVFFTPAPKKGAKKKA |
| | | | | PVEVMRK |
| | | | 71 | SDRYLADGGDPSKVWFVPGPRKRKKNAP |
| | | | | PLKKPPKPRELVKRS |
| | | | 72 | SDHILDHGGDPDTVFFMATKRGQNKPTL |
| | | | | HKRK |
| | | | 73 | SDLFLRMGGDRSRVFFEVPPKKAKKAPK |
| | | | | KPPKKPAGPRIVKRT |
| | | | 74 | ANLYLERGGDPSKVFFTRPLKKDSKSKK |
| | | | | PRKPTKRT |
| | | | 75 | VDAVLRKGGDESLVYFKNKEGETLKER |
| | | | 76 | ADHVQAAGGDVEWFFFKTCGKGKEIKTV |
| | | | | RRS |
| | | | 77 | AQAFVAKGGDPKVVTIKPNGKPKMFRK |
| | | | 78 | SDQLLQQGADPASLFFDGERDGKPCRHK |
| | | | | KK |
| | | | 79 | ADMLISHGRDDDAFLSHGEFPTLEKRKK |
| | | | | F |
| | | | 80 | SEALSAKGSPLALHDGAPIK |
| | | | 81 | RDCLKNKNIDESEFTFEKNGKKLDPT |
| | | | 82 | SDLAKERGVDDTLYMFPFFKGKGKKRKT |
| | | | | EIRKRW |
| | | | 83 | SRWLRDHGWNASDFTQITKGRKKVERLW |
| | | | | S |
| | | | 84 | SDLYLRRGGDPSRVFFVPQPSTPKKNAK |
| | | | | KPPAPRKPVKRT |
| | | | 85 | SDHLINHGGDTSAVFFQTIRKGTKKLET |
| | | | | IKRK |
| | | | 86 | ADELIKFENKDAFYPSGKKKF |
| | | | 87 | SEAILAQGVSRDQVFFTPNPKKGSKEPV |
| | | | | EVMRK |
| | | | 88 | ADEFINNGGDKSLVYFTAKDKKSKKEKL |
| | | | | VKLS |
| | | | 89 | SDLYLKNGGDESRVYFEINNKRIKRS |
| | | | 90 | AQHLMSLGFGESATHVRYRPKRKASERT |
| | | | | ILKY |
| | | | 91 | SDALIEVGKEEETNFVTSNGPRKRT |
| | | | 92 | SDCLVALGKTDDVFFVPKAKKGKKETGI |
| | | | | AVKRK |
| | | | 93 | SDYLIDNNITNDVYRTVNKAKYKTN |
| | | | 94 | SDYLIQIGRGTETEKTITTKKGKEKILT |
| | | | | IR |
| | | | 95 | SDLYIERGGDPRDVHQQVETKPKGKRKS |
| | | | | EIRILKIR |
| | | | 96 | SDYIVDHGGDPEKVFFETKSKKDKTKRY |
| | | | | KRR |
| | | | 97 | SDHLLAKGVTDQVFFEKKSKGKKKGTET |
| | | | | VKRK |
| | | | 98 | FNKHVENGGDPNVLLFTPTEDKKNKGKK |
| | | | | SKNKKGEYGDRVPHS |
| | | | 99 | SNYLLNNGLSPQEGASDQDWVVPSSTRE |
| | | | | VARVAYRISDSKGKGKKKPEIFKAS |
| | | | 100 | AKWVMRHGDPSLVEVLEYRKDNEIKLDK |
| | | | | NGVPKKVKLT |
| 1 | S479-S505 | Yes | 101 | DLYIERGGDPRDVHQQVETKPKGKRKSE |
| | (EKAQVSN | | | IRILKIR |
| | KSEIYFTS | | | |
| | TDKGKTKD | | | |
| | VMKS | | | |
| | (SEQ ID | | | |
| | NO: 16)) | | | |
| 1 | I489-S505 | Yes | 48 | GSSG |
| | (IYFTSTD | | | |
| | KGKTKDVM | | | |
| | KS (SEQ | | | |
| | ID NO: | | | |
| | 17)) | | | |
| 1 | V483-S505 | Yes | 48 | GSSG |
| | (VSNKSEI | | | |
| | YFTSTDKG | | | |
| | KTKDVMKS | | | |
| | (SEQ ID | | | |
| | NO: 18)) | | | |
| 1 | I489-F491 | Yes | 102 | SGS |
| | (IVF (SEQ | | | |
| | ID NO: | | | |
| | 19)) | | | |
| 1 | K496-K504 | Yes | 48 | GSSG |
| | (KGKTKDV | | | |
| | MK (SEQ | | | |
| | ID NO: | | | |
| | 20)) | | | |
| 1 | N568-R597 | Yes | 103 | DLDVKKKFNGRGIRDIGWDNFFSSRKEN |
| | (NLVKKNN | | | R |
| | FFGGSGKR | | | |
| | EPGWDNFY | | | |
| | KPKKENR | | | |
| | (SEQ ID | | | |
| | NO: 21)) | | | |
| 1 | T629-A657 | Yes | 104 | TCPDCGFCSKENRDGINFTCRKCGVSYH |
| | (TCPKCKY | | | A |
| | CDSKNRNG | | | |
| | EKFNCLKC | | | |
| | GIELNA) | | | |
| | (SEQ ID | | | |
| | NO: 22) | | | |
| 2 | R155-E176 | Yes | 48 | GSSG |
| | (RARLESI | | | |
| | NASRADEG | | | |
| | LPEIKAE | | | |
| | (SEQ ID | | | |
| | NO: 23)) | | | |
| 2 | S509-K536 | Yes | 48 | GSSG |
| | (SVSRDQV | | 18 | VSNKSEIYFTSTDKGKTKDVMKS |
| | FFTPAPKK | | | |
| | GAKKKAPV | | | |
| | EVMRK | | | |
| | (SEQ ID | | | |
| | NO: 24)) | | | |
| 2 | K522-K536 | Yes | 48 | GSSG |
| | (KKGAKKK | | | |
| | APVEVMRK | | | |
| | (SEQ ID | | | |
| | NO: 25)) | | | |
| 3 | E151-D172 | Yes | 48 | GSSG |
| | (EKKFNEI | | | |
| | NHKRSLEG | | | |
| | LPIITPD | | | |
| | (SEQ ID | | | |
| | NO: 26)) | | | |
| 3 | G530-S548 | Yes | 48 | GSSG |
| | (GGDESRV | | 18 | VSNKSEIYFTSTDKGKTKDVMKS |
| | YFEINNKR | | | |
| | IKRS (SEQ | | | |
| | ID NO: | | | |
| | 27)) | | | |
| 3 | K543-S548 | Yes | 48 | GSSG |
| | (KRIKRS | | | |
| | (SEQ ID | | | |
| | NO: 28)) | | | |
| 4 | F162-V183 | Yes | 48 | GSSG |
| | (FEKLSEK | | | |
| | NOLLIEEG | | | |
| | QPVKDYV | | | |
| | (SEQ ID | | | |
| | NO: 29)) | | | |
| 4 | G548-S583 | Yes | 48 | GSSG |
| | (GGDPSKV | | 18 | VSNKSEIYFTSTDKGKTKDVMKS |
| | WFVPGPRK | | | |
| | RKKNAPPL | | | |
| | KKPPKPRE | | | |
| | LVKRS | | | |
| | (SEQ ID | | | |
| | NO: 30)) | | | |
| 4 | R562-S583 | Yes | 48 | GSSG |
| | (RKRKKNA | | | |
| | PPLKKPPK | | | |
| | PRELVKRS | | | |
| | (SEQ ID | | | |
| | NO: 31)) | | | |
| 5 | R115-R136 | Yes | 48 | GSSG |
| | (RKKFEGI | | | |
| | NERRSKEG | | | |
| | MPLLEPR | | | |
| | (SEQ ID | | | |
| | NO: 32)) | | | |
| 5 | G475-T492 | Yes | 48 | GSSG |
| | (GKEEETN | | 18 | VSNKSEIYFTSTDKGKTKDVMKS |
| | FVTSNGPR | | | |
| | KRT (SEQ | | | |
| | ID NO: | | | |
| | 33)) | | | |
| 5 | P488-T492 | Yes | 48 | GSSG |
| | (PRKRT | | | |
| | (SEQ ID | | | |
| | NO: 34)) | | | |

TABLE 4 provides illustrative amino acid sequences of effector proteins that are useful in the compositions, systems and methods described herein.

TABLE 4
EXEMPLARY AMINO ACID SEQUENCE(S) OF EFFECTOR
PROTEIN(S)

| SEQ | |
|---|---|
| ID | |
| NO: | Amino Acid Sequence |
| 105 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQGSSG |
| | DYKWFQDYKPKLSKEVRDALSDIEWRLRRESLEFNKLSKSREQDARQLANWISS |
| | MCDVIGIENLVKKNNFFGGSGKREPGWDNFYKPKKENRWWINAIHKALTELSQN |
| | KGKRVILLPAMRTSITCPKCKYCDSKNRNGEKFNCLKCGIELNADIDVATENLA |
| | TVAITAQSMPKPTCERSGDAKKPVRARKAKAPEFHDKLAPSYTVVLREAV |
| 106 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEGSSGDYKWFQDYKPKLSKEVRDALSDIEWRLRRESLEFNKLSKSREQDARQL |
| | ANWISSMCDVIGIENLVKKNNFFGGSGKREPGWDNFYKPKKENRWWINAIHKAL |
| | TELSQNKGKRVILLPAMRTSITCPKCKYCDSKNRNGEKFNCLKCGIELNADIDV |
| | ATENLATVAITAQSMPKPTCERSGDAKKPVRARKAKAPEFHDKLAPSYTVVLRE |
| | AV |
| 107 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDGSSGSDYKWFQDYKPKLSKEVRDALSDIEWRLRRESLEFNKLSKS |
| | REQDARQLANWISSMCDVIGIENLVKKNNFFGGSGKREPGWDNFYKPKKENRWW |
| | INAIHKALTELSQNKGKRVILLPAMRTSITCPKCKYCDSKNRNGEKFNCLKCGI |
| | ELNADIDVATENLATVAITAQSMPKPTCERSGDAKKPVRARKAKAPEFHDKLAP |
| | SYTVVLREAV |
| 108 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SESGSTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 109 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTELSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SESSSTSTDKGKTKDSMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 110 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEAAATSTDKGKTKDAMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 111 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SESSFTSTDKGKTKDSMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 112 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEAAFTSTDKGKTKDAMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 113 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYGTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 114 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDKGKTKDVMKSAYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 115 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDAGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 116 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDKGATKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 117 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTELSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDKGKTADVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 118 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDKGKTKDVMASDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 119 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDSGSTSDVMSSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 120 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDAGATADVMASDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 121 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDAGATADVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 122 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTELSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDKGKTKRVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | DKLAPSYTVVLREAV |
| 123 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNGSSGEIKAFDDKGY |
| | LLQKPSPNKSIYCYQSVSPKPFITSKYHNVNLPEEYIGYYRKSNEPIVSPYQFD |
| | RLRIPIGEPGYVPKWQYTFLSKKENKRRKLSKRIKNVSPILGIICIKKDWCVED |
| | MRGLLRTNHWKKYHKPTDSINDLFDYFTGDPVIDTKANVVRFRYKMENGIVNYK |
| | PVREKKGKELLENICDQNGSCKLATVDVGQNNPVAIGLFELKKVNGELTKTLIS |
| | RHPTPIDFCNKITAYRERYDKLESSIKLDAIKQLTSEQKIEVDNYNNNFTPQNT |
| | KQIVCSKLNINPNDLPWDKMISGTHFISEKAQVSNKSEIYFTSTDKGKTKDVMK |
| | SDYKWFQDYKPKLSKEVRDALSDIEWRLRRESLEFNKLSKSREQDARQLANWIS |
| | SMCDVIGIENLVKKNNFFGGSGKREPGWDNFYKPKKENRWWINAIHKALTELSQ |
| | NKGKRVILLPAMRTSITCPKCKYCDSKNRNGEKFNCLKCGIELNADIDVATENL |
| | ATVAITAQSMPKPTCERSGDAKKPVRARKAKAPEFHDKLAPSYTVVLREAV |
| 124 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNGSSGEIKAFDDKGY |
| | LLQKPSPNKSIYCYQSVSPKPFITSKYHNVNLPEEYIGYYRKSNEPIVSPYQFD |
| | RLRIPIGEPGYVPKWQYTELSKKENKRRKLSKRIKNVSPILGIICIKKDWCVED |
| | MRGLLRTNHWKKYHKPTDSINDLEDYFTGDPVIDTKANVVRFRYKMENGIVNYK |
| | PVREKKGKELLENICDQNGSCKLATVDVGQNNPVAIGLFELKKVNGELTKTLIS |
| | RHPTPIDFCNKITAYRERYDKLESSIKLDAIKQLTSEQKIEVDNYNNNFTPQNT |
| | KQIVCSKLNINPNDLPWDKMISGTHFISEKAQVSNKSEIYFTSTDGSSGSDYKW |
| | FQDYKPKLSKEVRDALSDIEWRLRRESLEFNKLSKSREQDARQLANWISSMCDV |
| | IGIENLVKKNNFFGGSGKREPGWDNFYKPKKENRWWINAIHKALTELSQNKGKR |
| | VILLPAMRTSITCPKCKYCDSKNRNGEKFNCLKCGIELNADIDVATENLATVAI |
| | TAQSMPKPTCERSGDAKKPVRARKAKAPEFHDKLAPSYTVVLREAV |
| 125 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNGSSGEIKAFDDKGY |
| | LLQKPSPNKSIYCYQSVSPKPFITSKYHNVNLPEEYIGYYRKSNEPIVSPYQFD |
| | RLRIPIGEPGYVPKWQYTFLSKKENKRRKLSKRIKNVSPILGIICIKKDWCVED |
| | MRGLLRTNHWKKYHKPTDSINDLFDYFTGDPVIDTKANVVRFRYKMENGIVNYK |
| | PVREKKGKELLENICDQNGSCKLATVDVGQNNPVAIGLFELKKVNGELTKTLIS |
| | RHPTPIDFCNKITAYRERYDKLESSIKLDAIKQLTSEQKIEVDNYNNNFTPQNT |
| | KQIVCSKLNINPNDLPWDKMISGTHFISEKAQGSSGDYKWFQDYKPKLSKEVRD |
| | ALSDIEWRLRRESLEFNKLSKSREQDARQLANWISSMCDVIGIENLVKKNNFFG |
| | GSGKREPGWDNFYKPKKENRWWINAIHKALTELSQNKGKRVILLPAMRTSITCP |
| | KCKYCDSKNRNGEKFNCLKCGIELNADIDVATENLATVAITAQSMPKPTCERSG |
| | DAKKPVRARKAKAPEFHDKLAPSYTVVLREAV |
| 126 | MPKPAVESEFSKVLKKHFPGERFRSSYMKRGGKILAAQGEEAVVAYLQGKSEEE |
| | PPNFQPPAKCHVVTKSRDFAEWPIMKASEAIQRYIYALSTTERAACKPGKSSES |
| | HAAWFAATGVSNHGYSHVQGLNLIFDHTLGRYDGVLKKVQLRNEKARARLESIN |
| | ASRADEGLPEIKAEEEEVATNETGHLLQPPGINPSFYVYQTISPQAYRPRDEIV |
| | LPPEYAGYVRDPNAPIPLGVVRNRCDIQKGCPGYIPEWQREAGTAISPKTGKAV |
| | TVPGLSPKKNKRMRRYWRSEKEKAQDALLVTVRIGTDWVVIDVRGLLRNARWRT |
| | IAPKDISLNALLDLFTGDPVIDVRRNIVTFTYTLDACGTYARKWTLKGKQTKAT |
| | LDKLTATQTVALVAIDLGQTNPISAGISRVTQENGALQCEPLDRFTLPDDLLKD |
| | ISAYRIAWDRNEEELRARSVEALPEAQQAEVRALDGVSKETARTQLCADFGLDP |
| | KRLPWDKMSSNTTFISEALLSNGSSGDRTWARAYKPRLSVEAQKLKNEALWALK |
| | RTSPEYLKLSRRKEELCRRSINYVIEKTRRRTQCQIVIPVIEDLNVRFFHGSGK |
| | RLPGWDNFFTAKKENRWFIQGLHKAFSDLRTHRSFYVFEVRPERTSITCPKCGH |
| | CEVGNRDGEAFQCLSCGKTCNADLDVATHNLTQVALTGKTMPKREEPRDAQGTA |
| | PARKTKKASKSKAPPAEREDQTPAQEPSQTS |
| 127 | MPKPAVESEFSKVLKKHFPGERFRSSYMKRGGKILAAQGEEAVVAYLQGKSEEE |
| | PPNFQPPAKCHVVTKSRDFAEWPIMKASEAIQRYIYALSTTERAACKPGKSSES |
| | HAAWFAATGVSNHGYSHVQGLNLIFDHTLGRYDGVLKKVQLRNEKARARLESIN |
| | ASRADEGLPEIKAEEEEVATNETGHLLQPPGINPSFYVYQTISPQAYRPRDEIV |
| | LPPEYAGYVRDPNAPIPLGVVRNRCDIQKGCPGYIPEWQREAGTAISPKTGKAV |
| | TVPGLSPKKNKRMRRYWRSEKEKAQDALLVTVRIGTDWVVIDVRGLLRNARWRT |
| | IAPKDISLNALLDLFTGDPVIDVRRNIVTFTYTLDACGTYARKWTLKGKQTKAT |
| | LDKLTATQTVALVAIDLGQTNPISAGISRVTQENGALQCEPLDRFTLPDDLLKD |
| | ISAYRIAWDRNEEELRARSVEALPEAQQAEVRALDGVSKETARTQLCADFGLDP |
| | KRLPWDKMSSNTTFISEALLSNSVSRDQVFFTPAPGSSGDRTWARAYKPRLSVE |
| | AQKLKNEALWALKRTSPEYLKLSRRKEELCRRSINYVIEKTRRRTQCQIVIPVI |
| | EDLNVRFFHGSGKRLPGWDNFFTAKKENRWFIQGLHKAFSDLRTHRSFYVFEVR |
| | PERTSITCPKCGHCEVGNRDGEAFQCLSCGKTCNADLDVATHNLTQVALTGKTM |
| | PKREEPRDAQGTAPARKTKKASKSKAPPAEREDQTPAQEPSQTS |
| 128 | MPKPAVESEFSKVLKKHFPGERFRSSYMKRGGKILAAQGEEAVVAYLQGKSEEE |
| | PPNFQPPAKCHVVTKSRDFAEWPIMKASEAIQRYIYALSTTERAACKPGKSSES |
| | HAAWFAATGVSNHGYSHVQGLNLIFDHTLGRYDGVLKKVQLRNEKAGSSGEEEV |
| | ATNETGHLLQPPGINPSFYVYQTISPQAYRPRDEIVLPPEYAGYVRDPNAPIPL |
| | GVVRNRCDIQKGCPGYIPEWQREAGTAISPKTGKAVTVPGLSPKKNKRMRRYWR |
| | SEKEKAQDALLVTVRIGTDWVVIDVRGLLRNARWRTIAPKDISLNALLDLFTGD |
| | PVIDVRRNIVTFTYTLDACGTYARKWTLKGKQTKATLDKLTATQTVALVAIDLG |
| | QTNPISAGISRVTQENGALQCEPLDRFTLPDDLLKDISAYRIAWDRNEEELRAR |
| | SVEALPEAQQAEVRALDGVSKETARTQLCADFGLDPKRLPWDKMSSNTTFISEA |
| | LLSNSVSRDQVFFTPAPKKGAKKKAPVEVMRKDRTWARAYKPRLSVEAQKLKNE |
| | ALWALKRTSPEYLKLSRRKEELCRRSINYVIEKTRRRTQCQIVIPVIEDLNVRE |
| | FHGSGKRLPGWDNFFTAKKENRWFIQGLHKAFSDLRTHRSFYVFEVRPERTSIT |
| | CPKCGHCEVGNRDGEAFQCLSCGKTCNADLDVATHNLTQVALTGKTMPKREEPR |
| | DAQGTAPARKTKKASKSKAPPAEREDQTPAQEPSQTS |
| 129 | MPKPAVESEFSKVLKKHFPGERFRSSYMKRGGKILAAQGEEAVVAYLQGKSEEE |
| | PPNFQPPAKCHVVTKSRDFAEWPIMKASEAIQRYIYALSTTERAACKPGKSSES |
| | HAAWFAATGVSNHGYSHVQGLNLIFDHTLGRYDGVLKKVQLRNEKAGSSGEEEV |
| | ATNETGHLLQPPGINPSFYVYQTISPQAYRPRDEIVLPPEYAGYVRDPNAPIPL |
| | GVVRNRCDIQKGCPGYIPEWQREAGTAISPKTGKAVTVPGLSPKKNKRMRRYWR |
| | SEKEKAQDALLVTVRIGTDWVVIDVRGLLRNARWRTIAPKDISLNALLDLFTGD |
| | PVIDVRRNIVTFTYTLDACGTYARKWTLKGKQTKATLDKLTATQTVALVAIDLG |
| | QTNPISAGISRVTQENGALQCEPLDRFTLPDDLLKDISAYRIAWDRNEEELRAR |
| | SVEALPEAQQAEVRALDGVSKETARTQLCADFGLDPKRLPWDKMSSNTTFISEA |
| | LLSNSVSRDQVFFTPAPGSSGDRTWARAYKPRLSVEAQKLKNEALWALKRTSPE |
| | YLKLSRRKEELCRRSINYVIEKTRRRTQCQIVIPVIEDLNVRFFHGSGKRLPGW |
| | DNFFTAKKENRWFIQGLHKAFSDLRTHRSFYVFEVRPERTSITCPKCGHCEVGN |
| | RDGEAFQCLSCGKTCNADLDVATHNLTQVALTGKTMPKREEPRDAQGTAPARKT |
| | KKASKSKAPPAEREDQTPAQEPSQTS |
| 130 | MPKPAVESEFSKVLKKHFPGERFRSSYMKRGGKILAAQGEEAVVAYLQGKSEEE |
| | PPNFQPPAKCHVVTKSRDFAEWPIMKASEAIQRYIYALSTTERAACKPGKSSES |
| | HAAWFAATGVSNHGYSHVQGLNLIFDHTLGRYDGVLKKVQLRNEKAGSSGEEEV |
| | ATNETGHLLQPPGINPSFYVYQTISPQAYRPRDEIVLPPEYAGYVRDPNAPIPL |
| | GVVRNRCDIQKGCPGYIPEWQREAGTAISPKTGKAVTVPGLSPKKNKRMRRYWR |
| | SEKEKAQDALLVTVRIGTDWVVIDVRGLLRNARWRTIAPKDISLNALLDLFTGD |
| | PVIDVRRNIVTFTYTLDACGTYARKWTLKGKQTKATLDKLTATQTVALVAIDLG |
| | QTNPISAGISRVTQENGALQCEPLDRFTLPDDLLKDISAYRIAWDRNEEELRAR |
| | SVEALPEAQQAEVRALDGVSKETARTQLCADFGLDPKRLPWDKMSSNTTFISEA |
| | LLSNGSSGDRTWARAYKPRLSVEAQKLKNEALWALKRTSPEYLKLSRRKEELCR |
| | RSINYVIEKTRRRTQCQIVIPVIEDLNVRFFHGSGKRLPGWDNFFTAKKENRWF |
| | IQGLHKAFSDLRTHRSFYVFEVRPERTSITCPKCGHCEVGNRDGEAFQCLSCGK |
| | TCNADLDVATHNLTQVALTGKTMPKREEPRDAQGTAPARKTKKASKSKAPPAER |
| | EDQTPAQEPSQTS |
| 131 | MPKPAVESEFSKVLKKHFPGERFRSSYMKRGGKILAAQGEEAVVAYLQGKSEEE |
| | PPNFQPPAKCHVVTKSRDFAEWPIMKASEAIQRYIYALSTTERAACKPGKSSES |
| | HAAWFAATGVSNHGYSHVQGLNLIFDHTLGRYDGVLKKVQLRNEKARARLESIN |
| | ASRADEGLPEIKAEEEEVATNETGHLLQPPGINPSFYVYQTISPQAYRPRDEIV |
| | LPPEYAGYVRDPNAPIPLGVVRNRCDIQKGCPGYIPEWQREAGTAISPKTGKAV |
| | TVPGLSPKKNKRMRRYWRSEKEKAQDALLVTVRIGTDWVVIDVRGLLRNARWRT |
| | IAPKDISLNALLDLFTGDPVIDVRRNIVTFTYTLDACGTYARKWTLKGKQTKAT |
| | LDKLTATQTVALVAIDLGQTNPISAGISRVTQENGALQCEPLDRFTLPDDLLKD |
| | ISAYRIAWDRNEEELRARSVEALPEAQQAEVRALDGVSKETARTQLCADFGLDP |
| | KRLPWDKMSSNTTFISEALLSNSVSNKSEIYFTSTDKGKTKDVMKSDRTWARAY |
| | KPRLSVEAQKLKNEALWALKRTSPEYLKLSRRKEELCRRSINYVIEKTRRRTQC |
| | QIVIPVIEDLNVRFFHGSGKRLPGWDNFFTAKKENRWFIQGLHKAFSDLRTHRS |
| | FYVFEVRPERTSITCPKCGHCEVGNRDGEAFQCLSCGKTCNADLDVATHNLTQV |
| | ALTGKTMPKREEPRDAQGTAPARKTKKASKSKAPPAEREDQTPAQEPSQTS |
| 132 | MEKEITELTKIRREFPNKKFSSTDMKKAGKLLKAEGPDAVRDELNSCQEIIGDF |
| | KPPVKTNIVSISRPFEEWPVSMVGRAIQEYYFSLTKEELESVHPGTSSEDHKSF |
| | FNITGLSNYNYTSVQGLNLIFKNAKAIYDGTLVKANNKNKKLEKKENEINHKRS |
| | LEGLPIITPDFEEPFDENGHLNNPPGINRNIYGYQGCAAKVFVPSKHKMVSLPK |
| | EYEGYNRDPNLSLAGERNRLEIPEGEPGHVPWFQRMDIPEGQIGHVNKIQRENF |
| | VHGKNSGKVKFSDKTGRVKRYHHSKYKDATKPYKFLEESKKVSALDSILAIITI |
| | GDDWVVFDIRGLYRNVFYRELAQKGLTAVQLLDLFTGDPVIDPKKGVVTFSYKE |
| | GVVPVFSQKIVPRFKSRDTLEKLTSQGPVALLSVDLGQNEPVAARVCSLKNIND |
| | KITLDNSCRISFLDDYKKQIKDYRDSLDELEIKIRLEAINSLETNQQVEIRDLD |
| | VFSADRAKANTVDMFDIDPNLISWDSMSDARVSTQISDLYLKNGSSGDYNISQL |
| | VRPKLSDSTRKNLNDSIWKLKRTSEEYLKLSKRKLELSRAVVNYTIRQSKLLSG |
| | INDIVIILEDLDVKKKENGRGIRDIGWDNFFSSRKENRWFIPAFHKTFSELSSN |
| | RGLCVIEVNPAWTSATCPDCGFCSKENRDGINFTCRKCGVSYHADIDVATLNIA |
| | RVAVLGKPMSGPADRERLGDTKKPRVARSRKTMKRKDISNSTVEAMVTA |
| 133 | MEKEITELTKIRREFPNKKESSTDMKKAGKLLKAEGPDAVRDELNSCQEIIGDE |
| | KPPVKTNIVSISRPFEEWPVSMVGRAIQEYYFSLTKEELESVHPGTSSEDHKSF |
| | FNITGLSNYNYTSVQGLNLIFKNAKAIYDGTLVKANNKNKKLEKKFNEINHKRS |
| | LEGLPIITPDFEEPFDENGHLNNPPGINRNIYGYQGCAAKVFVPSKHKMVSLPK |
| | EYEGYNRDPNLSLAGFRNRLEIPEGEPGHVPWFQRMDIPEGQIGHVNKIQRENF |
| | VHGKNSGKVKFSDKTGRVKRYHHSKYKDATKPYKFLEESKKVSALDSILAIITI |
| | GDDWVVFDIRGLYRNVFYRELAQKGLTAVQLLDLFTGDPVIDPKKGVVTFSYKE |
| | GVVPVFSQKIVPRFKSRDTLEKLTSQGPVALLSVDLGQNEPVAARVCSLKNIND |
| | KITLDNSCRISFLDDYKKQIKDYRDSLDELEIKIRLEAINSLETNQQVEIRDLD |
| | VFSADRAKANTVDMFDIDPNLISWDSMSDARVSTQISDLYLKNGSSGDYNISQL |
| | VRPKLSDSTRKNLNDSIWKLKRTSEEYLKLSKRKLELSRAVVNYTIRQSKLLSG |
| | INDIVIILEDLDVKKKENGRGIRDIGWDNFFSSRKENRWFIPAFHKTFSELSSN |
| | RGLCVIEVNPAWTSATCPDCGFCSKENRDGINFTCRKCGVSYHADIDVATLNIA |
| | RVAVLGKPMSGPADRERLGDTKKPRVARSRKTMKRKDISNSTVEAMVTA |
| 134 | MEKEITELTKIRREFPNKKESSTDMKKAGKLLKAEGPDAVRDFLNSCQEIIGDF |
| | KPPVKTNIVSISRPFEEWPVSMVGRAIQEYYFSLTKEELESVHPGTSSEDHKSF |
| | FNITGLSNYNYTSVQGLNLIFKNAKAIYDGTLVKANNKNKKLGSSGFEEPFDEN |
| | GHLNNPPGINRNIYGYQGCAAKVFVPSKHKMVSLPKEYEGYNRDPNLSLAGERN |
| | RLEIPEGEPGHVPWFQRMDIPEGQIGHVNKIQRFNFVHGKNSGKVKFSDKTGRV |
| | KRYHHSKYKDATKPYKFLEESKKVSALDSILAIITIGDDWVVFDIRGLYRNVFY |
| | RELAQKGLTAVQLLDLFTGDPVIDPKKGVVTESYKEGVVPVFSQKIVPREKSRD |
| | TLEKLTSQGPVALLSVDLGQNEPVAARVCSLKNINDKITLDNSCRISFLDDYKK |
| | QIKDYRDSLDELEIKIRLEAINSLETNQQVEIRDLDVFSADRAKANTVDMFDID |
| | PNLISWDSMSDARVSTQISDLYLKNGGDESRVYFEINNKRIKRSDYNISQLVRP |
| | KLSDSTRKNLNDSIWKLKRTSEEYLKLSKRKLELSRAVVNYTIRQSKLLSGIND |
| | IVIILEDLDVKKKENGRGIRDIGWDNFFSSRKENRWFIPAFHKTFSELSSNRGL |
| | CVIEVNPAWTSATCPDCGFCSKENRDGINFTCRKCGVSYHADIDVATLNIARVA |
| | VLGKPMSGPADRERLGDTKKPRVARSRKTMKRKDISNSTVEAMVTA |
| 135 | MEKEITELTKIRREFPNKKESSTDMKKAGKLLKAEGPDAVRDELNSCQEIIGDE |
| | KPPVKTNIVSISRPFEEWPVSMVGRAIQEYYFSLTKEELESVHPGTSSEDHKSF |
| | FNITGLSNYNYTSVQGLNLIFKNAKAIYDGTLVKANNKNKKLGSSGFEEPFDEN |
| | GHLNNPPGINRNIYGYQGCAAKVFVPSKHKMVSLPKEYEGYNRDPNLSLAGERN |
| | RLEIPEGEPGHVPWFQRMDIPEGQIGHVNKIQRFNFVHGKNSGKVKFSDKTGRV |
| | KRYHHSKYKDATKPYKFLEESKKVSALDSILAIITIGDDWVVFDIRGLYRNVFY |
| | RELAQKGLTAVQLLDLFTGDPVIDPKKGVVTFSYKEGVVPVFSQKIVPRFKSRD |
| | TLEKLTSQGPVALLSVDLGQNEPVAARVCSLKNINDKITLDNSCRISFLDDYKK |
| | QIKDYRDSLDELEIKIRLEAINSLETNQQVEIRDLDVFSADRAKANTVDMFDID |
| | PNLISWDSMSDARVSTQISDLYLKNGSSGDYNISQLVRPKLSDSTRKNLNDSIW |
| | KLKRTSEEYLKLSKRKLELSRAVVNYTIRQSKLLSGINDIVIILEDLDVKKKEN |
| | GRGIRDIGWDNFFSSRKENRWFIPAFHKTFSELSSNRGLCVIEVNPAWTSATCP |
| | DCGFCSKENRDGINFTCRKCGVSYHADIDVATLNIARVAVLGKPMSGPADRERL |
| | GDTKKPRVARSRKTMKRKDISNSTVEAMVTA |
| 136 | MEKEITELTKIRREFPNKKFSSTDMKKAGKLLKAEGPDAVRDELNSCQEIIGDF |
| | KPPVKTNIVSISRPFEEWPVSMVGRAIQEYYFSLTKEELESVHPGTSSEDHKSF |
| | FNITGLSNYNYTSVQGLNLIFKNAKAIYDGTLVKANNKNKKLGSSGFEEPFDEN |
| | GHLNNPPGINRNIYGYQGCAAKVFVPSKHKMVSLPKEYEGYNRDPNLSLAGERN |
| | RLEIPEGEPGHVPWFQRMDIPEGQIGHVNKIQRFNFVHGKNSGKVKFSDKTGRV |
| | KRYHHSKYKDATKPYKFLEESKKVSALDSILAIITIGDDWVVFDIRGLYRNVFY |
| | RELAQKGLTAVQLLDLFTGDPVIDPKKGVVTFSYKEGVVPVFSQKIVPRFKSRD |
| | TLEKLTSQGPVALLSVDLGQNEPVAARVCSLKNINDKITLDNSCRISFLDDYKK |
| | QIKDYRDSLDELEIKIRLEAINSLETNQQVEIRDLDVESADRAKANTVDMFDID |
| | PNLISWDSMSDARVSTQISDLYLKNGGDESRVYFEINNGSSGDYNISQLVRPKL |
| | SDSTRKNLNDSIWKLKRTSEEYLKLSKRKLELSRAVVNYTIRQSKLLSGINDIV |
| | IILEDLDVKKKENGRGIRDIGWDNFFSSRKENRWFIPAFHKTFSELSSNRGLCV |
| | IEVNPAWTSATCPDCGFCSKENRDGINFTCRKCGVSYHADIDVATLNIARVAVL |
| | GKPMSGPADRERLGDTKKPRVARSRKTMKRKDISNSTVEAMVTA |
| 137 | MEKEITELTKIRREFPNKKFSSTDMKKAGKLLKAEGPDAVRDELNSCQEIIGDF |
| | KPPVKTNIVSISRPFEEWPVSMVGRAIQEYYFSLTKEELESVHPGTSSEDHKSF |
| | FNITGLSNYNYTSVQGLNLIFKNAKAIYDGTLVKANNKNKKLEKKENEINHKRS |
| | LEGLPIITPDFEEPFDENGHLNNPPGINRNIYGYQGCAAKVFVPSKHKMVSLPK |
| | EYEGYNRDPNLSLAGFRNRLEIPEGEPGHVPWFQRMDIPEGQIGHVNKIQRENF |
| | VHGKNSGKVKFSDKTGRVKRYHHSKYKDATKPYKFLEESKKVSALDSILAIITI |
| | GDDWVVFDIRGLYRNVFYRELAQKGLTAVQLLDLFTGDPVIDPKKGVVTFSYKE |
| | GVVPVFSQKIVPRFKSRDTLEKLTSQGPVALLSVDLGQNEPVAARVCSLKNIND |
| | KITLDNSCRISFLDDYKKQIKDYRDSLDELEIKIRLEAINSLETNQQVEIRDLD |
| | VFSADRAKANTVDMFDIDPNLISWDSMSDARVSTQISDLYLKNGVSNKSEIYFT |
| | STDKGKTKDVMKSDYNISQLVRPKLSDSTRKNLNDSIWKLKRTSEEYLKLSKRK |
| | LELSRAVVNYTIRQSKLLSGINDIVIILEDLDVKKKENGRGIRDIGWDNFFSSR |
| | KENRWFIPAFHKTFSELSSNRGLCVIEVNPAWTSATCPDCGFCSKENRDGINFT |
| | CRKCGVSYHADIDVATLNIARVAVLGKPMSGPADRERLGDTKKPRVARSRKTMK |
| | RKDISNSTVEAMVTA |
| 138 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPEWQHPLLNRRKNRRRRDWYSASL |
| | NKPKATCSKRSGTPNRKNSRTDQIQSGRFKGAIPVLMRFQDEWVIIDMRGLLRT |
| | NHWKKYHKPTDSINDLFDYFTGDPVIDTKANVVRFRYKMENGIVNYKPVREKKG |
| | KELLENICDQNGSCKLATVDVGQNNPVAIGLFELKKVNGELTKTLISRHPTPID |
| | FCNKITAYRERYDKLESSIKLDAIKQLTSEQKIEVDNYNNNFTPQNTKQIVCSK |
| | LNINPNDLPWDKMISGTHFISEKAQVSNKSEIYFTSTDKGKTKDVMKSDYKWFQ |
| | DYKPKLSKEVRDALSDIEWRLRRESLEFNKLSKSREQDARQLANWISSMCDVIG |
| | IENLVKKNNFFGGSGKREPGWDNFYKPKKENRWWINAIHKALTELSQNKGKRVI |
| | LLPAMRTSITCPKCKYCDSKNRNGEKFNCLKCGIELNADIDVATENLATVAITA |
| | QSMPKPTCERSGDAKKPVRARKAKAPEFHDKLAPSYTVVLREAV |
| 139 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPGHQRFADTGQNNSGKANPNKKGR |
| | MRKYYGHGTKYTQPGEYQEVFRKGHREGNKRRYWEEDFRSEAHDCILYVIHIGD |
| | DWVVCDMRGLLRTNHWKKYHKPTDSINDLFDYFTGDPVIDTKANVVRFRYKMEN |
| | GIVNYKPVREKKGKELLENICDQNGSCKLATVDVGQNNPVAIGLFELKKVNGEL |
| | TKTLISRHPTPIDFCNKITAYRERYDKLESSIKLDAIKQLTSEQKIEVDNYNNN |
| | FTPQNTKQIVCSKLNINPNDLPWDKMISGTHFISEKAQVSNKSEIYFTSTDKGK |
| | TKDVMKSDYKWFQDYKPKLSKEVRDALSDIEWRLRRESLEFNKLSKSREQDARQ |
| | LANWISSMCDVIGIENLVKKNNFFGGSGKREPGWDNFYKPKKENRWWINAIHKA |
| | LTELSQNKGKRVILLPAMRTSITCPKCKYCDSKNRNGEKENCLKCGIELNADID |
| | VATENLATVAITAQSMPKPTCERSGDAKKPVRARKAKAPEFHDKLAPSYTVVLR |
| | EAV |
| 140 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPEHQRDKLKKGGRVKRLRTTRVRV |
| | DATETVRAKAEALNAEKARLRGKEAILAVFQIEEDWALIDMRGLLRTNHWKKYH |
| | KPTDSINDLFDYFTGDPVIDTKANVVRFRYKMENGIVNYKPVREKKGKELLENI |
| | CDQNGSCKLATVDVGQNNPVAIGLFELKKVNGELTKTLISRHPTPIDFCNKITA |
| | YRERYDKLESSIKLDAIKQLTSEQKIEVDNYNNNFTPQNTKQIVCSKLNINPND |
| | LPWDKMISGTHFISEKAQVSNKSEIYFTSTDKGKTKDVMKSDYKWFQDYKPKLS |
| | KEVRDALSDIEWRLRRESLEFNKLSKSREQDARQLANWISSMCDVIGIENLVKK |
| | NNFFGGSGKREPGWDNFYKPKKENRWWINAIHKALTELSQNKGKRVILLPAMRT |
| | SITCPKCKYCDSKNRNGEKFNCLKCGIELNADIDVATENLATVAITAQSMPKPT |
| | CERSGDAKKPVRARKAKAPEFHDKLAPSYTVVLREAV |
| 141 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPEPHREGLTGRKDRRMRRYYETER |
| | GTKLKRPPLTAKGRADKANEALLVVVRIDSDWVVMDMRGLLRTNHWKKYHKPTD |
| | SINDLFDYFTGDPVIDTKANVVRFRYKMENGIVNYKPVREKKGKELLENICDQN |
| | GSCKLATVDVGQNNPVAIGLFELKKVNGELTKTLISRHPTPIDFCNKITAYRER |
| | YDKLESSIKLDAIKQLTSEQKIEVDNYNNNFTPQNTKQIVCSKLNINPNDLPWD |
| | KMISGTHFISEKAQVSNKSEIYFTSTDKGKTKDVMKSDYKWFQDYKPKLSKEVR |
| | DALSDIEWRLRRESLEFNKLSKSREQDARQLANWISSMCDVIGIENLVKKNNFF |
| | GGSGKREPGWDNFYKPKKENRWWINAIHKALTELSQNKGKRVILLPAMRTSITC |
| | PKCKYCDSKNRNGEKFNCLKCGIELNADIDVATENLATVAITAQSMPKPTCERS |
| | GDAKKPVRARKAKAPEFHDKLAPSYTVVLREAV |
| 142 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPEHQRKNLKKKGRVRLYRRTPPKT |
| | KALASILAVLQIGKDWVLFDMRGLLRTNHWKKYHKPTDSINDLFDYFTGDPVID |
| | TKANVVRFRYKMENGIVNYKPVREKKGKELLENICDQNGSCKLATVDVGQNNPV |
| | AIGLFELKKVNGELTKTLISRHPTPIDFCNKITAYRERYDKLESSIKLDAIKQL |
| | TSEQKIEVDNYNNNFTPQNTKQIVCSKLNINPNDLPWDKMISGTHFISEKAQVS |
| | NKSEIYFTSTDKGKTKDVMKSDYKWFQDYKPKLSKEVRDALSDIEWRLRRESLE |
| | FNKLSKSREQDARQLANWISSMCDVIGIENLVKKNNFFGGSGKREPGWDNFYKP |
| | KKENRWWINAIHKALTELSQNKGKRVILLPAMRTSITCPKCKYCDSKNRNGEKF |
| | NCLKCGIELNADIDVATENLATVAITAQSMPKPTCERSGDAKKPVRARKAKAPE |
| | FHDKLAPSYTVVLREAV |
| 143 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPEWQRPLLNRHKGRRHRSWYANSL |
| | NKPRKSRTEEAKDRQNAGKRTALIEAERLKGVLPVLMRFKEDWLIIDMRGLLRT |
| | NHWKKYHKPTDSINDLFDYFTGDPVIDTKANVVRFRYKMENGIVNYKPVREKKG |
| | KELLENICDQNGSCKLATVDVGQNNPVAIGLFELKKVNGELTKTLISRHPTPID |
| | FCNKITAYRERYDKLESSIKLDAIKQLTSEQKIEVDNYNNNFTPQNTKQIVCSK |
| | LNINPNDLPWDKMISGTHFISEKAQVSNKSEIYFTSTDKGKTKDVMKSDYKWFQ |
| | DYKPKLSKEVRDALSDIEWRLRRESLEFNKLSKSREQDARQLANWISSMCDVIG |
| | IENLVKKNNFFGGSGKREPGWDNFYKPKKENRWWINAIHKALTELSQNKGKRVI |
| | LLPAMRTSITCPKCKYCDSKNRNGEKFNCLKCGIELNADIDVATENLATVAITA |
| | QSMPKPTCERSGDAKKPVRARKAKAPEFHDKLAPSYTVVLREAV |
| 144 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPAWQREQGLVKPGGRRRRLSGSES |
| | NMRQKVDPSTGPRRSTRSGTVNRSNQRTGRNGDPLLVEIRMKEDWVLLDMRGLL |
| | RTNHWKKYHKPTDSINDLFDYFTGDPVIDTKANVVRFRYKMENGIVNYKPVREK |
| | KGKELLENICDQNGSCKLATVDVGQNNPVAIGLFELKKVNGELTKTLISRHPTP |
| | IDFCNKITAYRERYDKLESSIKLDAIKQLTSEQKIEVDNYNNNFTPQNTKQIVC |
| | SKLNINPNDLPWDKMISGTHFISEKAQVSNKSEIYFTSTDKGKTKDVMKSDYKW |
| | FQDYKPKLSKEVRDALSDIEWRLRRESLEFNKLSKSREQDARQLANWISSMCDV |
| | IGIENLVKKNNFFGGSGKREPGWDNFYKPKKENRWWINAIHKALTELSQNKGKR |
| | VILLPAMRTSITCPKCKYCDSKNRNGEKFNCLKCGIELNADIDVATENLATVAI |
| | TAQSMPKPTCERSGDAKKPVRARKAKAPEFHDKLAPSYTVVLREAV |
| 145 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVDEYGKLISKRRKERINKDDAILCV |
| | SNFGDDWIIFDMRGLLRTNHWKKYHKPTDSINDLFDYFTGDPVIDTKANVVRER |
| | YKMENGIVNYKPVREKKGKELLENICDQNGSCKLATVDVGQNNPVAIGLFELKK |
| | VNGELTKTLISRHPTPIDFCNKITAYRERYDKLESSIKLDAIKQLTSEQKIEVD |
| | NYNNNFTPQNTKQIVCSKLNINPNDLPWDKMISGTHFISEKAQVSNKSEIYFTS |
| | TDKGKTKDVMKSDYKWFQDYKPKLSKEVRDALSDIEWRLRRESLEFNKLSKSRE |
| | QDARQLANWISSMCDVIGIENLVKKNNFFGGSGKREPGWDNFYKPKKENRWWIN |
| | AIHKALTELSQNKGKRVILLPAMRTSITCPKCKYCDSKNRNGEKFNCLKCGIEL |
| | NADIDVATENLATVAITAQSMPKPTCERSGDAKKPVRARKAKAPEFHDKLAPSY |
| | TVVLREAV |
| 146 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPPWDRENLSVKKHRRKRASWARSR |
| | GGAIDDNMLLAVVRVADDWALLDMRGLLRTNHWKKYHKPTDSINDLFDYFTGDP |
| | VIDTKANVVRFRYKMENGIVNYKPVREKKGKELLENICDQNGSCKLATVDVGQN |
| | NPVAIGLFELKKVNGELTKTLISRHPTPIDFCNKITAYRERYDKLESSIKLDAI |
| | KQLTSEQKIEVDNYNNNFTPQNTKQIVCSKLNINPNDLPWDKMISGTHFISEKA |
| | QVSNKSEIYFTSTDKGKTKDVMKSDYKWFQDYKPKLSKEVRDALSDIEWRLRRE |
| | SLEFNKLSKSREQDARQLANWISSMCDVIGIENLVKKNNFFGGSGKREPGWDNF |
| | YKPKKENRWWINAIHKALTELSQNKGKRVILLPAMRTSITCPKCKYCDSKNRNG |
| | EKFNCLKCGIELNADIDVATENLATVAITAQSMPKPTCERSGDAKKPVRARKAK |
| | APEFHDKLAPSYTVVLREAV |
| 147 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPLHDREKLTSNKHRRMKLPKSLRA |
| | QGALPVCFRVFDDWAVVDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 148 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPEWMRTAGEKTNPRTQKKEMHPGL |
| | STRKNKRMRLPRSVRSAPLGALLVTIHLGEDWLVLDMRGLLRTNHWKKYHKPTD |
| | SINDLFDYFTGDPVIDTKANVVRFRYKMENGIVNYKPVREKKGKELLENICDQN |
| | GSCKLATVDVGQNNPVAIGLFELKKVNGELTKTLISRHPTPIDFCNKITAYRER |
| | YDKLESSIKLDAIKQLTSEQKIEVDNYNNNFTPQNTKQIVCSKLNINPNDLPWD |
| | KMISGTHFISEKAQVSNKSEIYFTSTDKGKTKDVMKSDYKWFQDYKPKLSKEVR |
| | DALSDIEWRLRRESLEFNKLSKSREQDARQLANWISSMCDVIGIENLVKKNNFF |
| | GGSGKREPGWDNFYKPKKENRWWINAIHKALTELSQNKGKRVILLPAMRTSITC |
| | PKCKYCDSKNRNGEKFNCLKCGIELNADIDVATENLATVAITAQSMPKPTCERS |
| | GDAKKPVRARKAKAPEFHDKLAPSYTVVLREAV |
| 149 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVNKIQRFNFVHGKNSGKVKFSDKTG |
| | RVKRYHHSKYKDATKPYKFLEESKKVSALDSILAIITIGDDWVVFDMRGLLRTN |
| | HWKKYHKPTDSINDLFDYFTGDPVIDTKANVVRFRYKMENGIVNYKPVREKKGK |
| | ELLENICDQNGSCKLATVDVGQNNPVAIGLFELKKVNGELTKTLISRHPTPIDE |
| | CNKITAYRERYDKLESSIKLDAIKQLTSEQKIEVDNYNNNFTPQNTKQIVCSKL |
| | NINPNDLPWDKMISGTHFISEKAQVSNKSEIYFTSTDKGKTKDVMKSDYKWFQD |
| | YKPKLSKEVRDALSDIEWRLRRESLEFNKLSKSREQDARQLANWISSMCDVIGI |
| | ENLVKKNNFFGGSGKREPGWDNFYKPKKENRWWINAIHKALTELSQNKGKRVIL |
| | LPAMRTSITCPKCKYCDSKNRNGEKFNCLKCGIELNADIDVATENLATVAITAQ |
| | SMPKPTCERSGDAKKPVRARKAKAPEFHDKLAPSYTVVLREAV |
| 150 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPWFQRMDIPEGQIGHVNKIQRENF |
| | VHGKNSGKVKFSDKTGRVKRYHHSKYKDATKPYKFLEESKKVSALDSILAIITI |
| | GDDWVVFDMRGLLRTNHWKKYHKPTDSINDLFDYFTGDPVIDTKANVVRFRYKM |
| | ENGIVNYKPVREKKGKELLENICDQNGSCKLATVDVGQNNPVAIGLFELKKVNG |
| | ELTKTLISRHPTPIDFCNKITAYRERYDKLESSIKLDAIKQLTSEQKIEVDNYN |
| | NNFTPQNTKQIVCSKLNINPNDLPWDKMISGTHFISEKAQVSNKSEIYFTSTDK |
| | GKTKDVMKSDYKWFQDYKPKLSKEVRDALSDIEWRLRRESLEFNKLSKSREQDA |
| | RQLANWISSMCDVIGIENLVKKNNFFGGSGKREPGWDNFYKPKKENRWWINAIH |
| | KALTELSQNKGKRVILLPAMRTSITCPKCKYCDSKNRNGEKENCLKCGIELNAD |
| | IDVATENLATVAITAQSMPKPTCERSGDAKKPVRARKAKAPEFHDKLAPSYTVV |
| | LREAV |
| 151 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPEKHRSQLSMAKHKRRRAWYALSQ |
| | NKPRPPKDGSKGRRSVRDLADLKAASLADAIPLVSRVGFDWVVIDMRGLLRTNH |
| | WKKYHKPTDSINDLFDYFTGDPVIDTKANVVRFRYKMENGIVNYKPVREKKGKE |
| | LLENICDQNGSCKLATVDVGQNNPVAIGLFELKKVNGELTKTLISRHPTPIDFC |
| | NKITAYRERYDKLESSIKLDAIKQLTSEQKIEVDNYNNNFTPQNTKQIVCSKLN |
| | INPNDLPWDKMISGTHFISEKAQVSNKSEIYFTSTDKGKTKDVMKSDYKWFQDY |
| | KPKLSKEVRDALSDIEWRLRRESLEFNKLSKSREQDARQLANWISSMCDVIGIE |
| | NLVKKNNFFGGSGKREPGWDNFYKPKKENRWWINAIHKALTELSQNKGKRVILL |
| | PAMRTSITCPKCKYCDSKNRNGEKFNCLKCGIELNADIDVATENLATVAITAQS |
| | MPKPTCERSGDAKKPVRARKAKAPEFHDKLAPSYTVVLREAV |
| 152 | MIKPTVSQFLIPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPYWQRPFLSKRRNRRVRAGWGKQV |
| | SSIQAWLTGALLVIVRLGNEAFLADMRGLLRTNHWKKYHKPTDSINDLFDYFTG |
| | DPVIDTKANVVRFRYKMENGIVNYKPVREKKGKELLENICDQNGSCKLATVDVG |
| | QNNPVAIGLFELKKVNGELTKTLISRHPTPIDFCNKITAYRERYDKLESSIKLD |
| | AIKQLTSEQKIEVDNYNNNFTPQNTKQIVCSKLNINPNDLPWDKMISGTHFISE |
| | KAQVSNKSEIYFTSTDKGKTKDVMKSDYKWFQDYKPKLSKEVRDALSDIEWRLR |
| | RESLEFNKLSKSREQDARQLANWISSMCDVIGIENLVKKNNFFGGSGKREPGWD |
| | NFYKPKKENRWWINAIHKALTELSQNKGKRVILLPAMRTSITCPKCKYCDSKNR |
| | NGEKFNCLKCGIELNADIDVATENLATVAITAQSMPKPTCERSGDAKKPVRARK |
| | AKAPEFHDKLAPSYTVVLREAV |
| 153 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPGHQRKESTTEGPKINFRKGRIRR |
| | SYTALYAKRDSRRVRQGKLALPSYRHHMMRLNSNAESAILAVIFFGKDWVVFDM |
| | RGLLRTNHWKKYHKPTDSINDLEDYFTGDPVIDTKANVVRFRYKMENGIVNYKP |
| | VREKKGKELLENICDQNGSCKLATVDVGQNNPVAIGLFELKKVNGELTKTLISR |
| | HPTPIDFCNKITAYRERYDKLESSIKLDAIKQLTSEQKIEVDNYNNNFTPQNTK |
| | QIVCSKLNINPNDLPWDKMISGTHFISEKAQVSNKSEIYFTSTDKGKTKDVMKS |
| | DYKWFQDYKPKLSKEVRDALSDIEWRLRRESLEFNKLSKSREQDARQLANWISS |
| | MCDVIGIENLVKKNNFFGGSGKREPGWDNFYKPKKENRWWINAIHKALTELSQN |
| | KGKRVILLPAMRTSITCPKCKYCDSKNRNGEKFNCLKCGIELNADIDVATENLA |
| | TVAITAQSMPKPTCERSGDAKKPVRARKAKAPEFHDKLAPSYTVVLREAV |
| 154 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLCTDYFSVQGLNLIFQNARKRYIGVQTKVTNRNEKRHKKLKRINAKRIAEG |
| | LPELTSDEPESALDETGHLIDPPGLNTNIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 155 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLPDRGLPVQAINKIAKAAVNRAFGVVRKVENRNEKRRSRDNRIAEHNRENG |
| | LTEVVREAPEVATNADGELLHPPGIDPSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | DKLAPSYTVVLREAV |
| 156 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLPNFGYMSAQGLNAIFSGALARYEGVVQKVENRNKKRFEKLSEKNQLLIEE |
| | GQPVKDYVPDTAYHTPETLQKLAENNHVRVEDLGDMIDRLVHPPGIHRSIYCYQ |
| | SVSPKPFITSKYHNVNLPEEYIGYYRKSNEPIVSPYQFDRLRIPIGEPGYVPKW |
| | QYTFLSKKENKRRKLSKRIKNVSPILGIICIKKDWCVFDMRGLLRTNHWKKYHK |
| | PTDSINDLFDYFTGDPVIDTKANVVRFRYKMENGIVNYKPVREKKGKELLENIC |
| | DQNGSCKLATVDVGQNNPVAIGLFELKKVNGELTKTLISRHPTPIDFCNKITAY |
| | RERYDKLESSIKLDAIKQLTSEQKIEVDNYNNNFTPQNTKQIVCSKLNINPNDL |
| | PWDKMISGTHFISEKAQVSNKSEIYFTSTDKGKTKDVMKSDYKWFQDYKPKLSK |
| | EVRDALSDIEWRLRRESLEFNKLSKSREQDARQLANWISSMCDVIGIENLVKKN |
| | NFFGGSGKREPGWDNFYKPKKENRWWINAIHKALTELSQNKGKRVILLPAMRTS |
| | ITCPKCKYCDSKNRNGEKFNCLKCGIELNADIDVATENLATVAITAQSMPKPTC |
| | ERSGDAKKPVRARKAKAPEFHDKLAPSYTVVLREAV |
| 157 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLSEDDLMALEAQLLETIMGNAISLHGGVLKKIDNANVKAAKRLSGRNEARL |
| | NKGLQELPPEQEGSAYGADGLLVNPPGLNLNIYCYQSVSPKPFITSKYHNVNLP |
| | EEYIGYYRKSNEPIVSPYQFDRLRIPIGEPGYVPKWQYTFLSKKENKRRKLSKR |
| | IKNVSPILGIICIKKDWCVFDMRGLLRTNHWKKYHKPTDSINDLFDYFTGDPVI |
| | DTKANVVRFRYKMENGIVNYKPVREKKGKELLENICDQNGSCKLATVDVGQNNP |
| | VAIGLFELKKVNGELTKTLISRHPTPIDFCNKITAYRERYDKLESSIKLDAIKQ |
| | LTSEQKIEVDNYNNNFTPQNTKQIVCSKLNINPNDLPWDKMISGTHFISEKAQV |
| | SNKSEIYFTSTDKGKTKDVMKSDYKWFQDYKPKLSKEVRDALSDIEWRLRRESL |
| | EFNKLSKSREQDARQLANWISSMCDVIGIENLVKKNNFFGGSGKREPGWDNFYK |
| | PKKENRWWINAIHKALTELSQNKGKRVILLPAMRTSITCPKCKYCDSKNRNGEK |
| | FNCLKCGIELNADIDVATENLATVAITAQSMPKPTCERSGDAKKPVRARKAKAP |
| | EFHDKLAPSYTVVLREAV |
| 158 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLSNYNYTSVQGLNLIFKNAKAIYDGTLVKANNKNKKLEKKENEINHKRSLE |
| | GLPIITPDFEEPFDENGHLNNPPGINRNIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 159 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLLTFPMNQVQMLNGIFNRAFSVYLGVEKKVANRNAEAREKLKNRGQEDQFV |
| | EEFAYSYCPVDFKDGMFVHDSAEEGETQGCLLHPPGIDPKIYCYQSVSPKPFIT |
| | SKYHNVNLPEEYIGYYRKSNEPIVSPYQFDRLRIPIGEPGYVPKWQYTELSKKE |
| | NKRRKLSKRIKNVSPILGIICIKKDWCVFDMRGLLRTNHWKKYHKPTDSINDLF |
| | DYFTGDPVIDTKANVVRFRYKMENGIVNYKPVREKKGKELLENICDQNGSCKLA |
| | TVDVGQNNPVAIGLFELKKVNGELTKTLISRHPTPIDFCNKITAYRERYDKLES |
| | SIKLDAIKQLTSEQKIEVDNYNNNFTPQNTKQIVCSKLNINPNDLPWDKMISGT |
| | HFISEKAQVSNKSEIYFTSTDKGKTKDVMKSDYKWFQDYKPKLSKEVRDALSDI |
| | EWRLRRESLEFNKLSKSREQDARQLANWISSMCDVIGIENLVKKNNFFGGSGKR |
| | EPGWDNFYKPKKENRWWINAIHKALTELSQNKGKRVILLPAMRTSITCPKCKYC |
| | DSKNRNGEKFNCLKCGIELNADIDVATENLATVAITAQSMPKPTCERSGDAKKP |
| | VRARKAKAPEFHDKLAPSYTVVLREAV |
| 160 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLNIEYQNVAGLNLIFNNVKNTYNGVILKVKNRNEKLKKKAIKNNYEFEEIK |
| | TENDDGCLINKPGINNVIYCYQSVSPKPFITSKYHNVNLPEEYIGYYRKSNEPI |
| | VSPYQFDRLRIPIGEPGYVPKWQYTFLSKKENKRRKLSKRIKNVSPILGIICIK |
| | KDWCVFDMRGLLRTNHWKKYHKPTDSINDLFDYFTGDPVIDTKANVVRFRYKME |
| | NGIVNYKPVREKKGKELLENICDQNGSCKLATVDVGQNNPVAIGLFELKKVNGE |
| | LTKTLISRHPTPIDFCNKITAYRERYDKLESSIKLDAIKQLTSEQKIEVDNYNN |
| | NFTPQNTKQIVCSKLNINPNDLPWDKMISGTHFISEKAQVSNKSEIYFTSTDKG |
| | KTKDVMKSDYKWFQDYKPKLSKEVRDALSDIEWRLRRESLEFNKLSKSREQDAR |
| | QLANWISSMCDVIGIENLVKKNNFFGGSGKREPGWDNFYKPKKENRWWINAIHK |
| | ALTELSQNKGKRVILLPAMRTSITCPKCKYCDSKNRNGEKFNCLKCGIELNADI |
| | DVATENLATVAITAQSMPKPTCERSGDAKKPVRARKAKAPEFHDKLAPSYTVVL |
| | REAV |
| 161 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFITDLFLARGG |
| | DPKKCMFTSEPKKKKNSKQVLYKIRDYKWFQDYKPKLSKEVRDALSDIEWRLRR |
| | ESLEFNKLSKSREQDARQLANWISSMCDVIGIENLVKKNNFFGGSGKREPGWDN |
| | FYKPKKENRWWINAIHKALTELSQNKGKRVILLPAMRTSITCPKCKYCDSKNRN |
| | GEKFNCLKCGIELNADIDVATENLATVAITAQSMPKPTCERSGDAKKPVRARKA |
| | KAPEFHDKLAPSYTVVLREAV |
| 162 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFIATAYLEKGG |
| | DRKVATLKPKNRPEMLRRDYKWFQDYKPKLSKEVRDALSDIEWRLRRESLEFNK |
| | LSKSREQDARQLANWISSMCDVIGIENLVKKNNFFGGSGKREPGWDNFYKPKKE |
| | NRWWINAIHKALTELSQNKGKRVILLPAMRTSITCPKCKYCDSKNRNGEKENCL |
| | KCGIELNADIDVATENLATVAITAQSMPKPTCERSGDAKKPVRARKAKAPEFHD |
| | KLAPSYTVVLREAV |
| 163 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISDDFLRRGG |
| | DPNIVHFDRQPKKGKVSKKSQRIKRSDYKWFQDYKPKLSKEVRDALSDIEWRLR |
| | RESLEFNKLSKSREQDARQLANWISSMCDVIGIENLVKKNNFFGGSGKREPGWD |
| | NFYKPKKENRWWINAIHKALTELSQNKGKRVILLPAMRTSITCPKCKYCDSKNR |
| | NGEKFNCLKCGIELNADIDVATENLATVAITAQSMPKPTCERSGDAKKPVRARK |
| | AKAPEFHDKLAPSYTVVLREAV |
| 164 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTELSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFIKQFLKKNGG |
| | NKSLIEYIPYQKKKSKKTPKAVLRSDYKWFQDYKPKLSKEVRDALSDIEWRLRR |
| | ESLEFNKLSKSREQDARQLANWISSMCDVIGIENLVKKNNFFGGSGKREPGWDN |
| | FYKPKKENRWWINAIHKALTELSQNKGKRVILLPAMRTSITCPKCKYCDSKNRN |
| | GEKFNCLKCGIELNADIDVATENLATVAITAQSMPKPTCERSGDAKKPVRARKA |
| | KAPEFHDKLAPSYTVVLREAV |
| 165 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEALLSNSV |
| | SRDQVFFTPAPKKGAKKKAPVEVMRKDYKWFQDYKPKLSKEVRDALSDIEWRLR |
| | RESLEFNKLSKSREQDARQLANWISSMCDVIGIENLVKKNNFFGGSGKREPGWD |
| | NFYKPKKENRWWINAIHKALTELSQNKGKRVILLPAMRTSITCPKCKYCDSKNR |
| | NGEKFNCLKCGIELNADIDVATENLATVAITAQSMPKPTCERSGDAKKPVRARK |
| | AKAPEFHDKLAPSYTVVLREAV |
| 166 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISDRYLADGG |
| | DPSKVWFVPGPRKRKKNAPPLKKPPKPRELVKRSDYKWFQDYKPKLSKEVRDAL |
| | SDIEWRLRRESLEFNKLSKSREQDARQLANWISSMCDVIGIENLVKKNNFFGGS |
| | GKREPGWDNFYKPKKENRWWINAIHKALTELSQNKGKRVILLPAMRTSITCPKC |
| | KYCDSKNRNGEKFNCLKCGIELNADIDVATENLATVAITAQSMPKPTCERSGDA |
| | KKPVRARKAKAPEFHDKLAPSYTVVLREAV |
| 167 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISDHILDHGG |
| | DPDTVFFMATKRGQNKPTLHKRKDYKWFQDYKPKLSKEVRDALSDIEWRLRRES |
| | LEFNKLSKSREQDARQLANWISSMCDVIGIENLVKKNNFFGGSGKREPGWDNFY |
| | KPKKENRWWINAIHKALTELSQNKGKRVILLPAMRTSITCPKCKYCDSKNRNGE |
| | KFNCLKCGIELNADIDVATENLATVAITAQSMPKPTCERSGDAKKPVRARKAKA |
| | PEFHDKLAPSYTVVLREAV |
| 168 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVEDMRGLLRTNHWKKYHKPTDSINDLEDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISDLFLRMGG |
| | DRSRVFFEVPPKKAKKAPKKPPKKPAGPRIVKRTDYKWFQDYKPKLSKEVRDAL |
| | SDIEWRLRRESLEFNKLSKSREQDARQLANWISSMCDVIGIENLVKKNNFFGGS |
| | GKREPGWDNFYKPKKENRWWINAIHKALTELSQNKGKRVILLPAMRTSITCPKC |
| | KYCDSKNRNGEKFNCLKCGIELNADIDVATENLATVAITAQSMPKPTCERSGDA |
| | KKPVRARKAKAPEFHDKLAPSYTVVLREAV |
| 169 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFIANLYLERGG |
| | DPSKVFFTRPLKKDSKSKKPRKPTKRTDYKWFQDYKPKLSKEVRDALSDIEWRL |
| | RRESLEFNKLSKSREQDARQLANWISSMCDVIGIENLVKKNNFFGGSGKREPGW |
| | DNFYKPKKENRWWINAIHKALTELSQNKGKRVILLPAMRTSITCPKCKYCDSKN |
| | RNGEKENCLKCGIELNADIDVATENLATVAITAQSMPKPTCERSGDAKKPVRAR |
| | KAKAPEFHDKLAPSYTVVLREAV |
| 170 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFIVDAVLRKGG |
| | DESLVYFKNKEGETLKFRDYKWFQDYKPKLSKEVRDALSDIEWRLRRESLEFNK |
| | LSKSREQDARQLANWISSMCDVIGIENLVKKNNFFGGSGKREPGWDNFYKPKKE |
| | NRWWINAIHKALTELSQNKGKRVILLPAMRTSITCPKCKYCDSKNRNGEKENCL |
| | KCGIELNADIDVATENLATVAITAQSMPKPTCERSGDAKKPVRARKAKAPEFHD |
| | KLAPSYTVVLREAV |
| 171 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFIADHVQAAGG |
| | DVEWFFFKTCGKGKEIKTVRRSDYKWFQDYKPKLSKEVRDALSDIEWRLRRESL |
| | EFNKLSKSREQDARQLANWISSMCDVIGIENLVKKNNFFGGSGKREPGWDNFYK |
| | PKKENRWWINAIHKALTELSQNKGKRVILLPAMRTSITCPKCKYCDSKNRNGEK |
| | FNCLKCGIELNADIDVATENLATVAITAQSMPKPTCERSGDAKKPVRARKAKAP |
| | EFHDKLAPSYTVVLREAV |
| 172 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFIAQAFVAKGG |
| | DPKVVTIKPNGKPKMFRKDYKWFQDYKPKLSKEVRDALSDIEWRLRRESLEFNK |
| | LSKSREQDARQLANWISSMCDVIGIENLVKKNNFFGGSGKREPGWDNFYKPKKE |
| | NRWWINAIHKALTELSQNKGKRVILLPAMRTSITCPKCKYCDSKNRNGEKENCL |
| | KCGIELNADIDVATENLATVAITAQSMPKPTCERSGDAKKPVRARKAKAPEFHD |
| | KLAPSYTVVLREAV |
| 173 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISDQLLQQGA |
| | DPASLFFDGERDGKPCRHKKKDYKWFQDYKPKLSKEVRDALSDIEWRLRRESLE |
| | FNKLSKSREQDARQLANWISSMCDVIGIENLVKKNNFFGGSGKREPGWDNFYKP |
| | KKENRWWINAIHKALTELSQNKGKRVILLPAMRTSITCPKCKYCDSKNRNGEKF |
| | NCLKCGIELNADIDVATENLATVAITAQSMPKPTCERSGDAKKPVRARKAKAPE |
| | FHDKLAPSYTVVLREAV |
| 174 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTELSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFIADMLISHGR |
| | DDDAFLSHGEFPTLEKRKKFDYKWFQDYKPKLSKEVRDALSDIEWRLRRESLEF |
| | NKLSKSREQDARQLANWISSMCDVIGIENLVKKNNFFGGSGKREPGWDNFYKPK |
| | KENRWWINAIHKALTELSQNKGKRVILLPAMRTSITCPKCKYCDSKNRNGEKEN |
| | CLKCGIELNADIDVATENLATVAITAQSMPKPTCERSGDAKKPVRARKAKAPEF |
| | HDKLAPSYTVVLREAV |
| 175 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEALSAKGS |
| | PLALHDGAPIKDYKWFQDYKPKLSKEVRDALSDIEWRLRRESLEFNKLSKSREQ |
| | DARQLANWISSMCDVIGIENLVKKNNFFGGSGKREPGWDNFYKPKKENRWWINA |
| | IHKALTELSQNKGKRVILLPAMRTSITCPKCKYCDSKNRNGEKENCLKCGIELN |
| | ADIDVATENLATVAITAQSMPKPTCERSGDAKKPVRARKAKAPEFHDKLAPSYT |
| | VVLREAV |
| 176 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFIRDCLKNKNI |
| | DESEFTFEKNGKKLDPTDYKWFQDYKPKLSKEVRDALSDIEWRLRRESLEENKL |
| | SKSREQDARQLANWISSMCDVIGIENLVKKNNFFGGSGKREPGWDNFYKPKKEN |
| | RWWINAIHKALTELSQNKGKRVILLPAMRTSITCPKCKYCDSKNRNGEKENCLK |
| | CGIELNADIDVATENLATVAITAQSMPKPTCERSGDAKKPVRARKAKAPEFHDK |
| | LAPSYTVVLREAV |
| 177 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTELSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISDLAKERGV |
| | DDTLYMFPFFKGKGKKRKTEIRKRWDYKWFQDYKPKLSKEVRDALSDIEWRLRR |
| | ESLEFNKLSKSREQDARQLANWISSMCDVIGIENLVKKNNFFGGSGKREPGWDN |
| | FYKPKKENRWWINAIHKALTELSQNKGKRVILLPAMRTSITCPKCKYCDSKNRN |
| | GEKFNCLKCGIELNADIDVATENLATVAITAQSMPKPTCERSGDAKKPVRARKA |
| | KAPEFHDKLAPSYTVVLREAV |
| 178 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISRWLRDHGW |
| | NASDFTQITKGRKKVERLWSDYKWFQDYKPKLSKEVRDALSDIEWRLRRESLEF |
| | NKLSKSREQDARQLANWISSMCDVIGIENLVKKNNFFGGSGKREPGWDNFYKPK |
| | KENRWWINAIHKALTELSQNKGKRVILLPAMRTSITCPKCKYCDSKNRNGEKEN |
| | CLKCGIELNADIDVATENLATVAITAQSMPKPTCERSGDAKKPVRARKAKAPEF |
| | HDKLAPSYTVVLREAV |
| 179 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISDLYLRRGG |
| | DPSRVFFVPQPSTPKKNAKKPPAPRKPVKRTDYKWFQDYKPKLSKEVRDALSDI |
| | EWRLRRESLEFNKLSKSREQDARQLANWISSMCDVIGIENLVKKNNFFGGSGKR |
| | EPGWDNFYKPKKENRWWINAIHKALTELSQNKGKRVILLPAMRTSITCPKCKYC |
| | DSKNRNGEKFNCLKCGIELNADIDVATENLATVAITAQSMPKPTCERSGDAKKP |
| | VRARKAKAPEFHDKLAPSYTVVLREAV |
| 180 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISDHLINHGG |
| | DTSAVFFQTIRKGTKKLETIKRKDYKWFQDYKPKLSKEVRDALSDIEWRLRRES |
| | LEFNKLSKSREQDARQLANWISSMCDVIGIENLVKKNNFFGGSGKREPGWDNFY |
| | KPKKENRWWINAIHKALTELSQNKGKRVILLPAMRTSITCPKCKYCDSKNRNGE |
| | KFNCLKCGIELNADIDVATENLATVAITAQSMPKPTCERSGDAKKPVRARKAKA |
| | PEFHDKLAPSYTVVLREAV |
| 181 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTELSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFIADELIKFEN |
| | KDAFYPSGKKKFDYKWFQDYKPKLSKEVRDALSDIEWRLRRESLEFNKLSKSRE |
| | QDARQLANWISSMCDVIGIENLVKKNNFFGGSGKREPGWDNFYKPKKENRWWIN |
| | AIHKALTELSQNKGKRVILLPAMRTSITCPKCKYCDSKNRNGEKFNCLKCGIEL |
| | NADIDVATENLATVAITAQSMPKPTCERSGDAKKPVRARKAKAPEFHDKLAPSY |
| | TVVLREAV |
| 182 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEAILAQGV |
| | SRDQVFFTPNPKKGSKEPVEVMRKDYKWFQDYKPKLSKEVRDALSDIEWRLRRE |
| | SLEFNKLSKSREQDARQLANWISSMCDVIGIENLVKKNNFFGGSGKREPGWDNF |
| | YKPKKENRWWINAIHKALTELSQNKGKRVILLPAMRTSITCPKCKYCDSKNRNG |
| | EKFNCLKCGIELNADIDVATENLATVAITAQSMPKPTCERSGDAKKPVRARKAK |
| | APEFHDKLAPSYTVVLREAV |
| 183 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTELSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFIADEFINNGG |
| | DKSLVYFTAKDKKSKKEKLVKLSDYKWFQDYKPKLSKEVRDALSDIEWRLRRES |
| | LEFNKLSKSREQDARQLANWISSMCDVIGIENLVKKNNFFGGSGKREPGWDNFY |
| | KPKKENRWWINAIHKALTELSQNKGKRVILLPAMRTSITCPKCKYCDSKNRNGE |
| | KFNCLKCGIELNADIDVATENLATVAITAQSMPKPTCERSGDAKKPVRARKAKA |
| | PEFHDKLAPSYTVVLREAV |
| 184 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISDLYLKNGG |
| | DESRVYFEINNKRIKRSDYKWFQDYKPKLSKEVRDALSDIEWRLRRESLEENKL |
| | SKSREQDARQLANWISSMCDVIGIENLVKKNNFFGGSGKREPGWDNFYKPKKEN |
| | RWWINAIHKALTELSQNKGKRVILLPAMRTSITCPKCKYCDSKNRNGEKFNCLK |
| | CGIELNADIDVATENLATVAITAQSMPKPTCERSGDAKKPVRARKAKAPEFHDK |
| | LAPSYTVVLREAV |
| 185 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFIAQHLMSLGF |
| | GESATHVRYRPKRKASERTILKYDYKWFQDYKPKLSKEVRDALSDIEWRLRRES |
| | LEFNKLSKSREQDARQLANWISSMCDVIGIENLVKKNNFFGGSGKREPGWDNFY |
| | KPKKENRWWINAIHKALTELSQNKGKRVILLPAMRTSITCPKCKYCDSKNRNGE |
| | KFNCLKCGIELNADIDVATENLATVAITAQSMPKPTCERSGDAKKPVRARKAKA |
| | PEFHDKLAPSYTVVLREAV |
| 186 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISDALIEVGK |
| | EEETNFVTSNGPRKRTDYKWFQDYKPKLSKEVRDALSDIEWRLRRESLEENKLS |
| | KSREQDARQLANWISSMCDVIGIENLVKKNNFFGGSGKREPGWDNFYKPKKENR |
| | WWINAIHKALTELSQNKGKRVILLPAMRTSITCPKCKYCDSKNRNGEKENCLKC |
| | GIELNADIDVATENLATVAITAQSMPKPTCERSGDAKKPVRARKAKAPEFHDKL |
| | APSYTVVLREAV |
| 187 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISDCLVALGK |
| | TDDVFFVPKAKKGKKETGIAVKRKDYKWFQDYKPKLSKEVRDALSDIEWRLRRE |
| | SLEFNKLSKSREQDARQLANWISSMCDVIGIENLVKKNNFFGGSGKREPGWDNF |
| | YKPKKENRWWINAIHKALTELSQNKGKRVILLPAMRTSITCPKCKYCDSKNRNG |
| | EKFNCLKCGIELNADIDVATENLATVAITAQSMPKPTCERSGDAKKPVRARKAK |
| | APEFHDKLAPSYTVVLREAV |
| 188 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISDYLIDNNI |
| | TNDVYRTVNKAKYKTNDYKWFQDYKPKLSKEVRDALSDIEWRLRRESLEENKLS |
| | KSREQDARQLANWISSMCDVIGIENLVKKNNFFGGSGKREPGWDNFYKPKKENR |
| | WWINAIHKALTELSQNKGKRVILLPAMRTSITCPKCKYCDSKNRNGEKFNCLKC |
| | GIELNADIDVATENLATVAITAQSMPKPTCERSGDAKKPVRARKAKAPEFHDKL |
| | APSYTVVLREAV |
| 189 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISDYLIQIGR |
| | GTETEKTITTKKGKEKILTIRDYKWFQDYKPKLSKEVRDALSDIEWRLRRESLE |
| | FNKLSKSREQDARQLANWISSMCDVIGIENLVKKNNFFGGSGKREPGWDNFYKP |
| | KKENRWWINAIHKALTELSQNKGKRVILLPAMRTSITCPKCKYCDSKNRNGEKF |
| | NCLKCGIELNADIDVATENLATVAITAQSMPKPTCERSGDAKKPVRARKAKAPE |
| | FHDKLAPSYTVVLREAV |
| 190 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISDLYIERGG |
| | DPRDVHQQVETKPKGKRKSEIRILKIRDYKWFQDYKPKLSKEVRDALSDIEWRL |
| | RRESLEFNKLSKSREQDARQLANWISSMCDVIGIENLVKKNNFFGGSGKREPGW |
| | DNFYKPKKENRWWINAIHKALTELSQNKGKRVILLPAMRTSITCPKCKYCDSKN |
| | RNGEKFNCLKCGIELNADIDVATENLATVAITAQSMPKPTCERSGDAKKPVRAR |
| | KAKAPEFHDKLAPSYTVVLREAV |
| 191 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISDYIVDHGG |
| | DPEKVFFETKSKKDKTKRYKRRDYKWFQDYKPKLSKEVRDALSDIEWRLRRESL |
| | EFNKLSKSREQDARQLANWISSMCDVIGIENLVKKNNFFGGSGKREPGWDNFYK |
| | PKKENRWWINAIHKALTELSQNKGKRVILLPAMRTSITCPKCKYCDSKNRNGEK |
| | FNCLKCGIELNADIDVATENLATVAITAQSMPKPTCERSGDAKKPVRARKAKAP |
| | EFHDKLAPSYTVVLREAV |
| 192 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISDHLLAKGV |
| | TDQVFFEKKSKGKKKGTETVKRKDYKWFQDYKPKLSKEVRDALSDIEWRLRRES |
| | LEFNKLSKSREQDARQLANWISSMCDVIGIENLVKKNNFFGGSGKREPGWDNFY |
| | KPKKENRWWINAIHKALTELSQNKGKRVILLPAMRTSITCPKCKYCDSKNRNGE |
| | KFNCLKCGIELNADIDVATENLATVAITAQSMPKPTCERSGDAKKPVRARKAKA |
| | PEFHDKLAPSYTVVLREAV |
| 193 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFIENKHVENGG |
| | DPNVLLFTPTEDKKNKGKKSKNKKGEYGDRVPHSDYKWFQDYKPKLSKEVRDAL |
| | SDIEWRLRRESLEFNKLSKSREQDARQLANWISSMCDVIGIENLVKKNNFFGGS |
| | GKREPGWDNFYKPKKENRWWINAIHKALTELSQNKGKRVILLPAMRTSITCPKC |
| | KYCDSKNRNGEKFNCLKCGIELNADIDVATENLATVAITAQSMPKPTCERSGDA |
| | KKPVRARKAKAPEFHDKLAPSYTVVLREAV |
| 194 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTELSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISNYLLNNGL |
| | SPQEGASDQDWVVPSSTREVARVAYRISDSKGKGKKKPEIFKASDYKWFQDYKP |
| | KLSKEVRDALSDIEWRLRRESLEFNKLSKSREQDARQLANWISSMCDVIGIENL |
| | VKKNNFFGGSGKREPGWDNFYKPKKENRWWINAIHKALTELSQNKGKRVILLPA |
| | MRTSITCPKCKYCDSKNRNGEKFNCLKCGIELNADIDVATENLATVAITAQSMP |
| | KPTCERSGDAKKPVRARKAKAPEFHDKLAPSYTVVLREAV |
| 195 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTELSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFIAKWVMRHGD |
| | PSLVEVLEYRKDNEIKLDKNGVPKKVKLTDYKWFQDYKPKLSKEVRDALSDIEW |
| | RLRRESLEFNKLSKSREQDARQLANWISSMCDVIGIENLVKKNNFFGGSGKREP |
| | GWDNFYKPKKENRWWINAIHKALTELSQNKGKRVILLPAMRTSITCPKCKYCDS |
| | KNRNGEKFNCLKCGIELNADIDVATENLATVAITAQSMPKPTCERSGDAKKPVR |
| | ARKAKAPEFHDKLAPSYTVVLREAV |
| 196 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLEDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIEDLDVKKKENGRGIRDIGWDNFFSSRKE |
| | NRWWINAIHKALTELSQNKGKRVILLPAMRTSITCPKCKYCDSKNRNGEKFNCL |
| | KCGIELNADIDVATENLATVAITAQSMPKPTCERSGDAKKPVRARKAKAPEFHD |
| | KLAPSYTVVLREAV |
| 197 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTELSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPDCGFCSKENRDGINFTC |
| | RKCGVSYHADIDVATENLATVAITAQSMPKPTCERSGDAKKPVRARKAKAPEFH |
| | DKLAPSYTVVLREAV |
| 198 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDGSSGSDYKWFQDYKPKLSKEVRDALSDIEWRLRRESLEFNKLSKS |
| | REQDARQLANWISSMCDVIGIENLVKKNNFFGGSGKREPGWDNFYKPKKENRWW |
| | INAIHKALTELSQNKGKRVILLPAMRTSITCPKCKYCDSKNRNGEKFNCLKCGI |
| | ELNADIDVATENLATVAITAQSMPKPTCERSGDAKKPVRARKAKAPEFHDKLAP |
| | SYTVVLREAV |
| 199 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLG |
| | SSGEIKAFDDKGYLLQKPSPNKSIYCYQSVSPKPFITSKYHNVNLPEEYIGYYR |
| | KSNEPIVSPYQFDRLRIPIGEPGYVPKWQYTFLSKKENKRRKLSKRIKNVSPIL |
| | GIICIKKDWCVFDMRGLLRTNHWKKYHKPTDSINDLFDYFTGDPVIDTKANVVR |
| | FRYKMENGIVNYKPVREKKGKELLENICDQNGSCKLATVDVGQNNPVAIGLFEL |
| | KKVNGELTKTLISRHPTPIDFCNKITAYRERYDKLESSIKLDAIKQLTSEQKIE |
| | VDNYNNNFTPQNTKQIVCSKLNINPNDLPWDKMISGTHFISEKAQVSNKSEIYF |
| | TSTDGSSGSDYKWFQDYKPKLSKEVRDALSDIEWRLRRESLEFNKLSKSREQDA |
| | RQLANWISSMCDVIGIENLVKKNNFFGGSGKREPGWDNFYKPKKENRWWINAIH |
| | KALTELSQNKGKRVILLPAMRTSITCPKCKYCDSKNRNGEKENCLKCGIELNAD |
| | IDVATENLATVAITAQSMPKPTCERSGDAKKPVRARKAKAPEFHDKLAPSYTVV |
| | LREAV |
| 200 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNGSSGLAKINRKNEIAKLNGEQEISFEEIKAFDD |
| | KGYLLQKPSPNKSIYCYQSVSPKPFITSKYHNVNLPEEYIGYYRKSNEPIVSPY |
| | QFDRLRIPIGEPGYVPKWQYTELSKKENKRRKLSKRIKNVSPILGIICIKKDWC |
| | VFDMRGLLRTNHWKKYHKPTDSINDLFDYFTGDPVIDTKANVVRFRYKMENGIV |
| | NYKPVREKKGKELLENICDQNGSCKLATVDVGQNNPVAIGLFELKKVNGELTKT |
| | LISRHPTPIDFCNKITAYRERYDKLESSIKLDAIKQLTSEQKIEVDNYNNNFTP |
| | QNTKQIVCSKLNINPNDLPWDKMISGTHFISEKAQVSNKSEIYFTSTDGSSGSD |
| | YKWFQDYKPKLSKEVRDALSDIEWRLRRESLEFNKLSKSREQDARQLANWISSM |
| | CDVIGIENLVKKNNFFGGSGKREPGWDNFYKPKKENRWWINAIHKALTELSQNK |
| | GKRVILLPAMRTSITCPKCKYCDSKNRNGEKFNCLKCGIELNADIDVATENLAT |
| | VAITAQSMPKPTCERSGDAKKPVRARKAKAPEFHDKLAPSYTVVLREAV |
| 201 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNAIAKLN |
| | GAQAISFAEIKAFDDKGYLLQKPSPNKSIYCYQSVSPKPFITSKYHNVNLPEEY |
| | IGYYRKSNEPIVSPYQFDRLRIPIGEPGYVPKWQYTELSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDGSSGSDYKWFQDYKPKLSKEVRDALSDIEWRLRRESLEFNKLSKS |
| | REQDARQLANWISSMCDVIGIENLVKKNNFFGGSGKREPGWDNFYKPKKENRWW |
| | INAIHKALTELSQNKGKRVILLPAMRTSITCPKCKYCDSKNRNGEKFNCLKCGI |
| | ELNADIDVATENLATVAITAQSMPKPTCERSGDAKKPVRARKAKAPEFHDKLAP |
| | SYTVVLREAV |
| 202 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNGSSGEEIKAFDDKG |
| | YLLQKPSPNKSIYCYQSVSPKPFITSKYHNVNLPEEYIGYYRKSNEPIVSPYQF |
| | DRLRIPIGEPGYVPKWQYTELSKKENKRRKLSKRIKNVSPILGIICIKKDWCVF |
| | DMRGLLRTNHWKKYHKPTDSINDLFDYFTGDPVIDTKANVVRFRYKMENGIVNY |
| | KPVREKKGKELLENICDQNGSCKLATVDVGQNNPVAIGLFELKKVNGELTKTLI |
| | SRHPTPIDFCNKITAYRERYDKLESSIKLDAIKQLTSEQKIEVDNYNNNFTPQN |
| | TKQIVCSKLNINPNDLPWDKMISGTHFISEKAQVSNKSEIYFTSTDKGKTKDVM |
| | KSDYKWFQDYKPKLSKEVRDALSDIEWRLRRESLEFNKLSKSREQDARQLANWI |
| | SSMCDVIGIENLVKKNNFFGGSGKREPGWDNFYKPKKENRWWINAIHKALTELS |
| | QNKGKRVILLPAMRTSITCPKCKYCDSKNRNGEKFNCLKCGIELNADIDVATEN |
| | LATVAITAQSMPKPTCERSGDAKKPVRARKAKAPEFHDKLAPSYTVVLREAV |
| 203 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNGSSGEIKAFDDKGY |
| | LLQKPSPNKSIYCYQSVSPKPFITSKYHNVNLPEEYIGYYRKSNEPIVSPYQFD |
| | RLRIPIGEPGYVPKWQYTFLSKKENKRRKLSKRIKNVSPILGIICIKKDWCVED |
| | MRGLLRTNHWKKYHKPTDSINDLFDYFTGDPVIDTKANVVRFRYKMENGIVNYK |
| | PVREKKGKELLENICDQNGSCKLATVDVGQNNPVAIGLFELKKVNGELTKTLIS |
| | RHPTPIDFCNKITAYRERYDKLESSIKLDAIKQLTSEQKIEVDNYNNNFTPQNT |
| | KQIVCSKLNINPNDLPWDKMISGTHFISEKAQVSNKSEIYFTSTDKGKTKDVMK |
| | SDYKWFQDYKPKLSKEVRDALSDIEWRLRRESLEFNKLSKSREQDARQLANWIS |
| | SMCDVIGIENLVKKNNFFGGSGKREPGWDNFYKPKKENRWWINAIHKALTELSQ |
| | NKGKRVILLPAMRTSITCPKCKYCDSKNRNGEKFNCLKCGIELNADIDVATENL |
| | ATVAITAQSMPKPTCERSGDAKKPVRARKAKAPEFHDKLAPSYTVVLREAV |
| 204 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQGSSG |
| | DYKWFQDYKPKLSKEVRDALSDIEWRLRRESLEFNKLSKSREQDARQLANWISS |
| | MCDVIGIENLVKKNNFFGGSGKREPGWDNFYKPKKENRWWINAIHKALTELSQN |
| | KGKRVILLPAMRTSITCPKCKYCDSKNRNGEKFNCLKCGIELNADIDVATENLA |
| | TVAITAQSMPKPTCERSGDAKKPVRARKAKAPEFHDKLAPSYTVVLREAV |
| 205 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEGSSGDYKWFQDYKPKLSKEVRDALSDIEWRLRRESLEFNKLSKSREQDARQL |
| | ANWISSMCDVIGIENLVKKNNFFGGSGKREPGWDNFYKPKKENRWWINAIHKAL |
| | TELSQNKGKRVILLPAMRTSITCPKCKYCDSKNRNGEKFNCLKCGIELNADIDV |
| | ATENLATVAITAQSMPKPTCERSGDAKKPVRARKAKAPEFHDKLAPSYTVVLRE |
| | AV |
| 206 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLG |
| | SSGEIKAFDDKGYLLQKPSPNKSIYCYQSVSPKPFITSKYHNVNLPEEYIGYYR |
| | KSNEPIVSPYQFDRLRIPIGEPGYVPKWQYTELSKKENKRRKLSKRIKNVSPIL |
| | GIICIKKDWCVFDMRGLLRTNHWKKYHKPTDSINDLFDYFTGDPVIDTKANVVR |
| | FRYKMENGIVNYKPVREKKGKELLENICDQNGSCKLATVDVGQNNPVAIGLFEL |
| | KKVNGELTKTLISRHPTPIDFCNKITAYRERYDKLESSIKLDAIKQLTSEQKIE |
| | VDNYNNNFTPQNTKQIVCSKLNINPNDLPWDKMISGTHFISEKAQGSSGDYKWF |
| | QDYKPKLSKEVRDALSDIEWRLRRESLEFNKLSKSREQDARQLANWISSMCDVI |
| | GIENLVKKNNFFGGSGKREPGWDNFYKPKKENRWWINAIHKALTELSQNKGKRV |
| | ILLPAMRTSITCPKCKYCDSKNRNGEKFNCLKCGIELNADIDVATENLATVAIT |
| | AQSMPKPTCERSGDAKKPVRARKAKAPEFHDKLAPSYTVVLREAV |
| 207 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNGSSGLAKINRKNEIAKLNGEQEISFEEIKAFDD |
| | KGYLLQKPSPNKSIYCYQSVSPKPFITSKYHNVNLPEEYIGYYRKSNEPIVSPY |
| | QFDRLRIPIGEPGYVPKWQYTFLSKKENKRRKLSKRIKNVSPILGIICIKKDWC |
| | VFDMRGLLRTNHWKKYHKPTDSINDLFDYFTGDPVIDTKANVVRFRYKMENGIV |
| | NYKPVREKKGKELLENICDQNGSCKLATVDVGQNNPVAIGLFELKKVNGELTKT |
| | LISRHPTPIDFCNKITAYRERYDKLESSIKLDAIKQLTSEQKIEVDNYNNNFTP |
| | QNTKQIVCSKLNINPNDLPWDKMISGTHFISEKAQGSSGDYKWFQDYKPKLSKE |
| | VRDALSDIEWRLRRESLEFNKLSKSREQDARQLANWISSMCDVIGIENLVKKNN |
| | FFGGSGKREPGWDNFYKPKKENRWWINAIHKALTELSQNKGKRVILLPAMRTSI |
| | TCPKCKYCDSKNRNGEKFNCLKCGIELNADIDVATENLATVAITAQSMPKPTCE |
| | RSGDAKKPVRARKAKAPEFHDKLAPSYTVVLREAV |
| 208 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNAIAKLN |
| | GAQAISFAEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQGSSG |
| | DYKWFQDYKPKLSKEVRDALSDIEWRLRRESLEFNKLSKSREQDARQLANWISS |
| | MCDVIGIENLVKKNNFFGGSGKREPGWDNFYKPKKENRWWINAIHKALTELSQN |
| | KGKRVILLPAMRTSITCPKCKYCDSKNRNGEKFNCLKCGIELNADIDVATENLA |
| | TVAITAQSMPKPTCERSGDAKKPVRARKAKAPEFHDKLAPSYTVVLREAV |
| 323 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNAIAKLN |
| | GLQIISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQGSSG |
| | DYKWFQDYKPKLSKEVRDALSDIEWRLRRESLEFNKLSKSREQDARQLANWISS |
| | MCDVIGIENLVKKNNFFGGSGKREPGWDNFYKPKKENRWWINAIHKALTELSQN |
| | KGKRVILLPAMRTSITCPKCKYCDSKNRNGEKFNCLKCGIELNADIDVATENLA |
| | TVAITAQSMPKPTCERSGDAKKPVRARKAKAPEFHDKLAPSYTVVLREAV |
| 210 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNGSSGEEIKAFDDKG |
| | YLLQKPSPNKSIYCYQSVSPKPFITSKYHNVNLPEEYIGYYRKSNEPIVSPYQF |
| | DRLRIPIGEPGYVPKWQYTELSKKENKRRKLSKRIKNVSPILGIICIKKDWCVF |
| | DMRGLLRTNHWKKYHKPTDSINDLFDYFTGDPVIDTKANVVRFRYKMENGIVNY |
| | KPVREKKGKELLENICDQNGSCKLATVDVGQNNPVAIGLFELKKVNGELTKTLI |
| | SRHPTPIDFCNKITAYRERYDKLESSIKLDAIKQLTSEQKIEVDNYNNNFTPQN |
| | TKQIVCSKLNINPNDLPWDKMISGTHFISEKAQGSSGDYKWFQDYKPKLSKEVR |
| | DALSDIEWRLRRESLEFNKLSKSREQDARQLANWISSMCDVIGIENLVKKNNFF |
| | GGSGKREPGWDNFYKPKKENRWWINAIHKALTELSQNKGKRVILLPAMRTSITC |
| | PKCKYCDSKNRNGEKFNCLKCGIELNADIDVATENLATVAITAQSMPKPTCERS |
| | GDAKKPVRARKAKAPEFHDKLAPSYTVVLREAV |
| 211 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNGSSGEIKAFDDKGY |
| | LLQKPSPNKSIYCYQSVSPKPFITSKYHNVNLPEEYIGYYRKSNEPIVSPYQFD |
| | RLRIPIGEPGYVPKWQYTFLSKKENKRRKLSKRIKNVSPILGIICIKKDWCVED |
| | MRGLLRTNHWKKYHKPTDSINDLFDYFTGDPVIDTKANVVRFRYKMENGIVNYK |
| | PVREKKGKELLENICDQNGSCKLATVDVGQNNPVAIGLFELKKVNGELTKTLIS |
| | RHPTPIDFCNKITAYRERYDKLESSIKLDAIKQLTSEQKIEVDNYNNNFTPQNT |
| | KQIVCSKLNINPNDLPWDKMISGTHFISEKAQGSSGDYKWFQDYKPKLSKEVRD |
| | ALSDIEWRLRRESLEFNKLSKSREQDARQLANWISSMCDVIGIENLVKKNNFFG |
| | GSGKREPGWDNFYKPKKENRWWINAIHKALTELSQNKGKRVILLPAMRTSITCP |
| | KCKYCDSKNRNGEKENCLKCGIELNADIDVATENLATVAITAQSMPKPTCERSG |
| | DAKKPVRARKAKAPEFHDKLAPSYTVVLREAV |
| 212 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKGSSGFEEIKAF |
| | DDKGYLLQKPSPNKSIYCYQSVSPKPFITSKYHNVNLPEEYIGYYRKSNEPIVS |
| | PYQFDRLRIPIGEPGYVPKWQYTFLSKKENKRRKLSKRIKNVSPILGIICIKKD |
| | WCVFDMRGLLRTNHWKKYHKPTDSINDLFDYFTGDPVIDTKANVVRFRYKMENG |
| | IVNYKPVREKKGKELLENICDQNGSCKLATVDVGQNNPVAIGLFELKKVNGELT |
| | KTLISRHPTPIDFCNKITAYRERYDKLESSIKLDAIKQLTSEQKIEVDNYNNNE |
| | TPQNTKQIVCSKLNINPNDLPWDKMISGTHFISEKAQGSSGDYKWFQDYKPKLS |
| | KEVRDALSDIEWRLRRESLEFNKLSKSREQDARQLANWISSMCDVIGIENLVKK |
| | NNFFGGSGKREPGWDNFYKPKKENRWWINAIHKALTELSQNKGKRVILLPAMRT |
| | SITCPKCKYCDSKNRNGEKFNCLKCGIELNADIDVATENLATVAITAQSMPKPT |
| | CERSGDAKKPVRARKAKAPEFHDKLAPSYTVVLREAV |
| 213 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKIGSSFEEIKAF |
| | DDKGYLLQKPSPNKSIYCYQSVSPKPFITSKYHNVNLPEEYIGYYRKSNEPIVS |
| | PYQFDRLRIPIGEPGYVPKWQYTELSKKENKRRKLSKRIKNVSPILGIICIKKD |
| | WCVFDMRGLLRTNHWKKYHKPTDSINDLFDYFTGDPVIDTKANVVRFRYKMENG |
| | IVNYKPVREKKGKELLENICDQNGSCKLATVDVGQNNPVAIGLFELKKVNGELT |
| | KTLISRHPTPIDFCNKITAYRERYDKLESSIKLDAIKQLTSEQKIEVDNYNNNE |
| | TPQNTKQIVCSKLNINPNDLPWDKMISGTHFISEKAQGSSGDYKWFQDYKPKLS |
| | KEVRDALSDIEWRLRRESLEFNKLSKSREQDARQLANWISSMCDVIGIENLVKK |
| | NNFFGGSGKREPGWDNFYKPKKENRWWINAIHKALTELSQNKGKRVILLPAMRT |
| | SITCPKCKYCDSKNRNGEKFNCLKCGIELNADIDVATENLATVAITAQSMPKPT |
| | CERSGDAKKPVRARKAKAPEFHDKLAPSYTVVLREAV |
| 214 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKGSISFEE |
| | IKAFDDKGYLLQKPSPNKSIYCYQSVSPKPFITSKYHNVNLPEEYIGYYRKSNE |
| | PIVSPYQFDRLRIPIGEPGYVPKWQYTELSKKENKRRKLSKRIKNVSPILGIIC |
| | IKKDWCVFDMRGLLRTNHWKKYHKPTDSINDLFDYFTGDPVIDTKANVVRFRYK |
| | MENGIVNYKPVREKKGKELLENICDQNGSCKLATVDVGQNNPVAIGLFELKKVN |
| | GELTKTLISRHPTPIDFCNKITAYRERYDKLESSIKLDAIKQLTSEQKIEVDNY |
| | NNNFTPQNTKQIVCSKLNINPNDLPWDKMISGTHFISEKAQGSSGDYKWFQDYK |
| | PKLSKEVRDALSDIEWRLRRESLEFNKLSKSREQDARQLANWISSMCDVIGIEN |
| | LVKKNNFFGGSGKREPGWDNFYKPKKENRWWINAIHKALTELSQNKGKRVILLP |
| | AMRTSITCPKCKYCDSKNRNGEKFNCLKCGIELNADIDVATENLATVAITAQSM |
| | PKPTCERSGDAKKPVRARKAKAPEFHDKLAPSYTVVLREAV |
| 215 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLG |
| | SSGEIKAFDDKGYLLQKPSPNKSIYCYQSVSPKPFITSKYHNVNLPEEYIGYYR |
| | KSNEPIVSPYQFDRLRIPIGEPGYVPKWQYTELSKKENKRRKLSKRIKNVSPIL |
| | GIICIKKDWCVFDMRGLLRTNHWKKYHKPTDSINDLFDYFTGDPVIDTKANVVR |
| | FRYKMENGIVNYKPVREKKGKELLENICDQNGSCKLATVDVGQNNPVAIGLFEL |
| | KKVNGELTKTLISRHPTPIDFCNKITAYRERYDKLESSIKLDAIKQLTSEQKIE |
| | VDNYNNNFTPQNTKQIVCSKLNINPNDLPWDKMISGTHFISEKAQVSNKSEIYF |
| | TSTDKGKTKDVMKSDYKWFQDYKPKLSKEVRDALSDIEWRLRRESLEFNKLSKS |
| | REQDARQLANWISSMCDVIGIENLVKKNNFFGGSGKREPGWDNFYKPKKENRWW |
| | INAIHKALTELSQNKGKRVILLPAMRTSITCPKCKYCDSKNRNGEKFNCLKCGI |
| | ELNADIDVATENLATVAITAQSMPKPTCERSGDAKKPVRARKAKAPEFHDKLAP |
| | SYTVVLREAV |
| 216 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNGSSGLAKINRKNEIAKLNGEQEISFEEIKAFDD |
| | KGYLLQKPSPNKSIYCYQSVSPKPFITSKYHNVNLPEEYIGYYRKSNEPIVSPY |
| | QFDRLRIPIGEPGYVPKWQYTELSKKENKRRKLSKRIKNVSPILGIICIKKDWC |
| | VFDMRGLLRTNHWKKYHKPTDSINDLFDYFTGDPVIDTKANVVRFRYKMENGIV |
| | NYKPVREKKGKELLENICDQNGSCKLATVDVGQNNPVAIGLFELKKVNGELTKT |
| | LISRHPTPIDFCNKITAYRERYDKLESSIKLDAIKQLTSEQKIEVDNYNNNFTP |
| | QNTKQIVCSKLNINPNDLPWDKMISGTHFISEKAQVSNKSEIYFTSTDKGKTKD |
| | VMKSDYKWFQDYKPKLSKEVRDALSDIEWRLRRESLEFNKLSKSREQDARQLAN |
| | WISSMCDVIGIENLVKKNNFFGGSGKREPGWDNFYKPKKENRWWINAIHKALTE |
| | LSQNKGKRVILLPAMRTSITCPKCKYCDSKNRNGEKFNCLKCGIELNADIDVAT |
| | ENLATVAITAQSMPKPTCERSGDAKKPVRARKAKAPEFHDKLAPSYTVVLREAV |
| 217 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNAIAKLN |
| | GAQAISFAEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 218 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNAIAKLN |
| | GLQIISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLEDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 219 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNGSSGEEIKAFDDKG |
| | YLLQKPSPNKSIYCYQSVSPKPFITSKYHNVNLPEEYIGYYRKSNEPIVSPYQF |
| | DRLRIPIGEPGYVPKWQYTFLSKKENKRRKLSKRIKNVSPILGIICIKKDWCVF |
| | DMRGLLRTNHWKKYHKPTDSINDLFDYFTGDPVIDTKANVVRFRYKMENGIVNY |
| | KPVREKKGKELLENICDQNGSCKLATVDVGQNNPVAIGLFELKKVNGELTKTLI |
| | SRHPTPIDFCNKITAYRERYDKLESSIKLDAIKQLTSEQKIEVDNYNNNFTPQN |
| | TKQIVCSKLNINPNDLPWDKMISGTHFISEKAQVSNKSEIYFTSTDKGKTKDVM |
| | KSDYKWFQDYKPKLSKEVRDALSDIEWRLRRESLEFNKLSKSREQDARQLANWI |
| | SSMCDVIGIENLVKKNNFFGGSGKREPGWDNFYKPKKENRWWINAIHKALTELS |
| | QNKGKRVILLPAMRTSITCPKCKYCDSKNRNGEKFNCLKCGIELNADIDVATEN |
| | LATVAITAQSMPKPTCERSGDAKKPVRARKAKAPEFHDKLAPSYTVVLREAV |
| 220 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNGSSGEIKAFDDKGY |
| | LLQKPSPNKSIYCYQSVSPKPFITSKYHNVNLPEEYIGYYRKSNEPIVSPYQFD |
| | RLRIPIGEPGYVPKWQYTELSKKENKRRKLSKRIKNVSPILGIICIKKDWCVFD |
| | MRGLLRTNHWKKYHKPTDSINDLFDYFTGDPVIDTKANVVRFRYKMENGIVNYK |
| | PVREKKGKELLENICDQNGSCKLATVDVGQNNPVAIGLFELKKVNGELTKTLIS |
| | RHPTPIDFCNKITAYRERYDKLESSIKLDAIKQLTSEQKIEVDNYNNNFTPQNT |
| | KQIVCSKLNINPNDLPWDKMISGTHFISEKAQVSNKSEIYFTSTDKGKTKDVMK |
| | SDYKWFQDYKPKLSKEVRDALSDIEWRLRRESLEFNKLSKSREQDARQLANWIS |
| | SMCDVIGIENLVKKNNFFGGSGKREPGWDNFYKPKKENRWWINAIHKALTELSQ |
| | NKGKRVILLPAMRTSITCPKCKYCDSKNRNGEKFNCLKCGIELNADIDVATENL |
| | ATVAITAQSMPKPTCERSGDAKKPVRARKAKAPEFHDKLAPSYTVVLREAV |
| 221 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKGSSGFEEIKAF |
| | DDKGYLLQKPSPNKSIYCYQSVSPKPFITSKYHNVNLPEEYIGYYRKSNEPIVS |
| | PYQFDRLRIPIGEPGYVPKWQYTFLSKKENKRRKLSKRIKNVSPILGIICIKKD |
| | WCVFDMRGLLRTNHWKKYHKPTDSINDLFDYFTGDPVIDTKANVVRFRYKMENG |
| | IVNYKPVREKKGKELLENICDQNGSCKLATVDVGQNNPVAIGLFELKKVNGELT |
| | KTLISRHPTPIDFCNKITAYRERYDKLESSIKLDAIKQLTSEQKIEVDNYNNNF |
| | TPQNTKQIVCSKLNINPNDLPWDKMISGTHFISEKAQVSNKSEIYFTSTDKGKT |
| | KDVMKSDYKWFQDYKPKLSKEVRDALSDIEWRLRRESLEFNKLSKSREQDARQL |
| | ANWISSMCDVIGIENLVKKNNFFGGSGKREPGWDNFYKPKKENRWWINAIHKAL |
| | TELSQNKGKRVILLPAMRTSITCPKCKYCDSKNRNGEKFNCLKCGIELNADIDV |
| | ATENLATVAITAQSMPKPTCERSGDAKKPVRARKAKAPEFHDKLAPSYTVVLRE |
| | AV |
| 222 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKIGSSFEEIKAF |
| | DDKGYLLQKPSPNKSIYCYQSVSPKPFITSKYHNVNLPEEYIGYYRKSNEPIVS |
| | PYQFDRLRIPIGEPGYVPKWQYTELSKKENKRRKLSKRIKNVSPILGIICIKKD |
| | WCVFDMRGLLRTNHWKKYHKPTDSINDLFDYFTGDPVIDTKANVVRFRYKMENG |
| | IVNYKPVREKKGKELLENICDQNGSCKLATVDVGQNNPVAIGLFELKKVNGELT |
| | KTLISRHPTPIDFCNKITAYRERYDKLESSIKLDAIKQLTSEQKIEVDNYNNNF |
| | TPQNTKQIVCSKLNINPNDLPWDKMISGTHFISEKAQVSNKSEIYFTSTDKGKT |
| | KDVMKSDYKWFQDYKPKLSKEVRDALSDIEWRLRRESLEFNKLSKSREQDARQL |
| | ANWISSMCDVIGIENLVKKNNFFGGSGKREPGWDNFYKPKKENRWWINAIHKAL |
| | TELSQNKGKRVILLPAMRTSITCPKCKYCDSKNRNGEKENCLKCGIELNADIDV |
| | ATENLATVAITAQSMPKPTCERSGDAKKPVRARKAKAPEFHDKLAPSYTVVLRE |
| | AV |
| 223 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKGSISFEE |
| | IKAFDDKGYLLQKPSPNKSIYCYQSVSPKPFITSKYHNVNLPEEYIGYYRKSNE |
| | PIVSPYQFDRLRIPIGEPGYVPKWQYTFLSKKENKRRKLSKRIKNVSPILGIIC |
| | IKKDWCVFDMRGLLRTNHWKKYHKPTDSINDLFDYFTGDPVIDTKANVVRFRYK |
| | MENGIVNYKPVREKKGKELLENICDQNGSCKLATVDVGQNNPVAIGLFELKKVN |
| | GELTKTLISRHPTPIDFCNKITAYRERYDKLESSIKLDAIKQLTSEQKIEVDNY |
| | NNNFTPQNTKQIVCSKLNINPNDLPWDKMISGTHFISEKAQVSNKSEIYFTSTD |
| | KGKTKDVMKSDYKWFQDYKPKLSKEVRDALSDIEWRLRRESLEFNKLSKSREQD |
| | ARQLANWISSMCDVIGIENLVKKNNFFGGSGKREPGWDNFYKPKKENRWWINAI |
| | HKALTELSQNKGKRVILLPAMRTSITCPKCKYCDSKNRNGEKFNCLKCGIELNA |
| | DIDVATENLATVAITAQSMPKPTCERSGDAKKPVRARKAKAPEFHDKLAPSYTV |
| | VLREAV |
| 224 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISDLYIERGG |
| | DPRDVHQQVETKPKGKRKSEIRILKIRDYKWFQDYKPKLSKEVRDALSDIEWRL |
| | RRESLEFNKLSKSREQDARQLANWISSMCDVIGIENLVKKNNFFGGSGKREPGW |
| | DNFYKPKKENRWWINAIHKALTELSQNKGKRVILLPAMRTSITCPKCKYCDSKN |
| | RNGEKFNCLKCGIELNADIDVATENLATVAITAQSMPKPTCERSGDAKKPVRAR |
| | KAKAPEFHDKLAPSYTVVLREAV |
| 225 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEVAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | DKLAPSYTVVLREAV |
| 226 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEQAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | DKLAPSYTVVLREAV |
| 227 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEFAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 228 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEWAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | DKLAPSYTVVLREAV |
| 229 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLFLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTELSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | DKLAPSYTVVLREAV |
| 230 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLYLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 231 | MIKPTVSQFLTPGFKLIRNHSRTAGKKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTELSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | DKLAPSYTVVLREAV |
| 232 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMISGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIQNLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 233 | MPKIKKPTEISLLRKEVFPDLHFAKDRMRAASLVLKNEGREAAIEYLRVNHEDK |
| | PPNFMPPAKTPYVALSRPLEQWPIAQASIAIQKYIFGLTKDEFSATKKLLYGDK |
| | STPNTESRKRWFEVTGVPNFGYMSAQGLNAIFSGALARYEGVVQKVENRNKKRF |
| | EKLSEKNQLLIEEGQPVKDYVPDTAYHTPETLQKLAENNHVRVEDLGDMIDRLV |
| | HPPGIHRSIYGYQQVPPFAYDPDNPKGIILPKAYAGYTRKPHDIIEAMPNRLNI |
| | PEGQAGYIPEHQRDKLKKGGRVKRLRTTRVRVDATETVRAKAEALNAEKARLRG |
| | KEAILAVFQIEEDWALIDMRGLLRNVYMRKLIAAGELTPTTLLGYFTETLTLDP |
| | RRTEATFCYHLRSEGALHAEYVRHGKNTRELLLDLTKDNEKIALVTIDLGQRNP |
| | LAAAIFRVGRDASGDLTENSLEPVSRMLLPQAYLDQIKAYRDAYDSFRQNIWDT |
| | ALASLTPEQQRQILAYEAYTPDDSKENVLRLLLGGNVMPDDLPWEDMTKNTHYI |
| | SDRYLADGSSGDHNISHLSEFRPQLLKETRDAFEKAKIDTERGHVGYQKLSTRK |
| | DQLCKEILNWLEAEAVRLTRCKTMVLGLEDLNGPFFNQGKGKVRGWVSFFRQKQ |
| | ENRWIVNGFRKNALARAHDKGKYILELWPSWTSQTCPKCKHVHADNRHGDDFVC |
| | LQCGARLHADAEVATWNLAVVAIQGHSLPGPVREKSNDRKKSGSARKSKKANES |
| | GKVVGAWAAQATPKRATSKKETGTARNPVYNPLETQASCPAP |
| 234 | MPKIKKPTEISLLRKEVFPDLHFAKDRMRAASLVLKNEGREAAIEYLRVNHEDK |
| | PPNFMPPAKTPYVALSRPLEQWPIAQASIAIQKYIFGLTKDEFSATKKLLYGDK |
| | STPNTESRKRWFEVTGVPNFGYMSAQGLNAIFSGALARYEGVVQKVENRNKKRF |
| | EKLSEKNQLLIEEGQPVKDYVPDTAYHTPETLQKLAENNHVRVEDLGDMIDRLV |
| | HPPGIHRSIYGYQQVPPFAYDPDNPKGIILPKAYAGYTRKPHDIIEAMPNRLNI |
| | PEGQAGYIPEHQRDKLKKGGRVKRLRTTRVRVDATETVRAKAEALNAEKARLRG |
| | KEAILAVFQIEEDWALIDMRGLLRNVYMRKLIAAGELTPTTLLGYFTETLTLDP |
| | RRTEATFCYHLRSEGALHAEYVRHGKNTRELLLDLTKDNEKIALVTIDLGQRNP |
| | LAAAIFRVGRDASGDLTENSLEPVSRMLLPQAYLDQIKAYRDAYDSFRQNIWDT |
| | ALASLTPEQQRQILAYEAYTPDDSKENVLRLLLGGNVMPDDLPWEDMTKNTHYI |
| | SDRYLADGGDPSKVWFVPGPGSSGDHNISHLSEFRPQLLKETRDAFEKAKIDTE |
| | RGHVGYQKLSTRKDQLCKEILNWLEAEAVRLTRCKTMVLGLEDLNGPFFNQGKG |
| | KVRGWVSFFRQKQENRWIVNGFRKNALARAHDKGKYILELWPSWTSQTCPKCKH |
| | VHADNRHGDDFVCLQCGARLHADAEVATWNLAVVAIQGHSLPGPVREKSNDRKK |
| | SGSARKSKKANESGKVVGAWAAQATPKRATSKKETGTARNPVYNPLETQASCPA |
| | P |
| 235 | MPKIKKPTEISLLRKEVFPDLHFAKDRMRAASLVLKNEGREAAIEYLRVNHEDK |
| | PPNFMPPAKTPYVALSRPLEQWPIAQASIAIQKYIFGLTKDEFSATKKLLYGDK |
| | STPNTESRKRWFEVTGVPNFGYMSAQGLNAIFSGALARYEGVVQKVENRNKKRG |
| | SSGPDTAYHTPETLQKLAENNHVRVEDLGDMIDRLVHPPGIHRSIYGYQQVPPF |
| | AYDPDNPKGIILPKAYAGYTRKPHDIIEAMPNRLNIPEGQAGYIPEHQRDKLKK |
| | GGRVKRLRTTRVRVDATETVRAKAEALNAEKARLRGKEAILAVFQIEEDWALID |
| | MRGLLRNVYMRKLIAAGELTPTTLLGYFTETLTLDPRRTEATFCYHLRSEGALH |
| | AEYVRHGKNTRELLLDLTKDNEKIALVTIDLGQRNPLAAAIFRVGRDASGDLTE |
| | NSLEPVSRMLLPQAYLDQIKAYRDAYDSFRQNIWDTALASLTPEQQRQILAYEA |
| | YTPDDSKENVLRLLLGGNVMPDDLPWEDMTKNTHYISDRYLADGGDPSKVWFVP |
| | GPRKRKKNAPPLKKPPKPRELVKRSDHNISHLSEFRPQLLKETRDAFEKAKIDT |
| | ERGHVGYQKLSTRKDQLCKEILNWLEAEAVRLTRCKTMVLGLEDLNGPFFNQGK |
| | GKVRGWVSFFRQKQENRWIVNGERKNALARAHDKGKYILELWPSWTSQTCPKCK |
| | HVHADNRHGDDFVCLQCGARLHADAEVATWNLAVVAIQGHSLPGPVREKSNDRK |
| | KSGSARKSKKANESGKVVGAWAAQATPKRATSKKETGTARNPVYNPLETQASCP |
| | AP |
| 236 | MPKIKKPTEISLLRKEVFPDLHFAKDRMRAASLVLKNEGREAAIEYLRVNHEDK |
| | PPNFMPPAKTPYVALSRPLEQWPIAQASIAIQKYIFGLTKDEFSATKKLLYGDK |
| | STPNTESRKRWFEVTGVPNFGYMSAQGLNAIFSGALARYEGVVQKVENRNKKRG |
| | SSGPDTAYHTPETLQKLAENNHVRVEDLGDMIDRLVHPPGIHRSIYGYQQVPPF |
| | AYDPDNPKGIILPKAYAGYTRKPHDIIEAMPNRLNIPEGQAGYIPEHQRDKLKK |
| | GGRVKRLRTTRVRVDATETVRAKAEALNAEKARLRGKEAILAVFQIEEDWALID |
| | MRGLLRNVYMRKLIAAGELTPTTLLGYFTETLTLDPRRTEATFCYHLRSEGALH |
| | AEYVRHGKNTRELLLDLTKDNEKIALVTIDLGQRNPLAAAIFRVGRDASGDLTE |
| | NSLEPVSRMLLPQAYLDQIKAYRDAYDSFRQNIWDTALASLTPEQQRQILAYEA |
| | YTPDDSKENVLRLLLGGNVMPDDLPWEDMTKNTHYISDRYLADGSSGDHNISHL |
| | SEFRPQLLKETRDAFEKAKIDTERGHVGYQKLSTRKDQLCKEILNWLEAEAVRL |
| | TRCKTMVLGLEDLNGPFFNQGKGKVRGWVSFFRQKQENRWIVNGFRKNALARAH |
| | DKGKYILELWPSWTSQTCPKCKHVHADNRHGDDFVCLQCGARLHADAEVATWNL |
| | AVVAIQGHSLPGPVREKSNDRKKSGSARKSKKANESGKVVGAWAAQATPKRATS |
| | KKETGTARNPVYNPLETQASCPAP |
| 237 | MPKIKKPTEISLLRKEVFPDLHFAKDRMRAASLVLKNEGREAAIEYLRVNHEDK |
| | PPNFMPPAKTPYVALSRPLEQWPIAQASIAIQKYIFGLTKDEFSATKKLLYGDK |
| | STPNTESRKRWFEVTGVPNFGYMSAQGLNAIFSGALARYEGVVQKVENRNKKRG |
| | SSGPDTAYHTPETLQKLAENNHVRVEDLGDMIDRLVHPPGIHRSIYGYQQVPPF |
| | AYDPDNPKGIILPKAYAGYTRKPHDIIEAMPNRLNIPEGQAGYIPEHQRDKLKK |
| | GGRVKRLRTTRVRVDATETVRAKAEALNAEKARLRGKEAILAVFQIEEDWALID |
| | MRGLLRNVYMRKLIAAGELTPTTLLGYFTETLTLDPRRTEATFCYHLRSEGALH |
| | AEYVRHGKNTRELLLDLTKDNEKIALVTIDLGQRNPLAAAIFRVGRDASGDLTE |
| | NSLEPVSRMLLPQAYLDQIKAYRDAYDSFRQNIWDTALASLTPEQQRQILAYEA |
| | YTPDDSKENVLRLLLGGNVMPDDLPWEDMTKNTHYISDRYLADGGDPSKVWFVP |
| | GPGSSGDHNISHLSEFRPQLLKETRDAFEKAKIDTERGHVGYQKLSTRKDQLCK |
| | EILNWLEAEAVRLTRCKTMVLGLEDLNGPFFNQGKGKVRGWVSFFRQKQENRWI |
| | VNGFRKNALARAHDKGKYILELWPSWTSQTCPKCKHVHADNRHGDDFVCLQCGA |
| | RLHADAEVATWNLAVVAIQGHSLPGPVREKSNDRKKSGSARKSKKANESGKVVG |
| | AWAAQATPKRATSKKETGTARNPVYNPLETQASCPAP |
| 238 | MPKIKKPTEISLLRKEVFPDLHFAKDRMRAASLVLKNEGREAAIEYLRVNHEDK |
| | PPNFMPPAKTPYVALSRPLEQWPIAQASIAIQKYIFGLTKDEFSATKKLLYGDK |
| | STPNTESRKRWFEVTGVPNFGYMSAQGLNAIFSGALARYEGVVQKVENRNKKRF |
| | EKLSEKNQLLIEEGQPVKDYVPDTAYHTPETLQKLAENNHVRVEDLGDMIDRLV |
| | HPPGIHRSIYGYQQVPPFAYDPDNPKGIILPKAYAGYTRKPHDIIEAMPNRLNI |
| | PEGQAGYIPEHQRDKLKKGGRVKRLRTTRVRVDATETVRAKAEALNAEKARLRG |
| | KEAILAVFQIEEDWALIDMRGLLRNVYMRKLIAAGELTPTTLLGYFTETLTLDP |
| | RRTEATFCYHLRSEGALHAEYVRHGKNTRELLLDLTKDNEKIALVTIDLGQRNP |
| | LAAAIFRVGRDASGDLTENSLEPVSRMLLPQAYLDQIKAYRDAYDSFRQNIWDT |
| | ALASLTPEQQRQILAYEAYTPDDSKENVLRLLLGGNVMPDDLPWEDMTKNTHYI |
| | SDRYLADVSNKSEIYFTSTDKGKTKDVMKSDHNISHLSEFRPQLLKETRDAFEK |
| | AKIDTERGHVGYQKLSTRKDQLCKEILNWLEAEAVRLTRCKTMVLGLEDLNGPF |
| | FNQGKGKVRGWVSFFRQKQENRWIVNGERKNALARAHDKGKYILELWPSWTSQT |
| | CPKCKHVHADNRHGDDFVCLQCGARLHADAEVATWNLAVVAIQGHSLPGPVREK |
| | SNDRKKSGSARKSKKANESGKVVGAWAAQATPKRATSKKETGTARNPVYNPLET |
| | QASCPAP |
| 239 | QAVIKYLSDKGAVDPPDFRPPAKCNIIAQSRPFDEWPICKASMAIQQHIYGLTK |
| | NEFDESSPGISSASHEQWFAKTGVDTHGFTHVQGLNLIFQHAKKRYEGVIKKVE |
| | NYNEKERKKFEGINERRSKEGMPLLEPRLRTAFGDDGKFAEKPGVNPSIYLYQQ |
| | TSPRPYDKTKHPYVHAPFELKEITTIPTQDDRLKIPFGAPGHVPEKHRSQLSMA |
| | KHKRRRAWYALSQNKPRPPKDGSKGRRSVRDLADLKAASLADAIPLVSRVGEDW |
| | VVIDGRGLLRNLRWRKLAHEGMTVEEMLGFFSGDPVIDPRRNVATFIYKAEHAT |
| | VKSRKPIGGAKRAREELLKATASSDGVIRQVGLISVDLGQTNPVAYEISRMHQA |
| | NGELVAEHLEYGLINDEQVNSIQRYRAAWDSMNESFRQKAIESLSMEAQDEIMQ |
| | ASTGAAKRTREAVLTMFGPNATLPWSRMSSNTTCISDALIEVGSSGDAQWAAYL |
| | RPRVNPETRALLNQAVWDLMKRSDEYERLSKRKLEMARQCVNFVVARAEKLTQC |
| | NNIGIVLENLVVRNFHGSGRRESGWEGFFEPKRENRWFMQVLHKAFSDLAQHRG |
| | VMVFEVHPAYSSQTCPACRYVDPKNRSSEDRERFKCLKCGRSENADREVATENI |
| | REIARTGVGLPKPDCERSRDVQTPGTARKSGRSLKSQDNLSEPKRVLQSKTRKK |
| | ITSTETQNEPLATDLKT |
| 240 | QAVIKYLSDKGAVDPPDFRPPAKCNIIAQSRPFDEWPICKASMAIQQHIYGLTK |
| | NEFDESSPGTSSASHEQWFAKTGVDTHGFTHVQGLNLIFQHAKKRYEGVIKKVE |
| | NYNEKERKKFEGINERRSKEGMPLLEPRLRTAFGDDGKFAEKPGVNPSIYLYQQ |
| | TSPRPYDKTKHPYVHAPFELKEITTIPTQDDRLKIPFGAPGHVPEKHRSQLSMA |
| | KHKRRRAWYALSQNKPRPPKDGSKGRRSVRDLADLKAASLADAIPLVSRVGEDW |
| | VVIDGRGLLRNLRWRKLAHEGMTVEEMLGFFSGDPVIDPRRNVATFIYKAEHAT |
| | VKSRKPIGGAKRAREELLKATASSDGVIRQVGLISVDLGQTNPVAYEISRMHQA |
| | NGELVAEHLEYGLINDEQVNSIQRYRAAWDSMNESFRQKAIESLSMEAQDEIMQ |
| | ASTGAAKRTREAVLTMFGPNATLPWSRMSSNTTCISDALIEVGKEEETNFVTSN |
| | GGSSGDAQWAAYLRPRVNPETRALLNQAVWDLMKRSDEYERLSKRKLEMARQCV |
| | NFVVARAEKLTQCNNIGIVLENLVVRNFHGSGRRESGWEGFFEPKRENRWEMQV |
| | LHKAFSDLAQHRGVMVFEVHPAYSSQTCPACRYVDPKNRSSEDRERFKCLKCGR |
| | SFNADREVATFNIREIARTGVGLPKPDCERSRDVQTPGTARKSGRSLKSQDNLS |
| | EPKRVLQSKTRKKITSTETQNEPLATDLKT |
| 241 | QAVIKYLSDKGAVDPPDERPPAKCNIIAQSRPFDEWPICKASMAIQQHIYGLTK |
| | NEFDESSPGTSSASHEQWFAKTGVDTHGFTHVQGLNLIFQHAKKRYEGVIKKVE |
| | NYNEKEGSSGLRTAFGDDGKFAEKPGVNPSIYLYQQTSPRPYDKTKHPYVHAPF |
| | ELKEITTIPTQDDRLKIPFGAPGHVPEKHRSQLSMAKHKRRRAWYALSQNKPRP |
| | PKDGSKGRRSVRDLADLKAASLADAIPLVSRVGFDWVVIDGRGLLRNLRWRKLA |
| | HEGMTVEEMLGFFSGDPVIDPRRNVATFIYKAEHATVKSRKPIGGAKRAREELL |
| | KATASSDGVIRQVGLISVDLGQTNPVAYEISRMHQANGELVAEHLEYGLINDEQ |
| | VNSIQRYRAAWDSMNESFRQKAIESLSMEAQDEIMQASTGAAKRTREAVLTMFG |
| | PNATLPWSRMSSNTTCISDALIEVGKEEETNFVTSNGPRKRTDAQWAAYLRPRV |
| | NPETRALLNQAVWDLMKRSDEYERLSKRKLEMARQCVNFVVARAEKLTQCNNIG |
| | IVLENLVVRNFHGSGRRESGWEGFFEPKRENRWEMQVLHKAFSDLAQHRGVMVE |
| | EVHPAYSSQTCPACRYVDPKNRSSEDRERFKCLKCGRSFNADREVATFNIREIA |
| | RTGVGLPKPDCERSRDVQTPGTARKSGRSLKSQDNLSEPKRVLQSKTRKKITST |
| | ETQNEPLATDLKT |
| 242 | QAVIKYLSDKGAVDPPDERPPAKCNIIAQSRPFDEWPICKASMAIQQHIYGLTK |
| | NEFDESSPGTSSASHEQWFAKTGVDTHGFTHVQGLNLIFQHAKKRYEGVIKKVE |
| | NYNEKEGSSGLRTAFGDDGKFAEKPGVNPSIYLYQQTSPRPYDKTKHPYVHAPF |
| | ELKEITTIPTQDDRLKIPFGAPGHVPEKHRSQLSMAKHKRRRAWYALSQNKPRP |
| | PKDGSKGRRSVRDLADLKAASLADAIPLVSRVGFDWVVIDGRGLLRNLRWRKLA |
| | HEGMTVEEMLGFFSGDPVIDPRRNVATFIYKAEHATVKSRKPIGGAKRAREELL |
| | KATASSDGVIRQVGLISVDLGQTNPVAYEISRMHQANGELVAEHLEYGLINDEQ |
| | VNSIQRYRAAWDSMNESFRQKAIESLSMEAQDEIMQASTGAAKRTREAVLTMFG |
| | PNATLPWSRMSSNTTCISDALIEVGSSGDAQWAAYLRPRVNPETRALLNQAVWD |
| | LMKRSDEYERLSKRKLEMARQCVNFVVARAEKLTQCNNIGIVLENLVVRNFHGS |
| | GRRESGWEGFFEPKRENRWFMQVLHKAFSDLAQHRGVMVFEVHPAYSSQTCPAC |
| | RYVDPKNRSSEDRERFKCLKCGRSFNADREVATENIREIARTGVGLPKPDCERS |
| | RDVQTPGTARKSGRSLKSQDNLSEPKRVLQSKTRKKITSTETQNEPLATDLKT |
| 243 | QAVIKYLSDKGAVDPPDFRPPAKCNIIAQSRPFDEWPICKASMAIQQHIYGLTK |
| | NEFDESSPGTSSASHEQWFAKTGVDTHGFTHVQGLNLIFQHAKKRYEGVIKKVE |
| | NYNEKEGSSGLRTAFGDDGKFAEKPGVNPSIYLYQQTSPRPYDKTKHPYVHAPF |
| | ELKEITTIPTQDDRLKIPFGAPGHVPEKHRSQLSMAKHKRRRAWYALSQNKPRP |
| | PKDGSKGRRSVRDLADLKAASLADAIPLVSRVGFDWVVIDGRGLLRNLRWRKLA |
| | HEGMTVEEMLGFFSGDPVIDPRRNVATFIYKAEHATVKSRKPIGGAKRAREELL |
| | KATASSDGVIRQVGLISVDLGQTNPVAYEISRMHQANGELVAEHLEYGLINDEQ |
| | VNSIQRYRAAWDSMNESFRQKAIESLSMEAQDEIMQASTGAAKRTREAVLTMFG |
| | PNATLPWSRMSSNTTCISDALIEVGKEEETNFVTSNGGSSGDAQWAAYLRPRVN |
| | PETRALLNQAVWDLMKRSDEYERLSKRKLEMARQCVNFVVARAEKLTQCNNIGI |
| | VLENLVVRNFHGSGRRESGWEGFFEPKRENRWFMQVLHKAFSDLAQHRGVMVFE |
| | VHPAYSSQTCPACRYVDPKNRSSEDRERFKCLKCGRSENADREVATENIREIAR |
| | TGVGLPKPDCERSRDVQTPGTARKSGRSLKSQDNLSEPKRVLQSKTRKKITSTE |
| | TQNEPLATDLKT |
| 244 | QAVIKYLSDKGAVDPPDERPPAKCNIIAQSRPFDEWPICKASMAIQQHIYGLTK |
| | NEFDESSPGTSSASHEQWFAKTGVDTHGFTHVQGLNLIFQHAKKRYEGVIKKVE |
| | NYNEKERKKFEGINERRSKEGMPLLEPRLRTAFGDDGKFAEKPGVNPSIYLYQQ |
| | TSPRPYDKTKHPYVHAPFELKEITTIPTQDDRLKIPFGAPGHVPEKHRSQLSMA |
| | KHKRRRAWYALSQNKPRPPKDGSKGRRSVRDLADLKAASLADAIPLVSRVGFDW |
| | VVIDGRGLLRNLRWRKLAHEGMTVEEMLGFFSGDPVIDPRRNVATFIYKAEHAT |
| | VKSRKPIGGAKRAREELLKATASSDGVIRQVGLISVDLGQTNPVAYEISRMHQA |
| | NGELVAEHLEYGLLNDEQVNSIQRYRAAWDSMNESFRQKAIESLSMEAQDEIMQ |
| | ASTGAAKRTREAVLTMFGPNATLPWSRMSSNTTCISDALIEVVSNKSEIYFTST |
| | DKGKTKDVMKSDAQWAAYLRPRVNPETRALLNQAVWDLMKRSDEYERLSKRKLE |
| | MARQCVNFVVARAEKLTQCNNIGIVLENLVVRNFHGSGRRESGWEGFFEPKREN |
| | RWFMQVLHKAFSDLAQHRGVMVFEVHPAYSSQTCPACRYVDPKNRSSEDRERFK |
| | CLKCGRSFNADREVATFNIREIARTGVGLPKPDCERSRDVQTPGTARKSGRSLK |
| | SQDNLSEPKRVLQSKTRKKITSTETQNEPLATDLKT |
| 271 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKPNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 324 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYRNVNLPEEY |
| | IGYYRKPNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 325 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNRAKINRKNEIAKLN |
| | GEQEISFEEIKAFDDKGYLLQKPSPNKSIYCYQSVSPKPFITSKYHNVNLPEEY |
| | IGYYRKPNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | GKLAPSYTVVLREAV |
| 326 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNRAKINRKNEIAKLN |
| | GEQEISFEEIKAFDDKGYLLQKPSPNKSIYCYQSVSPKPFITSKYRNVNLPEEY |
| | IGYYRKPNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 327 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKPNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GGLGPSYTVVLREAV |
| 328 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNRAKINRKNEIAKLN |
| | GEQEISFEEIKAFDDKGYLLQKPSPNKSIYCYQSVSPKPFITSKYRNVNLPEEY |
| | IGYYRKPNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GGLGPSYTVVLREAV |
| 329 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKPNEPIVSPYQFDRLRIPIGEPGYVPKWQYTELSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 330 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYRNVNLPEEY |
| | IGYYRKPNEPIVSPYQFDRLRIPIGEPGYVPKWQYTELSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 331 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNRAKINRKNEIAKLN |
| | GEQEISFEEIKAFDDKGYLLQKPSPNKSIYCYQSVSPKPFITSKYHNVNLPEEY |
| | IGYYRKPNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 332 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNRAKINRKNEIAKLN |
| | GEQEISFEEIKAFDDKGYLLQKPSPNKSIYCYQSVSPKPFITSKYRNVNLPEEY |
| | IGYYRKPNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | GKLAPSYTVVLREAV |
| 281 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKPNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GGLGPSYTVVLREAV |
| 282 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNRAKINRKNEIAKLN |
| | GEQEISFEEIKAFDDKGYLLQKPSPNKSIYCYQSVSPKPFITSKYRNVNLPEEY |
| | IGYYRKPNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | GKLAPSYTVVLREAV |
| 283 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNRIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTELSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 284 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNRIAKLN |
| | GEQEISFEEIKAFDDKGYLLQKPSPNKSIYCYQSVSPKPFITSKYHNVNLPEEY |
| | IGYYRKPNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 285 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNRIAKLN |
| | GEQEISFEEIKAFDDKGYLLQKPSPNKSIYCYQSVSPKPFITSKYHNVNLPEEY |
| | IGYYRKPNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKILISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 286 | IKPTVSQFLTPGFKLIRNHSRTAGRKLKNEGEEACKKFVRENEIPKDECPNFQG |
| | GPAIANIIAKSREFTEWEIYQSSLAIQEVIFTLPKDKLPEPILKEEWRAQWLSE |
| | HGLDTVPYKEAAGLNLIIKNAVNTYKGVQVKVDNKNKNNLAKINRKNEIAKLNG |
| | EQEISFEEIKAFDDKGYLLQKPSPNKSIYCYQSVSPKPFITSKYHNVNLPEEYI |
| | GYYRKSNEPIVSPYQFDRLRIPIGEPGYVPKWQYTELSKKENKRRKLSKRIKNV |
| | SPILGIICIKKDWCVFDMRGLLRTNHWKKYHKPTDSINDLFDYFTGDPVIDTKA |
| | NVVRFRYKMENGIVNYKPVREKKGKELLENICDQNGSCKLATVDVGQNNPVAIG |
| | LFELKKVNGELTKTLISRHPTPIDFCNKITAYRERYDKLESSIKLDAIKQLTSE |
| | QKIEVDNYNNNFTPQNTKQIVCSKLNINPNDLPWDKMTSGTHFISEKAQVSNKS |
| | EIYFTSTDKGKTKDVMKSDYKWFQDYKPKLSKEVRDALSDIEWRLRRESLEFNK |
| | LSKSREQDARQLANWISSMCDVIGIENLVKKNNFFGGSGKREPGWDNFYKPKKE |
| | NRWWINAIHKALTELSQNKGKRVILLPAMRTSITCPKCKYCDSKNRNGEKENCL |
| | KCGIELNADIDVATENLATVAITAQSMPKPTCERSGDAKKPVRARKAKAPEFHD |
| | KLAPSYTVVLREAV |
| 287 | KPTVSQFLTPGFKLIRNHSRTAGRKLKNEGEEACKKFVRENEIPKDECPNFQGG |
| | PAIANIIAKSREFTEWEIYQSSLAIQEVIFTLPKDKLPEPILKEEWRAQWLSEH |
| | GLDTVPYKEAAGLNLIIKNAVNTYKGVQVKVDNKNKNNLAKINRKNEIAKLNGE |
| | QEISFEEIKAFDDKGYLLQKPSPNKSIYCYQSVSPKPFITSKYHNVNLPEEYIG |
| | YYRKSNEPIVSPYQFDRLRIPIGEPGYVPKWQYTELSKKENKRRKLSKRIKNVS |
| | PILGIICIKKDWCVFDMRGLLRTNHWKKYHKPTDSINDLFDYFTGDPVIDTKAN |
| | VVRFRYKMENGIVNYKPVREKKGKELLENICDQNGSCKLATVDVGQNNPVAIGL |
| | FELKKVNGELTKTLISRHPTPIDFCNKITAYRERYDKLESSIKLDAIKQLTSEQ |
| | KIEVDNYNNNFTPQNTKQIVCSKLNINPNDLPWDKMTSGTHFISEKAQVSNKSE |
| | IYFTSTDKGKTKDVMKSDYKWFQDYKPKLSKEVRDALSDIEWRLRRESLEENKL |
| | SKSREQDARQLANWISSMCDVIGIENLVKKNNFFGGSGKREPGWDNFYKPKKEN |
| | RWWINAIHKALTELSQNKGKRVILLPAMRTSITCPKCKYCDSKNRNGEKENCLK |
| | CGIELNADIDVATENLATVAITAQSMPKPTCERSGDAKKPVRARKAKAPEFHDK |
| | LAPSYTVVLREAV |
| 288 | PTVSQFLTPGFKLIRNHSRTAGRKLKNEGEEACKKFVRENEIPKDECPNFQGGP |
| | AIANIIAKSREFTEWEIYQSSLAIQEVIFTLPKDKLPEPILKEEWRAQWLSEHG |
| | LDTVPYKEAAGLNLIIKNAVNTYKGVQVKVDNKNKNNLAKINRKNEIAKLNGEQ |
| | EISFEEIKAFDDKGYLLQKPSPNKSIYCYQSVSPKPFITSKYHNVNLPEEYIGY |
| | YRKSNEPIVSPYQFDRLRIPIGEPGYVPKWQYTELSKKENKRRKLSKRIKNVSP |
| | ILGIICIKKDWCVFDMRGLLRTNHWKKYHKPTDSINDLFDYFTGDPVIDTKANV |
| | VRFRYKMENGIVNYKPVREKKGKELLENICDQNGSCKLATVDVGQNNPVAIGLE |
| | ELKKVNGELTKTLISRHPTPIDFCNKITAYRERYDKLESSIKLDAIKQLTSEQK |
| | IEVDNYNNNFTPQNTKQIVCSKLNINPNDLPWDKMTSGTHFISEKAQVSNKSEI |
| | YFTSTDKGKTKDVMKSDYKWFQDYKPKLSKEVRDALSDIEWRLRRESLEFNKLS |
| | KSREQDARQLANWISSMCDVIGIENLVKKNNFFGGSGKREPGWDNFYKPKKENR |
| | WWINAIHKALTELSQNKGKRVILLPAMRTSITCPKCKYCDSKNRNGEKENCLKC |
| | GIELNADIDVATENLATVAITAQSMPKPTCERSGDAKKPVRARKAKAPEFHDKL |
| | APSYTVVLREAV |
| 289 | TVSQFLTPGFKLIRNHSRTAGRKLKNEGEEACKKFVRENEIPKDECPNFQGGPA |
| | IANIIAKSREFTEWEIYQSSLAIQEVIFTLPKDKLPEPILKEEWRAQWLSEHGL |
| | DTVPYKEAAGLNLIIKNAVNTYKGVQVKVDNKNKNNLAKINRKNEIAKLNGEQE |
| | ISFEEIKAFDDKGYLLQKPSPNKSIYCYQSVSPKPFITSKYHNVNLPEEYIGYY |
| | RKSNEPIVSPYQFDRLRIPIGEPGYVPKWQYTELSKKENKRRKLSKRIKNVSPI |
| | LGIICIKKDWCVFDMRGLLRTNHWKKYHKPTDSINDLFDYFTGDPVIDTKANVV |
| | RFRYKMENGIVNYKPVREKKGKELLENICDQNGSCKLATVDVGQNNPVAIGLFE |
| | LKKVNGELTKTLISRHPTPIDFCNKITAYRERYDKLESSIKLDAIKQLTSEQKI |
| | EVDNYNNNFTPQNTKQIVCSKLNINPNDLPWDKMTSGTHFISEKAQVSNKSEIY |
| | FTSTDKGKTKDVMKSDYKWFQDYKPKLSKEVRDALSDIEWRLRRESLEFNKLSK |
| | SREQDARQLANWISSMCDVIGIENLVKKNNFFGGSGKREPGWDNFYKPKKENRW |
| | WINAIHKALTELSQNKGKRVILLPAMRTSITCPKCKYCDSKNRNGEKENCLKCG |
| | IELNADIDVATENLATVAITAQSMPKPTCERSGDAKKPVRARKAKAPEFHDKLA |
| | PSYTVVLREAV |
| 290 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREA |
| 291 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLRE |
| 292 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLR |
| 293 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVL |
| 294 | MIKPTVSQFLTPGFKLIRNHSRTAWLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTELSKKENKRRKLSKRIKN |
| | VSPILGIICIKKDWIVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKWVILLPAMRTSITCPKCKYCDSKNRNGEKENC |
| | LKCGIELNADIDVATENLATVAITAQSMPKPTCERSGDAKKPVRARKAKAPEFH |
| | DKLAPSYTVVLREAV |
| 295 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQIARQLANWISSMCDVIGIENLVKKNNFFGGSGKREPGWDNFYKPKK |
| | ENRWWIYAIHKALTELSQNKGKWVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 296 | MIKPTVSQFLTPGFKLIRNHSRTAWLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVYQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWIYAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 297 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTELSKKENKRRKLSKRIKN |
| | VSPILGIICIKKDWIVEDMRGLLRTNHWKKYHKPTDSINDLFDYFTGDPVIDTK |
| | ANVVRFRYKMENGIVNYKPVREKKGKELLENICDQNGSCKLATVDVYQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQIARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 298 | MIKPTVSQFLTPGFKLIRNHSRTAYLKLKNEGEEACKKFVRENEIPKDECPNFQ |
| | GGPAIANIIALSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKYVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 299 | MIKPTVSQFLTPGFKLIRNHSRTAYLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHFVNLPEEY |
| | IGYYRKSNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMFTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 300 | MIKPTVSQFLTPGFKLIRNHSRTAFLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKYVILLPAMFTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 301 | MIKPTVSQFLTPGFKLIRNHSRTAFLKLKNEGEEACKKFVRENEIPKDECPNFQ |
| | GGPAIANIIALSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHFVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 302 | MIKPTVSQFLTPGFKLIRNHWRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLLTNHWKKYHKPTDSINDLFDYFTGDPVIDTK |
| | ANVVRFRYKMENGIVNYKPVREKKGKELLENICDQNGSCKLAIVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | DKLAPSYTVVLREAV |
| 303 | MIKPTVSQFLTPGFKLIRNHSRTAGLKLKNEGEEACKKFVRENEIPKDECPNFQ |
| | GGPAIANIIAKSREFTEWEIYQSMLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTELSKKENKWRKLSKRIKN |
| | VSPILGIIFIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 304 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKMRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLAIVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQWARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 305 | MIKPTVSQFLTPGFKLIRNHFRTAGLKLKNEGEEACKKFVRENEIPKDECPNFQ |
| | GGPAIANIIAKSREFTEWEIYQSMLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQYARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | DKLAPSYTVVLREAV |
| 306 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTELSKKENKRRKLSKRIKN |
| | VSPILGIIIIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQLARQLANWISSMCDVIGIENLVKKNNFFGGSGKREPGWDNFYKPKK |
| | ENRWWIFAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 307 | MIKPTVSQFLTPGFKLIRNHSRTAGLKLKNEGEEACKKFVRENEIPKDECPNFQ |
| | GGPAIANIIAKSREFTEWEIYQSFLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKLRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQLARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 308 | MIKPTVSQFLTPGFKLIRNHSRTAGLKLKNEGEEACKKFVRENEIPKDECPNFQ |
| | GGPAIANIIAKSREFTEWEIYQSFLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIIIIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLAWVAITAQSMPKPTCERSGDAKKPVRARKAKAPEFH |
| | DKLAPSYTVVLREAV |
| 309 | MIKPTVSQFLTPGFKLIRNHWRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQWARQLANWISSMCDVIGIENLVKKNNFFGGSGKREPGWDNFYKPKK |
| | ENRWWINAIHKALTELSQNKGKRVILLPAMYTSITCPKCKYCDSKNRNGEKENC |
| | LKCGIELNADIDVATENLATVAITAQSMPKPTCERSGDAKKPVRARKAKAPEFH |
| | DKLAPSYTVVLREAV |
| 310 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTELSKKENKLRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWIFAIHKALTELSQNKGKRVILLPAMRTSITCPKCKYCDSKNRNGEKFNC |
| | LKCGIELNADIDVATENLAWVAITAQSMPKPTCERSGDAKKPVRARKAKAPEFH |
| | DKLAPSYTVVLREAV |
| 311 | MIKPTVSQFLTPGFKLIRNHSRTALLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLAVVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMWTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 312 | MIKPTVSQFLTPGFKLIRNHSRTALLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKLWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQWTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | DKLAPSYTVVLREAV |
| 313 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKWRKLSKRIKN |
| | VSPILGIICIKKDWVVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQYARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | DKLAPSYTVVLREAV |
| 314 | MIKPTVSQFLTPGFKLIRNHSRTAGLKLKNEGEEACKKFVRENEIPKDECPNFQ |
| | GGPAIANIIAKSREFTEWEIYQSILAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVMYKPVREKKGKELLENICDQNGSCKLATVDVGQNNPVAI |
| | GLFELKKVNGELTKTLISRHPTPIWFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 315 | MIKPTVSQFLTPGFKLIRNHYRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHYVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCILATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | DKLAPSYTVVLREAV |
| 316 | MIKPTVSQFLTPGFKLIRNHSRTAVLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVFQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSIVCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 317 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKIAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVLYKPVREKKGKELLENICDQNGSCILATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 318 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIIWIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQWTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMWTSITCPKCKYCDSKNRNGEKENC |
| | LKCGIELNADIDVATENLATVAITAQSMPKPTCERSGDAKKPVRARKAKAPEFH |
| | DKLAPSYTVVLREAV |
| 319 | MIKPTVSQFLTPGFKLIRNHSRTAGLKLKNEGEEACKKFVRENEIPKDECPNFQ |
| | GGPAIANIIAKSREFTEWEIYQSILAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVLPYQFDRLRIPIGEPGYVPKWQYTELSKKENKRRKLSKRIKN |
| | VSPILGIIYIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 320 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKIAVNTYKGVQVKVDNKNKNNLAKINRKNEIAKLN |
| | GEQEISFEEIKAFDDKGYLLQKPSPNKSIYCYQSVSPKPFITSKYHYVNLPEEY |
| | IGYYRKSNEPIVSPYQFDRLRIPIGEPGYVPKWQYTELSKKENKRRKLSKRIKN |
| | VSPILGIICIKKDWCVFDMRGLLRTNHWWKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | DKLAPSYTVVLREAV |
| 321 | MIKPTVSQFLTPGFKLIRNHSRTAILKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVLPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVMYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 322 | MIKPTVSQFLTPGFKLIRNHSRTAILKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKLSKRIKN |
| | VSPILGIIYIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIWFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 333 | MIKPTVSQFLTPGFKLIRNHYRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTELSKKENKRRKLSKRIKN |
| | VSPILGIICIKKDWCVFDMRGLLRTNHWWKYHKPTDSINDLFDYFTGDPVIDTK |
| | ANVVRFRYKMENGIVLYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 334 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTELSKKENKRRKLSKRIKN |
| | VSPILGIICIKKDWCVEDMRGLLRTNHWKKYHKPTDSINDLEDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | DKLAPSYTVVLREAV |
| 335 | MIKPTVSQFLTPGFKLIRNHSRTAGLKLKNEGEEACKKFVRENEIPKDECPNFQ |
| | PPAKANIIAKSREFTEWEIYQSSLAIQEVIFTLPKDKLPEPILKEEWRAQWLSE |
| | HGLDTVPYKEAAGLNLIIKNAVNTYKGVQVKVDNKNKNNLAKINRKNEIAKLNG |
| | EQEISFEEIKAFDDKGYLLQKPSPNKSIYCYQSVSPKPFITSKYHNVNLPEEYI |
| | GYYRKSNEPIVSPYQFDRLRIPIGEPGYVPKWQYTELSKKENKRRKLSKRIKNV |
| | SPILGIICIKKDWCVFDMRGLLRTNHWKKYHKPTDSINDLFDYFTGDPVIDTKA |
| | NVVRFRYKMENGIVNYKPVREKKGKELLENICDQNGSCKLATVDVGQNNPVAIG |
| | LFELKKVNGELTKTLISRHPTPIDFCNKITAYRERYDKLESSIKLDAIKQLTSE |
| | QKIEVDNYNNNFTPQNTKQIVCSKLNINPNDLPWDKMTSGTHFISEKAQVSNKS |
| | EIYFTSTDKGKTKDVMKSDYKWFQDYKPKLSKEVRDALSDIEWRLRRESLEFNK |
| | LSKSREQDARQLANWISSMCDVIGIENLVKKNNFFGGSGKREPGWDNFYKPKKE |
| | NRWWINAIHKALTELSQNKGKRVILLPAMRTSITCPKCKYCDSKNRNGEKENCL |
| | KCGIELNADIDVATENLATVAITAQSMPKPTCERSGDAKKPVRARKAKAPEFHD |
| | KLAPSYTVVLREAV |
| 336 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPEKHRSQLSMAKHKRRRAWYALSQ |
| | NKPRPPKDGSKGRRSVRDLADLKAASLADAILGIICIKKDWCVEDMRGLLRTNH |
| | WKKYHKPTDSINDLEDYFTGDPVIDTKANVVRFRYKMENGIVNYKPVREKKGKE |
| | LLENICDQNGSCKLATVDVGQNNPVAIGLFELKKVNGELTKTLISRHPTPIDFC |
| | NKITAYRERYDKLESSIKLDAIKQLTSEQKIEVDNYNNNFTPQNTKQIVCSKLN |
| | INPNDLPWDKMTSGTHFISEKAQVSNKSEIYFTSTDKGKTKDVMKSDYKWFQDY |
| | KPKLSKEVRDALSDIEWRLRRESLEFNKLSKSREQDARQLANWISSMCDVIGIE |
| | NLVKKNNFFGGSGKREPGWDNFYKPKKENRWWINAIHKALTELSQNKGKRVILL |
| | PAMRTSITCPKCKYCDSKNRNGEKFNCLKCGIELNADIDVATENLATVAITAQS |
| | MPKPTCERSGDAKKPVRARKAKAPEFHDKLAPSYTVVLREAV |
| 337 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPEWMRTAGEKTNPRTQKKFMHPGL |
| | STRKNKRMRLPRSVRSAPLGAILGIICIKKDWCVEDMRGLLRTNHWKKYHKPTD |
| | SINDLEDYFTGDPVIDTKANVVRFRYKMENGIVNYKPVREKKGKELLENICDQN |
| | GSCKLATVDVGQNNPVAIGLFELKKVNGELTKTLISRHPTPIDFCNKITAYRER |
| | YDKLESSIKLDAIKQLTSEQKIEVDNYNNNFTPQNTKQIVCSKLNINPNDLPWD |
| | KMTSGTHFISEKAQVSNKSEIYFTSTDKGKTKDVMKSDYKWFQDYKPKLSKEVR |
| | DALSDIEWRLRRESLEFNKLSKSREQDARQLANWISSMCDVIGIENLVKKNNFF |
| | GGSGKREPGWDNFYKPKKENRWWINAIHKALTELSQNKGKRVILLPAMRTSITC |
| | PKCKYCDSKNRNGEKFNCLKCGIELNADIDVATENLATVAITAQSMPKPTCERS |
| | GDAKKPVRARKAKAPEFHDKLAPSYTVVLREAV |
| 338 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPEWQRPHLSMKCKRVRMWYARANW |
| | RRKPGRRSVLNEARLKEASAKGAILGIICIKKDWCVFDMRGLLRTNHWKKYHKP |
| | TDSINDLFDYFTGDPVIDTKANVVRFRYKMENGIVNYKPVREKKGKELLENICD |
| | QNGSCKLATVDVGQNNPVAIGLFELKKVNGELTKTLISRHPTPIDFCNKITAYR |
| | ERYDKLESSIKLDAIKQLTSEQKIEVDNYNNNFTPQNTKQIVCSKLNINPNDLP |
| | WDKMTSGTHFISEKAQVSNKSEIYFTSTDKGKTKDVMKSDYKWFQDYKPKLSKE |
| | VRDALSDIEWRLRRESLEFNKLSKSREQDARQLANWISSMCDVIGIENLVKKNN |
| | FFGGSGKREPGWDNFYKPKKENRWWINAIHKALTELSQNKGKRVILLPAMRTSI |
| | TCPKCKYCDSKNRNGEKFNCLKCGIELNADIDVATENLATVAITAQSMPKPTCE |
| | RSGDAKKPVRARKAKAPEFHDKLAPSYTVVLREAV |
| 339 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPEWQREAGTAISPKTGKAVTVPGL |
| | SPKKNKRMRRYWRSEKEKAQDAILGIICIKKDWCVEDMRGLLRTNHWKKYHKPT |
| | DSINDLFDYFTGDPVIDTKANVVRFRYKMENGIVNYKPVREKKGKELLENICDQ |
| | NGSCKLATVDVGQNNPVAIGLFELKKVNGELTKTLISRHPTPIDFCNKITAYRE |
| | RYDKLESSIKLDAIKQLTSEQKIEVDNYNNNFTPQNTKQIVCSKLNINPNDLPW |
| | DKMTSGTHFISEKAQVSNKSEIYFTSTDKGKTKDVMKSDYKWFQDYKPKLSKEV |
| | RDALSDIEWRLRRESLEFNKLSKSREQDARQLANWISSMCDVIGIENLVKKNNF |
| | FGGSGKREPGWDNFYKPKKENRWWINAIHKALTELSQNKGKRVILLPAMRTSIT |
| | CPKCKYCDSKNRNGEKFNCLKCGIELNADIDVATENLATVAITAQSMPKPTCER |
| | SGDAKKPVRARKAKAPEFHDKLAPSYTVVLREAV |
| 340 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPEWQRSQLTTQKHRRKRSWYSAQK |
| | WKPRTGRTSTFDPDRLNCARAQGAILGIICIKKDWCVFDMRGLLRTNHWKKYHK |
| | PTDSINDLFDYFTGDPVIDTKANVVRFRYKMENGIVNYKPVREKKGKELLENIC |
| | DQNGSCKLATVDVGQNNPVAIGLFELKKVNGELTKTLISRHPTPIDFCNKITAY |
| | RERYDKLESSIKLDAIKQLTSEQKIEVDNYNNNFTPQNTKQIVCSKLNINPNDL |
| | PWDKMTSGTHFISEKAQVSNKSEIYFTSTDKGKTKDVMKSDYKWFQDYKPKLSK |
| | EVRDALSDIEWRLRRESLEFNKLSKSREQDARQLANWISSMCDVIGIENLVKKN |
| | NFFGGSGKREPGWDNFYKPKKENRWWINAIHKALTELSQNKGKRVILLPAMRTS |
| | ITCPKCKYCDSKNRNGEKFNCLKCGIELNADIDVATENLATVAITAQSMPKPTC |
| | ERSGDAKKPVRARKAKAPEFHDKLAPSYTVVLREAV |
| 341 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPEPHREGLTGRKDRRMRRYYETER |
| | GTKLKRPPLTAKGRADKANEAILGIICIKKDWCVFDMRGLLRTNHWKKYHKPTD |
| | SINDLFDYFTGDPVIDTKANVVRFRYKMENGIVNYKPVREKKGKELLENICDQN |
| | GSCKLATVDVGQNNPVAIGLFELKKVNGELTKTLISRHPTPIDFCNKITAYRER |
| | YDKLESSIKLDAIKQLTSEQKIEVDNYNNNFTPQNTKQIVCSKLNINPNDLPWD |
| | KMTSGTHFISEKAQVSNKSEIYFTSTDKGKTKDVMKSDYKWFQDYKPKLSKEVR |
| | DALSDIEWRLRRESLEFNKLSKSREQDARQLANWISSMCDVIGIENLVKKNNFF |
| | GGSGKREPGWDNFYKPKKENRWWINAIHKALTELSQNKGKRVILLPAMRTSITC |
| | PKCKYCDSKNRNGEKFNCLKCGIELNADIDVATENLATVAITAQSMPKPTCERS |
| | GDAKKPVRARKAKAPEFHDKLAPSYTVVLREAV |
| 342 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPEWQRLKCSTNKHRRMRQWSNQDY |
| | KPKAGRRAKPLEFQAHLTRERAKGAILGIICIKKDWCVEDMRGLLRTNHWKKYH |
| | KPTDSINDLFDYFTGDPVIDTKANVVRFRYKMENGIVNYKPVREKKGKELLENI |
| | CDQNGSCKLATVDVGQNNPVAIGLFELKKVNGELTKTLISRHPTPIDFCNKITA |
| | YRERYDKLESSIKLDAIKQLTSEQKIEVDNYNNNFTPQNTKQIVCSKLNINPND |
| | LPWDKMTSGTHFISEKAQVSNKSEIYFTSTDKGKTKDVMKSDYKWFQDYKPKLS |
| | KEVRDALSDIEWRLRRESLEFNKLSKSREQDARQLANWISSMCDVIGIENLVKK |
| | NNFFGGSGKREPGWDNFYKPKKENRWWINAIHKALTELSQNKGKRVILLPAMRT |
| | SITCPKCKYCDSKNRNGEKFNCLKCGIELNADIDVATENLATVAITAQSMPKPT |
| | CERSGDAKKPVRARKAKAPEFHDKLAPSYTVVLREAV |
| 343 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQREAGTAISPKTGKAVTVPGL |
| | SPKKNKRMRRYWRSEKEKAQDAILGIICIKKDWCVEDMRGLLRTNHWKKYHKPT |
| | DSINDLFDYFTGDPVIDTKANVVRFRYKMENGIVNYKPVREKKGKELLENICDQ |
| | NGSCKLATVDVGQNNPVAIGLFELKKVNGELTKTLISRHPTPIDFCNKITAYRE |
| | RYDKLESSIKLDAIKQLTSEQKIEVDNYNNNFTPQNTKQIVCSKLNINPNDLPW |
| | DKMTSGTHFISEKAQVSNKSEIYFTSTDKGKTKDVMKSDYKWFQDYKPKLSKEV |
| | RDALSDIEWRLRRESLEFNKLSKSREQDARQLANWISSMCDVIGIENLVKKNNF |
| | FGGSGKREPGWDNFYKPKKENRWWINAIHKALTELSQNKGKRVILLPAMRTSIT |
| | CPKCKYCDSKNRNGEKFNCLKCGIELNADIDVATENLATVAITAQSMPKPTCER |
| | SGDAKKPVRARKAKAPEFHDKLAPSYTVVLREAV |
| 344 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKTGKAVTVPGL |
| | SPKKNKRMRRYWRSEKEKAQDAILGIICIKKDWCVFDMRGLLRTNHWKKYHKPT |
| | DSINDLFDYFTGDPVIDTKANVVRFRYKMENGIVNYKPVREKKGKELLENICDQ |
| | NGSCKLATVDVGQNNPVAIGLFELKKVNGELTKTLISRHPTPIDFCNKITAYRE |
| | RYDKLESSIKLDAIKQLTSEQKIEVDNYNNNFTPQNTKQIVCSKLNINPNDLPW |
| | DKMTSGTHFISEKAQVSNKSEIYFTSTDKGKTKDVMKSDYKWFQDYKPKLSKEV |
| | RDALSDIEWRLRRESLEFNKLSKSREQDARQLANWISSMCDVIGIENLVKKNNF |
| | FGGSGKREPGWDNFYKPKKENRWWINAIHKALTELSQNKGKRVILLPAMRTSIT |
| | CPKCKYCDSKNRNGEKFNCLKCGIELNADIDVATENLATVAITAQSMPKPTCER |
| | SGDAKKPVRARKAKAPEFHDKLAPSYTVVLREAV |
| 345 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPKWQYTFLSKKENKRRKAVTVPGL |
| | SPKKNKRMRRYWRSEKEKAQDAILGIICIKKDWCVFDMRGLLRTNHWKKYHKPT |
| | DSINDLFDYFTGDPVIDTKANVVRFRYKMENGIVNYKPVREKKGKELLENICDQ |
| | NGSCKLATVDVGQNNPVAIGLFELKKVNGELTKTLISRHPTPIDFCNKITAYRE |
| | RYDKLESSIKLDAIKQLTSEQKIEVDNYNNNFTPQNTKQIVCSKLNINPNDLPW |
| | DKMTSGTHFISEKAQVSNKSEIYFTSTDKGKTKDVMKSDYKWFQDYKPKLSKEV |
| | RDALSDIEWRLRRESLEFNKLSKSREQDARQLANWISSMCDVIGIENLVKKNNF |
| | FGGSGKREPGWDNFYKPKKENRWWINAIHKALTELSQNKGKRVILLPAMRTSIT |
| | CPKCKYCDSKNRNGEKFNCLKCGIELNADIDVATENLATVAITAQSMPKPTCER |
| | SGDAKKPVRARKAKAPEFHDKLAPSYTVVLREAV |
| 346 | MIKPTVSQFLTPGFKLIRNHSRTAGLKLKNEGEEACKKFVRENEIPKDECPNFQ |
| | PPAKANIIAKSREFTEWEIYQSSLAIQEVIFTLPKDKLPEPILKEEWRAQWLSE |
| | HGLDTVPYKEAAGLNLIIKNAVNTYKGVQVKVDNKNKNNLAKINRKNEIAKLNG |
| | EQEISFEEIKAFDDKGYLLQKPSPNKSIYCYQSVSPKPFITSKYHNVNLPEEYI |
| | GYYRKSNEPIVSPYQFDRLRIPIGEPGYVPEWQREAGTAISPKRRKAVTVPGLS |
| | PKKNKRMRRYWRSEKEKAQDAILGIICIKKDWCVFDMRGLLRTNHWKKYHKPTD |
| | SINDLFDYFTGDPVIDTKANVVRFRYKMENGIVNYKPVREKKGKELLENICDQN |
| | GSCKLATVDVGQNNPVAIGLFELKKVNGELTKTLISRHPTPIDFCNKITAYRER |
| | YDKLESSIKLDAIKQLTSEQKIEVDNYNNNFTPQNTKQIVCSKLNINPNDLPWD |
| | KMTSGTHFISEKAQVSNKSEIYFTSTDKGKTKDVMKSDYKWFQDYKPKLSKEVR |
| | DALSDIEWRLRRESLEFNKLSKSREQDARQLANWISSMCDVIGIENLVKKNNFF |
| | GGSGKREPGWDNFYKPKKENRWWINAIHKALTELSQNKGKRVILLPAMRTSITC |
| | PKCKYCDSKNRNGEKFNCLKCGIELNADIDVATENLATVAITAQSMPKPTCERS |
| | GDAKKPVRARKAKAPEFHDKLAPSYTVVLREAV |
| 347 | MIKPTVSQFLTPGFKLIRNHSRTAGLKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKSNEPIVSPYQFDRLRIPIGEPGYVPEWQREAGTAISPKRRKAVTVPGL |
| | SPKKNKRMRRYWRSEKEKAQDAILGIICIKKDWCVFDMRGLLRTNHWKKYHKPT |
| | DSINDLEDYFTGDPVIDTKANVVRFRYKMENGIVNYKPVREKKGKELLENICDQ |
| | NGSCKLATVDVGQNNPVAIGLFELKKVNGELTKTLISRHPTPIDFCNKITAYRE |
| | RYDKLESSIKLDAIKQLTSEQKIEVDNYNNNFTPQNTKQIVCSKLNINPNDLPW |
| | DKMTSGTHFISEKAQVSNKSEIYFTSTDKGKTKDVMKSDYKWFQDYKPKLSKEV |
| | RDALSDIEWRLRRESLEFNKLSKSREQDARQLANWISSMCDVIGIENLVKKNNF |
| | FGGSGKREPGWDNFYKPKKENRWWINAIHKALTELSQNKGKRVILLPAMRTSIT |
| | CPKCKYCDSKNRNGEKFNCLKCGIELNADIDVATENLATVAITAQSMPKPTCER |
| | SGDAKKPVRARKAKAPEFHDKLAPSYTVVLREAV |
| 379 | MIKPTVSQFLTPGFKLIRNHSRTAGRKLKNEGEEACKKFVRENEIPKDECPNFQ |
| | GGPAIANIIAKSREFTEWEIYQSSLAIQEVIFGLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPGPNKSIYCYQSVSPKPFITSKYRNVNLPEEY |
| | IGYYRKPNEPIVSPYQFDRLRIPIGEPGYVPKWQYTFLSKKENKRRKLSKRIKN |
| | VSPILGIICIKKDWCVEDMRGLLRTNHWKKYHKPTDSINDLEDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDELNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALNDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 380 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAQGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKPNEPIVSPYQFDRLRIPIGEPGYVPKWQYTFLSKKKNKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRKALNDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 381 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKKNLAKINRKNEIAKLN |
| | GEQEISFEEIKAFDDKGYLLQKPSPNKSIYCYQSVSPKPFITSKYRNVNLPEEY |
| | IGYYRKPNEPIVSPYQFDRLRIPIGEPGYVPKWQYTFLSKKKNKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVKKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 382 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKKNLAKINRKNEIAKLN |
| | GEQEISFEEIKAFDDKGYLLQKPGPNKSIYCYQSVSPKPFITSKYHNVNLPEEY |
| | IGYYRKPNEPIVSPYQFDRLRIPIGEPGYVPKWQYTFLSKKKNKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALNDIEWRLRRESLEFN |
| | KLSKSREQLARQLANWISSMCDVIGIENLVKKNNFFGGSGKREPGWDNFYKPKK |
| | ENRWWINAIHKALTELSQNKGKRVILLPAMRTSITCPKCKYCDKKNRNGEKFNC |
| | LKCGIELNADIDVATENLATVAITAQSMPKPTCERSGDAKKPVRARKAKAPEFH |
| | GKLAPSYTVVLREAV |
| 383 | MIKPTVSQFLTPGFKLIRNHLRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPGPNKSIYCYQSVSPKPFITSKYHNVNLPEEY |
| | IGYSRKPNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 384 | MIKPTVSQFLTPGFKLIRNHSRTAGRKLKNEGEEACKKFVRENEIPKDECPNFQ |
| | GGPAIANIIAKSREFTEWEIYQSSLAIQEVIFGLPKDKLPEPILKEEWRAQWLS |
| | EHGLDTVPYKEAQGLNLIIKNAVNTYKGVQVKVDNKNKNNLAKINRKNEIAKLN |
| | GEQEISFEEIKAFDDKGYLLQKPGPNKSIYCYQSVSPKPFITSKYRNVNLPEEY |
| | IGYSRKPNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDELNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRKALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | GKLAPSYTVVLREAV |
| 385 | MIKPTVSQFLTPGFKLIRNHLRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAQGLNLIIKNAVNTYKGVQVKVDNKNKKNLAKINRKNEIAKLN |
| | GEQEISFEEIKAFDDKGYLLQKPGPNKSIYCYQSVSPKPFITSKYHNVNLPEEY |
| | IGYSRKPNEPIVSPYQFDRLRIPIGEPGYVPKWQYTELSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALNDIEWRLRRESLEFN |
| | KLSKSREQLARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 386 | MIKPTVSQFLTPGFKLIRNHLRTAGRKLKNEGEEACKKFVRENEIPKDECPNFQ |
| | GGPAIANIIAKSREFTEWEIYRSSLAIQEVIFTLPKDKLPEPILKEEWRAQWLS |
| | EHGLDTVPYKEAAGLNLIIKNAVNTYKGVQVKVDNKNKKNRAKINRKNEIAKLN |
| | GEQEISFEEIKAFDDKGYLLQKPSPNKSIYCYQSVSPKPFITSKYHNVNLPEEY |
| | IGYSRKPNEPIVSPYQFDRLRIPIGEPGYVPKWQRTFLSKKKNKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVKKSDYKWFQDYKPKLSKEVRDALSDIEWRLRRESLEFN |
| | KLSKSREQDARRLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 387 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAQGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYSRKPNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDELNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRKALSDIEWRLRRESLEFN |
| | KLSKSREQDARRLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 388 | MIKPTVSQFLTPGFKLIRNHLRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAQGLNLIIKNAVNTYKGVQVKVDNKNKKNLAKINRKNEIAKLN |
| | GEQEISFEEIKAFDDKGYLLQKPSPNKSIYCYQSVSPKPFITSKYHNVNLPEEY |
| | IGYSRKPNEPIVSPYQFDRLRIPIGEPGYVPKWQRTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDELNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 389 | MIKPTVSQFLTPGFKLIRNHSRTAGRKLKNEGEEACKKFVRENEIPKDECPNFQ |
| | GGPAIANIIAKSREFTEWEIYRSSLAIQEVIFGLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKPNEPIVSPYQFDRLRIPIGEPGYVPKWQYTFLSKKKNKRRKLSKRIKN |
| | VSPILGIIRIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVKKSDYKWFQDYKPKLSKEVRKALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 390 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKKNLAKINRKNEIAKLN |
| | GEQEISFEEIKAFDDKGYLLQKPGPNKSIYCYQSVSPKPFITSKYHNVNLPEEY |
| | IGYYRKPNEPIVSPYQFDRLRIPIGEPGYVPKWQYTELSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVKKSDYKWFQDYKPKLSKEVRDALNDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 391 | MIKPTVSQFLTPGFKLIRNHLRTAGRKLKNEGEEACKKFVRENEIPKDECPNFQ |
| | GGPAIANIIAKSREFTEWEIYQSSLAIQEVIFGLPKDKLPEPILKEEWRAQWLS |
| | EHGLDTVPYKEAAGLNLIIKNAVNTYKGVQVKVDNKNKKNLAKINRKNEIAKLN |
| | GEQEISFEEIKAFDDKGYLLQKPSPNKSIYCYQSVSPKPFITSKYRNVNLPEEY |
| | IGYSRKPNEPIVSPYQFDRLRIPIGEPGYVPKWQYTELSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 392 | MIKPTVSQFLTPGFKLIRNHLRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAQGLNLIIKNAVNTYKGVQVKVDNKNKKNLAKINRKNEIAKLN |
| | GEQEISFEEIKAFDDKGYLLQKPGPNKSIYCYQSVSPKPFITSKYHNVNLPEEY |
| | IGYYRKPNEPIVSPYQFDRLRIPIGEPGYVPKWQYTELSKKKNKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRKALSDIEWRLRRESLEFN |
| | KLSKSREQDARRLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 393 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAQGLNLIIKNAVNTYKGVQVKVDNKNKNNRAKINRKNEIAKLN |
| | GEQEISFEEIKAFDDKGYLLQKPGPNKSIYCYQSVSPKPFITSKYHNVNLPEEY |
| | IGYSRKPNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | GKLAPSYTVVLREAV |
| 394 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAQGLNLIIKNAVNTYKGVQVKVDNKNKKNLAKINRKNEIAKLN |
| | GEQEISFEEIKAFDDKGYLLQKPSPNKSIYCYQSVSPKPFITSKYHNVNLPEEY |
| | IGYSRKPNEPIVSPYQFDRLRIPIGEPGYVPKWQYTELSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVKKSDYKWFQDYKPKLSKEVRDALNDIEWRLRRESLEEN |
| | KLSKSREQLARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | GKLAPSYTVVLREAV |
| 395 | MIKPTVSQFLTPGFKLIRNHSRTAGRKLKNEGEEACKKFVRENEIPKDECPNFQ |
| | GGPAIANIIAKSREFTEWEIYQSSLAIQEVIFGLPKDKLPEPILKEEWRAQWLS |
| | EHGLDTVPYKEAQGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYSRKPNEPIVSPYQFDRLRIPIGEPGYVPKWQYTFLSKKKNKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFLNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 396 | MIKPTVSQFLTPGFKLIRNHSRTAGRKLKNEGEEACKKFVRENEIPKDECPNFQ |
| | GGPAIANIIAKSREFTEWEIYQSSLAIQEVIFGLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPGPNKSIYCYQSVSPKPFITSKYHNVNLPEEY |
| | IGYSRKPNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVKKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 397 | MIKPTVSQFLTPGFKLIRNHLRTAGRKLKNEGEEACKKFVRENEIPKDECPNFQ |
| | GGPAIANIIAKSREFTEWEIYRSSLAIQEVIFGLPKDKLPEPILKEEWRAQWLS |
| | EHGLDTVPYKEAAGLNLIIKNAVNTYKGVQVKVDNKNKKNLAKINRKNEIAKLN |
| | GEQEISFEEIKAFDDKGYLLQKPGPNKSIYCYQSVSPKPFITSKYHNVNLPEEY |
| | IGYYRKPNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALNDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDKKNRNGEKENC |
| | LKCGIELNADIDVATENLATVAITAQSMPKPTCERSGDAKKPVRARKAKAPEFH |
| | GKLAPSYTVVLREAV |
| 398 | MIKPTVSQFLTPGFKLIRNHLRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAQGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYSRKPNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFLNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVKKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 399 | MIKPTVSQFLTPGFKLIRNHSRTAGRKLKNEGEEACKKFVRENEIPKDECPNFQ |
| | GGPAIANIIAKSREFTEWEIYQSSLAIQEVIFGLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKPNEPIVSPYQFDRLRIPIGEPGYVPKWQYTELSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALNDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 400 | MIKPTVSQFLTPGFKLIRNHSRTAGRKLKNEGEEACKKFVRENEIPKDECPNFQ |
| | GGPAIANIIAKSREFTEWEIYQSSLAIQEVIFGLPKDKLPEPILKEEWRAQWLS |
| | EHGLDTVPYKEAAGLNLIIKNAVNTYKGVQVKVDNKNKKNLAKINRKNEIAKLN |
| | GEQEISFEEIKAFDDKGYLLQKPSPNKSIYCYQSVSPKPFITSKYHNVNLPEEY |
| | IGYYRKPNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALNDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | GKLAPSYTVVLREAV |
| 401 | MIKPTVSQFLTPGFKLIRNHSRTAGRKLKNEGEEACKKFVRENEIPKDECPNFQ |
| | GGPAIANIIAKSREFTEWEIYQSSLAIQEVIFGLPKDKLPEPILKEEWRAQWLS |
| | EHGLDTVPYKEAAGLNLIIKNAVNTYKGVQVKVDNKNKKNLAKINRKNEIAKLN |
| | GEQEISFEEIKAFDDKGYLLQKPSPNKSIYCYQSVSPKPFITSKYHNVNLPEEY |
| | IGYYRKPNEPIVSPYQFDRLRIPIGEPGYVPKWQYTFLSKKKNKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALNDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 402 | MIKPTVSQFLTPGFKLIRNHSRTAGRKLKNEGEEACKKFVRENEIPKDECPNFQ |
| | GGPAIANIIAKSREFTEWEIYQSSLAIQEVIFGLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYSRKPNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALNDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 403 | MIKPTVSQFLTPGFKLIRNHSRTAGRKLKNEGEEACKKFVRENEIPKDECPNFQ |
| | GGPAIANIIAKSREFTEWEIYQSSLAIQEVIFGLPKDKLPEPILKEEWRAQWLS |
| | EHGLDTVPYKEAAGLNLIIKNAVNTYKGVQVKVDNKNKKNLAKINRKNEIAKLN |
| | GEQEISFEEIKAFDDKGYLLQKPSPNKSIYCYQSVSPKPFITSKYHNVNLPEEY |
| | IGYSRKPNEPIVSPYQFDRLRIPIGEPGYVPKWQYTFLSKKKNKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALNDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | GKLAPSYTVVLREAV |
| 404 | MIKPTVSQFLTPGFKLIRNHSRTAGRKLKNEGEEACKKFVRENEIPKDECPNFQ |
| | GGPAIANIIAKSREFTEWEIYRSSLAIQEVIFGLPKDKLPEPILKEEWRAQWLS |
| | EHGLDTVPYKEAAGLNLIIKNAVNTYKGVQVKVDNKNKKNLAKINRKNEIAKLN |
| | GEQEISFEEIKAFDDKGYLLQKPSPNKSIYCYQSVSPKPFITSKYHNVNLPEEY |
| | IGYSRKPNEPIVSPYQFDRLRIPIGEPGYVPKWQYTELSKKKNKRRKLSKRIKN |
| | VSPILGIIRIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVKKSDYKWFQDYKPKLSKEVRKALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 405 | MIKPTVSQFLTPGFKLIRNHSRTAGRKLKNEGEEACKKFVRENEIPKDECPNFQ |
| | GGPAIANIIAKSREFTEWEIYRSSLAIQEVIFGLPKDKLPEPILKEEWRAQWLS |
| | EHGLDTVPYKEAAGLNLIIKNAVNTYKGVQVKVDNKNKKNLAKINRKNEIAKLN |
| | GEQEISFEEIKAFDDKGYLLQKPSPNKSIYCYQSVSPKPFITSKYHNVNLPEEY |
| | IGYSRKPNEPIVSPYQFDRLRIPIGEPGYVPKWQYTFLSKKKNKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVKKSDYKWFQDYKPKLSKEVRKALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 406 | MIKPTVSQFLTPGFKLIRNHSRTAGRKLKNEGEEACKKFVRENEIPKDECPNFQ |
| | GGPAIANIIAKSREFTEWEIYRSSLAIQEVIFGLPKDKLPEPILKEEWRAQWLS |
| | EHGLDTVPYKEAAGLNLIIKNAVNTYKGVQVKVDNKNKKNLAKINRKNEIAKLN |
| | GEQEISFEEIKAFDDKGYLLQKPSPNKSIYCYQSVSPKPFITSKYHNVNLPEEY |
| | IGYSRKPNEPIVSPYQFDRLRIPIGEPGYVPKWQYTFLSKKKNKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVKKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | GKLAPSYTVVLREAV |
| 407 | MIKPTVSQFLTPGFKLIRNHLRTAGRKLKNEGEEACKKFVRENEIPKDECPNFQ |
| | GGPAIANIIAKSREFTEWEIYRSSLAIQEVIFGLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKPNEPIVSPYQFDRLRIPIGEPGYVPKWQYTFLSKKKNKRRKLSKRIKN |
| | VSPILGIIRIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDELNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVKKSDYKWFQDYKPKLSKEVRKALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | GKLAPSYTVVLREAV |
| 408 | MIKPTVSQFLTPGFKLIRNHSRTAGRKLKNEGEEACKKFVRENEIPKDECPNFQ |
| | GGPAIANIIAKSREFTEWEIYRSSLAIQEVIFGLPKDKLPEPILKEEWRAQWLS |
| | EHGLDTVPYKEAAGLNLIIKNAVNTYKGVQVKVDNKNKKNLAKINRKNEIAKLN |
| | GEQEISFEEIKAFDDKGYLLQKPGPNKSIYCYQSVSPKPFITSKYHNVNLPEEY |
| | IGYSRKPNEPIVSPYQFDRLRIPIGEPGYVPKWQYTFLSKKKNKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDELNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVKKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | GKLAPSYTVVLREAV |
| 409 | MIKPTVSQFLTPGFKLIRNHSRTAGRKLKNEGEEACKKFVRENEIPKDECPNFQ |
| | GGPAIANIIAKSREFTEWEIYQSSLAIQEVIFGLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYSRKPNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | GKLAPSYTVVLREAV |
| 410 | MIKPTVSQFLTPGFKLIRNHSRTAGRKLKNEGEEACKKFVRENEIPKDECPNFQ |
| | GGPAIANIIAKSREFTEWEIYQSSLAIQEVIFGLPKDKLPEPILKEEWRAQWLS |
| | EHGLDTVPYKEAAGLNLIIKNAVNTYKGVQVKVDNKNKKNLAKINRKNEIAKLN |
| | GEQEISFEEIKAFDDKGYLLQKPSPNKSIYCYQSVSPKPFITSKYHNVNLPEEY |
| | IGYSRKPNEPIVSPYQFDRLRIPIGEPGYVPKWQYTFLSKKKNKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | GKLAPSYTVVLREAV |
| 411 | MIKPTVSQFLTPGFKLIRNHLRTAGRKLKNEGEEACKKFVRENEIPKDECPNFQ |
| | GGPAIANIIAKSREFTEWEIYQSSLAIQEVIFGLPKDKLPEPILKEEWRAQWLS |
| | EHGLDTVPYKEAAGLNLIIKNAVNTYKGVQVKVDNKNKKNLAKINRKNEIAKLN |
| | GEQEISFEEIKAFDDKGYLLQKPSPNKSIYCYQSVSPKPFITSKYHNVNLPEEY |
| | IGYSRKPNEPIVSPYQFDRLRIPIGEPGYVPKWQYTFLSKKKNKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 412 | MIKPTVSQFLTPGFKLIRNHLRTAGRKLKNEGEEACKKFVRENEIPKDECPNFQ |
| | GGPAIANIIAKSREFTEWEIYQSSLAIQEVIFGLPKDKLPEPILKEEWRAQWLS |
| | EHGLDTVPYKEAAGLNLIIKNAVNTYKGVQVKVDNKNKKNLAKINRKNEIAKLN |
| | GEQEISFEEIKAFDDKGYLLQKPSPNKSIYCYQSVSPKPFITSKYHNVNLPEEY |
| | IGYSRKPNEPIVSPYQFDRLRIPIGEPGYVPKWQYTFLSKKKNKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDELNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVKKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 413 | MIKPTVSQFLTPGFKLIRNHLRTAGRKLKNEGEEACKKFVRENEIPKDECPNFQ |
| | GGPAIANIIAKSREFTEWEIYQSSLAIQEVIFGLPKDKLPEPILKEEWRAQWLS |
| | EHGLDTVPYKEAQGLNLIIKNAVNTYKGVQVKVDNKNKKNLAKINRKNEIAKLN |
| | GEQEISFEEIKAFDDKGYLLQKPSPNKSIYCYQSVSPKPFITSKYHNVNLPEEY |
| | IGYSRKPNEPIVSPYQFDRLRIPIGEPGYVPKWQYTFLSKKKNKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFLNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVKKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDKKNRNGEKFNC |
| | LKCGIELNADIDVATENLATVAITAQSMPKPTCERSGDAKKPVRARKAKAPEFH |
| | GKLAPSYTVVLREAV |
| 414 | MIKPTVSQFLTPGFKLIRNHLRTAGRKLKNEGEEACKKFVRENEIPKDECPNFQ |
| | GGPAIANIIAKSREFTEWEIYQSSLAIQEVIFGLPKDKLPEPILKEEWRAQWLS |
| | EHGLDTVPYKEAQGLNLIIKNAVNTYKGVQVKVDNKNKKNLAKINRKNEIAKLN |
| | GEQEISFEEIKAFDDKGYLLQKPGPNKSIYCYQSVSPKPFITSKYHNVNLPEEY |
| | IGYSRKPNEPIVSPYQFDRLRIPIGEPGYVPKWQYTFLSKKKNKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDELNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVKKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDKKNRNGEKFNC |
| | LKCGIELNADIDVATENLATVAITAQSMPKPTCERSGDAKKPVRARKAKAPEFH |
| | GKLAPSYTVVLREAV |
| 415 | MIKPTVSQFLTPGFKLIRNHSRTAGRKLKNEGEEACKKFVRENEIPKDECPNFQ |
| | GGPAIANIIAKSREFTEWEIYQSSLAIQEVIFGLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKPNEPIVSPYQFDRLRIPIGEPGYVPKWQYTFLSKKENKRRKLSKRIKN |
| | VSPILGIICIKKDWCVEDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKENC |
| | LKCGIELNADIDVATENLATVAITAQSMPKPTCERSGDAKKPVRARKAKAPEFH |
| | GKLAPSYTVVLREAV |
| 416 | MIKPTVSQFLTPGFKLIRNHSRTAGRKLKNEGEEACKKFVRENEIPKDECPNFQ |
| | GGPAIANIIAKSREFTEWEIYQSSLAIQEVIFGLPKDKLPEPILKEEWRAQWLS |
| | EHGLDTVPYKEAAGLNLIIKNAVNTYKGVQVKVDNKNKKNLAKINRKNEIAKLN |
| | GEQEISFEEIKAFDDKGYLLQKPSPNKSIYCYQSVSPKPFITSKYHNVNLPEEY |
| | IGYYRKPNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 417 | MIKPTVSQFLTPGFKLIRNHSRTAGRKLKNEGEEACKKFVRENEIPKDECPNFQ |
| | GGPAIANIIAKSREFTEWEIYQSSLAIQEVIFGLPKDKLPEPILKEEWRAQWLS |
| | EHGLDTVPYKEAAGLNLIIKNAVNTYKGVQVKVDNKNKKNLAKINRKNEIAKLN |
| | GEQEISFEEIKAFDDKGYLLQKPSPNKSIYCYQSVSPKPFITSKYHNVNLPEEY |
| | IGYYRKPNEPIVSPYQFDRLRIPIGEPGYVPKWQYTELSKKKNKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 418 | MIKPTVSQFLTPGFKLIRNHSRTAGRKLKNEGEEACKKFVRENEIPKDECPNFQ |
| | GGPAIANIIAKSREFTEWEIYQSSLAIQEVIFGLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPGPNKSIYCYQSVSPKPFITSKYRNVNLPEEY |
| | IGYYRKPNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFLNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 419 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKKNLAKINRKNEIAKLN |
| | GEQEISFEEIKAFDDKGYLLQKPGPNKSIYCYQSVSPKPFITSKYHNVNLPEEY |
| | IGYYRKPNEPIVSPYQFDRLRIPIGEPGYVPKWQYTFLSKKENKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVKKSDYKWFQDYKPKLSKEVRDALSDIEWRLRRESLEEN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |
| 420 | MIKPTVSQFLTPGFKLIRNHSRTAGRKLKNEGEEACKKFVRENEIPKDECPNFQ |
| | GGPAIANIIAKSREFTEWEIYQSSLAIQEVIFTLPKDKLPEPILKEEWRAQWLS |
| | EHGLDTVPYKEAAGLNLIIKNAVNTYKGVQVKVDNKNKNNLAKINRKNEIAKLN |
| | GEQEISFEEIKAFDDKGYLLQKPSPNKSIYCYQSVSPKPFITSKYHNVNLPEEY |
| | IGYYRKPNEPIVSPYQFDRLRIPIGEPGYVPKWQYTFLSKKKNKRRKLSKRIKN |
| | VSPILGIICIKKDWCVFDMRGLLRTNHWKKYHKPTDSINDLFDYFTGDPVIDTK |
| | ANVVRFRYKMENGIVNYKPVREKKGKELLENICDQNGSCKLATVDVGQNNPVAI |
| | GLFELKKVNGELTKTLISRHPTPIDFCNKITAYRERYDKLESSIKLDAIKQLTS |
| | EQKIEVDNYNNNFTPQNTKQIVCSKLNINPNDLPWDKMTSGTHFISEKAQVSNK |
| | SEIYFTSTDKGKTKDVMKSDYKWFQDYKPKLSKEVRDALSDIEWRLRRESLEFN |
| | KLSKSREQDARQLANWISSMCDVIGIENLVKKNNFFGGSGKREPGWDNFYKPKK |
| | ENRWWINAIHKALTELSQNKGKRVILLPAMRTSITCPKCKYCDSKNRNGEKFNC |
| | LKCGIELNADIDVATENLATVAITAQSMPKPTCERSGDAKKPVRARKAKAPEFH |
| | GKLAPSYTVVLREAV |

TABLE 5 provides illustrative sequences of exemplary heterologous peptide modifications of effector protein(s) that are useful in the compositions, systems and methods described herein.

TABLE 5
SEQUENCES OF EXEMPLARY HETEROLOGOUS
PEPTIDE MODIFICATIONS

| SEQ ID | | |
|---|---|---|
| NO: | Description | Sequence |
| 245 | NLS | KR(K/R)R |
| 246 | NLS | P/R)XXKR(^{\hat{ }}D/E )(K/R) |
| 247 | NLS | KRX(W/F/Y)XXAF |
| 248 | NLS | (R/P)XXKR(K/R)(^{\hat{ }}D/E) |
| 249 | NLS | LGKR(K/R)(W/F/Y) |
| 250 | NLS | KRX₁₀K(K/R)(K/R) |
| 251 | EEP | GLFXALLXLLXSLWXLLLXA |
| 252 | NLS | K(K/R)RK |
| 253 | NLS | KRX₁₁K(K/R)(K/R) |
| 254 | NLS | KRX₁₂K(K/R)(K/R) |
| 255 | NLS | KRX₁₀K(K/R)X(K/R) |
| 256 | NLS | KRX₁₁K(K/R)X(K/R) |
| 257 | NLS | KRX₁₂K(K/R)X(K/R) |
| 258 | NLS | APKKKRKVGIHGVPAA |

*wherein X is any naturally occurring amino acid; and ^{\hat{ }}D/E is any naturally occurring amino acid except Asp or Glu

TABLE 6 provides illustrative PAM sequences that are useful in the compositions, systems and methods described herein.

TABLE 6
EXEMPLARY PAM SEQUENCES

| Effector Protein SEQ ID NO: | PAM Sequence* (5′→3′) |
|---|---|
| 259 | NTTN |
| 260 | NTTA |
| 261 | NTTC |
| 262 | NTTG |
| 263 | NTTT |
| 264 | GTTG |

*wherein each N is independently any one of A, G, C, or T

TABLE 7 provides an illustrative repeat sequence for use with the compositions, systems and methods of the disclosure.

TABLE 7
EXEMPLARY REPEAT SEQUENCES FOR USE
IN GUIDE NUCLEIC ACIDS

| Seq ID | |
|---|---|
| No. | Repeat sequence (5′→ 3′), shown as RNA |
| 265 | AUAGAUUGCUCCUUACGAGGAGAC |
| 348 | AUUGCUCCUUACGAGGAGAC |

TABLE 8 provides illustrative target nucleic acids that are useful in the compositions, systems and methods described herein.

TABLE 8
EXEMPLARY TARGET NUCLEIC ACIDS
Exemplary targets

AAVS1, ABCA4, ABCB11, ABCC8, ABCD1, ABCG5, ABCG8, ACAD9, ACADM,
ACADVL, ACAT1, ACTA1, ACOX1, ACSF3, ADA, ADAMTS2, ADGRG1, AGA, AGL,
AGPS, AGXT, AHI1, AIRE, ALDH3A2, ALDOB, ALG6, ALK, ALKBH5, ALMS1, ALPL,
AMRC9, AMT, ANAPC10, ANAPC11, ANGPTL3, ANGPTL4, APC, Apo(a), APOCIII,
APOEε4, APOL1, APP, AQP2, AR, ARFRP1, ARG1, ARH, ARL13B, ARL6, ARSA, ARSB,
ASL, ASNS, ASPA, ASS1, ATM, ATP6V1B1, ATP7A, ATP7B, ATRX, ATXN1, ATXN10,
ATXN2, ATXN3, ATXN7, ATXN8OS, AXIN1, AXIN2, B2M, BACE-1, BAK1, BAP1,
BARD1, BAX2, BBS1, BBS10, BBS12, BBS2, BCKDHA, BCKDHB, BCL2L2, BCS1L,
BEST1, Betaglobin gene, BLM, BMPR1A, BRAF, BRAFV600E, BRCA1, BRCA2, BRIP1,
BSND, C9orf72, CA4, CACNA1A, CAH1, CAPN3, CASR, CBS, CCNB1 CC2D2A, CCR5,
CD1, CD2, CD3, CD3D, CD3Z, CD4, CD5, CD6, CD7, CD8A, CD8B, CD9, CD14,
CD18, CD19, CD21, CD22, CD23, CD27, CD28, CD30, CD33, CD34, CD36, CD38,
CD40, CD40L, CD44, CD46, CD47, CD48, CD52, CD55, CD57, CD58, CD59, CD68,
CD69, CD72, CD73, CD74, CD79A, CD80, CD81, CD83, CD84, CD86, CD90, CD93,
CD96, CD99, CD100, CD123, CD160, CD163, CD164, CD164L2, CD166, CD200,
CD204, CD207, CD209, CD226, CD244, CD247, CD274, CD276, CD300, CD320,
CDC73, CDH1, CDH23, CDK11, CDK4, CDKN1A, CDKN1B, CDKN1C, CDKN2A,
CDKN2B, CEBPA, CELA3B, CEP290, CERKL, CFB, CFTR, CHCHD10, CHEK2, CHM,
CHRNE, CIDEB, CIITA, CLN3, CLN5, CLN6, CLN8, CLRN1, CLTA, CMT1A, CNBP,
CNGB1, CNGB3, COL1A1, COL1A2, COL27A1, COL4A3, COL4A4, COL4A5,
COL6A1, COL6A2, COL6A3, COL7A1, CPS1, CPT1A, CPT2, CRB1, CREBBP, CRX,
CRYAA, CTNNA1, CTNNB1, CTNND2, CTNS, CTSK, CXCL12, CYBA, CYBB,
CYP11B1, CYP11B2, CYP17A1, CYP19A1, CYP21A2, CYP27A1, DBT, DCC,
DCLRE1C, DERL2, DFNA36, DFNB31, DGAT2, DHCR7, DHDDS, DICER1, DIS3L2,
DLD, DMD, DMPK, DNAH5, DNAI1, DNAI2, DNM2, DNMT1, DPC4, DYSF, EDA,
EDN3, EDNRB, EGFR, EIF2B5, EMC2, EMC3, EMD, EMX1, EN1, EPCAM, ERCC6,
ERCC8, ESCO2, ETFA, ETFDH, ETHE1, EVC, EVC2, EYS, F5, F9, FXI, FAH,
FAM161A, FANCA, FANCB, FANCC, FANCD1, FANCD2, FANCE, FANCF, FANCG,
FANCI, FANCJ, FANCL, FANCM, FANCN, FANCP, FANCS, FBN1, FGF14, FGFR2,
FGFR3, FGA, FGB, FGG, FH, FHL1, FIX, FKRP, FKTN, FLCN, FMR1, FOXP3,
FSCN2, FSHD1, FUS, FUT8, FVIII, FXII, FXN, G6PC, GAA, GALC, GALK1, GALT,
GAMT, GATA2, GATA-4, GBA, GBE1, GCDH, GCGR, GDNF, GFAP, GFM1, GHR,
GJB1, GJB2, GLA, GLB1, GLDC, GLE1, GNE, GNPTAB, GNPTG, GNS, GPAM, GPC3,
GPR98, GREM1, GRHPR, GRIN2B, H2AFX, H2AX, HADHA, HAX1, HBA1, HBA2,
HBB, HBV cccDNA, HER2, HEXA, HEXB, HFE, HGSNAT, HLCS, HMGCL, HAO1,
HOGA1, HOXB13, HPRPF3, HPRT1, HPS1, HPS3, HRAS, HRD1, HSD3B2, HSD17B4,
HSD17B13, HTT, HUS1, HYAL1, HYLS1, IDS, IDUA, IFITM5, IFN, IFN-γ, IKBKAP,
IL2RG, IL7R, IMPDH1, INPP5E, IRF4, ITGB2, ITPR1, IVD, JAG1, JAK1, JAK3,
KCNC3, KCND3, KCNJ11, KLKB1, KLHL7, KRAS, LAMA1, LAMA2, LAMA3, LAMB3,
LAMC2, LCA5, LDHA, LDLR, LDLRAP1, LHX3, LIFR, LIPA, LMNA, LMOD3, LOR,
LOXHD1, LPA, LPL, LRAT, LRP6, LRPPRC, LRRK2, MADR2, MAN2B1, MAPT,
MARC1, MAX, MCM6, MCOLN1, MECP2, MED17, MEFV, MEN1, MERTK, MESP2,
MET, METex14, MFN2, MFSD8, MIA3, MITF, MKL2, MKS1, MLC1, MLH1, MLH3,
MMAA, MMAB, MMACHC, MMADHC, MMD, MPI, MPL, MPV17, MSH2, MSH3,
MSH6, MTHFD1L, MTHFR, MTM1, MTRR, MTTP, MUT, MUTYH, MYC, MYH7,
MYO7A, NAGLU, NAGS, NBN, NDRG1, NDUFAF5, NDUFS6, NEB, NF1, NF2, NKX2-
5, NOG, NOTCH1, NOTCH2, NPC1, NPC2, NPHP1, NPHS1, NPHS2, NRAS, NR2E3,
NTHL1, NTRK, NTRK1, OAT, OCT4, OFD1, OPA3, OTC, PAH, PALB2, PAQR8, PAX3,
PC, PCCA, PCCB, PCDH15, PCSK9, PD1, PDCD1, PDE6B, PDGFRA, PDHA1,
PDHB, PEX1, PEX10, PEX12, PEX13, PEX14, PEX16, PEX19, PEX2, PEX26, PEX3,
PEX5, PEX6, PEX7, PFKM, PHGDH, PHOX2B, PKD1, PKD2, PKHD1, PKK,
PLEKHG4, PMM2, PMP22, PMS1, PMS2, PNPLA3, POLD1, POLE, POMGNT1,
POT1, POU5F1, PPM1A, PPP2R2B, PPT1, PRCD, PRKAG2, PRKAR1A, PRKCG,
PRNP, PROM1, PROP1, PRPF31, PRPF8, PRPH2, PRPS1, PSAP, PSD3, PSD95,
PSEN1, PSEN2, PSRC1, PTCH1, PTEN, PTS, PUS1, PYGM, RAB23, RAD50, RAD51C,
RAD51D, RAG1, RAG2, RAPSN, RARS2, RB1, RDH12, RECQL4, RET, RHO, RICTOR,
RMRP, ROS1, RP1, RP2, RPE65, RPGR, RPGRIP1L, RPL32P3, RS1, RTCA, RTEL1,
RUNX1, SACS, SAMHD1, SCN1A, SCN2A, SDHA, SDHAF2, SDHB, SDHC, SDHD,
SEL1L, SEPSECS, SERPINA1, SERPINC1, SERPING1, SGCA, SGCB, SGCG, SGSH,
SIRT1, SLC12A3, SLC12A6, SLC17A5, SLC22A5, SLC25A13, SLC25A15, SLC26A2,
SLC26A4, SLC35A3, SLC35B4, SLC37A4, SLC39A4, SLC4A11, SLC6A8, SLC7A7,
SMAD3, SMAD4, SMARCA4, SMARCAL1, SMARCB1, SMARCE1, SMN1, SMPD1,
SNAI2, SNCA, SNRNP200, SOD1, SOX10, SPARA7, SPTBN2, STAR, STAT3, STK11,
SUFU, SUMF1, SYNE1, SYNE2, SYS1, TARDBP, TAT, TBK1, TBP, TCIRG1, TCTN3,
TECPR2, TERC, TERT, TFR2, TGFBR2, TGM1, TH, TLE3, TMEM127, TMEM138,
TMEM216, TMEM43, TMEM67, TMPRSS6, TNNT1, TNNT3, TNN12, TOP1, TOPORS,
TP53, TPM2, TPM3, TPP1, TRAC, TRMU, TSC1, TSC2, TSFM, TSPAN14, TTBK2,
TTC8, TTPA, TTR, TULP1, TYMP, UBE2G2, UBE2J1, UBE3A, USH1C, USH1G,
USH2A, VEGF, VHL, VPS13A, VPS13B, VPS35, VPS45, VRK1, VSX2, VWF, WAS,
WDR19, WDR48, WNT10A, WRN, WS2B, WS2C, WT1, XPA, XPC, XPF, XRCC3, YAP1,
ZAC1, ZEB1, ZFYVE26 and ZNF423

TABLE 9 provides illustrative diseases and syndromes for compositions, systems and methods described herein.

TABLE 9
DISEASES AND SYNDROMES
Exemplary Diseases and Syndromes

11-hydroxylase deficiency; 17,20-desmolase deficiency; 17-hydroxylase deficiency; 3-
hydroxyisobutyrate aciduria; 3-hydroxysteroid dehydrogenase deficiency; 46,XY gonadal
dysgenesis; AAA syndrome; ABCA3 deficiency; ABCC8-associated hyperinsulinism;
aceruloplasminemia; acromegaly; achondrogenesis type 2; acral peeling skin syndrome;
acrodermatitis enteropathica; acute bacterial infection; adrenocortical micronodular
hyperplasia; adrenoleukodystrophies; adrenomyeloneuropathies; Aicardi-Goutieres
syndrome; AIDS; Alagille disease (also called Alagille Syndrome); Alexander Disease;
Alpers syndrome; alpha-1 antitrypsin deficiency (AATD); alpha-mannosidosis; Alstrom
syndrome; Alzheimer's disease; amebic dysentery; amelogenesis imperfecta; amish type
microcephaly; amyotrophic lateral sclerosis (ALS); anaplastic large cell lymphoma;
anauxetic dysplasia; androgen insensitivity syndrome; angiopathic thrombosis;
antiphospholipid syndrome; Antley-Bixler syndrome; APECED; Apert syndrome; aplasia
of lacrimal and salivary glands; arginase-1 deficiency; argininosuccinic aciduria;
argininemia; arrhythmogenic right ventricular dysplasia; Arts syndrome; ARVD2;
arylsulfatase deficiency type metachromatic leukodystrophy; ataxia telangiectasia;
atherosclerotic cardiovascular disease; autoimmune lymphoproliferative syndrome;
autoimmune polyglandular syndrome type 1; autosomal dominant anhidrotic ectodermal
dysplasia; autosomal dominant deafness; autosomal dominant polycystic kidney disease;
autosomal recessive microtia; autosomal recessive renal glucosuria; autosomal visceral
heterotaxy; babesiosis; bacterial vaginosis; balantidial dysentery; Bardet-Biedl syndrome;
Bartter syndrome; basal cell nevus syndrome; Batten disease; benign recurrent intrahepatic
cholestasis; beta-mannosidosis; β-thalassemia; Bethlem myopathy; Blackfan-Diamond
anemia; bleeding disorder (coagulation); blepharophimosis; Byler disease; C syndrome;
CADASIL; calcific aortic stenosis; calcification of joints and arteries; carbamoyl
phosphate synthetase I deficiency; carcinoid syndrome diarrhea; cardiofaciocutaneous
syndrome; cardiovascular disease (CVD); Carney triad; carnitine palmitoyltransferase
deficiencies; cartilage-hair hypoplasia; cblC type of combined methylmalonic aciduria;
CD18 deficiency; CD3Z-associated primary T-cell immunodeficiency; CD40L
deficiency; CDAGS syndrome; CDG1A; CDG1B; CDG1M; CDG2C; CEDNIK
syndrome; central core disease; centronuclear myopathy; cerebral capillary malformation;
cerebrooculofacioskeletal syndrome type 4; cerebrooculogacioskeletal syndrome;
cerebrotendinous xanthomatosis; Chagas Disease; Charcot Marie Tooth Disesase;
chemotherapy; cherubism; CHILD syndrome; chronic granulomatous disease; chronic
recurrent multifocal osteomyelitis; cirrhosis; citrin deficiency; citrullinemia type I;
citrullinemia type II; classic hemochromatosis; CNPPB syndrome; cobalamin C disease;
Cockayne syndrome; coenzyme Q10 deficiency; Coffin-Lowry syndrome; Cohen
syndrome; combined deficiency of coagulation factors V; common variable immune
deficiency 3; complement hyperactivation; complete androgen insentivity; cone rod
dystrophies; conformational diseases; congenital adrenal hyperplasia; congenital bile acid
synthesis defect type 1; congenital bile acid synthesis defect type 2; congenital defect in
bile acid synthesis type; congenital erythropoietic porphyria; congenital generalized
osteosclerosis; congenital hyperplasia (CAH); congenital muscular dystrophy 1A
(MDC1A); Cornelia de Lange syndrome; coronary heart disease; Cousin syndrome;
Cowden disease; COX deficiency; Cri du chat syndrome; Crigler-Najjar disease; Crigler-
Najjar syndrome type 1; Crisponi syndrome; Crouzon syndrome; Currarino syndrome;
Curth-Macklin type ichthyosis hystrix; cutaneous T-cell lymphoma; cutis laxa; cystic
fibrosis; cystinosis; d-2-hydroxyglutaric aciduria; DDP syndrome; Dejerine-Sottas
disease; Denys-Drash syndrome; Dercum disease; desmin cardiomyopathy; desmin
myopathy; DGUOK-associated mitochondrial DNA depletion; diabetes Type I; diabetes
Type II; disorders of glutamate metabolism; distal spinal muscular atrophy type 5; DNA
repair diseases; dominant optic atrophy; Doyne honeycomb retinal dystrophy; Dravet
Syndrome; Duchenne muscular dystrophy; dyskeratosis congenita; Ehlers-Danlos
syndrome type 4; Ehlers-Danlos syndromes; Elejalde disease; Ellis-van Creveld disease;
Emery-Dreifuss muscular dystrophies; encephalomyopathic mtDNA depletion syndrome;
encephalitis; enzymatic diseases; EPCAM-associated congenital tufting enteropathy;
epidermolysis bullosa with pyloric atresia; epilepsy; fabry disease; facioscapulohumeral
muscular dystrophy; Factor V Leiden thrombophilia; Faisalabad histiocytosis; familial
atypical mycobacteriosis; familial capillary malformation-arteriovenous; Familial
Creutzfeld-Jakob disease; familial esophageal achalasia; familial glomuvenous
malformation; familial hemophagocytic lymphohistiocytosis; familial mediterranean
fever; familial megacalyces; familial schwannomatosis; familial spina bifida; familial
splenic asplenia/hypoplasia; familial thrombotic thrombocytopeniaurpura; Fanconi
disease (Fanconi anemia); Feingold syndrome; FENIB; fibrodysplasia ossificans
progressiva; FKTN; Fragile X syndrome; Francois-Neetens fleck corneal dystrophy;
Frasier syndrome; Friedreich's ataxia; FTDP-17; Fuchs corneal dystrophy; fucosidosis;
G6PD deficiency; galactosialidosis; Galloway syndrome; Gardner syndrome; Gaucher
disease; Gitelman syndrome; glaucoma; GLUT1 deficiency; GM2- Gangliosidoses (e.g.,
Tay Sachs Disease, Sandhoff Disease) glycogen storage disease type 1b; glycogen storage
disease type 2; glycogen storage disease type 3; glycogen storage disease type 4; glycogen
storage disease type 9a; glycogen storage diseases; GM1-gangliosidosis; Greenberg
syndrome; Greig cephalopolysyndactyly syndrome; hair genetic diseases; hairy cell
leukemia; HANAC syndrome; harlequin type ichtyosis congenita; HDR syndrome;
hearing loss; heart failure; hemochromatosis type 3; hemochromatosis type 4; hemolytic
anemia; hemolytic uremic syndrome; hemophilia A; hemophilia B; hepatitis C infection;
hereditary angioedema type 3; hereditary angioedemas; hereditary hemorrhagic
telangiectasia; hereditary hypofibrinogenemia; hereditary intraosseous vascular
malformation; hereditary leiomyomatosis and renal cell cancer; hereditary neuralgic
amyotrophy; hereditary orotic aciduria; hereditary sensory and autonomic neuropathy
type; Hermansky-Pudlak disease; HHH syndrome; HHT2; hidrotic ectodermal dysplasia
type 1; hidrotic ectodermal dysplasias; histiocytic sarcoma; HNF4A-associated
hyperinsulinism; HNPCC; homozygous familial hypercholesterolemia; hormone
refractory prostate cancer; human immunodeficiency with microcephaly; Human
monkeypox (MPX); human papilloma virus (HPV) infection; Huntington's disease; hyper-
IgD syndrome; hyperinsulinism-hyperammonemia syndrome; hypercholesterolemia;
hypertension; hypertrophy of the retinal pigment epithelium; hypochondrogenesis;
hypohidrotic ectodermal dysplasia; hypotension; ICF syndrome; idiopathic congenital
intestinal pseudo-obstruction; immunodeficiency 13; immunodeficiency 17;
immunodeficiency 25; immunodeficiency with hyper-IgM type 1; immunodeficiency with
hyper-IgM type 3; immunodeficiency with hyper-IgM type 4; immunodeficiency with
hyper-IgM type 5; immunoglobulin alpha deficiency; inborn errors of thyroid metabolism;
infantile myofibromatosis; infantile visceral myopathy; infantile X-linked spinal muscular
atrophy; influenza A; influenza B; intradialytic hypotension; intrahepatic cholestasis of
pregnancy; invasive aspergillosis; invasive mucormycosis; IPEX syndrome; IRAK4
deficiency; isolated congenital asplenia; Jeune syndrome; Johanson-Blizzard syndrome;
Joubert syndrome; JP-HHT syndrome; juvenile hemochromatosis; juvenile hyalin
fibromatosis; juvenile nephronophthisis; Kabuki mask syndrome; Kallmann syndromes;
Kartagener syndrome; KCNJ11-associated hyperinsulinism; Kearns-Sayre syndrome;
Kostmann disease; Kozlowski type of spondylometaphyseal dysplasia; Krabbe disease;
LADD syndrome; late infantile-onset neuronal ceroid lipofuscinosis; LCK deficiency;
LDHCP syndrome; Leber Congenital Amaurosis Teyp 10; Legius syndrome; Leigh
syndrome; lethal congenital contracture syndrome 2; lethal congenital contracture
syndromes; lethal contractural syndrome type 3; lethal neonatal CPT deficiency type 2;
lethal osteosclerotic bone dysplasia; leukocyte adhesion deficiency; Li Fraumeni
syndrome; LIG4 syndrome; limb girdle muscular dystrophies (LGMD1B, LGMD2A,
LGMD2B); lipodystrophy; lissencephaly type 1; lissencephaly type 3; Loeys-Dietz
syndrome; low phospholipid-associated cholelithiasis; Lynch Syndrome; lysinuric protein
intolerance; a lysosomal storage disease (e.g., Hunter syndrome, Hurler syndrome);
macular dystrophy; Maffucci syndrome; Majeed syndrome; malaria; mannose-binding
protein deficiency; mantle cell lymphoma; Marfan disease; Marshall syndrome; MASA
syndrome; mastocytosis; MCAD deficiency; McCune-Albright syndrome; MCKD2;
Meckel syndrome; MECP2 Duplication Syndrome; Meesmann corneal dystrophy;
megacystis-microcolon-intestinal hypoperistalsis; megaloblastic anemia type 1; MEHMO;
MELAS; Melnick-Needles syndrome; MEN2s; meningitis; Menkes disease;
metachromatic leukodystrophies; methymalonic acidemia due to transcobalamin receptor
defect; methylmalonic acidurias; methylvalonic aciduria; microcoria-congenital nephrosis
syndrome; microvillous atrophy; migraine; mitochondrial neurogastrointestinal
encephalomyopathy; monilethrix; monosomy X; mosaic trisomy 9 syndrome; Mowat-
Wilson syndrome; mucolipidosis type 2; mucolipidosis type Ma; mucolipidosis type IV;
mucopolysaccharidoses; mucopolysaccharidosis type 3A; mucopolysaccharidosis type
3C; mucopolysaccharidosis type 4B; multiminicore disease; multiple acyl-CoA
dehydrogenation deficiency; multiple cutaneous and mucosal venous malformations;
multiple endocrine neoplasia type 1; multiple myeloma; multiple sclerosis; multiple
sulfatase deficiency; mycosis fungoides; myotonic dystrophy; NAIC; nail-patella
syndrome; nemaline myopathies; neonatal diabetes mellitus; neonatal surfactant
deficiency; nephronophtisis; Netherton disease; neurofibromatoses; neurofibromatosis
type 1; Niemann-Pick disease type A; Niemann-Pick disease type B; Niemann-Pick
disease type C; NKX2E; non-alcoholic fatty liver disease (NAFLD); non-alcoholic
steatohepatitis (NASH); Noonan syndrome; North American Indian childhood cirrhosis;
NROB1 duplication-associated DSD; ocular genetic diseases; oculo-auricular syndrome;
OLEDAID; oligomeganephronia; oligomeganephronic renal hypolasia; Ollier disease;
Opitz-Kaveggia syndrome; ornithine transcarbamylase deficiency (OTCD);
orofaciodigital syndrome type 1; orofaciodigital syndrome type 2; osseous Paget disease;
osteogenesis imperfecta; otopalatodigital syndrome type 2; orthostatic hypotension;
overactive bladder; OXPHOS diseases; palmoplantar hyperkeratosis; panlobar
nephroblastomatosis; Parkes-Weber syndrome; Parkinson's disease; partial deletion of
21q22.2-q22.3; Pearson syndrome; Pelizaeus-Merzbacher disease; Pendred syndrome;
pentalogy of Cantrell; peroxisomal acyl-CoA-oxidase deficiency; Peutz-Jeghers
syndrome; Pfeiffer syndrome; Pierson syndrome; pigmented nodular adrenocortical
disease; pipecolic acidemia; Pitt-Hopkins syndrome; plasmalogens deficiency; platelet
glycoprotein IV deficiency; pleuropulmonary blastoma and cystic nephroma; pneumonia;
polycystic kidney disease; polycystic ovarian disease; polycystic lipomembranous
osteodysplasia; Pompe disease; including infantile onset Pompe disease (IOPD) and late
onset Pompe disease (LOPD); porphyrias; post-herpetic neuralgia; PRKAG2 cardiac
syndrome; premature ovarian failure; primary erythermalgia; primary hemochromatoses;
primary hyperoxaluria; progressive familial intrahepatic cholestasis; propionic acidemia;
prostate cancer; protein-losing enteropathy; pulmonary arterial hypertension; pyruvate
decarboxylase deficiency; RAPADILINO syndrome; renal cystinosis; restless leg
syndrome; retinitis pigmentosa; Rett Syndrome; rhabdoid tumor predisposition syndrome;
Rieger syndrome; ring chromosome 4; Roberts syndrome; Robinow-Sorauf syndrome;
Rothmund-Thomson syndrome; severe combined immunodeficiency disorder (SCID);
Saethre-Chotzen syndrome; Sandhoff disease; SC phocomelia syndrome; SCAS; Schinzel
phocomelia syndrome; schizophrenia; severe hypertriglyceridemia; short rib-polydactyly
syndrome type 1; short rib-polydactyly syndrome type 4; short-rib polydactyly syndrome
type 2; short-rib polydactyly syndrome type 3; Shwachman disease; Shwachman-Diamond
disease; sickle cell anemia; Silver-Russell syndrome; Simpson-Golabi-Behmel syndrome;
skin infection; Smith-Lemli-Opitz syndrome; SPG7-associated hereditary spastic
paraplegia; spherocytosis; spinocerebellar ataxia; spinal muscular atrophy; split-hand/foot
malformation with long bone deficiencies; spondylocostal dysostosis; sporadic
amyotrophic lateral sclerosis; sporadic visceral myopathy with inclusion bodies; storage
diseases; Stargardt macular dystrophy; STRA6-associated syndrome; stroke; tardive
dyskinesia; Tay-Sachs disease; thanatophoric dysplasia; thromboembolism; thrombosis;
thrombophilia due to antithrombin III deficiency; thyroid metabolism diseases; Tourette
syndrome; transcarbamylase deficiency; transthyretin-associated amyloidosis; trisomy 13;
trisomy 22; trisomy 2p syndrome; tuberous sclerosis; tufting enteropathy; ullrich
congenital muscular dystrophy (UCMD); urea cycle diseases; Usher Syndrome; Van Den
Ende-Gupta syndrome; Van der Woude syndrome; variegated mosaic aneuploidy
syndrome; VLCAD deficiency; von Hippel-Lindau disease; von Willebrand disease;
Waardenburg syndrome; WAGR syndrome; Walker-Warburg syndrome; Werner
syndrome; Wilson's disease; Wiskott-Aldrich Syndrome; Wolcott-Rallison syndrome;
Wolfram syndrome; X-linked agammaglobulinemia; X-linked chronic idiopathic
intestinal pseudo-obstruction; X-linked cleft palate with ankyloglossia; X-linked dominant
chondrodysplasia punctata; X-linked ectodermal dysplasia; X-linked Emery-Dreifuss
muscular dystrophy; X-linked lissencephaly; X-linked lymphoproliferative disease; X-
linked visceral heterotaxy; xanthinuria type 1; xanthinuria type 2; xeroderma
pigmentosum; XPV; and Zellweger disease.

### EXAMPLES

The following examples are included for illustrative purposes only and are not intended to limit the scope of the invention.

### Example 1. In Vitro Cis Cleavage Activity of Fourteen Effector Proteins

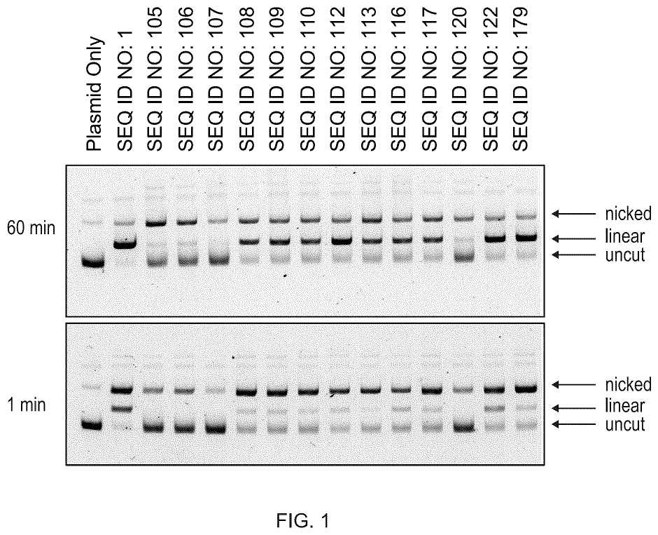

An in vitro screening was carried out to identify DNA-targeting cis cleavage activity (e.g., nicking activity). Specifically, compositions comprising an isolated effector protein, a guide nucleic acid (e.g., crRNA) and a target nucleic acid were tested in a cis cleavage assay. Briefly, compositions comprising an effector protein (e.g., 50 nM of effector protein selected from SEQ ID NO: 105-110, 112-113, 116-117, 120, 122 and 179), a guide nucleic acid (e.g., 50 nM of crRNA), a supercoiled double stranded target nucleic acid and a buffer (e.g., 200 mM HEPES pH 7.5 at 37° C., 3 mM Mg(OAc)₂, 1 mM TCEP and 0.2 mg/mL BSA, or other suitable 10× cleavage buffer) were incubated at 37° C. for different time intervals (e.g., 1 minute and 1 hour). In some experiments, incubated compositions further comprised a stop mix (e.g., proteinase K+EDTA, or formamide dye). Cis cleavage activity of the effector proteins (e.g., nickase activity) were confirmed by gel electrophoresis. FIG. 1 shows the results of the cis cleavage assay. At least some effector proteins were found to have enhanced nickase activity. For example, the effector protein comprising SEQ ID NO: 105-106 appears to have preference for nicking the double stranded target nucleic acid over double strand break.

### Example 2. In Vitro Assay for Determining Modified Activity of Effector Proteins

An in vitro screen is carried out to determine change in nuclease activity of effector proteins (e.g., as set forth in TABLE 4) relative to corresponding wildtype effector protein (e.g., as set forth in TABLE 1). Specifically, the assays is designed to determine if effector proteins comprised a nickase activity which are capable of specifically cutting only one strand of a double stranded target nucleic acid. Briefly, eukaryotic cells (e.g., immune cell, T cell, HEK29 cell, or any other eukaryotic cell) are treated (e.g., transfection, electroporation, or lipofection) with plasmid pairs co-expressing the effector protein and a guide nucleic acid in the presence or absence of nickase variant of SpyCas9. Transfected cells are first incubated for 72 hours. The incubated cells are then lysed and subjected to PCR amplification. Indels are detected by next generation sequencing (NGS) of PCR amplicons at the targeted loci, and indel percentage is calculated as the fraction of sequencing reads containing insertions or deletions relative to an unedited reference sequence. The effector proteins having nickase activity shows low indel when transfected alone but shows high indels when co-transfected with nickase variant of SpyCas9. The effector proteins having nuclease activity shows high indel irrespective of the presence of nickase variant of SpyCas9. The effector protein having no catalytic activity show low indel irrespective of the presence of nickase variant of SpyCas9.

### Example 3. In Vitro Cis Cleavage Activity of Thirty-One Effector Proteins

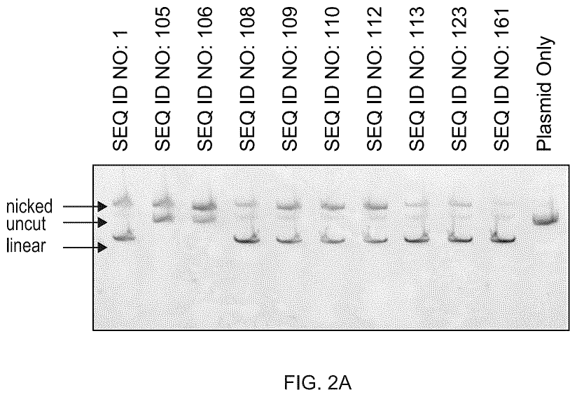

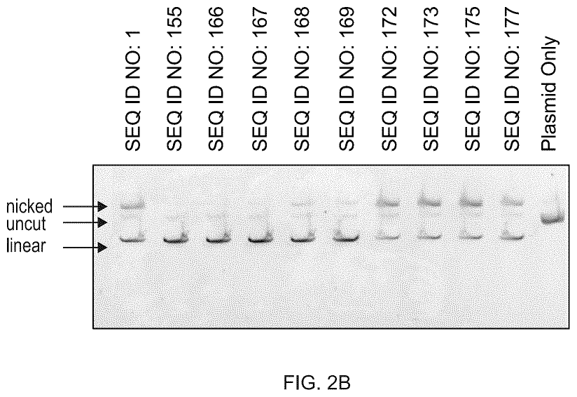

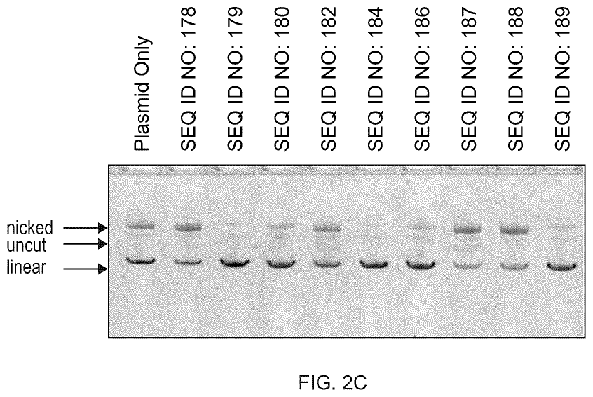

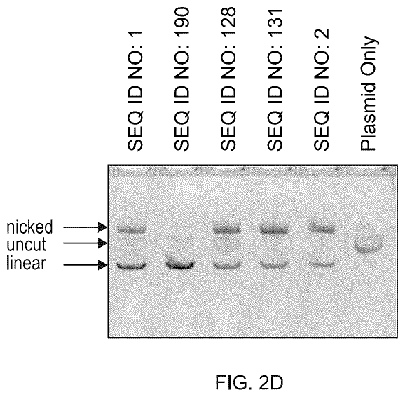

An in vitro screening was carried out to identify DNA-targeting cis cleavage activity (e.g., nickase activity). Specifically, compositions comprising an isolated effector protein, a guide nucleic acid (e.g., crRNA) and a target nucleic acid were tested in a cis cleavage assay. Briefly, compositions comprising an effector protein (e.g., 50 nM of effector protein selected from SEQ ID NO: 105-106, 108-110, 112-113, 123, 128, 131, 155, 161, 166-169, 172-173, 175, 177-180, 182, 184, 186, and 187-190), a guide nucleic acid (e.g., 50 nM of crRNA), various concentrations of a supercoiled double stranded target nucleic acid and a buffer (e.g., 200 mM HEPES pH 7.5 at 37° C., 3 mM Mg(OAc)₂, 1 mM TCEP and 0.2 mg/mL BSA, or other suitable 10× cleavage buffer) were incubated at 37° C. For positive control, the effector proteins of SEQ ID NO: 1 and 2 were used. For a negative control, only target nucleic acid (plasmid) was used. In some experiments, incubated compositions further comprised a stop mix (e.g., proteinase K+EDTA, or formamide dye). Cis cleavage activity of the effector proteins (e.g., nickase activity) were confirmed by gel electrophoresis. FIGS. 2A-2D show the results of the cis cleavage assay. At least some effector proteins were found to have enhanced nickase activity. For example, the effector protein comprising SEQ ID NO: 105-106 appears to have preference for nicking the double stranded target nucleic acid over double strand break.

### Example 4. In Vitro Determination of Cut Sites of Effector Proteins

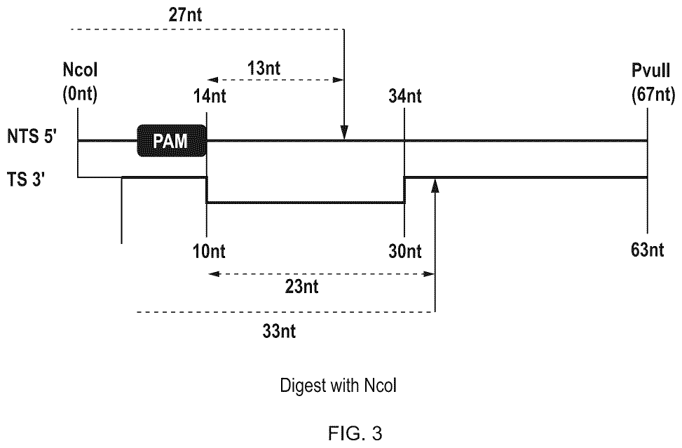

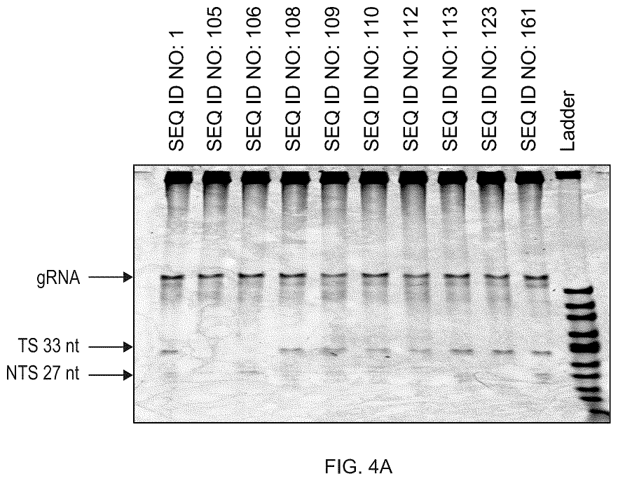

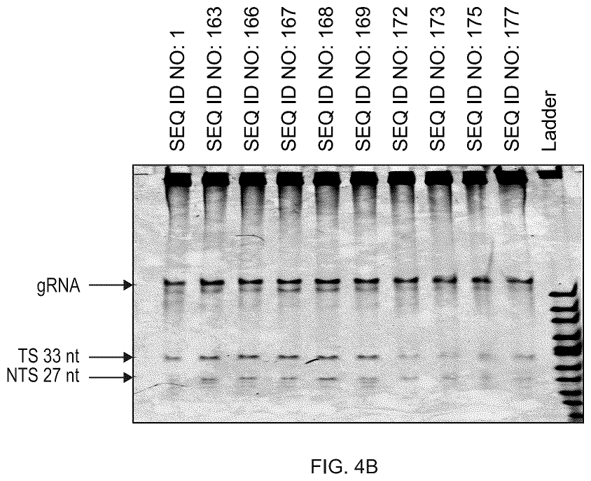

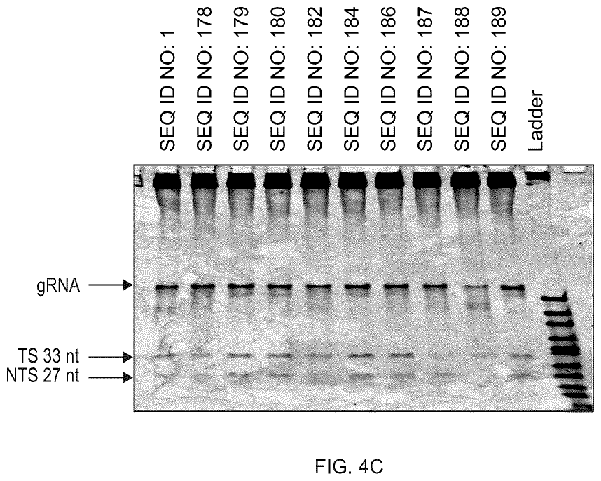

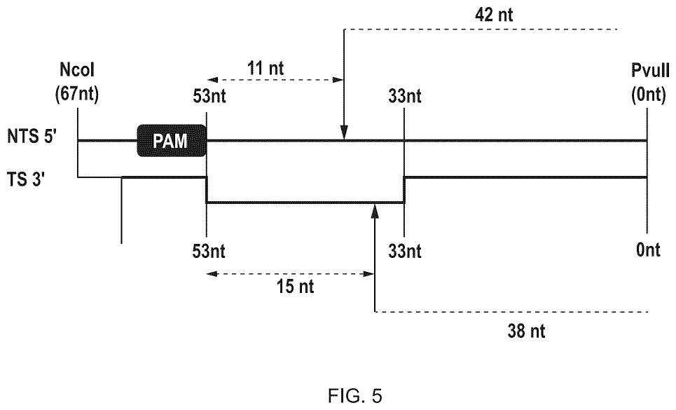

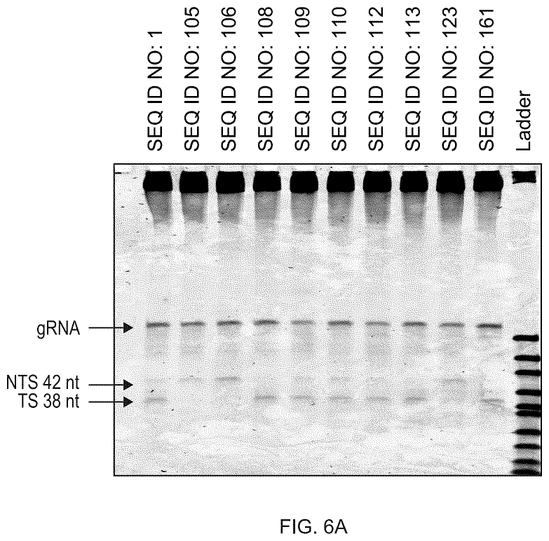

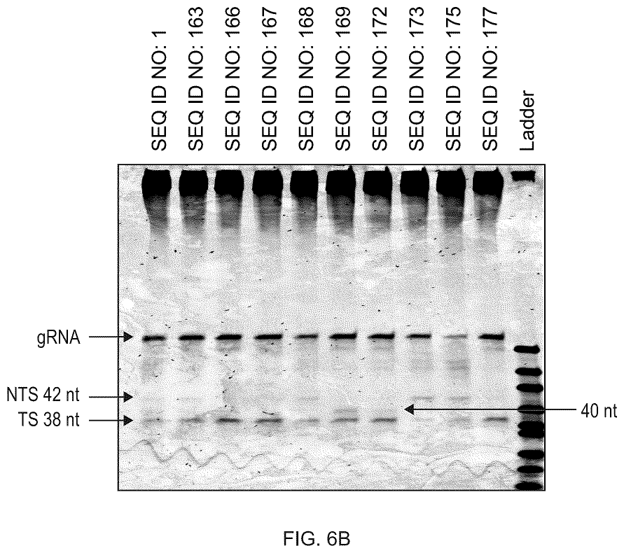

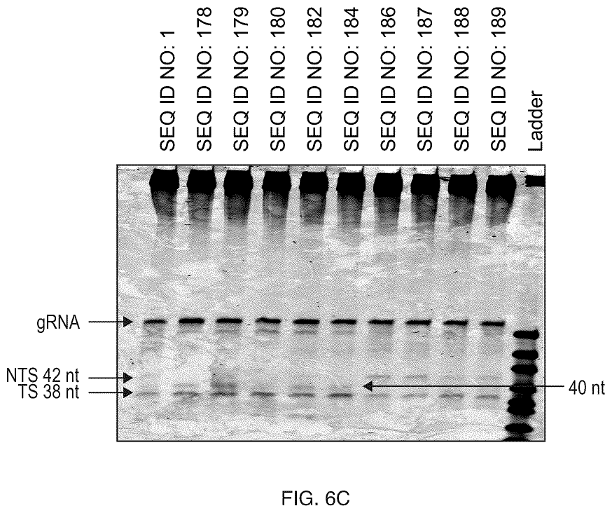

An in vitro determination of change in cut site of effector proteins was carried out. Briefly, a target nucleic acid was engineered to have two unique restriction enzyme recognition sites. The target nucleic acid comprises a target strand and a non-target strand. One restriction enzyme recognition site is 5′ to the PAM sequence (e.g., NcoI recognition site) and the other is 3′ to the PAM sequence (e.g., PvuII recognition site). The target nucleic acid was incubated with 50 nm of effector protein (e.g., effector protein having an amino acid sequence selected from SEQ ID NO: 105-106, 108-110, 112-113, 123, 128, 131, 155, 161, 166-169, 172-173, 175, 177-180, 182, 184, 186, and 187-190), 50 nm guide nucleic acid (e.g., crRNA) and a buffer (e.g., 200 mM HEPES pH 7.5 at 37° C., 3 mM Mg(OAc)₂, 1 mM TCEP and 0.2 mg/mL BSA, or other suitable 10× cleavage buffer) at 37° C. for cis cleavage. For positive control, WT effector protein of SEQ ID NO: 1 was used. In some experiments, incubated compositions further comprised a stop mix (e.g., proteinase K+EDTA, or formamide dye). Following the cis cleavage, the target nucleic acid was subjected to NcoI or PvuII restriction enzyme digestion resulting in specific sizes of digested polynucleotides. FIG. 3 shows expected sizes of nucleotide fragments following digestion with NcoI restriction enzyme. As shown in FIG. 3, the digestion with the WT effector protein would have produced 27 and 33 nucleotide containing fragments from the non-target strand and the target strand, respectively. Similarly, FIG. 5 shows expected sizes of nucleotide fragments following digestion with PvuII restriction enzyme. As shown in FIG. 5, the digestion with the WT effector protein would have produced 42 and 38 nucleotide containing fragments from the non-target strand and the target strand, respectively. Change is the cut site was determined by gel electrophoresis. The results of NcoI digested target nucleic acids for effector proteins are shown in FIGS. 4A-4C. Similarly, the results of PvuII digested target nucleic acids for effector proteins are shown in FIGS. 6A-6C. An analysis of FIGS. 4A-4C and 6A-6C confirms the results of Example 3 indicating at least some effector proteins were found to have enhanced nickase activity. For example, the effector protein comprising SEQ ID NO: 105-106 appears to have preference for nicking the double stranded target nucleic acid over double strand break.

### Example 5. Modulation of In Vitro Cis Cleavage Activity of Effector Proteins by Varying Spacer Length

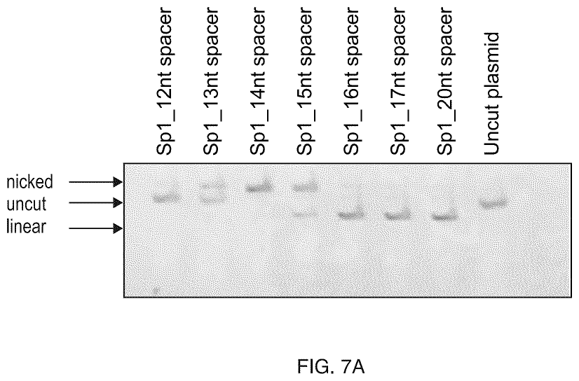

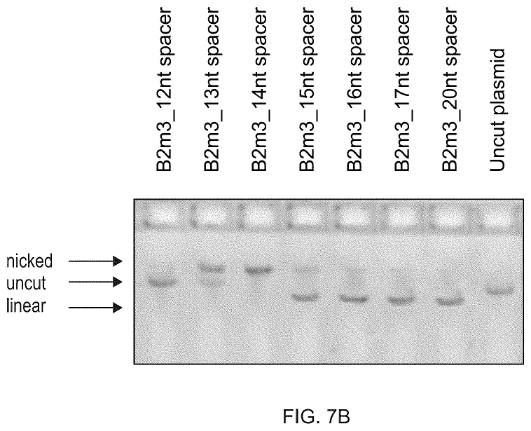

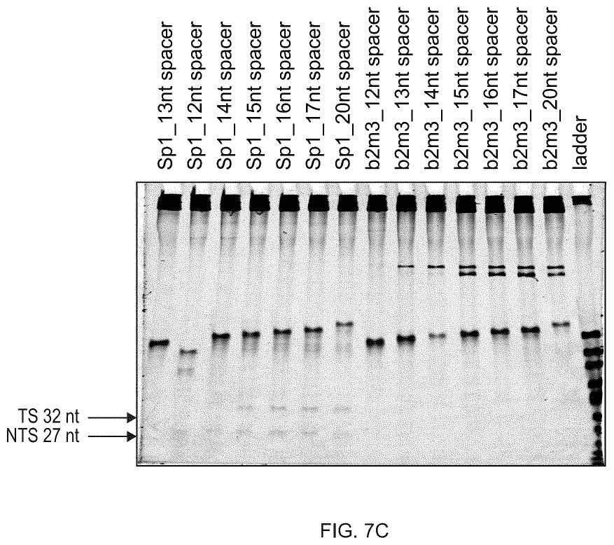

An in vitro screening was carried out to determine the effect of the length of a spacer sequence of a guide nucleic acid on an effector protein's cis cleavage activity (e.g., nickase activity). Specifically, an isolated WT effector protein (SEQ ID NO: 1) was incubated with a target nucleic acid (e.g., a supercoiled double stranded) and guide nucleic acids (e.g., crRNA) having different spacer lengths. Two sets of guide nucleic acids, one targeting SP1 gene and the other targeting B2M gene, having the lengths of the spacer sequence of 12, 13, 14, 15, 16, 17 and 20 nucleotides, were tested for a cis cleavage assay. Briefly, 50 nm of the WT effector protein, 50 nM of the guide nucleic acid, the target nucleic acid and a buffer (e.g., 200 mM HEPES pH 7.5 at 37° C., 3 mM Mg(OAc)₂, 1 mM TCEP and 0.2 mg/mL BSA, or other suitable 10× cleavage buffer) were incubated at 37° C. In some experiments, incubated compositions further comprised a stop mix (e.g., proteinase K+EDTA, or formamide dye). Cis cleavage activity of the effector proteins (e.g., nickase activity) were confirmed by gel electrophoresis. FIGS. 7A and 7B show the results of the cis cleavage activity against SP1 gene and B2M gene, respectively.

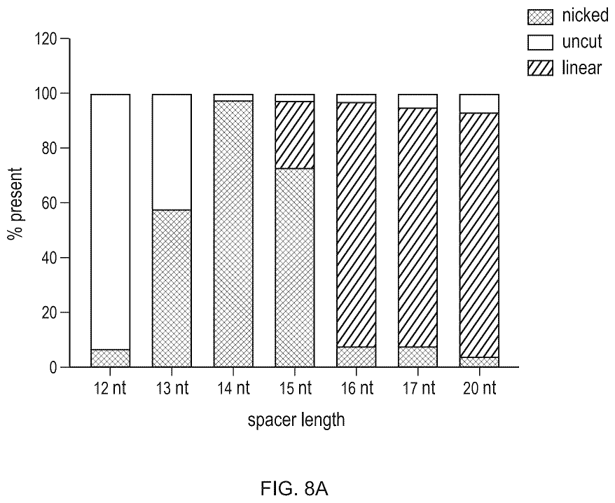

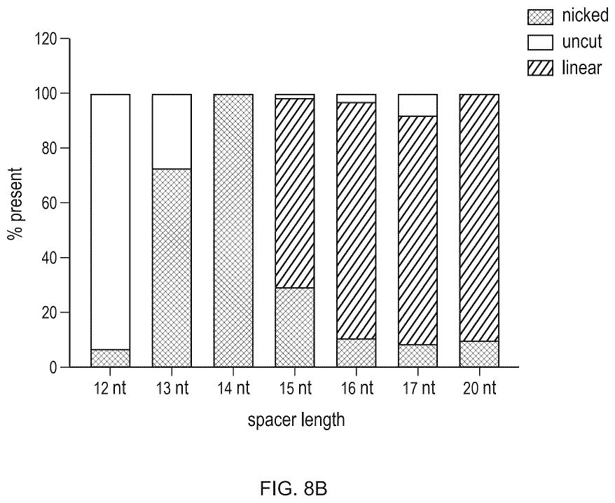

Additionally, the target nucleic acid was subjected to NcoI restriction enzyme digestion following the cis cleavage. The restriction enzyme mediated digestion resulted in specific sizes of digested polynucleotides. FIG. 3 shows expected sizes of digested polynucleotides for NcoI treated target nucleic acid. Change is the cut site was determined by gel electrophoresis. The results of NcoI digested target nucleic acids for the guide nucleic acids are shown in FIG. 7C. FIGS. 8A and 8B show quantitative analysis of FIGS. 7A and 7B data, respectively. An analysis of FIGS. 7A-7C and 8A-8B indicates that the catalytic activity of the effector proteins can be modified by changing the length of the spacer sequence of the guide nucleic acid. For example, a spacer length of 14 nucleotides appears to have preference for nicking the double stranded target nucleic acid over double strand break.

### Example 6: Exemplary CasPhi.12 Variant Testing in Mice

The lipid nanoparticles (LNPs) of the present disclosure were formulated using I471T variant of CasPhi.12 effector or protein (SEQ ID NO: 1) and guide RNA (mA*mU*mA*GAUUGCUCCUUACGAGGAGACGAGCAACGGCGGAAmG*mG*mU (SEQ ID NO: 266)) that is complementary to mouse PCSK9 gene.

In this example, WT C57BL/6J male mice (n=3-5), aged 6-8 weeks, were administered via intravenous (IV) injection via tail vein with 2 mg/kg of mRNA encoding nuclease and the guide RNA (1:3 ratio) formulated with an LNP comprising 35 mol % CKK-E12, 16 mol % DOPE, 46.5 mol % cholesterol and 2.5 mol % DMG-PEG 2000, with a CKK-E12/RNA w/w 10:1.

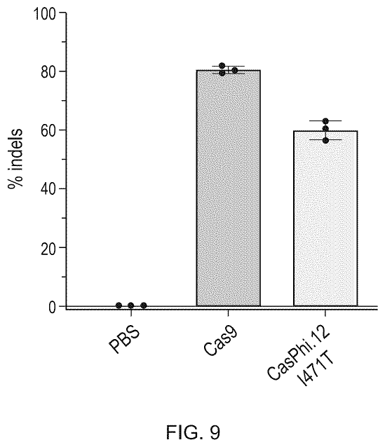

Cas9 mRNA and guide R8217 were injected as a control. The study was repeated, and each study ended 2-7 days post injection. Liver samples were collected for indel analysis by NGS. Data representative of multiple experiments, as provided in FIG. 9, show that a CasPhi.12 I147T variant produces indels comparable with Cas9 in WT mice in repeat studies with different mRNA lots and formulation runs.

### Example 7: Lipid Nanoparticle Formulations Comprising mRNA Encoding Effector Protein

In order to assess lipofection of mRNAs encoding various effector proteins, lipid nanoparticles (LNPs) are formulated using an mRNA having a nucleotide sequence encoding any one of the effector proteins of TABLE 1, a variant thereof or a fusion protein thereof, and two different guide RNAs. Cas9 system is used as a positive control. The guide RNAs each target different portion of the target gene. The mRNA and guide RNAs are mixed and diluted to various concentrations, as shown in TABLE 10 below, at a pH of about 3 to about 5 in acetate buffer or citrate buffer. Ionizable lipids, phospholipids, cholesterol and PEG lipids are dissolved in ethanol at various molar ratios. The lipid mixtures and RNA mixtures are further mixed in a microfluidic device at the various ionizable/RNA weight to weight ratios. The formulations are then further dialyzed against a Tris-saline buffer or phosphate buffered saline (PBS) at a pH of about 7.4 and concentrated to a desired concentration by Amicon filtration. The isolated LNPs are characterized to determine the encapsulation efficiency (EE), polydispersity index (PDI) and average particle size.

TABLE 10
LNP FORMULATIONS

| | Ionizable | Phospholipid | Cholesterol | PEG lipid |
|---|---|---|---|---|
| | lipid molar | molar | molar | molar |
| No. | ratio | ratio | ratio | ratio |
| 1 | 50 | 10 | 38.5 | 1.5 |
| 2 | 45 | 9 | 44 | 2 |
| 3 | 46.3 | 9.4 | 42.7 | 1.6 |
| 4 | 35 | 16 | 46.5 | 2.5 |
| 5 | 52.5 | 7.5 | 40 | 3 |
| 6 | 30 | 10 | 58.5 | 1.5 |
| 7 | 50 | 7 | 40 | 3 |
| 8 | 54.6 | 10.9 | 32.8 | 1.6 |
| 9 | 40 | 17 | 42 | 1 |

LNPs prepared as described above are administered to male C57BL/6J mice in a 6 day study (n=5) to test for mRNA delivery to animals in vivo. LNPs are dosed intravenously via the tail vein at doses of 0.5 mg/kg and 2 mg/kg. After 6 days, livers and serum are harvested for NGS and ELISA analysis, respectively.

### Example 8: CasPhi.12 Variants Testing in Mice

Lipid nanoparticles (LNPs) were formulated using variants of CasPhi.12 effector protein (SEQ ID NO: 1) and two guide RNA. One guide was engineered for targeting mPCSK9 gene (R8860), wherein the guide comprised a repeat sequence of (SEQ ID NO: 265) and a spacer sequence of GAGCAACGGCGGAAGGU (SEQ ID NO: 267). The other guide RNA was engineered for targeting mAPOC3 gene (R16962) wherein the guide comprised a repeat sequence of (SEQ ID NO: 265) and a spacer sequence of AGGUGAGAUCUAGGGAG (SEQ ID NO: 268). Five variants of CasPhi. 12 effector protein (SEQ ID NO: 1), one single mutant, one double mutant and three triple mutants were tested. The single mutant was I471T variant. The double mutant was L26R+I471T variant. The triple mutants were: (1) L26K+H208R+I471T; (2) L26K+L149R+I471T; and (3) L26K+D703G+I471T. In this example, mice were injected with a dose of 0.5 mg/kg, 1 mg/kg and 3 mg/kg of mRNA encoding effector protein and the guide RNA (1:3 ratio) formulated with an LNP.

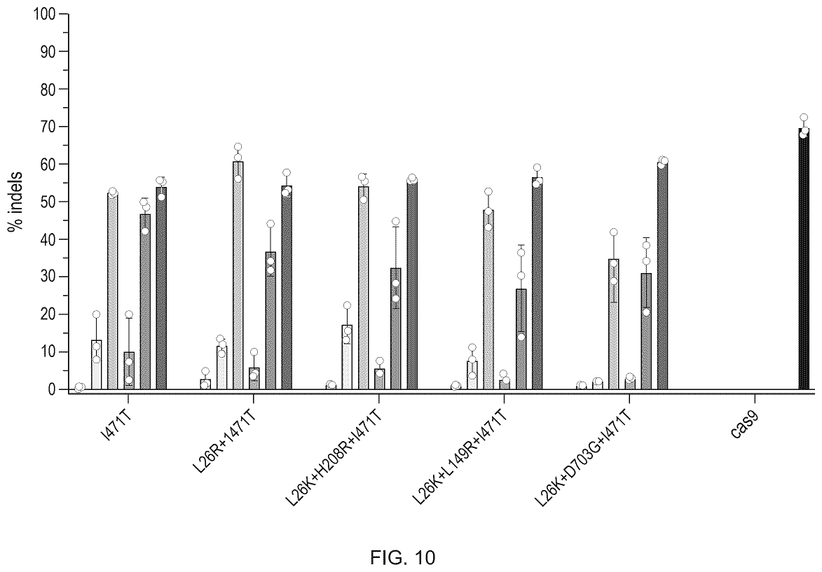

Cas9 mRNA and guide were injected as a control. Liver was harvested four days post injection and NGS analysis was performed. The results for each variant, % indel generated by (from left to right) R16962 at 0.5 mg/kg dose, R16962 at 1.0 mg/kg dose, R16962 at 3.0 mg/kg dose, R8860 at 0.5 mg/kg dose, R8860 at 1.0 mg/kg dose and R8860 at 3.0 mg/kg dose, are shown in FIG. 10. Cas9 was used as a positive control. An analysis of FIG. 10 indicates that, while editing is relatively low for R16962, the double mutant was able to rescue editing. This experiment suggests that, for some guide/dose regimes, the double mutant may be superior to the single mutant.

### Example 9: CasPhi.12 System Mediated Editing of Target Nucleic Acid Using Chemically Modified Guide Nucleic Acids

Two sets of chemical modifications were designed to identify compatible modifications for a guide nucleic acid in combination with L26R variant of WT CasPhi. 12 (SEQ ID NO: 1). The guide nucleic acid comprised a nucleotide sequence of

(SEQ ID NO: 269)
AUAGAUUGCUCCUUACGAGGAGACCUUUGCACUUUCCUUAG,

which was modified by one or more 2′OMe sugar modifications, and one or more PS backbone modifications. The two sets included the following modifications: (1) Unbiased tiling of modifications; and (2) Combinatorial modifications rationally designed based on predicted structure of the effector protein. FIGS. 11A-11F show various guide nucleic acid modifications that were tested. The system was then evaluated in primary human hepatocytes (PHH) for integration of the AAV-nLuc reporter.

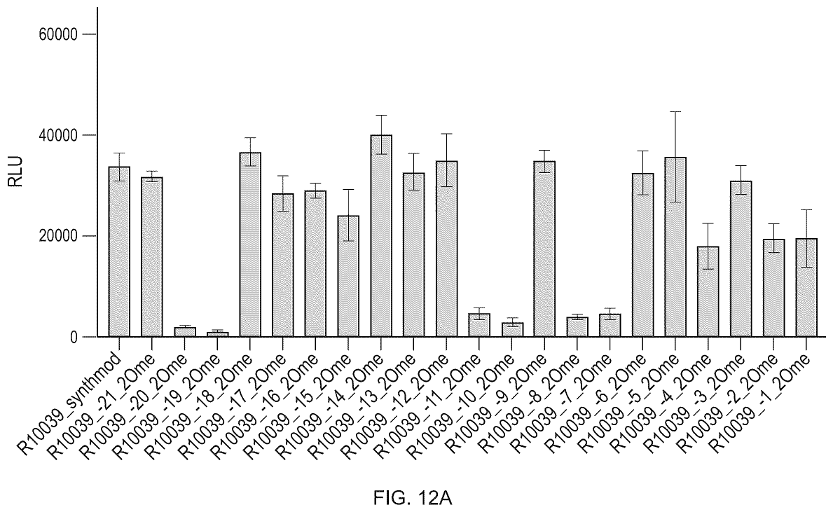

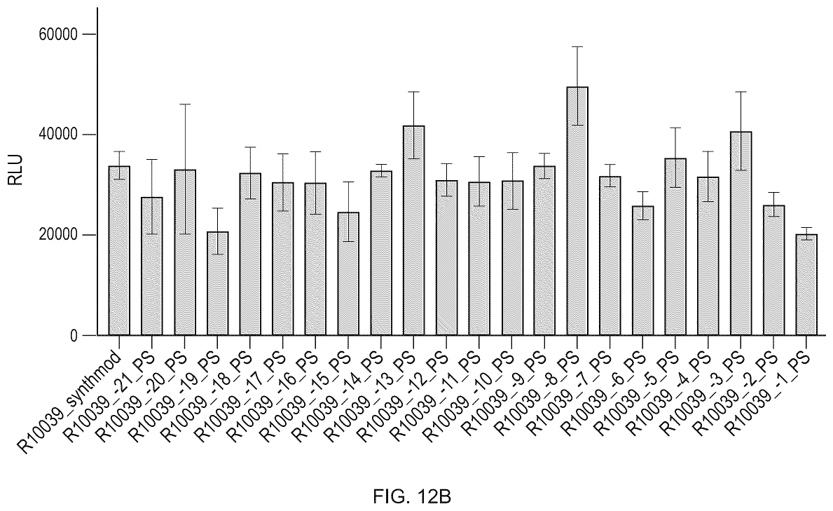

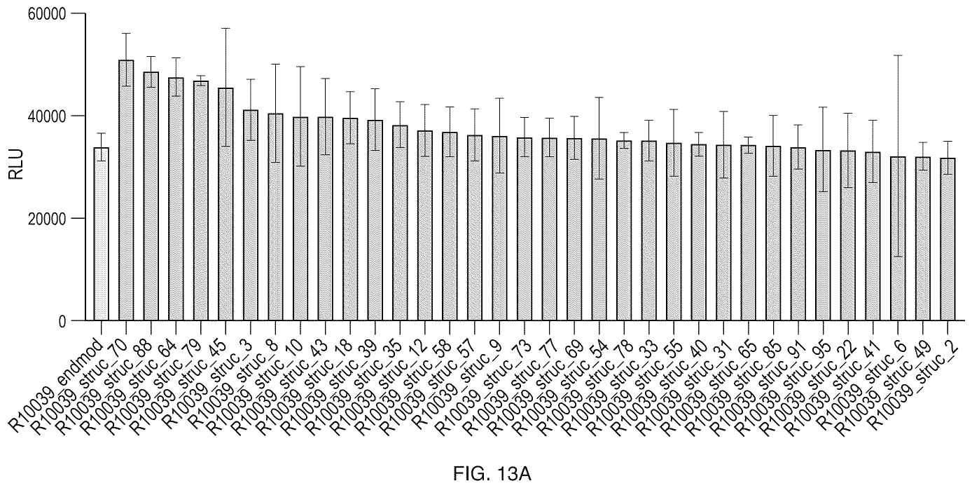

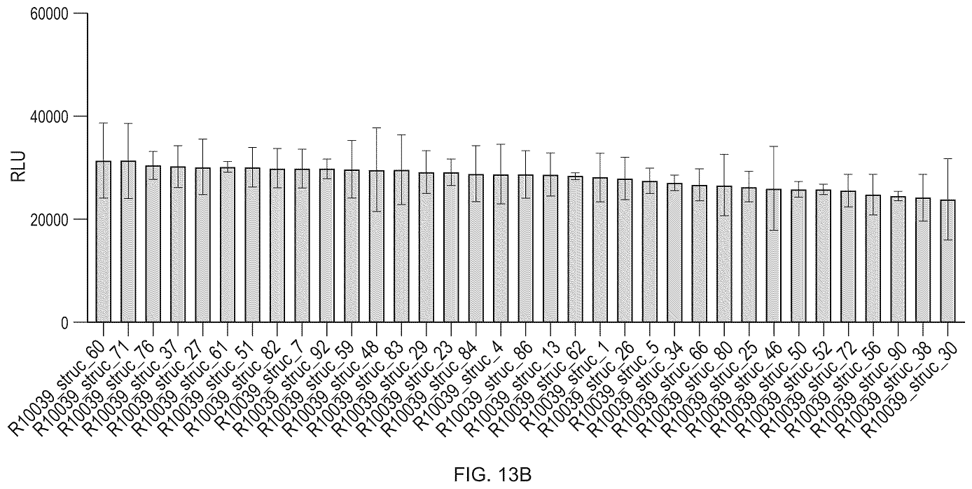

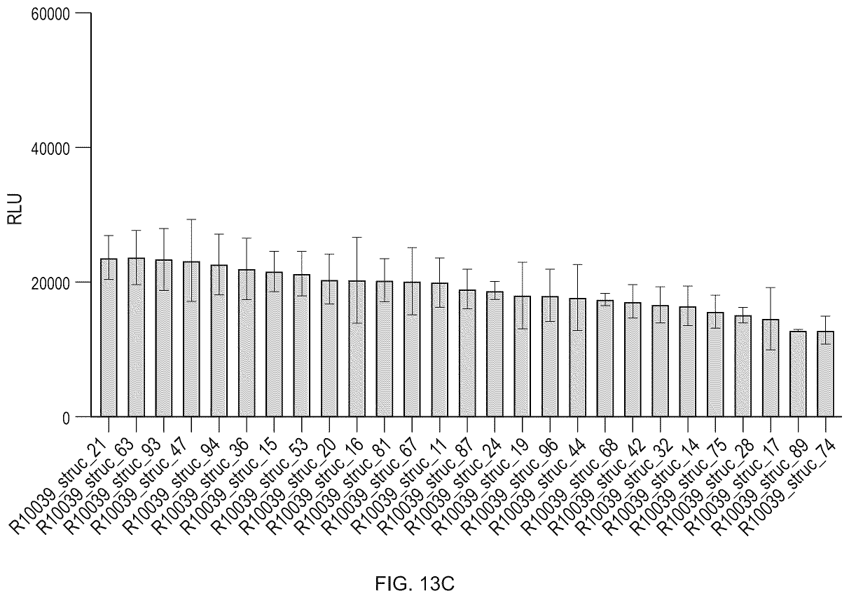

Briefly, 100,000 PHH were transfected with LNPs comprising 400 ng total RNA (1:1 mRNA/gRNA ratio) at 96-well scale by MessengerMax and co-transduced with 1E4 MOI AAVDJ-nLuc reporter. After 72 hours, integration was quantified by luciferase assay. FIGS. 12A-12B show results of luciferase assay for unbiased modifications. FIGS. 13A-13C show results of luciferase assay for combinatorial modifications, which are divided into three parts, FIGS. 13A-13C, for legibility.

An analysis of FIGS. 12A-12B and FIGS. 13A-13C indicate that PS backbone modifications are tolerated at all tested positions, whereas 2′OMe sugar modifications at positions −20, −19, −11, −10, −8, and −7 eliminate nuclease activity.

### Example 10: CasPhi.12 Variant Mediated Editing of Target Nucleic Acid

Lipid nanoparticles (LNPs) were formulated using variants of WT CasPhi.12 effector protein (SEQ ID NO: 1) and seven guide nucleic acids. The seven guide nucleic acids included:

(1) R10039
(AUAGAUUGCUCCUUACGAGGAGACCUUUGCACUUUCCUUAG
(SEQ ID NO: 269));
(2) R10006
(AUAGAUUGCUCCUUACGAGGAGACGUAUUUGUGAAGUCUUA
(SEQ ID NO: 270));
(3) R10059
(AUAGAUUGCUCCUUACGAGGAGACAAUAAAGCAUAGUGCAA
(SEQ ID NO: 272));
(4) R10067
(AUAGAUUGCUCCUUACGAGGAGACUGAGAUCAACAGCACAG
(SEQ ID NO: 273));
(5) R10081
(AUAGAUUGCUCCUUACGAGGAGACCAUUUUAGUCUGUCUUC
(SEQ ID NO: 274));
(6) R10131
(AUAGAUUGCUCCUUACGAGGAGACGGCUCUGAUUCCUACAG
(SEQ ID NO: 275));
and
(7) R10144
(AUAGAUUGCUCCUUACGAGGAGACGACUUAGAUUAUGC AUU
(SEQ ID NO: 276)).

The variants of WT CasPhi. 12 effector protein (SEQ ID NO: 1) that were tested included L26R substitution, I471T substitution and both, L26R and I471T, substitutions. Cas9 mRNA and corresponding guide nucleic acid were used as a positive control.

Briefly, 500,000 PHH were transfected with LNPs comprising 2 μg total RNA (1:1 mRNA/gRNA ratio) at 24-well scale by MessengerMax and co-transduced with 1E4 MOI AAVDJ-nLuc reporter. After 72 hours, integration was quantified by luciferase assay.

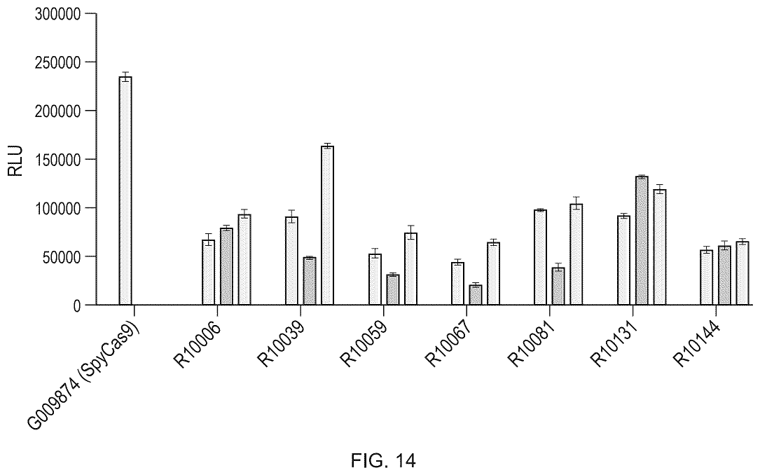

FIG. 14 shows results of the luciferase assay. An analysis of FIG. 14 indicates that CasPhi.12 I471T variant has variable activity across targets, increasing integration for some guide nucleic acids but decreasing for others. Also, CasPhi.12 L26R/I471T variant has improved integration activity across all guide nucleic acids, with nearly 2-fold activity increase for at least one guide nucleic acid, R10039.

### Example 11: CasPhi.12 Variant Mediated Editing of Target Nucleic Acid

Lipid nanoparticles (LNPs) were formulated using variants of WT CasPhi. 12 and tested for their capacity to generate indels in PCSK9 gene. The variants included L26R substitution, I471T substitution and both, L26R and I471T, substitutions. Four guide nucleic acids were which included:

(1) R8860
(mA*mU*mA*GAUUGCUCCUUACGAGGAGACGAGCAACGGCGGAAmG*
mG*mU (SEQ ID NO: 277));
(2) R8866
(mA*mU*mA*GAUUGCUCCUUACGAGGAGACAUGUCACAGAGUGGmG*
mA*mC (SEQ ID NO: 278));
(3) R16962
(auagauugcuccuuacgaggagacAGGUG AGAUCUAGGGAG
(SEQ ID NO: 279));
and
(4) R17005
(auagauugcuccuuacgaggagacAUCCCUAG AAGCAGCUA
(SEQ ID NO: 280)).

Briefly, mice were injected with a single dose (2 mpk) of LNPs comprising mRNA encoding effector protein and the guide nucleic acid (1:3 ratio).

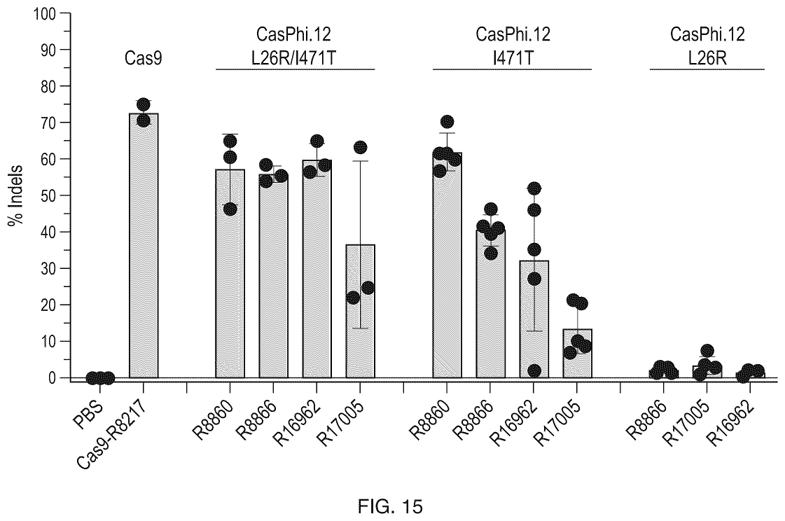

Cas9 mRNA and guide nucleic acid were injected as a control. Liver was harvested four days post injection and NGS analysis was performed. The results of the experiment are provided in FIG. 15. An analysis of FIG. 15 indicates that, while editing is relatively low for R16962, the double mutant was able to rescue editing. This suggests that, for some guide nucleic acid/dose regimes, the double mutant may be superior to the single mutant.

### Example 12: CasPhi.12 Variant Editing in Mammalian Cells

Mammalian HEK293T cells were transected with 100 ng of plasmid encoding a variant of CasPhi. 12 (SEQ ID NO: 1) and 100 ng of plasmid encoding a guide nucleic acid. Cells were harvested 72 hours later and target nucleic acid editing (Mod %) analyzed with NGS. The variants tested are provided in TABLE 11, which correspond to variants comprising the amino acid sequences of SEQ ID NOS: 271, 281-293, 324-332 and 334. Some of the variants comprise a truncation at the N terminus or C terminus. For example, 1Ctruncation indicates that the protein has a truncation of one nucleotide from the C terminus relative to SEQ ID NO: 1.

TABLE 11
List of Variants Tested for Editing of Mammalian Cells

| SEQ | | SEQ | | SEQ | |
|---|---|---|---|---|---|
| ID NO: | Description | ID NO: | Description | ID NO: | Description |
| 271 | L26R, I471T, | 331 | I471T, S223P, | 287 | L26R, I471T and |
| | S223P and D703G | | D703G and L149R | | 2Ntruncation |
| 324 | L26R, I471T, | 332 | I471T, S223P, | 288 | L26R, I471T and |
| | S223P, D703G and | | D703G, L149R and | | 3Ntruncation |
| | H208R | | H208R | | |
| 325 | L26R, I471T, | 281 | I471T, S223P, | 289 | L26R, I471T and |
| | S223P, D703G and | | D703G, D704G | | 4Ntruncation |
| | L149R | | and A706G | | |
| 326 | L26R, I471T, | 282 | I471T, S223P, | 290 | L26R, I471T and |
| | S223P, D703G, | | D703G, L149R, | | 1Ctruncation |
| | L149R and H208R | | H208R, D704G | | |
| | | | and A706G | | |
| 327 | L26R, I471T, | 283 | I471T and E157R | 291 | L26R, I471T and |
| | S223P, D703G, | | | | 2Ctruncation |
| | D704G and A706G | | | | |
| 328 | L26R, I471T, | 284 | I471T, E157R, | 292 | L26R, I471T and |
| | S223P, D703G, | | S223P and D703G | | 3Ctruncation |
| | L149R, H208R, | | | | |
| | D704G and A706G | | | | |
| 329 | I471T, S223P and | 285 | L26R, I471T, | 293 | L26R, I471T and |
| | D703G | | E157R, S223P and | | 4Ctruncation |
| | | | D703G | | |
| 330 | I471T, S223P, | 286 | L26R, I471T and | 334 | L26R and I471T |
| | D703G and H208R | | 1Ntruncation | | |

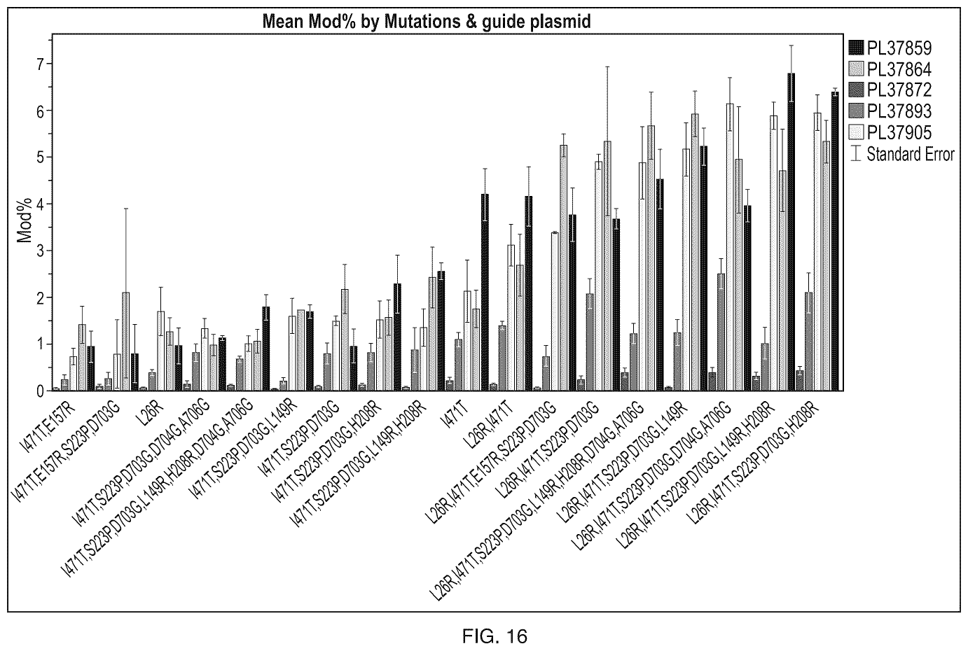

Variants were tested with 5 different guide nucleic acids (PL37859, PL37864, PL37872, PL37893 and PL37905), each having a repeat sequence of SEQ ID NO: 348. Data are provided in FIGS. 16-18.

FIG. 16 shows CasPhi.12 variants and guide nucleic acids induced modifications (Mod %) in a target nucleic acid.

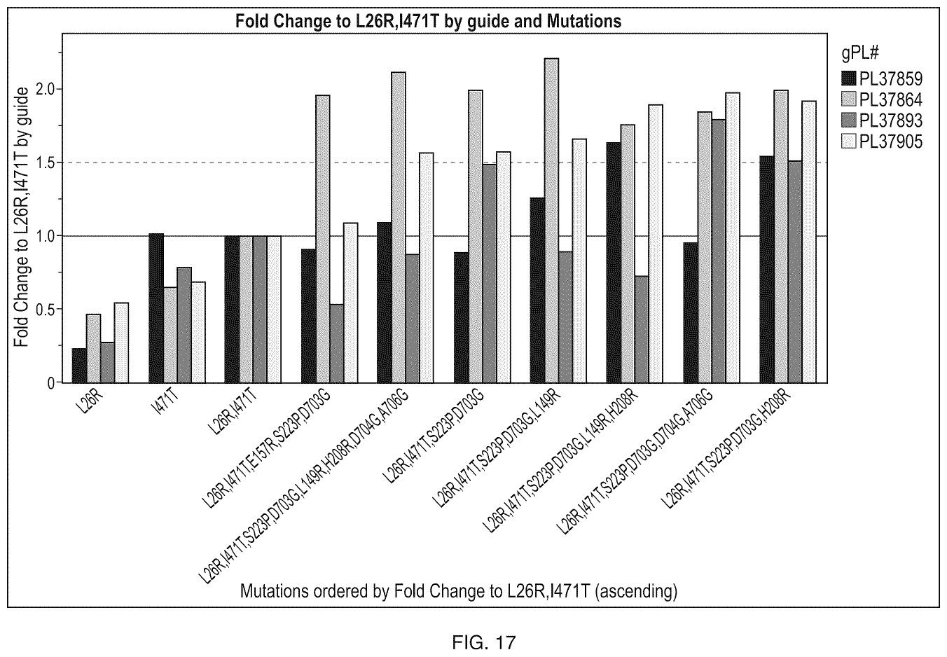

FIG. 17 shows fold change relative to CasPhi. 12 variant L26R, I471T. It is notable that CasPhi.12 variant L26R, I471T, S223P, D703G and H208R (SEQ ID NO: 324) has a positive fold change of at least 1.5 for every guide screened. Three other variants have at least a 1.5 fold change at three of the guides tested. Common residues changed between the top four performers in this experiment were L26R, I471T, S223P and D703G. L26R, I471T, S223P, D703G, D704G and A706G (SEQ ID NO: 327), which showed higher fold changes than L26R, I471T, S223P and D703G (SEQ ID NO: 271).

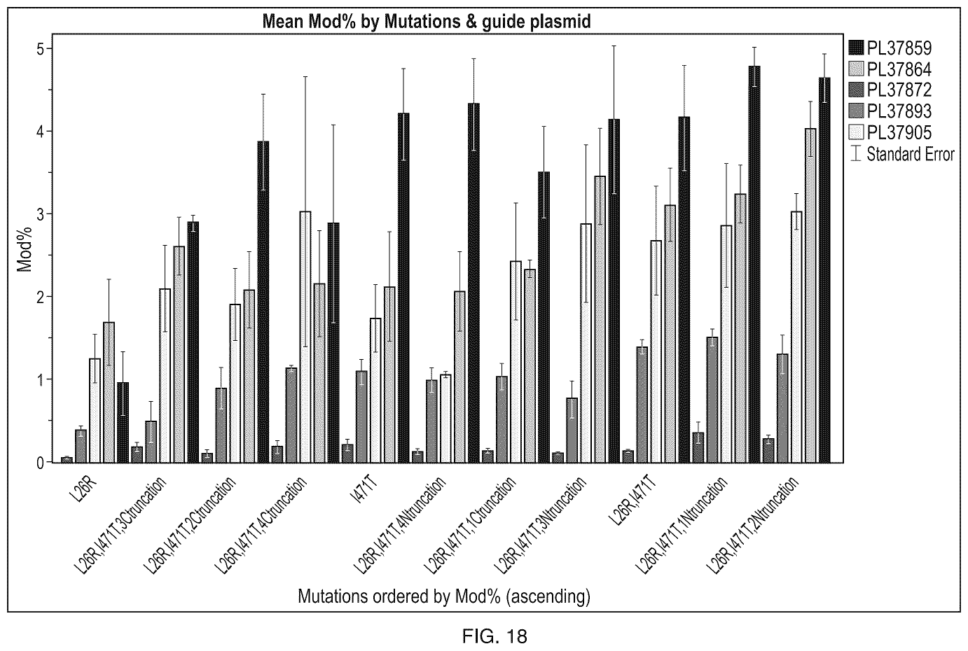

FIG. 18 shows induced modifications (Mod %) in a target nucleic acid for variants having truncations of 1-4 nucleotides at the N or C terminus. These results shows that such truncations did not dramatically change the editing outcomes when compared to CasPhi.12 variant L26R and I471T (SEQ ID NO: 334). This suggests that these truncations could be combined with other variants described herein.

### Example 13: Editing in Mammalian Cells by Effector Protein Variant

Effector protein variants and guide nucleic acid targeting APOC3 were cloned into two separate plasmids. The variants tested are provided in TABLE 12, which correspond to variants comprising the amino acid sequences of SEQ ID NOS: 271, 379-394 and 396-398. The guide nucleic acid comprised a nucleotide sequence of

(SEQ ID NO: 421)
AUUGCUCCUUACGAGGAGACUUGCCCUCCUGGCGCUCCUG.

TABLE 12
List of Variants Tested

| SEQ | |
|---|---|
| ID NO: | Description |
| 271 | L26R, I471T, S223P and D703G |
| 379 | L26R-T87G-S186G-H208R- |
| | S223P-C405L-I471T-S526N- |
| | D703G |
| 380 | L26R-A121Q-S223P-E258K- |
| | I471T-D523K-S526N-D703G |
| 381 | L26R-N147K-H208R-S223P- |
| | E258K-I471T-M503K-D703G |
| 382 | L26R-N147K-S186G-S223P- |
| | E258K-I471T-S526N-D549L- |
| | S638K-D703G |
| 383 | S21L-L26R-S186G-Y220S- |
| | S223P-I471T-D703G |
| 384 | L26R-T87G-A121Q-S186G- |
| | H208R-Y220S-S223P-C405L- |
| | I471T-D523K-D703G |
| 385 | S21L-L26R-A121Q-N147K- |
| | S186G-Y220S-S223P-I471T- |
| | S526N-D549L-D703G |
| 386 | S21L-L26R-Q76R-N147K- |
| | L149R-Y220S-S223P-Y251R- |
| | E258K-I471T-M503K-Q552R- |
| | D703G |
| 387 | L26R-A121Q-Y220S-S223P- |
| | C405L-I471T-D523K-Q552R- |
| | D703G |
| 388 | S21L-L26R-A121Q-N147K- |
| | Y220S-S223P-Y251R-C405L- |
| | I471T-D703G |
| 389 | L26R-Q76R-T87G-S223P- |
| | E258K-C279R-I471T-M503K- |
| | D523K-D703G |
| 390 | L26R-N147K-S186G-S223P- |
| | I471T-M503K-S526N-D703G |
| 391 | S21L-L26R-T87G-N147K- |
| | H208R-Y220S-S223P-I471T- |
| | D703G |
| 392 | S21L-L26R-A121Q-N147K- |
| | S186G-S223P-E258K-I471T- |
| | D523K-Q552R-D703G |
| 393 | L26R-A121Q-L149R-S186G- |
| | Y220S-S223P-I471T-D703G |
| 394 | L26R-A121Q-N147K-Y220S- |
| | S223P-I471T-M503K-S526N- |
| | D549L-D703G |
| 396 | L26R-T87G-S186G-Y220S- |
| | S223P-I471T-M503K-D703G |
| 397 | S21L-L26R-Q76R-T87G- |
| | N147K-S186G-S223P-I471T- |
| | S526N-S638K-D703G |
| 398 | S21L-L26R-A121Q-Y220S- |
| | S223P-C405L-I471T-M503K- |
| | D703G |

### Cell Culture Conditions

Mammalian HEK293T cells were cultured in Dulbecco's modified Eagle's medium (DMEM) supplemented with 10% (v/v) fetal bovine serum and 1% penicillin streptomycin. All cells were incubated, maintained, and cultured at 37° C. with 5% CO₂.

### HEK293T Tissue Culture Transfection Protocol and Genomic DNA Preparation

HEK293T cells were seeded on 96-well microplate. After 24 hours at 37° C., the cells were transfected at approximately 60% confluency with 0.72 μl TransIT-293 transfection reagent (Mirus Bio) using 50 ng plasmid comprising effector protein variant and 50 ng plasmid encoding the guide nucleic acid. The cells were then cultured for 3 days following transfection at 37° C., after which the medium was removed, and genomic DNA was extracted by the addition of 100 μl of freshly prepared Quick Extract DNA extraction solution (BioSearch Technologies) directly into each well of the tissue culture plate. The genomic DNA mixture was incubated at 65° C. for 5 min followed by incubation at 90° C. for another 5 min prior to subsequent PCR steps.

### High-Throughput DNA Sequencing of Genomic DNA Samples

Genomic sites of interest were amplified from genomic DNA samples and sequenced on an Illumina MiSeq. In brief, amplification primers containing Illumina forward and reverse adapters were used for a first round of PCR (adapter PCR) to amplify the genomic region of interest. Adapter PCR reactions (20 μl) were performed with 0.5 μM of each forward and reverse primer, 1 μl genomic DNA extract, Accuprime Taq Hifi polymerase, and 10x Accuprime buffer II (Invitrogen). PCR reactions were carried out as follows: 94° C. for 2 min, then 35 cycles of 94° C. for 30 s, 61° C. for 30 s, and 68° C. for 30 s, followed by a final 68° C. extension for 2 min. Unique Illumina barcoding primer pairs were added to each sample in a secondary PCR reaction (index PCR). Specifically, 10 μl of a given index PCR reaction contained 0.5 μM of each unique forward and reverse Illumina barcoding primer pair, 1 μl unpurified PCR reaction mixture from the adapter PCR, and Accuprime Taq Hifi polymerase, and 10× Accuprime buffer II (Invitrogen). The index PCR reactions were carried out as follows: 94° C. for 2 min, then 12 cycles of 94° C. for 30 s, 61° C. for 30 s, and 68° C. for 30 s, followed by a final 68° C. extension for 2 min. PCR products were evaluated analytically by electrophoresis in a 1.5% agarose gel. The PCR products were then purified using SPRIselect Beads-Based Reagent (Beckman Coulter). DNA concentration was measured by Lunatic UV/Vis spectrophotometer (Unchained Labs) and sequenced on an Illumina MiSeq instrument according to the manufacturer's protocols.

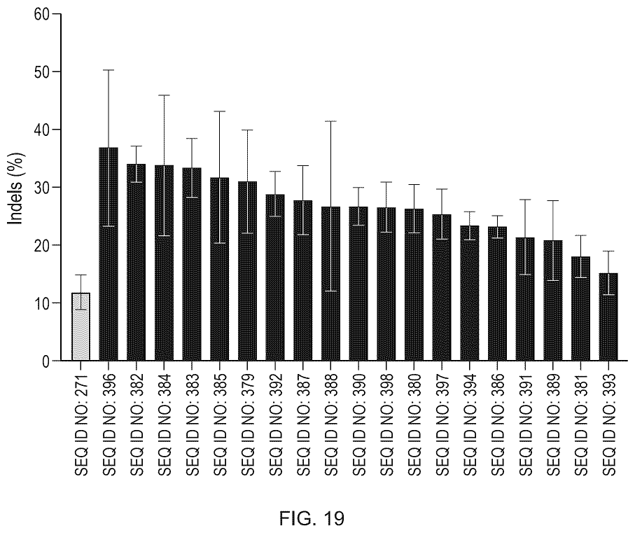

Sequencing reads were demultiplexed using MiSeq Reporter (Illumina). Alignment of amplicon sequences to a reference sequence was performed using CRISPResso2. The indel percentage was calculated by aligning the sequencing reads with a reference sequence and indel yields were calculated as: (number of indel-containing reads)/(total reads). The results are shown in FIG. 19. These results showed that all the tested effector protein variants produced at least 10% indel. Among the tested variants, the effector protein variant comprising an amino acid sequence of SEQ ID NO: 271 produced the lowest indel. Moreover, at least 12 effector protein variants (SEQ ID NOS: 379, 380, 382, 383, 384, 385, 387, 388, 390, 392, 396 and 398) produced at least twice the number of indels that the effector protein variant having an amino acid sequence of SEQ ID NO: 271.

1-48. (canceled)

49. A nucleic acid encoding an effector protein, wherein the effector protein is at least 90% identical to SEQ ID NO: 1, and wherein the effector protein comprises a combination of amino acid substitutions described in TABLE 2.

50. The nucleic acid of claim 49, wherein the combination of amino acid substitutions is selected from: (a) L26R, I471T, S223P and D703G; (b) L26R, I471T, S223P, D703G and H208R; (c) L26R, I471T, S223P, D703G and L149R; (d) L26R, I471T, S223P, D703G, L149R and H208R; (e) L26R, I471T, S223P, D703G, D704G and A706G; (f) L26R, I471T, S223P, D703G, L149R, H208R, D704G and A706G; (g) I471T, S223P and D703G; (h) I471T, S223P, D703G and H208R; (i) I471T, S223P, D703G and L149R; (j) I471T, S223P, D703G, L149R and H208R; (k) I471T, S223P, D703G, D704G and A706G; (l) I471T, S223P, D703G, L149R, H208R, D704G and A706G; (m) I471T and E157R; (n) I471T, E157R, S223P and D703G; (o) L26R, I471T, E157R, S223P and D703G; (p) L26R, T87G, S186G, H208R, S223P, C405L, I471T, S526N and D703G; (q) L26R, A121Q, S223P, E258K, I471T, D523K, S526N and D703G; (r) L26R, N147K, H208R, S223P, E258K, I471T, M503K and D703G; (s) L26R, N147K, S186G, S223P, E258K, I471T, S526N, D549L, S638K and D703G; (t) S21L, L26R, S186G, Y220S, S223P, I471T and D703G; (u) L26R, T87G, A121Q, S186G, H208R, Y220S, S223P, C405L, I471T, D523K and D703G; (v) S21L, L26R, A121Q, N147K, S186G, Y220S, S223P, I471T, S526N, D549L and D703G; (w) S21L, L26R, Q76R, N147K, L149R, Y220S, S223P, Y251R, E258K, I471T, M503K, Q552R and D703G; (x) L26R, A121Q, Y220S, S223P, C405L, I471T, D523K, Q552R and D703G; (y) S21L, L26R, A121Q, N147K, Y220S, S223P, Y251R, C405L, I471T and D703G; (z) L26R, Q76R, T87G, S223P, E258K, C279R, I471T, M503K, D523K and D703G; (aa) L26R, N147K, S186G, S223P, I471T, M503K, S526N and D703G; (bb) S21L, L26R, T87G, N147K, H208R, Y220S, S223P, I471T and D703G; (cc) S21L, L26R, A121Q, N147K, S186G, S223P, E258K, I471T, D523K, Q552R and D703G; (dd) L26R, A121Q, L149R, S186G, Y220S, S223P, I471T and D703G; (ee) L26R, A121Q, N147K, Y220S, S223P, I471T, M503K, S526N, D549L and D703G; (ff) L26R, T87G, A121Q, Y220S, S223P, E258K, C405L, I471T and D703G; (gg) L26R, T87G, S186G, Y220S, S223P, I471T, M503K and D703G; (hh) S21L, L26R, Q76R, T87G, N147K, S186G, S223P, I471T, S526N, S638K and D703G; (ii) S21L, L26R, A121Q, Y220S, S223P, C405L, I471T, M503K and D703G; (jj) L26R, S223P, I471T and D703G; (kk) L26R, T87G, S223P, I471T, S526N and D703G; (ll) L26R, T87G, N147K, S223P, I471T, S526N and D703G; (mm) L26R, T87G, N147K, S223P, E258K, I471T, S526N and D703G; (nn) L26R, T87G, Y220S, S223P, I471T, S526N and D703G; (oo) L26R, T87G, N147K, Y220S, S223P, E258K, I471T, S526N and D703G; (pp) L26R, Q76R, T87G, N147K, Y220S, S223P, E258K, C279R, I471T, M503K, D523K and D703G; (qq) L26R, Q76R, T87G, N147K, Y220S, S223P, E258K, I471T, M503K, D523K and D703G; (rr) L26R, Q76R, T87G, N147K, Y220S, S223P, E258K, I471T, M503K and D703G; (ss) S21L, L26R, Q76R, T87G, S223P, E258K, C279R, C405L, I471T, M503K, D523K and D703G; (tt) L26R, Q76R, T87G, N147K, S186G, Y220S, S223P, E258K, C405L, I471T, M503K and D703G; (uu) L26R, T87G, Y220S, S223P, I471T and D703G; (vv) L26R, T87G, N147K, Y220S, S223P, E258K, I471T and D703G; (ww) S21L, L26R, T87G, N147K, Y220S, S223P, E258K, I471T and D703G; (xx) S21L, L26R, T87G, N147K, Y220S, S223P, E258K, C405L, I471T, M503K and D703G; (yy) S21L, L26R, T87G, A121Q, N147K, Y220S, S223P, E258K, C405L, I471T, M503K, S638K and D703G; (zz) S21L, L26R, T87G, A121Q, N147K, S186G, Y220S, S223P, E258K, C405L, I471T, M503K, S638K and D703G; (aaa) L26R, T87G, S223P, I471T and D703G; (bbb) L26R, T87G, N147K, S223P, I471T and D703G; (ccc) L26R, T87G, N147K, S223P, E258K, I471T and D703G; (ddd) L26R, T87G, S186G, H208R, S223P, C405L, I471T and D703G; (eee) L26R, N147K, S186G, S223P, I471T, M503K and D703G; and (fff) L26R, S223P, E258K, I471T and D703G.

51. The nucleic acid of claim 49, wherein the combination of amino acid substitutions is L26R, I471T, S223P and D703G.

52. The nucleic acid of claim 49, wherein the effector protein comprises or consists of any one of the amino acid sequences described in TABLE 4.

53. The nucleic acid of claim 49, wherein the nucleic acid encoding the effector protein comprises a messenger RNA.

54. The nucleic acid of claim 49, wherein the nucleic acid encoding the effector protein is an adeno associated viral (AAV) vector.

55. The nucleic acid of claim 54, wherein the AAV vector encodes a guide RNA, an effector partner, or a combination thereof.

56. The nucleic acid of claim 55, wherein the effector partner is selected from a methyltransferase, a reverse transcriptase, and a deaminase.

57. A fusion protein or nucleic acid encoding the fusion protein, wherein the fusion protein comprises the effector protein of claim 49 and a heterologous peptide or protein, wherein the effector protein is covalently linked to the heterologous peptide or protein, optionally via a linker molecule.

58. A system comprising one or more components, wherein the one or more components individually comprise:
a) the nucleic acid of claim 49; and
b) a guide RNA or a nucleic acid that encodes the guide RNA,
wherein the guide RNA comprises a repeat sequence and a spacer sequence, wherein the repeat sequence, at least partially, interacts with the effector protein, and wherein the spacer sequence comprises a nucleic acid sequence that hybridizes to a target sequence in a target nucleic acid.

59. The system of claim 58, wherein the repeat sequence comprises a nucleotide sequence that is at least 90% identical to the nucleotide sequence recited in TABLE 7.

60. The system of claim 58, wherein the system comprises an expression vector, wherein the expression vector comprises the nucleic acid and the nucleic acid that encodes the guide RNA.

61. The system of claim 60, wherein the expression vector is an adeno associated viral (AAV) vector.

62. The system of claim 58, comprising a lipid nanoparticle (LNP), wherein the LNP encapsulates the nucleic acid and the guide RNA.

63. A composition comprising the nucleic acid of claim 49; and a guide RNA or nucleic acid encoding the guide RNA.

64. A pharmaceutical composition comprising the nucleic acid of claim 49, a guide RNA or nucleic acid encoding the guide RNA; and a pharmaceutically acceptable excipient.

65. A cell comprising the nucleic acid of claim 49; and a guide RNA or nucleic acid encoding the guide RNA.

66. A method of modifying a target nucleic acid, comprising contacting the target nucleic acid with the effector protein encoded by the nucleic acid of claim 49 and a guide RNA.

67. The method of claim 66, wherein the target nucleic acid is in a cell, and the method comprises delivering to the cell:
(a) the nucleic acid encoding the effector protein; and
(b) the guide RNA or a nucleic acid encoding the guide RNA.

68. A method of treating, preventing, or inhibiting a disease or syndrome in a subject, the method comprising administering to the subject the (a) nucleic acid of claim 49; and (b) a guide RNA or a nucleic acid encoding the guide RNA.
